SEQUENCE LISTING (538 sequences)

SEQ ID NO: 1 - DNA, 4273 bp, Homo sapiens
gccaataggagccgcgctggctggagagtaatgttacagagcggagagagtgaggaggctgcgtctggctcccgctctcacagccattgcagtacattgagctccatagagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaagcgcccatgtaacacaaactgccattcaacaggtatttcccttactacctaaggaattgtaaccattgctcagacattgtaggatttaactatgttgaaaactacaggagaggccgggcgcagtggctcacgcctgtaatcccagcactttgggaggccaaggcgggcagatcacgaggtcaggagattgagaccatcctggctaacgtggtgaaaccccgcctctactaaaaatacaaaaaattagccaagcgtggtgctgggcgcctgtagtcccagtaactcaggaggctgaggcaggagaatggcgtgaacccgggaggcggaggttgcagtgagccgagattgtgccactgcactccagcctgggtgacagagcaagactccatctcaaaaaaaaaaaaaaaacacaggagagacaactggtttttgaatgaaatacatgggtactgccttgcttgacatcacatagtccttgatgaaagttcacatttaggtctgcttggtacaatacgcctcctaaaaaggtccttgatgaaagttcacatttaggtctgcttggtacaacacgcctcctgaaagggtctgatagctttcagtagcagtaagacacttgcatgtgatggtaaggtatctgcaaatttgcacacaccgtacacagcttaagtcttagaattaacttgctaaaatgtgagcctttggtaattaggctgttttattagggagtgtgataatatttgaatttcttttcatatttgtgctttgtgtcattttcaaatgacccttgaaatgtattttaaaagtagataaaagccagaaagtga

SEQ ID NO: 2 - DNA, 4273 bp, Homo sapiens
tcactttctggcttttatctacttttaaaatacatttcaagggtcatttgaaaatgacacaaagcacaaatatgaaaagaaattcaaatattatcacactccctaataaaacagcctaattaccaaaggctcacattttagcaagttaattctaagacttaagctgtgtacggtgtgtgcaaatttgcagataccttaccatcacatgcaagtgtcttactgctactgaaagctatcagaccctttcaggaggcgtgttgtaccaagcagacctaaatgtgaactttcatcaaggacctttttaggaggcgtattgtaccaagcagacctaaatgtgaactttcatcaaggactatgtgatgtcaagcaaggcagtacccatgtatttcattcaaaaaccagttgtctctcctgtgttttttttttttttttgagatggagtcttgctctgtcacccaggctggagtgcagtggcacaatctcggctcactgcaacctccgcctcccgggttcacgccattctcctgcctcagcctcctgagttactgggactacaggcgcccagcaccacgcttggctaattttttgtatttttagtagaggcggggtttcaccacgttagccaggatggtctcaatctcctgacctcgtgatctgcccgccttggcctcccaaagtgctgggattacaggcgtgagccactgcgcccggcctctcctgtagttttcaacatagttaaatcctacaatgtctgagcaatggttacaattccttaggtagtaagggaaatacctgttgaatggcagtttgtgttacatgggcgcttttcttatctttatcatttaatcattacccctcgggtacacaggacacacaaatttcatttttcagatttcactttaagaaaattaataaatacagcaaacattaacaacactgtgatgggacgatcatcagaactgtggtaactaatcagcaatagctctcaacctttgcacagcaaagttaggaaatttgactcgttaatcaatatgccatatgcctatctttaaaatacaaaaaaactaagatttatacatcttagttcattagttttaaaaagcatcttcattttaaaagttggcccaattaattatagatttagtcttttcttgatgtcaacaaagtctttaaaccccacagcactgtaactatcttggcattaaaatagtttctatacactttctaaaaaaaatcatcttcagttacaaggctttacacttatctaaattgcctcccacttccatttacttgagttttagacatccaacttctaggggatactgttagaacaccaaaccatactaatcttcagattgaagaggggttttgtccattttcttaagatctgtggtcaagaataaaccctaaaaaaactgttcccattctcaacatcttaaaaaaatggtagttgtcatgacgtacctactaaagttacaaattctccttgagccagaattcattttaaatggatcagaattattaatgttggtgattaatggtgatttctatacagtagaaacttccatctaaatcagattgagtcatttgctcctcttaacaaaaaatctgatgttcgacacaattgcagcctatcactaaccctgctgttcgcttgcatgtatcttgtttttaaaaatcttgtttaaatcattctgaaagtaaaactagtttttcctcctttatatatagcaccgaatacttgcaatgtttgacaaaaccttttatcagcccattaacctatttataagcataaagtgagtatttttaaagggaaaaactttgccatcccttatacttatttaaaaggtactgctaagaggtattattagaaacaagatttaaaaatatgtaacaaaatcttaagttcttaagtgaaagccatttaactagtatttaaaacctctgcaattattagtttattagtatcatccaggactcagatgttcagtattcctcctgaaattacataaacaaatgcaaatggaaagaatccaagtctaaattatataacaaaacagcactccatcacaaaagcgtgtaaaattacaagaacgctattttaaaatactggcactttaagaaaacgataatctcgaaaaccacaaaattgccaaattgttccctaaactcctaagcagataaacatgactaatgaatgagtttgttttgtaaagaaaaatcattcaaataaattgaataattcatactgagatgcaaagtttgtcttctttcttcctatctacttggatttataaaggggcaaaccgtaatataggaaaatataccctattttgaatgtggcatctttgtttgaaaagctggcccaattaattaaaaatacttgtaatggaaagtctcgtttcctgagcagtccatatttagataaatgggaaaagacatctatagccagcattttcatagtcttcactgagactaatgtcaacaaaaaatttaatatggcagtcttgtattttaagctcacgcttttggggatacattctcaggtcttctttaatgtgggaactgcaaaatataactgccattacatggtaatgggagttaaagaatagagtcctcatgtaaaggttagaacatacttttctattagtccttcaaggacagactttcaaaatgtttgattctaataatcccatgctttgagtagattgattactcttcagtttgtaaatgtaagtgggctatgtgaaatttcttaactgatcaagattttggatgttcagttatggatgaaaactatctcaacttaactttacccccattatgatatgcagggtgtgtagacaaaagctttttattagctaactatatgaaaggataaacactgtaataattcatgtgattcaaaatgggcaaaagcaaaagactagcttcccctcaagaagaaaaatttcagacctatgaaaaggacaacaatactaataaaaaaaattgtatttacttagaagcattcagaatgtcaacaaaacagctgcaactttttttttttttttgcaattacagagtggtattcagttaacagaacaacaattatttcgtataagctgcatcagagacaactgaagatgaaaaaactaccatccccatatataactaatttgtgctgtgcaccaacaagaacctgctttaaatttccatgccaatttacaacccccatactgtaccaggcaaggttagtggctattgaaaataccaccaggacagggctatctaaagacacattcggtagtgtgttaactatacaaaaaaagacactgtacagtttaaaaacaaatcttacacagccttacatttcaatttttttctttaaaaggagtgagttgtgtacaggggggttaaatgctttatagacaagaaaaaaaaaactgcgctagaaccaacttattcatcatcatcatcttcttcttcatcttcatcttcttcatcttcctcctcctcctcatcctcttcatcttcctcatcttcctcctcttccttctttttcttgcttttttcagccttgacaactcccttttttgctgcatcaggctttcctttagctcgatatgcagcaatatccttttcgtatttttccttcagcttcgcagccttcttttcataaggctgcttgtcatctgcagcagtgttattccacatctctcccagtttcttcgcaacatcaccaatggacaggccaggatgttctcctttgatttttgggcgatactcagagcagaagaggaagaaggccgaaggaggcctcttgggtgcattgggatccttgaacttcttttttgtctcccctttgggagggatataggttttcatttctctttcataacgggccttgtccgcttttgccatatcttcaaattttcctttctctttagcagacatggtcttccacctctctgagcacttcttagaaaactctgagaagttgactgaagcatctgggtgcttcttcttatgctcctcccgacaagtttgcacaaaaaatgcatatgatgacattttgcctctcggcttcttaggatctcctttgcccatgtttagttatttttcctcagcgaggcacagagtcgcccagtgcccgtccggctctcacttgccccggcgctgtctctatggagctcaatgtactgcaatggctgtgagagcgggagccagacgcagcctcctcactctctccgctctgtaacattactctccagccagcgcggctcctattggc

SEQ ID NO: 3 - DNA, 2855 bp, Mus musculus
cgcgctggctggagagtaatgttacagagcggagagagtgaggaggctgcgtctggctcccgctctcacagccattgcagtacattgagctccatagagacagcgccggggcaagcgcgagccggacgggcactgggcgactctgtgcctcgcggaggaaaatcaactaaacatgggcaaaggagatcctaaaaagccgagaggcaaaatgtcctcatatgcattctttgtgcaaacttgccgggaggagcacaagaagaagcacccggatgcttctgtcaacttctcagagttctccaagaagtgctcagagaggtggaagaccatgtctgctaaagaaaaggggaaatttgaagatatggcaaaggctgacaaggctcgttatgaaagagaaatgaaaacctacatcccccccaaaggggagaccaaaaagaagttcaaggaccccaatgcacccaagaggcctccttcggccttcttcttgttctgttctgagtaccgccccaaaatcaaaggcgagcatcctggcttatccattggtgatgttgcaaagaaactaggagagatgtggaacaacactgcagcagatgacaagcagccctatgagaagaaagctgccaagctgaaggagaagtatgagaaggatattgctgcctacagagctaaaggaaaacctgatgcagcgaaaaagggggtggtcaaggctgaaaagagcaagaaaaagaaggaagaggaagatgatgaggaggatgaagaggatgaggaagaggaggaagaagaggaagacgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtctgggggttgtaaattggcatggaaatttaaagcaggttcttgttggtgcacagcacaaattagttatatatggggacagtagtttggttttttgtttttttttttttttcttttggttttctttttgggttttatttttttcatcttcagttgtctctgatgcagcttatacgaagataattgttgttctgttaactgaataccactctgtaattgcaaaaaaaaaattgcggctgttttgttgacattctgaatgcttctaagtaaatacaattttttttattagtattgttgtccttttcataggtctgaaagttttcttctcaaggggaagctagtcttttgctttgcccattttgggtcacatggattattagtgtgttatctttcatctagttagctggaagagagcttttgtccacatgccctgccattgtggtagggtaacattttcatccatagttgaagaatctcctaaatcgtgatagttggataagagatattatataacctacttggcaaagcaaggagtgatcaatactgtcacaccgtgggactattaggatcaagcaatctgaacgtctgtccttgaaggactgatagaaaagtaccttctaatccttacacgaggactctcctttaaccgccattactgtgtaatgacagttatattttgcagtttcccctactaaagaagacctgagaatgtatccccaaaagtgtgagcttaaaatacaagactgctgtactatttgttgaccttagtcccagcgaaggctatcacaagaacgctggctgtaaagcctttgcccttctatctagatatggattgctcaggaaacttgactgtttaaaggtatttttaattacttgagccagcttttaaaattatgccacatttaaaatgaagggtatattttcctatactgtggtttgtccctttatgaatcagatacaagaggataaactttgcatattagtaccatttgtccaatacatttgctttttctttataaaacccaaactcattcattaatcaggtttaatctgcttagtttagggaacaatttggcaattttgtggatttttttttgagattatcgttctcttaaagtgccagtgttttaaatagcgttcttgtaatttcacgcgcttttgtgatggagtgctgttatataattttgacttgggttctttacatttgcgttgttaatgtaatttgaggaggaatactgaacatgagtcctggatgatactaataaactaataattacagaggttttaaatattagttaaatgactttcacttaagaatttaagcttttggtcacactttataatagtgccttatagtataaacaactgaaaggctctttcccattaacaacccttgatgctggggccagtgagatagtgggtaaaaaggcagttggctgccaaccctgacaaccgatggcaaaaggagggaaccagcttccaaaatgctttgaccaaatgctccctccattcatgaacacagttttaaaatgttaaataggctagagggcagtaaaaacaggtttttttatcgagcatccctaatctatacatatgaggagccataatctgaatgttaagtgaaaagcgaggttggtcttaaagattgcacgtgtgttcttaagcctgtagaggacctccgcaggccgtaatggtctcgattaccaacttaagaacaagtgactggcttggaaacttgtactgttgctttagaactaccattgtggacatctgttgttagtaagtgatccatttaaaagtgaactctgcctcaa

SEQ ID NO: 4 - DNA, 2855 bp, Mus musculus
ttgaggcagagttcacttttaaatggatcacttactaacaacagatgtccacaatggtagttctaaagcaacagtacaagtttccaagccagtcacttgttcttaagttggtaatcgagaccattacggcctgcggaggtcctctacaggcttaagaacacacgtgcaatctttaagaccaacctcgcttttcacttaacattcagattatggctcctcatatgtatagattagggatgctcgataaaaaaacctgtttttactgccctctagcctatttaacattttaaaactgtgttcatgaatggagggagcatttggtcaaagcattttggaagctggttccctccttttgccatcggttgtcagggttggcagccaactgcctttttacccactatctcactggccccagcatcaagggttgttaatgggaaagagcctttcagttgtttatactataaggcactattataaagtgtgaccaaaagcttaaattcttaagtgaaagtcatttaactaatatttaaaacctctgtaattattagtttattagtatcatccaggactcatgttcagtattcctcctcaaattacattaacaacgcaaatgtaaagaacccaagtcaaaattatataacagcactccatcacaaaagcgcgtgaaattacaagaacgctatttaaaacactggcactttaagagaacgataatctcaaaaaaaaatccacaaaattgccaaattgttccctaaactaagcagattaaacctgattaatgaatgagtttgggttttataaagaaaaagcaaatgtattggacaaatggtactaatatgcaaagtttatcctcttgtatctgattcataaagggacaaaccacagtataggaaaatatacccttcattttaaatgtggcataattttaaaagctggctcaagtaattaaaaatacctttaaacagtcaagtttcctgagcaatccatatctagatagaagggcaaaggctttacagccagcgttcttgtgatagccttcgctgggactaaggtcaacaaatagtacagcagtcttgtattttaagctcacacttttggggatacattctcaggtcttctttagtaggggaaactgcaaaatataactgtcattacacagtaatggcggttaaaggagagtcctcgtgtaaggattagaaggtacttttctatcagtccttcaaggacagacgttcagattgcttgatcctaatagtcccacggtgtgacagtattgatcactccttgctttgccaagtaggttatataatatctcttatccaactatcacgatttaggagattcttcaactatggatgaaaatgttaccctaccacaatggcagggcatgtggacaaaagctctcttccagctaactagatgaaagataacacactaataatccatgtgacccaaaatgggcaaagcaaaagactagcttccccttgagaagaaaactttcagacctatgaaaaggacaacaatactaataaaaaaaattgtatttacttagaagcattcagaatgtcaacaaaacagccgcaattttttttttgcaattacagagtggtattcagttaacagaacaacaattatcttcgtataagctgcatcagagacaactgaagatgaaaaaaataaaacccaaaaagaaaaccaaaagaaaaaaaaaaaaaaacaaaaaaccaaactactgtccccatatataactaatttgtgctgtgcaccaacaagaacctgctttaaatttccatgccaatttacaacccccagactgtaccaggcaaggttagtggctattgaaaataccaccaggacagggctatctaaagacacattcggtagtgtgttaactatacaaaaaaagacactgtacagtttaaaaacaaatcttacacagccttacatttcaatttttttctttaaaaggagtgagttgtgtacaggggggttaaatgctttatagacaagaaaaaaaaactgcgctagaaccaacttattcatcatcatcatcttcttcttcatcttcgtcttcctcttcttcctcctcttcctcatcctcttcatcctcctcatcatcttcctcttccttctttttcttgctcttttcagccttgaccaccccctttttcgctgcatcaggttttcctttagctctgtaggcagcaatatccttctcatacttctccttcagcttggcagctttcttctcatagggctgcttgtcatctgctgcagtgttgttccacatctctcctagtttctttgcaacatcaccaatggataagccaggatgctcgcctttgattttggggcggtactcagaacagaacaagaagaaggccgaaggaggcctcttgggtgcattggggtccttgaacttctttttggtctcccctttgggggggatgtaggttttcatttctctttcataacgagccttgtcagcctttgccatatcttcaaatttccccttttctttagcagacatggtcttccacctctctgagcacttcttggagaactctgagaagttgacagaagcatccgggtgcttcttcttgtgctcctcccggcaagtttgcacaaagaatgcatatgaggacattttgcctctcggctttttaggatctcctttgcccatgtttagttgattttcctccgcgaggcacagagtcgcccagtgcccgtccggctcgcgcttgccccggcgctgtctctatggagctcaatgtactgcaatggctgtgagagcgggagccagacgcagcctcctcactctctccgctctgtaacattactctccagccagcgcg

SEQ ID NO: 5 - DNA, 2256 bp, Rattus norvegicus
cgagagccggacgggcactgggcgactctgtgcctcgcggaggaaaatcaactaaacatgggcaaaggagatcctaagaagccgagaggcaaaatgtcctcatatgcattctttgtgcaaacctgccgggaggagcacaagaagaagcacccggatgcttctgtcaacttctcagagttctccaagaagtgctcagagaggtggaagaccatgtctgctaaagaaaaggggaaatttgaagatatggcaaaggctgacaaggctcgttatgaaagagaaatgaaaacctacatcccccccaaaggggagaccaaaaagaagttcaaggaccccaatgcccccaagaggcctccttcggccttcttcttgttctgttctgagtaccgcccaaaaatcaaaggcgagcatcctggcttatccattggtgatgttgcgaagaaactaggagagatgtggaacaacactgctgcggatgacaagcagccctatgaaaagaaggccgccaagctgaaggagaagtatgagaaggatattgctgcctacagagctaaaggaaaacctgatgcagcgaaaaagggggtggtcaaggctgagaagagcaagaaaaagaaggaagaggaagacgacgaggaggatgaagaggatgaggaagaggaggaagaggaggaagacgaagatgaagaagaagatgatgatgatgaataagttggttctagcgcagtttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagctagccctgtcctggtggtattttcaatagccactaaccttgcctggtacagtctgggggttgtaaattggcatggaaattaaagcaggttcttgttggtgcacagcacaaattagttatatatggggacagtagtttggtttttggtttctttttttttttttttttggttttgttttttttccttttgttttttttttccatcttcagttgtctctgatgcagcttatacgaaggtaattgttgttctgttaactgaataccactctgtaattgcaaaaaaatggcggctgttttgttgacattctgaatgcttctaagtaaatacaattttttttattagtactgttgtccttttcataggtctgaaagttttcttctcaaggggaagctagtcttttgcttttgcccattttgggtcacatggattattagtgtgtatcttcatatagttggaaaaatcttttgtccacacaccctgcatattgtggtaggggtaacattttcatctacagttgaagaattctccaaaattgtgatcagttggataagagatactctacttagcaaagcaaggagtgatcaattctgtcacaccatgggattattagatcaaacagtctgaaagtctgtccttgaaggactaatagaaaagtatgttctgctccttacctgaggactcctttaactgccattactgtgtaatgacagttatattttgcagtttcccctactaaagacctgagaatgtatccccaaaagcgtgagcttaaaatacaagattgctgtactatttgttgaccttagtcccagcgaaggctatcatgagaagctggctgtaatgcctttgcccttctatctaaatacggattgctcaggaaacttgactgtttaaaggtatatttaattagttgagccagcttttaaaattatgccacatttaaaatgaagggtatattttcctatattgtggtttgtccctttataaatcagatacaaggaaagcggataaactttgcatattagtaccagttgtccaagccatttgctttttctttataaagcccgaactcattcattaatcatgtttaatctgcttagtttagggaacaatttggcaattttgtgggttttctttttttctttttcttttttttttttttttgagattatcattctcttaaagtgccagtgttttaaaatagcgttcttgtaattttccgcgcttttgtgatggagtgctgttatatcattttgacttgggttctttacagttgcctttgttctgtaatttgaggaggaatactgaacatgagtcctggatgatactaataaactaataattgcagaggttttaaaaaaaaaaaaaaaaaaaaaaaaa

SEQ ID NO: 6 - DNA, 2256 bp, Rattus norvegicus
tttttttttttttttttttttttttaaaacctctgcaattattagtttattagtatcatccaggactcatgttcagtattcctcctcaaattacagaacaaaggcaactgtaaagaacccaagtcaaaatgatataacagcactccatcacaaaagcgcggaaaattacaagaacgctattttaaaacactggcactttaagagaatgataatctcaaaaaaaaaaaaaaaagaaaaagaaaaaaagaaaacccacaaaattgccaaattgttccctaaactaagcagattaaacatgattaatgaatgagttcgggctttataaagaaaaagcaaatggcttggacaactggtactaatatgcaaagtttatccgctttccttgtatctgatttataaagggacaaaccacaatataggaaaatatacccttcattttaaatgtggcataattttaaaagctggctcaactaattaaatatacctttaaacagtcaagtttcctgagcaatccgtatttagatagaagggcaaaggcattacagccagcttctcatgatagccttcgctgggactaaggtcaacaaatagtacagcaatcttgtattttaagctcacgcttttggggatacattctcaggtctttagtaggggaaactgcaaaatataactgtcattacacagtaatggcagttaaaggagtcctcaggtaaggagcagaacatacttttctattagtccttcaaggacagactttcagactgtttgatctaataatcccatggtgtgacagaattgatcactccttgctttgctaagtagagtatctcttatccaactgatcacaattttggagaattcttcaactgtagatgaaaatgttacccctaccacaatatgcagggtgtgtggacaaaagatttttccaactatatgaagatacacactaataatccatgtgacccaaaatgggcaaaagcaaaagactagcttccccttgagaagaaaactttcagacctatgaaaaggacaacagtactaataaaaaaaattgtatttacttagaagcattcagaatgtcaacaaaacagccgccatttttttgcaattacagagtggtattcagttaacagaacaacaattaccttcgtataagctgcatcagagacaactgaagatggaaaaaaaaaacaaaaggaaaaaaaacaaaaccaaaaaaaaaaaaaaaaagaaaccaaaaaccaaactactgtccccatatataactaatttgtgctgtgcaccaacaagaacctgctttaatttccatgccaatttacaacccccagactgtaccaggcaaggttagtggctattgaaaataccaccaggacagggctagctaaagacacattcggtagtgtgttaactatacaaaaaaagacactgtacagtttaaaaacaaatcttacacagccttacatttcaatttttttctttaaaaggagtgagttgtgtacaggggggttaaatgctttatagacaagaaaaaaaaactgcgctagaaccaacttattcatcatcatcatcttcttcttcatcttcgtcttcctcctcttcctcctcttcctcatcctcttcatcctcctcgtcgtcttcctcttccttctttttcttgctcttctcagccttgaccaccccctttttcgctgcatcaggttttcctttagctctgtaggcagcaatatccttctcatacttctccttcagcttggcggccttcttttcatagggctgcttgtcatccgcagcagtgttgttccacatctctcctagtttcttcgcaacatcaccaatggataagccaggatgctcgcctttgatttttgggcggtactcagaacagaacaagaagaaggccgaaggaggcctcttgggggcattggggtccttgaacttctttttggtctcccctttgggggggatgtaggttttcatttctctttcataacgagccttgtcagcctttgccatatcttcaaatttccccttttctttagcagacatggtcttccacctctctgagcacttcttggagaactctgagaagttgacagaagcatccgggtgcttcttcttgtgctcctcccggcaggtttgcacaaagaatgcatatgaggacattttgcctctcggcttcttaggatctcctttgcccatgtttagttgattttcctccgcgaggcacagagtcgcccagtgcccgtccggctctcg

SEQ ID NO: 7 - DNA, 2323 bp, Macaca fascicularis
aatgttacagagcggagagagtgaggaggctgcgtctggctcccgctctcacagccattgcagtacattgagctccatagagacagcgccggggcaagtgagagccggacgggcactgggcgactctgtgcctcgctgaggaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaggcggacaaggcccgttacgaaagagaaatgaaaacctatatccctcccaaaggggagacaaaaaagaagttcaaggatcccaatgcacccaagaggcctccttcggccttcttcctgttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttacatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcataccataatgggggtaaagttaagttgagatagttttcatccataactgaacatcgaaaatcttgatcagttaagaaatttcacatagcccacttacatttacaaactgaagagtaatcagtctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagattgccatattaaattttttgttgacattagtctcagtgaagactatgaaaatgctggctatagatgtcttttcccatttatctcaatatggactgctcaggaaacgagactttccattacaagtatttttaattaattgggccagcttttaaaatgaagatgccacattcaaaatagggtgtattttcctatattatggtttgcccctttataaatcgaagtagataggaggaaagaagacacttaaactttgcatctcagtatgaattattcaattgatttgaatgatttttctttacaaaacaaactcattagtcattatctgcttagcagtttagggaacaatttggcaattttgtggtttttcgagattatcgttttcttaaagtgccagtattttaaaatagcgttcttgtaattttacacgcttttgtgatggagtgctgttttgttatataattttgacttggattctttccatttgcatttgtttatgtaatttcaggaggaatactgaacatctgagtcctggatgatactaataaactaataattgcagaggttttaaaaaaaaaaaaaaaaa

SEQ ID NO: 8 - DNA, 2323 bp, Macaca fascicularis
tttttttttttttttttaaaacctctgcaattattagtttattagtatcatccaggactcagatgttcagtattcctcctgaaattacataaacaaatgcaaatggaaagaatccaagtcaaaattatataacaaaacagcactccatcacaaaagcgtgtaaaattacaagaacgctattttaaaatactggcactttaagaaaacgataatctcgaaaaaccacaaaattgccaaattgttccctaaactgctaagcagataatgactaatgagtttgttttgtaaagaaaaatcattcaaatcaattgaataattcatactgagatgcaaagtttaagtgtcttctttcctcctatctacttcgatttataaaggggcaaaccataatataggaaaatacaccctattttgaatgtggcatcttcattttaaaagctggcccaattaattaaaaatacttgtaatggaaagtctcgtttcctgagcagtccatattgagataaatgggaaaagacatctatagccagcattttcatagtcttcactgagactaatgtcaacaaaaaatttaatatggcaatcttgtattttaagctcacgcttttggggatacattctcaggtcttctttaatgtgggaactgcaaaatataactgccattacatggtaatgggagttaaagaatagagtcctcatgtaaaggttagaacatacttttctattagtccttcaaggacagactttcaaaatgtttgattctaataatcccatgctttgagtagactgattactcttcagtttgtaaatgtaagtgggctatgtgaaatttcttaactgatcaagattttcgatgttcagttatggatgaaaactatctcaacttaactttacccccattatggtatgcagggtgtgtagacaaaagctttttattagctaactatatgaaaggataaacactgtaataattcatgtgattcaaaatgggcaaaagcaaaagactagcttcccctcaagaagaaaaatttcagacctatgaaaaggacaacaatactaataaaaaaaattgtatttacttagaagcattcagaatgtcaacaaaacagctgcaacttttttttttttttttgcaattacagagtggtattcagttaacagaacaacaattatttcgtataagctgcatcagagacaactgaagatgaaaaaactaccatccccatatataactaatttgtgctgtgcaccaacaagaacctgctttaaatttccatgccaatttacaacccccatactgtaccaggcaaggttagtggctattgaaaataccaccaggacagggctatgtaaagacacattcggtagtgtgttaactatacaaaaaaagacactgtacagtttaaaaacaaatcttacacagccttacatttcaatttttttctttaaaaggagtgagttgtgtacaggggggttaaatgctttatagacaagaaaaaaaaaactgcgctagaaccaacttattcatcatcatcatcttcttcttcatcttcatcttcttcatcttcctcctcctcctcatcctcttcatcttcctcatcttcctcctcttccttctttttcttgcttttttcagccttgacaactcccttttttgctgcatcaggctttcctttagctcgatatgcagcaatatccttttcgtatttttccttcagcttcgcagccttcttttcataaggctgcttgtcatctgcagcagtgttattccacatctctcccagtttcttcgcaacatcaccaatggacaggccaggatgttctcctttgatttttgggcgatactcagagcagaacaggaagaaggccgaaggaggcctcttgggtgcattgggatccttgaacttcttttttgtctcccctttgggagggatataggttttcatttctctttcgtaacgggccttgtccgcctttgccatatcttcaaattttcctttctctttagcagacatggtcttccacctctctgagcacttcttagaaaactctgagaagttgactgaagcatctgggtgcttcttcttatgctcctcccgacaagtttgcacaaaaaatgcatatgatgacattttgcctctcggcttcttaggatctcctttgcccatgtttagttatttttcctcagcgaggcacagagtcgcccagtgcccgtccggctctcacttgccccggcgctgtctctatggagctcaatgtactgcaatggctgtgagagcgggagccagacgcagcctcctcactctctccgctctgtaacatt

SEQ ID NO: 9 - DNA, 4270 bp, Homo sapiens
agtcagaacagaaatacatctcagggccaaaccgataggaaacgaggctgcctcgcggtggcaccgccaccccccaaccgggttccgagcaccggagctggctgctgctccctctttggagcaaagttttatgcaaagagggtgttttttgaaactttcggtgcacgaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaagcgcccatgtaacacaaactgccattcaacaggtatttcccttactacctaaggaattgtaaccattgctcagacattgtaggatttaactatgttgaaaactacaggagaggccgggcgcagtggctcacgcctgtaatcccagcactttgggaggccaaggcgggcagatcacgaggtcaggagattgagaccatcctggctaacgtggtgaaaccccgcctctactaaaaatacaaaaaattagccaagcgtggtgctgggcgcctgtagtcccagtaactcaggaggctgaggcaggagaatggcgtgaacccgggaggcggaggttgcagtgagccgagattgtgccactgcactccagcctgggtgacagagcaagactccatctcaaaaaaaaaaaaaaaacacaggagagacaactggtttttgaatgaaatacatgggtactgccttgcttgacatcacatagtccttgatgaaagttcacatttaggtctgcttggtacaatacgcctcctaaaaaggtccttgatgaaagttcacatttaggtctgcttggtacaacacgcctcctgaaagggtctgatagctttcagtagcagtaagacacttgcatgtgatggtaaggtatctgcaaatttgcacacaccgtacacagcttaagtcttagaattaacttgctaaaatgtgagcctttggtaattaggctgttttattagggagtgtgataatatttgaatttcttttcatatttgtgctttgtgtcattttcaaatgacccttgaaatgtattttaaaagtagataaaagccagaaagtga

SEQ ID NO: 10 - DNA, 4270 bp, Homo sapiens
tcactttctggcttttatctacttttaaaatacatttcaagggtcatttgaaaatgacacaaagcacaaatatgaaaagaaattcaaatattatcacactccctaataaaacagcctaattaccaaaggctcacattttagcaagttaattctaagacttaagctgtgtacggtgtgtgcaaatttgcagataccttaccatcacatgcaagtgtcttactgctactgaaagctatcagaccctttcaggaggcgtgttgtaccaagcagacctaaatgtgaactttcatcaaggacctttttaggaggcgtattgtaccaagcagacctaaatgtgaactttcatcaaggactatgtgatgtcaagcaaggcagtacccatgtatttcattcaaaaaccagttgtctctcctgtgttttttttttttttttgagatggagtcttgctctgtcacccaggctggagtgcagtggcacaatctcggctcactgcaacctccgcctcccgggttcacgccattctcctgcctcagcctcctgagttactgggactacaggcgcccagcaccacgcttggctaattttttgtatttttagtagaggcggggtttcaccacgttagccaggatggtctcaatctcctgacctcgtgatctgcccgccttggcctcccaaagtgctgggattacaggcgtgagccactgcgcccggcctctcctgtagttttcaacatagttaaatcctacaatgtctgagcaatggttacaattccttaggtagtaagggaaatacctgttgaatggcagtttgtgttacatgggcgcttttcttatctttatcatttaatcattacccctcgggtacacaggacacacaaatttcatttttcagatttcactttaagaaaattaataaatacagcaaacattaacaacactgtgatgggacgatcatcagaactgtggtaactaatcagcaatagctctcaacctttgcacagcaaagttaggaaatttgactcgttaatcaatatgccatatgcctatctttaaaatacaaaaaaactaagatttatacatcttagttcattagttttaaaaagcatcttcattttaaaagttggcccaattaattatagatttagtcttttcttgatgtcaacaaagtctttaaaccccacagcactgtaactatcttggcattaaaatagtttctatacactttctaaaaaaaatcatcttcagttacaaggctttacacttatctaaattgcctcccacttccatttacttgagttttagacatccaacttctaggggatactgttagaacaccaaaccatactaatcttcagattgaagaggggttttgtccattttcttaagatctgtggtcaagaataaaccctaaaaaaactgttcccattctcaacatcttaaaaaaatggtagttgtcatgacgtacctactaaagttacaaattctccttgagccagaattcattttaaatggatcagaattattaatgttggtgattaatggtgatttctatacagtagaaacttccatctaaatcagattgagtcatttgctcctcttaacaaaaaatctgatgttcgacacaattgcagcctatcactaaccctgctgttcgcttgcatgtatcttgtttttaaaaatcttgtttaaatcattctgaaagtaaaactagtttttcctcctttatatatagcaccgaatacttgcaatgtttgacaaaaccttttatcagcccattaacctatttataagcataaagtgagtatttttaaagggaaaaactttgccatcccttatacttatttaaaaggtactgctaagaggtattattagaaacaagatttaaaaatatgtaacaaaatcttaagttcttaagtgaaagccatttaactagtatttaaaacctctgcaattattagtttattagtatcatccaggactcagatgttcagtattcctcctgaaattacataaacaaatgcaaatggaaagaatccaagtctaaattatataacaaaacagcactccatcacaaaagcgtgtaaaattacaagaacgctattttaaaatactggcactttaagaaaacgataatctcgaaaaccacaaaattgccaaattgttccctaaactcctaagcagataaacatgactaatgaatgagtttgttttgtaaagaaaaatcattcaaataaattgaataattcatactgagatgcaaagtttgtcttctttcttcctatctacttggatttataaaggggcaaaccgtaatataggaaaatataccctattttgaatgtggcatctttgtttgaaaagctggcccaattaattaaaaatacttgtaatggaaagtctcgtttcctgagcagtccatatttagataaatgggaaaagacatctatagccagcattttcatagtcttcactgagactaatgtcaacaaaaaatttaatatggcagtcttgtattttaagctcacgcttttggggatacattctcaggtcttctttaatgtgggaactgcaaaatataactgccattacatggtaatgggagttaaagaatagagtcctcatgtaaaggttagaacatacttttctattagtccttcaaggacagactttcaaaatgtttgattctaataatcccatgctttgagtagattgattactcttcagtttgtaaatgtaagtgggctatgtgaaatttcttaactgatcaagattttggatgttcagttatggatgaaaactatctcaacttaactttacccccattatgatatgcagggtgtgtagacaaaagctttttattagctaactatatgaaaggataaacactgtaataattcatgtgattcaaaatgggcaaaagcaaaagactagcttcccctcaagaagaaaaatttcagacctatgaaaaggacaacaatactaataaaaaaaattgtatttacttagaagcattcagaatgtcaacaaaacagctgcaactttttttttttttttgcaattacagagtggtattcagttaacagaacaacaattatttcgtataagctgcatcagagacaactgaagatgaaaaaactaccatccccatatataactaatttgtgctgtgcaccaacaagaacctgctttaaatttccatgccaatttacaacccccatactgtaccaggcaaggttagtggctattgaaaataccaccaggacagggctatctaaagacacattcggtagtgtgttaactatacaaaaaaagacactgtacagtttaaaaacaaatcttacacagccttacatttcaatttttttctttaaaaggagtgagttgtgtacaggggggttaaatgctttatagacaagaaaaaaaaaactgcgctagaaccaacttattcatcatcatcatcttcttcttcatcttcatcttcttcatcttcctcctcctcctcatcctcttcatcttcctcatcttcctcctcttccttctttttcttgcttttttcagccttgacaactcccttttttgctgcatcaggctttcctttagctcgatatgcagcaatatccttttcgtatttttccttcagcttcgcagccttcttttcataaggctgcttgtcatctgcagcagtgttattccacatctctcccagtttcttcgcaacatcaccaatggacaggccaggatgttctcctttgatttttgggcgatactcagagcagaagaggaagaaggccgaaggaggcctcttgggtgcattgggatccttgaacttcttttttgtctcccctttgggagggatataggttttcatttctctttcataacgggccttgtccgcttttgccatatcttcaaattttcctttctctttagcagacatggtcttccacctctctgagcacttcttagaaaactctgagaagttgactgaagcatctgggtgcttcttcttatgctcctcccgacaagtttgcacaaaaaatgcatatgatgacattttgcctctcggcttcttaggatctcctttgcccatgtttagttatttttcgtgcaccgaaagtttcaaaaaacaccctctttgcataaaactttgctccaaagagggagcagcagccagctccggtgctcggaacccggttggggggtggcggtgccaccgcgaggcagcctcgtttcctatcggtttggccctgagatgtatttctgttctgact

SEQ ID NO: 11 - DNA, 5030 bp, Homo sapiens
tttctgcggagggattacgctgacgaaagagacctgcttgcgcgtcgctgttccgtggtccgcgcgagcgtggtcgggagccgctggttcctggggtgacccgcggaggtgggagagggaagggcttccgaagccggcgggggtgccatggaccctctccgccggcgcggccttcacagctgggccgcgccgggcatccgtagtccgctctcccaaagcctcggtggagctgaagctgccacagagtgcatgttcacaaagggtcatcacacacggagctgcccctccctgtctccctagagcccatcttcgaggccaggggcttttctaccaggattctggggtgtttctcctcctttcctccctcccagatcttctcacggtaaggggagcagcgaaagcgcagggactttgcattccacgacccgttctgactagtcaacagccgatctgtccctgctgctctaattccagctgccctgccttgttttaacttcagagaaagggggagttctcatttgataagtttaagcctttgctttcgtaggaaggtcatgtggcttaagggacatcgtgaccgcgtatgctatttctgcctgtgcattctaaatcttgggggcagcatattccagaagtccttttggtcgattggttctgtgtcctaggataacaatttgtagtttctgaccatttctttacagaaaaaccacaattggtattttggactgcggggttttttagtggtctcaaactaaatgattatattctggaataatgctgacaatttggatagggtggtgtggaggaaacaagtctcgtgtagaagaaattatttagtaaaaaggattttagtttttggtacttctgaatgcaaatggccaaggaatccagcagtttgttggggtttctccagacaaaaataggctgaaaaataactaaacatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagacaaaaaagaagttcaaggatcccaatgcacccaagaggcctccttcggccttcttcctcttctgctctgagtatcgcccaaaaatcaaaggagaacatcctggcctgtccattggtgatgttgcgaagaaactgggagagatgtggaataacactgctgcagatgacaagcagccttatgaaaagaaggctgcgaagctgaaggaaaaatacgaaaaggatattgctgcatatcgagctaaaggaaagcctgatgcagcaaaaaagggagttgtcaaggctgaaaaaagcaagaaaaagaaggaagaggaggaagatgaggaagatgaagaggatgaggaggaggaggaagatgaagaagatgaagatgaagaagaagatgatgatgatgaataagttggttctagcgcagttttttttttcttgtctataaagcatttaacccccctgtacacaactcactccttttaaagaaaaaaattgaaatgtaaggctgtgtaagatttgtttttaaactgtacagtgtctttttttgtatagttaacacactaccgaatgtgtctttagatagccctgtcctggtggtattttcaatagccactaaccttgcctggtacagtatgggggttgtaaattggcatggaaatttaaagcaggttcttgttggtgcacagcacaaattagttatatatggggatggtagttttttcatcttcagttgtctctgatgcagcttatacgaaataattgttgttctgttaactgaataccactctgtaattgcaaaaaaaaaaaaaaagttgcagctgttttgttgacattctgaatgcttctaagtaaatacaattttttttattagtattgttgtccttttcataggtctgaaatttttcttcttgaggggaagctagtcttttgcttttgcccattttgaatcacatgaattattacagtgtttatcctttcatatagttagctaataaaaagcttttgtctacacaccctgcatatcataatgggggtaaagttaagttgagatagttttcatccataactgaacatccaaaatcttgatcagttaagaaatttcacatagcccacttacatttacaaactgaagagtaatcaatctactcaaagcatgggattattagaatcaaacattttgaaagtctgtccttgaaggactaatagaaaagtatgttctaacctttacatgaggactctattctttaactcccattaccatgtaatggcagttatattttgcagttcccacattaaagaagacctgagaatgtatccccaaaagcgtgagcttaaaatacaagactgccatattaaattttttgttgacattagtctcagtgaagactatgaaaatgctggctatagatgtcttttcccatttatctaaatatggactgctcaggaaacgagactttccattacaagtatttttaattaattgggccagcttttcaaacaaagatgccacattcaaaatagggtatattttcctatattacggtttgcccctttataaatccaagtagataggaagaaagaagacaaactttgcatctcagtatgaattattcaatttatttgaatgatttttctttacaaaacaaactcattcattagtcatgtttatctgcttaggagtttagggaacaatttggcaattttgtggttttcgagattatcgttttcttaaagtgccagtattttaaaatagcgttcttgtaattttacacgcttttgtgatggagtgctgttttgttatataatttagacttggattctttccatttgcatttgtttatgtaatttcaggaggaatactgaacatctgagtcctggatgatactaataaactaataattgcagaggttttaaatactagttaaatggctttcacttaagaacttaagattttgttacatatttttaaatcttgtttctaataatacctcttagcagtaccttttaaataagtataagggatggcaaagtttttccctttaaaaatactcactttatgcttataaataggttaatgggctgataaaaggttttgtcaaacattgcaagtattcggtgctatatataaaggaggaaaaactagttttactttcagaatgatttaaacaagatttttaaaaacaagatacatgcaagcgaacagcagggttagtgataggctgcaattgtgtcgaacatcagattttttgttaagaggagcaaatgactcaatctgatttagatggaagtttctactgtatagaaatcaccattaatcaccaacattaataattctgatccatttaaaatgaattctggctcaaggagaatttgtaactttagtaggtacgtcatgacaactaccatttttttaagatgttgagaatgggaacagtttttttagggtttattcttgaccacagatcttaagaaaatggacaaaacccctcttcaatctgaagattagtatggtttggtgttctaacagtatcccctagaagttggatgtctaaaactcaagtaaatggaagtgggaggcaatttagataagtgtaaagccttgtaactgaagatgattttttttagaaagtgtatagaaactattttaatgccaagatagttacagtgctgtggggtttaaagactttgttgacatcaagaaaagactaaatctataattaattgggccaacttttaaaatgaagatgctttttaaaactaatgaactaagatgtataaatcttagtttttttgtattttaaagataggcatatggcatattgattaacgagtcaaatttcctaactttgctgtgcaaaggttgagagctattgctgattagttaccacagttctgatgatcgtcccatcacagtgttgttaatgtttgctgtatttattaattttcttaaagtgaaatctgaaaaatgaaatttgtgtgtcctgtgtacccgaggggtaatgattaaatgataaagataagaaaagcgcccatgtaacacaaactgccattcaacaggtatttcccttactacctaaggaattgtaaccattgctcagacattgtaggatttaactatgttgaaaactacaggagaggccgggcgcagtggctcacgcctgtaatcccagcactttgggaggccaaggcgggcagatcacgaggtcaggagattgagaccatcctggctaacgtggtgaaaccccgcctctactaaaaatacaaaaaattagccaagcgtggtgctgggcgcctgtagtcccagtaactcaggaggctgaggcaggagaatggcgtgaacccgggaggcggaggttgcagtgagccgagattgtgccactgcactccagcctgggtgacagagcaagactccatctcaaaaaaaaaaaaaaaacacaggagagacaactggtttttgaatgaaatacatgggtactgccttgcttgacatcacatagtccttgatgaaagttcacatttaggtctgcttggtacaatacgcctcctaaaaaggtccttgatgaaagttcacatttaggtctgcttggtacaacacgcctcctgaaagggtctgatagctttcagtagcagtaagacacttgcatgtgatggtaaggtatctgcaaatttgcacacaccgtacacagcttaagtcttagaattaacttgctaaaatgtgagcctttggtaattaggctgttttattagggagtgtgataatatttgaatttcttttcatatttgtgctttgtgtcattttcaaatgacccttgaaatgtattttaaaagtagataaaagccagaaagtga

SEQ ID NO: 12 - DNA, 5030 bp, Homo sapiens
tcactttctggcttttatctacttttaaaatacatttcaagggtcatttgaaaatgacacaaagcacaaatatgaaaagaaattcaaatattatcacactccctaataaaacagcctaattaccaaaggctcacattttagcaagttaattctaagacttaagctgtgtacggtgtgtgcaaatttgcagataccttaccatcacatgcaagtgtcttactgctactgaaagctatcagaccctttcaggaggcgtgttgtaccaagcagacctaaatgtgaactttcatcaaggacctttttaggaggcgtattgtaccaagcagacctaaatgtgaactttcatcaaggactatgtgatgtcaagcaaggcagtacccatgtatttcattcaaaaaccagttgtctctcctgtgttttttttttttttttgagatggagtcttgctctgtcacccaggctggagtgcagtggcacaatctcggctcactgcaacctccgcctcccgggttcacgccattctcctgcctcagcctcctgagttactgggactacaggcgcccagcaccacgcttggctaattttttgtatttttagtagaggcggggtttcaccacgttagccaggatggtctcaatctcctgacctcgtgatctgcccgccttggcctcccaaagtgctgggattacaggcgtgagccactgcgcccggcctctcctgtagttttcaacatagttaaatcctacaatgtctgagcaatggttacaattccttaggtagtaagggaaatacctgttgaatggcagtttgtgttacatgggcgcttttcttatctttatcatttaatcattacccctcgggtacacaggacacacaaatttcatttttcagatttcactttaagaaaattaataaatacagcaaacattaacaacactgtgatgggacgatcatcagaactgtggtaactaatcagcaatagctctcaacctttgcacagcaaagttaggaaatttgactcgttaatcaatatgccatatgcctatctttaaaatacaaaaaaactaagatttatacatcttagttcattagttttaaaaagcatcttcattttaaaagttggcccaattaattatagatttagtcttttcttgatgtcaacaaagtctttaaaccccacagcactgtaactatcttggcattaaaatagtttctatacactttctaaaaaaaatcatcttcagttacaaggctttacacttatctaaattgcctcccacttccatttacttgagttttagacatccaacttctaggggatactgttagaacaccaaaccatactaatcttcagattgaagaggggttttgtccattttcttaagatctgtggtcaagaataaaccctaaaaaaactgttcccattctcaacatcttaaaaaaatggtagttgtcatgacgtacctactaaagttacaaattctccttgagccagaattcattttaaatggatcagaattattaatgttggtgattaatggtgatttctatacagtagaaacttccatctaaatcagattgagtcatttgctcctcttaacaaaaaatctgatgttcgacacaattgcagcctatcactaaccctgctgttcgcttgcatgtatcttgtttttaaaaatcttgtttaaatcattctgaaagtaaaactagtttttcctcctttatatatagcaccgaatacttgcaatgtttgacaaaaccttttatcagcccattaacctatttataagcataaagtgagtatttttaaagggaaaaactttgccatcccttatacttatttaaaaggtactgctaagaggtattattagaaacaagatttaaaaatatgtaacaaaatcttaagttcttaagtgaaagccatttaactagtatttaaaacctctgcaattattagtttattagtatcatccaggactcagatgttcagtattcctcctgaaattacataaacaaatgcaaatggaaagaatccaagtctaaattatataacaaaacagcactccatcacaaaagcgtgtaaaattacaagaacgctattttaaaatactggcactttaagaaaacgataatctcgaaaaccacaaaattgccaaattgttccctaaactcctaagcagataaacatgactaatgaatgagtttgttttgtaaagaaaaatcattcaaataaattgaataattcatactgagatgcaaagtttgtcttctttcttcctatctacttggatttataaaggggcaaaccgtaatataggaaaatataccctattttgaatgtggcatctttgtttgaaaagctggcccaattaattaaaaatacttgtaatggaaagtctcgtttcctgagcagtccatatttagataaatgggaaaagacatctatagccagcattttcatagtcttcactgagactaatgtcaacaaaaaatttaatatggcagtcttgtattttaagctcacgcttttggggatacattctcaggtcttctttaatgtgggaactgcaaaatataactgccattacatggtaatgggagttaaagaatagagtcctcatgtaaaggttagaacatacttttctattagtccttcaaggacagactttcaaaatgtttgattctaataatcccatgctttgagtagattgattactcttcagtttgtaaatgtaagtgggctatgtgaaatttcttaactgatcaagattttggatgttcagttatggatgaaaactatctcaacttaactttacccccattatgatatgcagggtgtgtagacaaaagctttttattagctaactatatgaaaggataaacactgtaataattcatgtgattcaaaatgggcaaaagcaaaagactagcttcccctcaagaagaaaaatttcagacctatgaaaaggacaacaatactaataaaaaaaattgtatttacttagaagcattcagaatgtcaacaaaacagctgcaactttttttttttttttgcaattacagagtggtattcagttaacagaacaacaattatttcgtataagctgcatcagagacaactgaagatgaaaaaactaccatccccatatataactaatttgtgctgtgcaccaacaagaacctgctttaaatttccatgccaatttacaacccccatactgtaccaggcaaggttagtggctattgaaaataccaccaggacagggctatctaaagacacattcggtagtgtgttaactatacaaaaaaagacactgtacagtttaaaaacaaatcttacacagccttacatttcaatttttttctttaaaaggagtgagttgtgtacaggggggttaaatgctttatagacaagaaaaaaaaaactgcgctagaaccaacttattcatcatcatcatcttcttcttcatcttcatcttcttcatcttcctcctcctcctcatcctcttcatcttcctcatcttcctcctcttccttctttttcttgcttttttcagccttgacaactcccttttttgctgcatcaggctttcctttagctcgatatgcagcaatatccttttcgtatttttccttcagcttcgcagccttcttttcataaggctgcttgtcatctgcagcagtgttattccacatctctcccagtttcttcgcaacatcaccaatggacaggccaggatgttctcctttgatttttgggcgatactcagagcagaagaggaagaaggccgaaggaggcctcttgggtgcattgggatccttgaacttcttttttgtctcccctttgggagggatataggttttcatttctctttcataacgggccttgtccgcttttgccatatcttcaaattttcctttctctttagcagacatggtcttccacctctctgagcacttcttagaaaactctgagaagttgactgaagcatctgggtgcttcttcttatgctcctcccgacaagtttgcacaaaaaatgcatatgatgacattttgcctctcggcttcttaggatctcctttgcccatgtttagttatttttcagcctatttttgtctggagaaaccccaacaaactgctggattccttggccatttgcattcagaagtaccaaaaactaaaatcctttttactaaataatttcttctacacgagacttgtttcctccacaccaccctatccaaattgtcagcattattccagaatataatcatttagtttgagaccactaaaaaaccccgcagtccaaaataccaattgtggtttttctgtaaagaaatggtcagaaactacaaattgttatcctaggacacagaaccaatcgaccaaaaggacttctggaatatgctgcccccaagatttagaatgcacaggcagaaatagcatacgcggtcacgatgtcccttaagccacatgaccttcctacgaaagcaaaggcttaaacttatcaaatgagaactccccctttctctgaagttaaaacaaggcagggcagctggaattagagcagcagggacagatcggctgttgactagtcagaacgggtcgtggaatgcaaagtccctgcgctttcgctgctccccttaccgtgagaagatctgggagggaggaaaggaggagaaacaccccagaatcctggtagaaaagcccctggcctcgaagatgggctctagggagacagggaggggcagctccgtgtgtgatgaccctttgtgaacatgcactctgtggcagcttcagctccaccgaggctttgggagagcggactacggatgcccggcgcggcccagctgtgaaggccgcgccggcggagagggtccatggcacccccgccggcttcggaagcccttccctctcccacctccgcgggtcaccccaggaaccagcggctcccgaccacgctcgcgcggaccacggaacagcgacgcgcaagcaggtctctttcgtcagcgtaatccctccgcagaaa

SEQ ID NO: 13 - protein, 16 aa, Unknown
  source: /note=“Description of Unknown RFGF peptide”
AAVALLPAVLLALLAP

SEQ ID NO: 14 - protein, 11 aa, Unknown
  source: /note=“Description of Unknown RFGF analogue peptide”
AALLPVLLAAP

SEQ ID NO: 15 - protein, 13 aa, Human immunodeficiency virus
GRKKRRQRRRPPQ

SEQ ID NO: 16 - protein, 16 aa, Drosophila sp.
RQIKIWFQNRRMKWKK

SEQ ID NO: 17 - RNA, 21 nt, Artificial Sequence
  source: /note=“Description of Artificial Sequence Synthetic oligonucleotide”
gugcaaacuugucgggaggaa

SEQ ID NO: 18 - RNA, 21 nt, Artificial Sequence
  source: /note=“Description of Artificial Sequence Synthetic oligonucleotide”
uuuuuaaacuguacagugucu

SEQ ID NO: 19 - RNA, 21 nt, Artificial Sequence
  source: /note=“Description of Artificial Sequence Synthetic oligonucleotide”
uuuguauaguuaacacacuaa

SEQ ID NO: 20 - RNA, 21 nt, Artificial Sequence
  source: /note=“Description of Artificial Sequence Synthetic oligonucleotide”
uuguauaguuaacacacuaca

SEQ ID NO: 21 - RNA, 21 nt, Artificial Sequence
  source: /note=“Description of Artificial Sequence Synthetic oligonucleotide”
uguauaguuaacacacuacca

SEQ ID NO: 22 - RNA, 21 nt, Artificial Sequence
  source: /note=“Description of Artificial Sequence Synthetic oligonucleotide”
guauaguuaacacacuaccga

SEQ ID NO: 23 - RNA, 21 nt, Artificial Sequence
  source: /note=“Description of Artificial Sequence Synthetic oligonucleotide”
uauaguuaacacacuaccgaa

SEQ ID NO: 24 - RNA, 21 nt, Artificial Sequence
  source: /note=“Description of Artificial Sequence Synthetic oligonucleotide”
auaguuaacacacuaccgaau

SEQ ID NO: 25 - RNA, 21 nt, Artificial Sequence
  source: /note=“Description of Artificial Sequence Synthetic oligonucleotide”
uaguuaacacacuaccgaaua

SEQ ID NO: 26 - RNA, 21 nt, Artificial Sequence
  source: /note=“Description of Artificial Sequence Synthetic oligonucleotide”
aguuaacacacuaccgaaugu

SEQ ID NO: 27 - RNA, 21 nt, Artificial Sequence
  source: /note=“Description of Artificial Sequence Synthetic oligonucleotide”
guuaacacacuaccgaaugua

SEQ ID NO: 28 - RNA, 21 nt, Artificial Sequence
  source: /note=“Description of Artificial Sequence Synthetic oligonucleotide”
uuaacacacuaccgaaugugu

SEQ ID NO: 29 - RNA, 21 nt, Artificial Sequence
  source: /note=“Description of Artificial Sequence Synthetic oligonucleotide”
ucucugaugcagcuuauacga

SEQ ID NO: 30 - RNA, 21 nt, Artificial Sequence
  source: /note=“Description of Artificial Sequence Synthetic oligonucleotide”
cucugaugcagcuuauacgaa

SEQ ID NO: 31 - RNA, 21 nt, Artificial Sequence
  source: /note=“Description of Artificial Sequence Synthetic oligonucleotide”
ucugaugcagcuuauacgaaa

SEQ ID NO: 32 - RNA, 21 nt, Artificial Sequence
  source: /note=“Description of Artificial Sequence Synthetic oligonucleotide”
cugaugcagcuuauacgaaau

SEQ ID NO: 33 - RNA, 21 nt, Artificial Sequence
  source: /note=“Description of Artificial Sequence Synthetic oligonucleotide”
ugaugcagcuuauacgaaaua

SEQ ID NO: 34 - RNA, 21 nt, Artificial Sequence
  source: /note=“Description of Artificial Sequence Synthetic oligonucleotide”
uacgaaauaauuguuguucua

SEQ ID NO: 35 - RNA, 21 nt, Artificial Sequence
  source: /note=“Description of Artificial Sequence Synthetic oligonucleotide”
uuguuguccuuuucauaggua

SEQ ID NO: 36 - RNA, 21 nt, Artificial Sequence
  source: /note=“Description of Artificial Sequence Synthetic oligonucleotide”
gggaagcuagucuuuugcuuu

SEQ ID NO: 37 - RNA, 21 nt, Artificial Sequence
  source: /note=“Description of Artificial Sequence Synthetic oligonucleotide”
auccuuucauauaguuagcua

SEQ ID NO: 38 - RNA, 21 nt, Artificial Sequence
  source: /note=“Description of Artificial Sequence Synthetic oligonucleotide”
uccuuucauauaguuagcuaa

SEQ ID NO: 39 - RNA, 21 nt, Artificial Sequence
  source: /note=“Description of Artificial Sequence Synthetic oligonucleotide”
aaaaagcuuuugucuacacaa

SEQ ID NO: 40 - RNA, 21 nt, Artificial Sequence
  source: /note=“Description of Artificial Sequence Synthetic oligonucleotide”
uuugucuacacacccugcaua

SEQ ID NO: 41 - RNA, 21 nt, Artificial Sequence
  source: /note=“Description of Artificial Sequence Synthetic oligonucleotide”
uugucuacacacccugcauau

SEQ ID NO: 42 - RNA, 21 nt, Artificial Sequence
  source: /note=“Description of Artificial Sequence Synthetic oligonucleotide”
aaguuaaguugagauaguuuu

SEQ ID NO: 43 - RNA, 21 nt, Artificial Sequence
  source: /note=“Description of Artificial Sequence Synthetic oligonucleotide”
guuaaguugagauaguuuuca

SEQ ID NO: 44 - RNA, 21 nt, Artificial Sequence
  source: /note=“Description of Artificial Sequence Synthetic oligonucleotide”
aaguugagauaguuuucauca

SEQ ID NO: 45 - RNA, 21 nt, Artificial Sequence
  source: /note=“Description of Artificial Sequence Synthetic oligonucleotide”
agauaguuuucauccauaacu

SEQ ID NO: 46 - RNA, 21 nt, Artificial Sequence
  source: /note=“Description of Artificial Sequence Synthetic oligonucleotide”
gauaguuuucauccauaacua

SEQ ID NO: 47 - RNA, 21 nt, Artificial Sequence
  source: /note=“Description of Artificial Sequence Synthetic oligonucleotide”
auaguuuucauccauaacuga

SEQ ID NO: 48 - RNA, 21 nt, Artificial Sequence
  source: /note=“Description of Artificial Sequence Synthetic oligonucleotide”
aaaaucuugaucaguuaagaa

SEQ ID NO: 49 - RNA, 21 nt, Artificial Sequence
  source: /note=“Description of Artificial Sequence Synthetic oligonucleotide”
uaagaaauuucacauagccca

SEQ ID NO: 50 - RNA, 21 nt, Artificial Sequence
  source: /note=“Description of Artificial Sequence Synthetic oligonucleotide”
uucacauagcccacuuacauu

SEQ ID NO: 51 - RNA, 21 nt, Artificial Sequence
  source: /note=“Description of Artificial Sequence Synthetic oligonucleotide”
ucacauagcccacuuacauuu

SEQ ID NO: 52 - RNA, 21 nt, Artificial Sequence
  source: /note=“Description of Artificial Sequence Synthetic oligonucleotide”
cacauagcccacuuacauuua

SEQ ID NO: 53 - RNA, 21 nt, Artificial Sequence
  source: /note=“Description of Artificial Sequence Synthetic oligonucleotide”
cauagcccacuuacauuuaca

SEQ ID NO: 54 - RNA, 21 nt, Artificial Sequence
  source: /note=“Description of Artificial Sequence Synthetic oligonucleotide”
agcccacuuacauuuacaaac

SEQ ID NO: 55 - RNA, 21 nt, Artificial Sequence
  source: /note=“Description of Artificial Sequence Synthetic oligonucleotide”
aaucuacucaaagcaugggau

SEQ ID NO: 56 - RNA, 21 nt, Artificial Sequence
  source: /note=“Description of Artificial Sequence Synthetic oligonucleotide”
uacucaaagcaugggauuauu

SEQ ID NO: 57 - RNA, 21 nt, Artificial Sequence
  source: /note=“Description of Artificial Sequence Synthetic oligonucleotide”
acucaaagcaugggauuauua

SEQ ID NO: 58 - RNA, 21 nt, Artificial Sequence
  source: /note=“Description of Artificial Sequence Synthetic oligonucleotide”
caaagcaugggauuauuagaa

SEQ ID NO: 59 - RNA, 21 nt, Artificial Sequence
  source: /note=“Description of Artificial Sequence Synthetic oligonucleotide”
aagcaugggauuauuagaaua

SEQ ID NO: 60 - RNA, 21 nt, Artificial Sequence
  source: /note=“Description of Artificial Sequence Synthetic oligonucleotide”
ugggauuauuagaaucaaaca

SEQ ID NO: 61 - RNA, 21 nt, Artificial Sequence
  source: /note=“Description of Artificial Sequence Synthetic oligonucleotide”
aaagucuguccuugaaggacu

SEQ ID NO: 62 - RNA, 21 nt, Artificial Sequence
  source: /note=“Description of Artificial Sequence Synthetic oligonucleotide”
ucuguccuugaaggacuaaua

SEQ ID NO: 63 - RNA, 21 nt, Artificial Sequence
  source: /note=“Description of Artificial Sequence Synthetic oligonucleotide”
cuguccuugaaggacuaauaa

SEQ ID NO: 64 - RNA, 21 nt, Artificial Sequence
  source: /note=“Description of Artificial Sequence Synthetic oligonucleotide”
guccuugaaggacuaauagaa

SEQ ID NO: 65 - RNA, 21 nt, Artificial Sequence
  source: /note=“Description of Artificial Sequence Synthetic oligonucleotide”
aaguauguucuaaccuuuaca

SEQ ID NO: 66 - RNA, 21 nt, Artificial Sequence
  source: /note=“Description of Artificial Sequence Synthetic oligonucleotide”
uucuaaccuuuacaugaggaa

SEQ ID NO: 67 - RNA, 21 nt, Artificial Sequence
  source: /note=“Description of Artificial Sequence Synthetic oligonucleotide”
accuuuacaugaggacucuau

SEQ ID NO: 68 - RNA, 21 nt, Artificial Sequence
  source: /note=“Description of Artificial Sequence Synthetic oligonucleotide”
uuuacaugaggacucuauucu

SEQ ID NO: 69 - RNA, 21 nt, Artificial Sequence
  source: /note=“Description of Artificial Sequence Synthetic oligonucleotide”
uuacaugaggacucuauucuu

SEQ ID NO: 70 - RNA, 21 nt, Artificial Sequence
  source: /note=“Description of Artificial Sequence Synthetic oligonucleotide”
uacaugaggacucuauucuuu

SEQ ID NO: 71 - RNA, 21 nt, Artificial Sequence
  source: /note=“Description of Artificial Sequence Synthetic oligonucleotide”
ugaggacucuauucuuuaacu

SEQ ID NO: 72 - RNA, 21 nt, Artificial Sequence
  source: /note=“Description of Artificial Sequence Synthetic oligonucleotide”
aggacucuauucuuuaacuca

SEQ ID NO: 73 - RNA, 21 nt, Artificial Sequence
  source: /note=“Description of Artificial Sequence Synthetic oligonucleotide”
cucuauucuuuaacucccauu

SEQ ID NO: 74 - RNA, 21 nt, Artificial Sequence
  source: /note=“Description of Artificial Sequence Synthetic oligonucleotide”
uuuaacucccauuaccaugua

SEQ ID NO: 75 - RNA, 21 nt, Artificial Sequence
  source: /note=“Description of Artificial Sequence Synthetic oligonucleotide”
uuaacucccauuaccauguaa

SEQ ID NO: 76 - RNA, 21 nt, Artificial Sequence
  source: /note=“Description of Artificial Sequence Synthetic oligonucleotide”
uaacucccauuaccauguaau

SEQ ID NO: 77 - RNA, 21 nt, Artificial Sequence
  source: /note=“Description of Artificial Sequence Synthetic oligonucleotide”
acucccauuaccauguaauga

SEQ ID NO: 78 - RNA, 21 nt, Artificial Sequence
  source: /note=“Description of Artificial Sequence Synthetic oligonucleotide”
ucccauuaccauguaauggca

SEQ ID NO: 79 - RNA, 21 nt, Artificial Sequence
  source: /note=“Description of Artificial Sequence Synthetic oligonucleotide”
uaccauguaauggcaguuaua

SEQ ID NO: 80 - RNA, 21 nt, Artificial Sequence
  source: /note=“Description of Artificial Sequence Synthetic oligonucleotide”
uaauggcaguuauauuuugca

SEQ ID NO: 81 - RNA, 21 nt, Artificial Sequence
  source: /note=“Description of Artificial Sequence Synthetic oligonucleotide”
aaagcgugagcuuaaaauaca

SEQ ID NO: 82 - RNA, 21 nt, Artificial Sequence
  source: /note=“Description of Artificial Sequence Synthetic oligonucleotide”
aagcgugagcuuaaaauacaa

SEQ ID NO: 83 - RNA, 21 nt, Artificial Sequence
  source: /note=“Description of Artificial Sequence Synthetic oligonucleotide”
uuuguugacauuagucucagu

SEQ ID NO: 84 - RNA, 21 nt, Artificial Sequence
  source: /note=“Description of Artificial Sequence Synthetic oligonucleotide”
uuguugacauuagucucagua

SEQ ID NO: 85 - RNA, 21 nt, Artificial Sequence
  source: /note=“Description of Artificial Sequence Synthetic oligonucleotide”
ugaaaaugcuggcuauagaua

SEQ ID NO: 86 - RNA, 21 nt, Artificial Sequence
  source: /note=“Description of Artificial Sequence Synthetic oligonucleotide”
cuggcuauagaugucuuuuca

SEQ ID NO: 87 - RNA, 21 nt, Artificial Sequence
  source: /note=“Description of Artificial Sequence Synthetic oligonucleotide”
uaaauauggacugcucaggaa

SEQ ID NO: 88 - RNA, 21 nt, Artificial Sequence
  source: /note=“Description of Artificial Sequence Synthetic oligonucleotide”
auggacugcucaggaaacgaa

SEQ ID NO: 89 - RNA, 21 nt, Artificial Sequence
  source: /note=“Description of Artificial Sequence Synthetic oligonucleotide”
ggacugcucaggaaacgagaa

SEQ ID NO: 90 - RNA, 21 nt, Artificial Sequence
  source: /note=“Description of Artificial Sequence Synthetic oligonucleotide”
ugcucaggaaacgagacuuua

SEQ ID NO: 91 - RNA, 21 nt, Artificial Sequence
  source: /note=“Description of Artificial Sequence Synthetic oligonucleotide”
ggaaacgagacuuuccauuaa

SEQ ID NO: 92 - RNA, 21 nt, Artificial Sequence
  source: /note=“Description of Artificial Sequence Synthetic oligonucleotide”
gaaacgagacuuuccauuaca

SEQ ID NO: 93 - RNA, 21 nt, Artificial Sequence
  source: /note=“Description of Artificial Sequence Synthetic oligonucleotide”
uuuaauuaauugggccagcuu

SEQ ID NO: 94 - RNA, 21 nt, Artificial Sequence
  source: /note=“Description of Artificial Sequence Synthetic oligonucleotide”
uaauuaauugggccagcuuuu

SEQ ID NO: 95 - RNA, 21 nt, Artificial Sequence
  source: /note=“Description of Artificial Sequence Synthetic oligonucleotide”
gguuuucgagauuaucguuuu

SEQ ID NO: 96 - RNA, 21 nt, Artificial Sequence
  source: /note=“Description of Artificial Sequence Synthetic oligonucleotide”
guuuucgagauuaucguuuua

SEQ ID NO: 97 - RNA, 21 nt, Artificial Sequence
  source: /note=“Description of Artificial Sequence Synthetic oligonucleotide”
uuuucgagauuaucguuuucu

SEQ ID NO: 98 - RNA, 21 nt, Artificial Sequence
  source: /note=“Description of Artificial Sequence Synthetic oligonucleotide”
uuucgagauuaucguuuucuu

SEQ ID NO: 99 - RNA, 21 nt, Artificial Sequence
  source: /note=“Description of Artificial Sequence Synthetic oligonucleotide”
guauuuuaaaauagcguucuu

SEQ ID NO: 100 - RNA, 21 nt, Artificial Sequence
  source: /note=“Description of Artificial Sequence Synthetic oligonucleotide”
cguucuuguaauuuuacacga

SEQ ID NO: 101 - RNA, 21 nt, Artificial Sequence
  source: /note=“Description of Artificial Sequence Synthetic oligonucleotide”
guucuuguaauuuuacacgcu

SEQ ID NO: 102 - RNA, 21 nt, Artificial Sequence
  source: /note=“Description of Artificial Sequence Synthetic oligonucleotide”
uucuuguaauuuuacacgcuu

SEQ ID NO: 103 - RNA, 21 nt, Artificial Sequence
  source: /note=“Description of Artificial Sequence Synthetic oligonucleotide”
ucuuguaauuuuacacgcuuu

SEQ ID NO: 104 - RNA, 21 nt, Artificial Sequence
  source: /note=“Description of Artificial Sequence Synthetic oligonucleotide”
uguaauuuuacacgcuuuugu

SEQ ID NO: 105 - RNA, 21 nt, Artificial Sequence
  source: /note=“Description of Artificial Sequence Synthetic oligonucleotide”
uggagugcuguuuuguuauau

SEQ ID NO: 106 - RNA, 21 nt, Artificial Sequence
  source: /note=“Description of Artificial Sequence Synthetic oligonucleotide”
aggaggaauacugaacaucua

SEQ ID NO: 107 - RNA, 21 nt, Artificial Sequence
  source: /note=“Description of Artificial Sequence Synthetic oligonucleotide”
aucugaguccuggaugauacu

SEQ ID NO: 108 - RNA, 21 nt, Artificial Sequence
  source: /note=“Description of Artificial Sequence Synthetic oligonucleotide”
gaguccuggaugauacuaaua

SEQ ID NO: 109 - RNA, 21 nt, Artificial Sequence
  source: /note=“Description of Artificial Sequence Synthetic oligonucleotide”
aguccuggaugauacuaauaa

SEQ ID NO: 110 - RNA, 21 nt, Artificial Sequence
  source: /note=“Description of Artificial Sequence Synthetic oligonucleotide”
guccuggaugauacuaauaaa

SEQ ID NO: 111 - RNA, 23 nt, Artificial Sequence
  source: /note=“Description of Artificial Sequence Synthetic oligonucleotide”
uuccucccgacaaguuugcacaa

SEQ ID NO: 112 - RNA, 23 nt, Artificial Sequence
  source: /note=“Description of Artificial Sequence Synthetic oligonucleotide”
agacacuguacaguuuaaaaaca

SEQ ID NO: 113 - RNA, 23 nt, Artificial Sequence
  source: /note=“Description of Artificial Sequence Synthetic oligonucleotide”
uuaguguguuaacuauacaaaaa

SEQ ID NO: 114 - RNA, 23 nt, Artificial Sequence
  source: /note=“Description of Artificial Sequence Synthetic oligonucleotide”
uguaguguguuaacuauacaaaa

SEQ ID NO: 115 - RNA, 23 nt, Artificial Sequence
  source: /note=“Description of Artificial Sequence Synthetic oligonucleotide”
ugguaguguguuaacuauacaaa

SEQ ID NO: 116 - RNA, 23 nt, Artificial Sequence
  source: /note=“Description of Artificial Sequence Synthetic oligonucleotide”
ucgguaguguguuaacuauacaa

SEQ ID NO: 117 - RNA, 23 nt, Artificial Sequence
  source: /note=“Description of Artificial Sequence Synthetic oligonucleotide”
uucgguaguguguuaacuauaca

SEQ ID NO: 118 - RNA, 23 nt, Artificial Sequence
  source: /note=“Description of Artificial Sequence Synthetic oligonucleotide”
auucgguaguguguuaacuauac

SEQ ID NO: 119 - RNA, 23 nt, Artificial Sequence
  source: /note=“Description of Artificial Sequence Synthetic oligonucleotide”
uauucgguaguguguuaacuaua

SEQ ID NO: 120 - RNA, 23 nt, Artificial Sequence
  source: /note=“Description of Artificial Sequence Synthetic oligonucleotide”
acauucgguaguguguuaacuau

SEQ ID NO: 121 - RNA, 23 nt, Artificial Sequence
  source: /note=“Description of Artificial Sequence Synthetic oligonucleotide”
uacauucgguaguguguuaacua

SEQ ID NO: 122 - RNA, 23 nt, Artificial Sequence
  source: /note=“Description of Artificial Sequence Synthetic oligonucleotide”
acacauucgguaguguguuaacu

SEQ ID NO: 123 - RNA, 23 nt, Artificial Sequence
  source: /note=“Description of Artificial Sequence Synthetic oligonucleotide”
ucguauaagcugcaucagagaca

SEQ ID NO: 124 - RNA, 23 nt, Artificial Sequence
  source: /note=“Description of Artificial Sequence Synthetic oligonucleotide”
uucguauaagcugcaucagagac

SEQ ID NO: 125 - RNA, 23 nt, Artificial Sequence
  source: /note=“Description of Artificial Sequence Synthetic oligonucleotide”
uuucguauaagcugcaucagaga

SEQ ID NO: 126 - RNA, 23 nt, Artificial Sequence
  source: /note=“Description of Artificial Sequence Synthetic oligonucleotide”
auuucguauaagcugcaucagag

SEQ ID NO: 127 - RNA, 23 nt, Artificial Sequence
  source: /note=“Description of Artificial Sequence Synthetic oligonucleotide”
uauuucguauaagcugcaucaga

SEQ ID NO: 128 - RNA, 23 nt, Artificial Sequence
  source: /note=“Description of Artificial Sequence Synthetic oligonucleotide”
uagaacaacaauuauuucguaua

SEQ ID NO: 129 - RNA, 23 nt, Artificial Sequence
  source: /note=“Description of Artificial Sequence Synthetic oligonucleotide”
uaccuaugaaaaggacaacaaua

SEQ ID NO: 130 - RNA, 23 nt, Artificial Sequence
  source: /note=“Description of Artificial Sequence Synthetic oligonucleotide”
aaagcaaaagacuagcuuccccu

SEQ ID NO: 131 - RNA, 23 nt, Artificial Sequence
  source: /note=“Description of Artificial Sequence Synthetic oligonucleotide”
uagcuaacuauaugaaaggauaa

SEQ ID NO: 132 - RNA, 23 nt, Artificial Sequence
  source: /note=“Description of Artificial Sequence Synthetic oligonucleotide”
uuagcuaacuauaugaaaggaua

SEQ ID NO: 133 - RNA, 23 nt, Artificial Sequence
  source: /note=“Description of Artificial Sequence Synthetic oligonucleotide”
uuguguagacaaaagcuuuuuau

SEQ ID NO: 134 - RNA, 23 nt, Artificial Sequence
  source: /note=“Description of Artificial Sequence Synthetic oligonucleotide”
uaugcaggguguguagacaaaag

SEQ ID NO: 135 - RNA, 23 nt, Artificial Sequence
  source: /note=“Description of Artificial Sequence Synthetic oligonucleotide”
auaugcaggguguguagacaaaa

SEQ ID NO: 136 - RNA, 23 nt, Artificial Sequence
  source: /note=“Description of Artificial Sequence Synthetic oligonucleotide”
aaaacuaucucaacuuaacuuua

SEQ ID NO: 137 - RNA, 23 nt, Artificial Sequence
  source: /note=“Description of Artificial Sequence Synthetic oligonucleotide”
ugaaaacuaucucaacuuaacuu

SEQ ID NO: 138 - RNA, 23 nt, Artificial Sequence
  source: /note=“Description of Artificial Sequence Synthetic oligonucleotide”
ugaugaaaacuaucucaacuuaa

SEQ ID NO: 139 - RNA, 23 nt, Artificial Sequence
  source: /note=“Description of Artificial Sequence Synthetic oligonucleotide”
aguuauggaugaaaacuaucuca

SEQ ID NO: 140 - RNA, 23 nt, Artificial Sequence
  source: /note=“Description of Artificial Sequence Synthetic oligonucleotide”
uaguuauggaugaaaacuaucuc

SEQ ID NO: 141 - RNA, 23 nt, Artificial Sequence
  source: /note=“Description of Artificial Sequence Synthetic oligonucleotide”
ucaguuauggaugaaaacuaucu

SEQ ID NO: 142 - RNA, 23 nt, Artificial Sequence
  source: /note=“Description of Artificial Sequence Synthetic oligonucleotide”
uucuuaacugaucaagauuuugg

SEQ ID NO: 143 - RNA, 23 nt, Artificial Sequence
  source: /note=“Description of Artificial Sequence Synthetic oligonucleotide”
ugggcuaugugaaauuucuuaac

SEQ ID NO: 144 - RNA, 23 nt, Artificial Sequence
  source: /note=“Description of Artificial Sequence Synthetic oligonucleotide”
aauguaagugggcuaugugaaau

SEQ ID NO: 145 - RNA, 23 nt, Artificial Sequence
  source: /note=“Description of Artificial Sequence Synthetic oligonucleotide”
aaauguaagugggcuaugugaaa

SEQ ID NO: 146 - RNA, 23 nt, Artificial Sequence
  source: /note=“Description of Artificial Sequence Synthetic oligonucleotide”
uaaauguaagugggcuaugugaa

SEQ ID NO: 147 - RNA, 23 nt, Artificial Sequence
  source: /note=“Description of Artificial Sequence Synthetic oligonucleotide”
uguaaauguaagugggcuaugug

SEQ ID NO: 148 - RNA, 23 nt, Artificial Sequence
  source: /note=“Description of Artificial Sequence Synthetic oligonucleotide”
guuuguaaauguaagugggcuau

SEQ ID NO: 149 - RNA, 23 nt, Artificial Sequence
  source: /note=“Description of Artificial Sequence Synthetic oligonucleotide”
aucccaugcuuugaguagauuga

SEQ ID NO: 150 - RNA, 23 nt, Artificial Sequence
  source: /note=“Description of Artificial Sequence Synthetic oligonucleotide”
aauaaucccaugcuuugaguaga

SEQ ID NO: 151 - RNA, 23 nt, Artificial Sequence
  source: /note=“Description of Artificial Sequence Synthetic oligonucleotide”
uaauaaucccaugcuuugaguag

SEQ ID NO: 152 - RNA, 23 nt, Artificial Sequence
  source: /note=“Description of Artificial Sequence Synthetic oligonucleotide”
uucuaauaaucccaugcuuugag

SEQ ID NO: 153 - RNA, 23 nt, Artificial Sequence
  source: /note=“Description of Artificial Sequence Synthetic oligonucleotide”
uauucuaauaaucccaugcuuug

SEQ ID NO: 154 - RNA, 23 nt, Artificial Sequence
  source: /note=“Description of Artificial Sequence Synthetic oligonucleotide”
uguuugauucuaauaaucccaug

SEQ ID NO: 155 - RNA, 23 nt, Artificial Sequence
  source: /note=“Description of Artificial Sequence Synthetic oligonucleotide”
aguccuucaaggacagacuuuca

SEQ ID NO: 156 - RNA, 23 nt, Artificial Sequence
  source: /note=“Description of Artificial Sequence Synthetic oligonucleotide”
uauuaguccuucaaggacagacu

SEQ ID NO: 157 - RNA, 23 nt, Artificial Sequence
  source: /note=“Description of Artificial Sequence Synthetic oligonucleotide”
uuauuaguccuucaaggacagac

SEQ ID NO: 158 - RNA, 23 nt, Artificial Sequence
  source: /note=“Description of Artificial Sequence Synthetic oligonucleotide”
uucuauuaguccuucaaggacag

SEQ ID NO: 159 - RNA, 23 nt, Artificial Sequence
  source: /note=“Description of Artificial Sequence Synthetic oligonucleotide”
uguaaagguuagaacauacuuuu

SEQ ID NO: 160 - RNA, 23 nt, Artificial Sequence
  source: /note=“Description of Artificial Sequence Synthetic oligonucleotide”
uuccucauguaaagguuagaaca

SEQ ID NO: 161 - RNA, 23 nt, Artificial Sequence
  source: /note=“Description of Artificial Sequence Synthetic oligonucleotide”
auagaguccucauguaaagguua

SEQ ID NO: 162 - RNA, 23 nt, Artificial Sequence
  source: /note=“Description of Artificial Sequence Synthetic oligonucleotide”
agaauagaguccucauguaaagg

SEQ ID NO: 163 - RNA, 23 nt, Artificial Sequence
  source: /note=“Description of Artificial Sequence Synthetic oligonucleotide”
aagaauagaguccucauguaaag

SEQ ID NO: 164 - RNA, 23 nt, Artificial Sequence
  source: /note=“Description of Artificial Sequence Synthetic oligonucleotide”
aaagaauagaguccucauguaaa

SEQ ID NO: 165 - RNA, 23 nt, Artificial Sequence
  source: /note=“Description of Artificial Sequence Synthetic oligonucleotide”
aguuaaagaauagaguccucaug

SEQ ID NO: 166 - RNA, 23 nt, Artificial Sequence
  source: /note=“Description of Artificial Sequence Synthetic oligonucleotide”
ugaguuaaagaauagaguccuca

SEQ ID NO: 167 - RNA, 23 nt, Artificial Sequence
  source: /note=“Description of Artificial Sequence Synthetic oligonucleotide”
aaugggaguuaaagaauagaguc

SEQ ID NO: 168 - RNA, 23 nt, Artificial Sequence
  source: /note=“Description of Artificial Sequence Synthetic oligonucleotide”
uacaugguaaugggaguuaaaga

SEQ ID NO: 169 - RNA, 23 nt, Artificial Sequence
  source: /note=“Description of Artificial Sequence Synthetic oligonucleotide”
uuacaugguaaugggaguuaaag

SEQ ID NO: 170 - RNA, 23 nt, Artificial Sequence
  source: /note=“Description of Artificial Sequence Synthetic oligonucleotide”
auuacaugguaaugggaguuaaa

SEQ ID NO: 171 - RNA, 23 nt, Artificial Sequence
  source: /note=“Description of Artificial Sequence Synthetic oligonucleotide”
ucauuacaugguaaugggaguua

SEQ ID NO: 172 - RNA, 23 nt, Artificial Sequence
  source: /note=“Description of Artificial Sequence Synthetic oligonucleotide”
ugccauuacaugguaaugggagu

SEQ ID NO: 173 - RNA, 23 nt, Artificial Sequence
  source: /note=“Description of Artificial Sequence Synthetic oligonucleotide”
uauaacugccauuacaugguaau

SEQ ID NO: 174 - RNA, 23 nt, Artificial Sequence
  source: /note=“Description of Artificial Sequence Synthetic oligonucleotide”
ugcaaaauauaacugccauuaca

SEQ ID NO: 175 - RNA, 23 nt, Artificial Sequence
  source: /note=“Description of Artificial Sequence Synthetic oligonucleotide”
uguauuuuaagcucacgcuuuug

SEQ ID NO: 176 - RNA, 23 nt, Artificial Sequence
  source: /note=“Description of Artificial Sequence Synthetic oligonucleotide”
uuguauuuuaagcucacgcuuuu

SEQ ID NO: 177 - RNA, 23 nt, Artificial Sequence
  source: /note=“Description of Artificial Sequence Synthetic oligonucleotide”
acugagacuaaugucaacaaaaa

SEQ ID NO: 178 - RNA, 23 nt, Artificial Sequence
  source: /note=“Description of Artificial Sequence Synthetic oligonucleotide”
uacugagacuaaugucaacaaaa

SEQ ID NO: 179 - RNA, 23 nt, Artificial Sequence
  source: /note=“Description of Artificial Sequence Synthetic oligonucleotide”
uaucuauagccagcauuuucaua

SEQ ID NO: 180 - RNA, 23 nt, Artificial Sequence
  source: /note=“Description of Artificial Sequence Synthetic oligonucleotide”
ugaaaagacaucuauagccagca

SEQ ID NO: 181 - RNA, 23 nt, Artificial Sequence
  source: /note=“Description of Artificial Sequence Synthetic oligonucleotide”
uuccugagcaguccauauuuaga

SEQ ID NO: 182 - RNA, 23 nt, Artificial Sequence
  source: /note=“Description of Artificial Sequence Synthetic oligonucleotide”
uucguuuccugagcaguccauau

SEQ ID NO: 183 - RNA, 23 nt, Artificial Sequence
  source: /note=“Description of Artificial Sequence Synthetic oligonucleotide”
uucucguuuccugagcaguccau

SEQ ID NO: 184 - RNA, 23 nt, Artificial Sequence
  source: /note=“Description of Artificial Sequence Synthetic oligonucleotide”
uaaagucucguuuccugagcagu

SEQ ID NO: 185 - RNA, 23 nt, Artificial Sequence
  source: /note=“Description of Artificial Sequence Synthetic oligonucleotide”
uuaauggaaagucucguuuccug

SEQ ID NO: 186 - RNA, 23 nt, Artificial Sequence
  source: /note=“Description of Artificial Sequence Synthetic oligonucleotide”
uguaauggaaagucucguuuccu

SEQ ID NO: 187 - RNA, 23 nt, Artificial Sequence
  source: /note=“Description of Artificial Sequence Synthetic oligonucleotide”
aagcuggcccaauuaauuaaaaa

SEQ ID NO: 188 - RNA, 23 nt, Artificial Sequence
  source: /note=“Description of Artificial Sequence Synthetic oligonucleotide”
aaaagcuggcccaauuaauuaaa

SEQ ID NO: 189 - RNA, 23 nt, Artificial Sequence
  source: /note=“Description of Artificial Sequence Synthetic oligonucleotide”
aaaacgauaaucucgaaaaccac

SEQ ID NO: 190 - RNA, 23 nt, Artificial Sequence
  source: /note=“Description of Artificial Sequence Synthetic oligonucleotide”
uaaaacgauaaucucgaaaacca

SEQ ID NO: 191 - RNA, 23 nt, Artificial Sequence
  source: /note=“Description of Artificial Sequence Synthetic oligonucleotide”
agaaaacgauaaucucgaaaacc

SEQ ID NO: 192 - RNA, 23 nt, Artificial Sequence
  source: /note=“Description of Artificial Sequence Synthetic oligonucleotide”
aagaaaacgauaaucucgaaaac

SEQ ID NO: 193 - RNA, 23 nt, Artificial Sequence
  source: /note=“Description of Artificial Sequence Synthetic oligonucleotide”
aagaacgcuauuuuaaaauacug

SEQ ID NO: 194 - RNA, 23 nt, Artificial Sequence
  source: /note=“Description of Artificial Sequence Synthetic oligonucleotide”
ucguguaaaauuacaagaacgcu

SEQ ID NO: 195 - RNA, 23 nt, Artificial Sequence
  source: /note=“Description of Artificial Sequence Synthetic oligonucleotide”
agcguguaaaauuacaagaacgc

SEQ ID NO: 196 - RNA, 23 nt, Artificial Sequence
  source: /note=“Description of Artificial Sequence Synthetic oligonucleotide”
aagcguguaaaauuacaagaacg

SEQ ID NO: 197 - RNA, 23 nt, Artificial Sequence
  source: /note=“Description of Artificial Sequence Synthetic oligonucleotide”
aaagcguguaaaauuacaagaac

SEQ ID NO: 198 - RNA, 23 nt, Artificial Sequence
  source: /note=“Description of Artificial Sequence Synthetic oligonucleotide”
acaaaagcguguaaaauuacaag

SEQ ID NO: 199 - RNA, 23 nt, Artificial Sequence
  source: /note=“Description of Artificial Sequence Synthetic oligonucleotide”
auauaacaaaacagcacuccauc

SEQ ID NO: 200 - RNA, 23 nt, Artificial Sequence
  source: /note=“Description of Artificial Sequence Synthetic oligonucleotide”
uagauguucaguauuccuccuga

SEQ ID NO: 201 - RNA, 23 nt, Artificial Sequence
  source: /note=“Description of Artificial Sequence Synthetic oligonucleotide”
aguaucauccaggacucagaugu

SEQ ID NO: 202 - RNA, 23 nt, Artificial Sequence
  source: /note=“Description of Artificial Sequence Synthetic oligonucleotide”
uauuaguaucauccaggacucag

SEQ ID NO: 203 - RNA, 23 nt, Artificial Sequence
  source: /note=“Description of Artificial Sequence Synthetic oligonucleotide”
uuauuaguaucauccaggacuca

SEQ ID NO: 204 - RNA, 23 nt, Artificial Sequence
  source: /note=“Description of Artificial Sequence Synthetic oligonucleotide”
uuuauuaguaucauccaggacuc

SEQ ID NO: 205 - RNA, 21 nt, Artificial Sequence
  source: /note=“Description of Artificial Sequence Synthetic oligonucleotide”
uacgaaauaauuguuguucua

SEQ ID NO: 206 - RNA, 21 nt, Artificial Sequence
  source: /note=“Description of Artificial Sequence Synthetic oligonucleotide”
auccuuucauauaguuagcua

SEQ ID NO: 207 - RNA, 21 nt, Artificial Sequence
  source: /note=“Description of Artificial Sequence Synthetic oligonucleotide”
cacauagcccacuuacauuua

SEQ ID NO: 208 - RNA, 21 nt, Artificial Sequence
  source: /note=“Description of Artificial Sequence Synthetic oligonucleotide”
guccuggaugauacuaauaaa

SEQ ID NO: 209 - RNA, 21 nt, Artificial Sequence
  source: /note=“Description of Artificial Sequence Synthetic oligonucleotide”
uucacauagcccacuuacauu

SEQ ID NO: 210 - RNA, 21 nt, Artificial Sequence
  source: /note=“Description of Artificial Sequence Synthetic oligonucleotide”
cauagcccacuuacauuuaca

SEQ ID NO: 211 - RNA, 21 nt, Artificial Sequence
  source: /note=“Description of Artificial Sequence Synthetic oligonucleotide”
uacaugaggacucuauucuuu

SEQ ID NO: 212 - RNA, 21 nt, Artificial Sequence
  source: /note=“Description of Artificial Sequence Synthetic oligonucleotide”
ugaggacucuauucuuuaacu

SEQ ID NO: 213 - RNA, 21 nt, Artificial Sequence
  source: /note=“Description of Artificial Sequence Synthetic oligonucleotide”
aagcgugagcuuaaaauacaa

SEQ ID NO: 214 - RNA, 21 nt, Artificial Sequence
  source: /note=“Description of Artificial Sequence Synthetic oligonucleotide”
gguuuucgagauuaucguuuu

SEQ ID NO: 215 - RNA, 21 nt, Artificial Sequence
  source: /note=“Description of Artificial Sequence Synthetic oligonucleotide”
guauuuuaaaauagcguucuu

SEQ ID NO: 216 - RNA, 21 nt, Artificial Sequence
  source: /note=“Description of Artificial Sequence Synthetic oligonucleotide”
uuguuguccuuuucauaggua

SEQ ID NO: 217 - RNA, 21 nt, Artificial Sequence
  source: /note=“Description of Artificial Sequence Synthetic oligonucleotide”
ggaaacgagacuuuccauuaa

SEQ ID NO: 218 - RNA, 21 nt, Artificial Sequence
  source: /note=“Description of Artificial Sequence Synthetic oligonucleotide”
guuuucgagauuaucguuuua

SEQ ID NO: 219 - RNA, 21 nt, Artificial Sequence
  source: /note=“Description of Artificial Sequence Synthetic oligonucleotide”
uggagugcuguuuuguuauau

SEQ ID NO: 220 - RNA, 21 nt, Artificial Sequence
  source: /note=“Description of Artificial Sequence Synthetic oligonucleotide”
ucugaugcagcuuauacgaaa

SEQ ID NO: 221 - RNA, 21 nt, Artificial Sequence
  source: /note=“Description of Artificial Sequence Synthetic oligonucleotide”
cugaugcagcuuauacgaaau

SEQ ID NO: 222 - RNA, 21 nt, Artificial Sequence
  source: /note=“Description of Artificial Sequence Synthetic oligonucleotide”
gauaguuuucauccauaacua

SEQ ID NO: 223 - RNA, 21 nt, Artificial Sequence
  source: /note=“Description of Artificial Sequence Synthetic oligonucleotide”
ucacauagcccacuuacauuu

SEQ ID NO: 224 - RNA, 21 nt, Artificial Sequence
  source: /note=“Description of Artificial Sequence Synthetic oligonucleotide”
uuacaugaggacucuauucuu

SEQ ID NO: 225 - RNA, 21 nt, Artificial Sequence
  source: /note=“Description of Artificial Sequence Synthetic oligonucleotide”
cuggcuauagaugucuuuuca

SEQ ID NO: 226 - RNA, 21 nt, Artificial Sequence
  source: /note=“Description of Artificial Sequence Synthetic oligonucleotide”
uuucgagauuaucguuuucuu

SEQ ID NO: 227 - RNA, 21 nt, Artificial Sequence
  source: /note=“Description of Artificial Sequence Synthetic oligonucleotide”
uucuuguaauuuuacacgcuu

SEQ ID NO: 228 - RNA, 21 nt, Artificial Sequence
  source: /note=“Description of Artificial Sequence Synthetic oligonucleotide”
aguuaacacacuaccgaaugu

SEQ ID NO: 229 - RNA, 21 nt, Artificial Sequence
  source: /note=“Description of Artificial Sequence Synthetic oligonucleotide”
aaguuaaguugagauaguuuu

SEQ ID NO: 230 - RNA, 21 nt, Artificial Sequence
  source: /note=“Description of Artificial Sequence Synthetic oligonucleotide”
aagcaugggauuauuagaaua

SEQ ID NO: 231 - RNA, 21 nt, Artificial Sequence
  source: /note=“Description of Artificial Sequence Synthetic oligonucleotide”
cucuauucuuuaacucccauu

SEQ ID NO: 232 - RNA, 21 nt, Artificial Sequence
  source: /note=“Description of Artificial Sequence Synthetic oligonucleotide”
ucuuguaauuuuacacgcuuu

SEQ ID NO: 233 - RNA, 21 nt, Artificial Sequence
  source: /note=“Description of Artificial Sequence Synthetic oligonucleotide”
uaguuaacacacuaccgaaua

SEQ ID NO: 234 - RNA, 21 nt, Artificial Sequence
  source: /note=“Description of Artificial Sequence Synthetic oligonucleotide”
guuaacacacuaccgaaugua

SEQ ID NO: 235 - RNA, 21 nt, Artificial Sequence
  source: /note=“Description of Artificial Sequence Synthetic oligonucleotide”
cucugaugcagcuuauacgaa

SEQ ID NO: 236 - RNA, 21 nt, Artificial Sequence
  source: /note=“Description of Artificial Sequence Synthetic oligonucleotide”
uuugucuacacacccugcaua

SEQ ID NO: 237 - RNA, 21 nt, Artificial Sequence
  source: /note=“Description of Artificial Sequence Synthetic oligonucleotide”
agauaguuuucauccauaacu

SEQ ID NO: 238 - RNA, 21 nt, Artificial Sequence
  source: /note=“Description of Artificial Sequence Synthetic oligonucleotide”
uacucaaagcaugggauuauu

SEQ ID NO: 239 - RNA, 21 nt, Artificial Sequence
  source: /note=“Description of Artificial Sequence Synthetic oligonucleotide”
uuaacucccauuaccauguaa

SEQ ID NO: 240 - RNA, 21 nt, Artificial Sequence
  source: /note=“Description of Artificial Sequence Synthetic oligonucleotide”
uaacucccauuaccauguaau

SEQ ID NO: 241 - RNA, 21 nt, Artificial Sequence
  source: /note=“Description of Artificial Sequence Synthetic oligonucleotide”
aaagcgugagcuuaaaauaca

SEQ ID NO: 242 - RNA, 21 nt, Artificial Sequence
  source: /note=“Description of Artificial Sequence Synthetic oligonucleotide”
uuguugacauuagucucagua

SEQ ID NO: 243 - RNA, 21 nt, Artificial Sequence
  source: /note=“Description of Artificial Sequence Synthetic oligonucleotide”
uugucuacacacccugcauau

SEQ ID NO: 244 - RNA, 21 nt, Artificial Sequence
  source: /note=“Description of Artificial Sequence Synthetic oligonucleotide”
agcccacuuacauuuacaaac

SEQ ID NO: 245 - RNA, 21 nt, Artificial Sequence
  source: /note=“Description of Artificial Sequence Synthetic oligonucleotide”
uucuaaccuuuacaugaggaa

SEQ ID NO: 246 - RNA, 21 nt, Artificial Sequence
  source: /note=“Description of Artificial Sequence Synthetic oligonucleotide”
accuuuacaugaggacucuau

SEQ ID NO: 247 - RNA, 21 nt, Artificial Sequence
  source: /note=“Description of Artificial Sequence Synthetic oligonucleotide”
uuuaacucccauuaccaugua

SEQ ID NO: 248 - RNA, 21 nt, Artificial Sequence
  source: /note=“Description of Artificial Sequence Synthetic oligonucleotide”
ugcucaggaaacgagacuuua

SEQ ID NO: 249 - RNA, 21 nt, Artificial Sequence
  source: /note=“Description of Artificial Sequence Synthetic oligonucleotide”
auaguuaacacacuaccgaau

SEQ ID NO: 250 - RNA, 21 nt, Artificial Sequence
  source: /note=“Description of Artificial Sequence Synthetic oligonucleotide”
ugaugcagcuuauacgaaaua

SEQ ID NO: 251 - RNA, 21 nt, Artificial Sequence
  source: /note=“Description of Artificial Sequence Synthetic oligonucleotide”
ucuguccuugaaggacuaaua

SEQ ID NO: 252 - RNA, 21 nt, Artificial Sequence
  source: /note=“Description of Artificial Sequence Synthetic oligonucleotide”
gaaacgagacuuuccauuaca

SEQ ID NO: 253 - RNA, 21 nt, Artificial Sequence
  source: /note=“Description of Artificial Sequence Synthetic oligonucleotide”
guucuuguaauuuuacacgcu

SEQ ID NO: 254 - RNA, 21 nt, Artificial Sequence
  source: /note=“Description of Artificial Sequence Synthetic oligonucleotide”
uccuuucauauaguuagcuaa

SEQ ID NO: 255 - RNA, 21 nt, Artificial Sequence
  source: /note=“Description of Artificial Sequence Synthetic oligonucleotide”
aaagucuguccuugaaggacu

SEQ ID NO: 256 - RNA, 21 nt, Artificial Sequence
  source: /note=“Description of Artificial Sequence Synthetic oligonucleotide”
guccuugaaggacuaauagaa

SEQ ID NO: 257 - RNA, 21 nt, Artificial Sequence
  source: /note=“Description of Artificial Sequence Synthetic oligonucleotide”
cguucuuguaauuuuacacga

SEQ ID NO: 258 - RNA, 21 nt, Artificial Sequence
  source: /note=“Description of Artificial Sequence Synthetic oligonucleotide”
uguauaguuaacacacuacca

SEQ ID NO: 259 - RNA, 21 nt, Artificial Sequence
  source: /note=“Description of Artificial Sequence Synthetic oligonucleotide”
aggacucuauucuuuaacuca

SEQ ID NO: 260 - RNA, 21 nt, Artificial Sequence
  source: /note=“Description of Artificial Sequence Synthetic oligonucleotide”
uguaauuuuacacgcuuuugu

SEQ ID NO: 261 - RNA, 21 nt, Artificial Sequence
  source: /note=“Description of Artificial Sequence Synthetic oligonucleotide”
aguccuggaugauacuaauaa

SEQ ID NO: 262 - RNA, 21 nt, Artificial Sequence
  source: /note=“Description of Artificial Sequence Synthetic oligonucleotide”
uauaguuaacacacuaccgaa

SEQ ID NO: 263 - RNA, 21 nt, Artificial Sequence
  source: /note=“Description of Artificial Sequence Synthetic oligonucleotide”
aaaaucuugaucaguuaagaa

SEQ ID NO: 264 - RNA, 21 nt, Artificial Sequence
  source: /note=“Description of Artificial Sequence Synthetic oligonucleotide”
acucaaagcaugggauuauua

SEQ ID NO: 265 - RNA, 21 nt, Artificial Sequence
  source: /note=“Description of Artificial Sequence Synthetic oligonucleotide”
ucccauuaccauguaauggca

SEQ ID NO: 266 - RNA, 21 nt, Artificial Sequence
  source: /note=“Description of Artificial Sequence Synthetic oligonucleotide”
uuuguugacauuagucucagu

SEQ ID NO: 267 - RNA, 21 nt, Artificial Sequence
  source: /note=“Description of Artificial Sequence Synthetic oligonucleotide”
gaguccuggaugauacuaaua

SEQ ID NO: 268 - RNA, 21 nt, Artificial Sequence
  source: /note=“Description of Artificial Sequence Synthetic oligonucleotide”
uuuuuaaacuguacagugucu

SEQ ID NO: 269 - RNA, 21 nt, Artificial Sequence
  source: /note=“Description of Artificial Sequence Synthetic oligonucleotide”
uuguauaguuaacacacuaca

SEQ ID NO: 270 - RNA, 21 nt, Artificial Sequence
  source: /note=“Description of Artificial Sequence Synthetic oligonucleotide”
uuaacacacuaccgaaugugu

SEQ ID NO: 271 - RNA, 21 nt, Artificial Sequence
  source: /note=“Description of Artificial Sequence Synthetic oligonucleotide”
guuaaguugagauaguuuuca

SEQ ID NO: 272 - RNA, 21 nt, Artificial Sequence
  source: /note=“Description of Artificial Sequence Synthetic oligonucleotide”
ugaaaaugcuggcuauagaua

SEQ ID NO: 273 - RNA, 21 nt, Artificial Sequence
  source: /note=“Description of Artificial Sequence Synthetic oligonucleotide”
aggaggaauacugaacaucua

SEQ ID NO: 274 - RNA, 21 nt, Artificial Sequence
  source: /note=“Description of Artificial Sequence Synthetic oligonucleotide”
uuuguauaguuaacacacuaa

SEQ ID NO: 275 - RNA, 21 nt, Artificial Sequence
  source: /note=“Description of Artificial Sequence Synthetic oligonucleotide”
aaguauguucuaaccuuuaca

SEQ ID NO: 276 - RNA, 21 nt, Artificial Sequence
  source: /note=“Description of Artificial Sequence Synthetic oligonucleotide”
uaauuaauugggccagcuuuu

SEQ ID NO: 277 - RNA, 21 nt, Artificial Sequence
  source: /note=“Description of Artificial Sequence Synthetic oligonucleotide”
caaagcaugggauuauuagaa

SEQ ID NO: 278 - RNA, 21 nt, Artificial Sequence
  source: /note=“Description of Artificial Sequence Synthetic oligonucleotide”
cuguccuugaaggacuaauaa

SEQ ID NO: 279 - RNA, 21 nt, Artificial Sequence
  source: /note=“Description of Artificial Sequence Synthetic oligonucleotide”
uaccauguaauggcaguuaua

SEQ ID NO: 280 - RNA, 21 nt, Artificial Sequence
  source: /note=“Description of Artificial Sequence Synthetic oligonucleotide”
uaaauauggacugcucaggaa

SEQ ID NO: 281 - RNA, 21 nt, Artificial Sequence
  source: /note=“Description of Artificial Sequence Synthetic oligonucleotide”
guauaguuaacacacuaccga

SEQ ID NO: 282 - RNA, 21 nt, Artificial Sequence
  source: /note=“Description of Artificial Sequence Synthetic oligonucleotide”
uuuacaugaggacucuauucu

SEQ ID NO: 283 - RNA, 21 nt, Artificial Sequence
  source: /note=“Description of Artificial Sequence Synthetic oligonucleotide”
acucccauuaccauguaauga

SEQ ID NO: 284 - RNA, 21 nt, Artificial Sequence
  source: /note=“Description of Artificial Sequence Synthetic oligonucleotide”
aaaaagcuuuugucuacacaa

SEQ ID NO: 285 - RNA, 21 nt, Artificial Sequence
  source: /note=“Description of Artificial Sequence Synthetic oligonucleotide”
auaguuuucauccauaacuga

SEQ ID NO: 286 - RNA, 21 nt, Artificial Sequence
  source: /note=“Description of Artificial Sequence Synthetic oligonucleotide”
ugggauuauuagaaucaaaca

SEQ ID NO: 287 - RNA, 21 nt, Artificial Sequence
  source: /note=“Description of Artificial Sequence Synthetic oligonucleotide”
gggaagcuagucuuuugcuuu

SEQ ID NO: 288 - RNA, 21 nt, Artificial Sequence
  source: /note=“Description of Artificial Sequence Synthetic oligonucleotide”
aaguugagauaguuuucauca

SEQ ID NO: 289 - RNA, 21 nt, Artificial Sequence
  source: /note=“Description of Artificial Sequence Synthetic oligonucleotide”
uaagaaauuucacauagccca

SEQ ID NO: 290 - RNA, 21 nt, Artificial Sequence
  source: /note=“Description of Artificial Sequence Synthetic oligonucleotide”
uuuaauuaauugggccagcuu

SEQ ID NO: 291 - RNA, 21 nt, Artificial Sequence
  source: /note=“Description of Artificial Sequence Synthetic oligonucleotide”
uuuucgagauuaucguuuucu

SEQ ID NO: 292 - RNA, 21 nt, Artificial Sequence
  source: /note=“Description of Artificial Sequence Synthetic oligonucleotide”
aaucuacucaaagcaugggau

SEQ ID NO: 293 - RNA, 21 nt, Artificial Sequence
  source: /note=“Description of Artificial Sequence Synthetic oligonucleotide”
auggacugcucaggaaacgaa

SEQ ID NO: 294 - RNA, 21 nt, Artificial Sequence
  source: /note=“Description of Artificial Sequence Synthetic oligonucleotide”
aucugaguccuggaugauacu

SEQ ID NO: 295 - RNA, 21 nt, Artificial Sequence
  source: /note=“Description of Artificial Sequence Synthetic oligonucleotide”
ucucugaugcagcuuauacga

SEQ ID NO: 296 - RNA, 21 nt, Artificial Sequence
  source: /note=“Description of Artificial Sequence Synthetic oligonucleotide”
uaauggcaguuauauuuugca

SEQ ID NO: 297 - RNA, 21 nt, Artificial Sequence
  source: /note=“Description of Artificial Sequence Synthetic oligonucleotide”
ggacugcucaggaaacgagaa

SEQ ID NO: 298 - RNA, 21 nt, Artificial Sequence
  source: /note=“Description of Artificial Sequence Synthetic oligonucleotide”
gugcaaacuugucgggaggaa

SEQ ID NO: 299 - RNA, 23 nt, Artificial Sequence
  source: /note=“Description of Artificial Sequence Synthetic oligonucleotide”
uagaacaacaauuauuucguaua

SEQ ID NO: 300 - RNA, 23 nt, Artificial Sequence
  source: /note=“Description of Artificial Sequence Synthetic oligonucleotide”
uagcuaacuauaugaaaggauaa

SEQ ID NO: 301 - RNA, 23 nt, Artificial Sequence
  source: /note=“Description of Artificial Sequence Synthetic oligonucleotide”
uaaauguaagugggcuaugugaa

SEQ ID NO: 302 - RNA, 23 nt, Artificial Sequence
  source: /note=“Description of Artificial Sequence Synthetic oligonucleotide”
uuuauuaguaucauccaggacuc

SEQ ID NO: 303 - RNA, 23 nt, Artificial Sequence
  source: /note=“Description of Artificial Sequence Synthetic oligonucleotide”
aauguaagugggcuaugugaaau

SEQ ID NO: 304 - RNA, 23 nt, Artificial Sequence
  source: /note=“Description of Artificial Sequence Synthetic oligonucleotide”
uguaaauguaagugggcuaugug

SEQ ID NO: 305 - RNA, 23 nt, Artificial Sequence
  source: /note=“Description of Artificial Sequence Synthetic oligonucleotide”
aaagaauagaguccucauguaaa

SEQ ID NO: 306 - RNA, 23 nt, Artificial Sequence
  source: /note=“Description of Artificial Sequence Synthetic oligonucleotide”
aguuaaagaauagaguccucaug

SEQ ID NO: 307 - RNA, 23 nt, Artificial Sequence
  source: /note=“Description of Artificial Sequence Synthetic oligonucleotide”
uuguauuuuaagcucacgcuuuu

SEQ ID NO: 308 - RNA, 23 nt, Artificial Sequence
  source: /note=“Description of Artificial Sequence Synthetic oligonucleotide”
aaaacgauaaucucgaaaaccac

SEQ ID NO: 309 - RNA, 23 nt, Artificial Sequence
  source: /note=“Description of Artificial Sequence Synthetic oligonucleotide”
aagaacgcuauuuuaaaauacug

SEQ ID NO: 310 - RNA, 23 nt, Artificial Sequence
  source: /note=“Description of Artificial Sequence Synthetic oligonucleotide”
uaccuaugaaaaggacaacaaua

SEQ ID NO: 311 - RNA, 23 nt, Artificial Sequence
  source: /note=“Description of Artificial Sequence Synthetic oligonucleotide”
uuaauggaaagucucguuuccug

SEQ ID NO: 312 - RNA, 23 nt, Artificial Sequence
  source: /note=“Description of Artificial Sequence Synthetic oligonucleotide”
uaaaacgauaaucucgaaaacca

SEQ ID NO: 313 - RNA, 23 nt, Artificial Sequence
  source: /note=“Description of Artificial Sequence Synthetic oligonucleotide”
auauaacaaaacagcacuccauc

SEQ ID NO: 314 - RNA, 23 nt, Artificial Sequence
  source: /note=“Description of Artificial Sequence Synthetic oligonucleotide”
uuucguauaagcugcaucagaga

SEQ ID NO: 315 - RNA, 23 nt, Artificial Sequence
  source: /note=“Description of Artificial Sequence Synthetic oligonucleotide”
auuucguauaagcugcaucagag

SEQ ID NO: 316 - RNA, 23 nt, Artificial Sequence
  source: /note=“Description of Artificial Sequence Synthetic oligonucleotide”
uaguuauggaugaaaacuaucuc

SEQ ID NO: 317 - RNA, 23 nt, Artificial Sequence
  source: /note=“Description of Artificial Sequence Synthetic oligonucleotide”
aaauguaagugggcuaugugaaa

SEQ ID NO: 318 - RNA, 23 nt, Artificial Sequence
  source: /note=“Description of Artificial Sequence Synthetic oligonucleotide”
aagaauagaguccucauguaaag

SEQ ID NO: 319 - RNA, 23 nt, Artificial Sequence
  source: /note=“Description of Artificial Sequence Synthetic oligonucleotide”
ugaaaagacaucuauagccagca

SEQ ID NO: 320 - RNA, 23 nt, Artificial Sequence
  source: /note=“Description of Artificial Sequence Synthetic oligonucleotide”
aagaaaacgauaaucucgaaaac

SEQ ID NO: 321 - RNA, 23 nt, Artificial Sequence
  source: /note=“Description of Artificial Sequence Synthetic oligonucleotide”
aagcguguaaaauuacaagaacg

SEQ ID NO: 322 - RNA, 23 nt, Artificial Sequence
  source: /note=“Description of Artificial Sequence Synthetic oligonucleotide”
acauucgguaguguguuaacuau

SEQ ID NO: 323 - RNA, 23 nt, Artificial Sequence
  source: /note=“Description of Artificial Sequence Synthetic oligonucleotide”
aaaacuaucucaacuuaacuuua

SEQ ID NO: 324 - RNA, 23 nt, Artificial Sequence
  source: /note=“Description of Artificial Sequence Synthetic oligonucleotide”
uauucuaauaaucccaugcuuug

SEQ ID NO: 325 - RNA, 23 nt, Artificial Sequence
  source: /note=“Description of Artificial Sequence Synthetic oligonucleotide”
aaugggaguuaaagaauagaguc

SEQ ID NO: 326 - RNA, 23 nt, Artificial Sequence
  source: /note=“Description of Artificial Sequence Synthetic oligonucleotide”
aaagcguguaaaauuacaagaac

SEQ ID NO: 327 - RNA, 23 nt, Artificial Sequence
  source: /note=“Description of Artificial Sequence Synthetic oligonucleotide”
uauucgguaguguguuaacuaua

SEQ ID NO: 328 - RNA, 23 nt, Artificial Sequence
  source: /note=“Description of Artificial Sequence Synthetic oligonucleotide”
uacauucgguaguguguuaacua

SEQ ID NO: 329 - RNA, 23 nt, Artificial Sequence
  source: /note=“Description of Artificial Sequence Synthetic oligonucleotide”
uucguauaagcugcaucagagac

SEQ ID NO: 330 - RNA, 23 nt, Artificial Sequence
  source: /note=“Description of Artificial Sequence Synthetic oligonucleotide”
uaugcaggguguguagacaaaag

SEQ ID NO: 331 - RNA, 23 nt, Artificial Sequence
  source: /note=“Description of Artificial Sequence Synthetic oligonucleotide”
aguuauggaugaaaacuaucuca

SEQ ID NO: 332 - RNA, 23 nt, Artificial Sequence
  source: /note=“Description of Artificial Sequence Synthetic oligonucleotide”
aauaaucccaugcuuugaguaga

SEQ ID NO: 333 - RNA, 23 nt, Artificial Sequence
  source: /note=“Description of Artificial Sequence Synthetic oligonucleotide”
uuacaugguaaugggaguuaaag

SEQ ID NO: 334 - RNA, 23 nt, Artificial Sequence
  source: /note=“Description of Artificial Sequence Synthetic oligonucleotide”
auuacaugguaaugggaguuaaa

SEQ ID NO: 335 - RNA, 23 nt, Artificial Sequence
  source: /note=“Description of Artificial Sequence Synthetic oligonucleotide”
uguauuuuaagcucacgcuuuug

SEQ ID NO: 336 - RNA, 23 nt, Artificial Sequence
  source: /note=“Description of Artificial Sequence Synthetic oligonucleotide”
uacugagacuaaugucaacaaaa

SEQ ID NO: 337 - RNA, 23 nt, Artificial Sequence
  source: /note=“Description of Artificial Sequence Synthetic oligonucleotide”
auaugcaggguguguagacaaaa

SEQ ID NO: 338 - RNA, 23 nt, Artificial Sequence
  source: /note=“Description of Artificial Sequence Synthetic oligonucleotide”
guuuguaaauguaagugggcuau

SEQ ID NO: 339 - RNA, 23 nt, Artificial Sequence
  source: /note=“Description of Artificial Sequence Synthetic oligonucleotide”
uuccucauguaaagguuagaaca

SEQ ID NO: 340 - RNA, 23 nt, Artificial Sequence
  source: /note=“Description of Artificial Sequence Synthetic oligonucleotide”
auagaguccucauguaaagguua

SEQ ID NO: 341 - RNA, 23 nt, Artificial Sequence
  source: /note=“Description of Artificial Sequence Synthetic oligonucleotide”
uacaugguaaugggaguuaaaga

SEQ ID NO: 342 - RNA, 23 nt, Artificial Sequence
  source: /note=“Description of Artificial Sequence Synthetic oligonucleotide”
uaaagucucguuuccugagcagu

SEQ ID NO: 343 - RNA, 23 nt, Artificial Sequence
  source: /note=“Description of Artificial Sequence Synthetic oligonucleotide”
auucgguaguguguuaacuauac

SEQ ID NO: 344 - RNA, 23 nt, Artificial Sequence
  source: /note=“Description of Artificial Sequence Synthetic oligonucleotide”
uauuucguauaagcugcaucaga

SEQ ID NO: 345 - RNA, 23 nt, Artificial Sequence
  source: /note=“Description of Artificial Sequence Synthetic oligonucleotide”
uauuaguccuucaaggacagacu

SEQ ID NO: 346 - RNA, 23 nt, Artificial Sequence
  source: /note=“Description of Artificial Sequence Synthetic oligonucleotide”
uguaauggaaagucucguuuccu

SEQ ID NO: 347 - RNA, 23 nt, Artificial Sequence
  source: /note=“Description of Artificial Sequence Synthetic oligonucleotide”
agcguguaaaauuacaagaacgc

SEQ ID NO: 348 - RNA, 23 nt, Artificial Sequence
  source: /note=“Description of Artificial Sequence Synthetic oligonucleotide”
uuagcuaacuauaugaaaggaua

SEQ ID NO: 349 - RNA, 23 nt, Artificial Sequence
  source: /note=“Description of Artificial Sequence Synthetic oligonucleotide”
aguccuucaaggacagacuuuca

SEQ ID NO: 350 - RNA, 23 nt, Artificial Sequence
  source: /note=“Description of Artificial Sequence Synthetic oligonucleotide”
uucuauuaguccuucaaggacag

SEQ ID NO: 351 - RNA, 23 nt, Artificial Sequence
  source: /note=“Description of Artificial Sequence Synthetic oligonucleotide”
ucguguaaaauuacaagaacgcu

SEQ ID NO: 352 - RNA, 23 nt, Artificial Sequence
  source: /note=“Description of Artificial Sequence Synthetic oligonucleotide”
ugguaguguguuaacuauacaaa

SEQ ID NO: 353 - RNA, 23 nt, Artificial Sequence
  source: /note=“Description of Artificial Sequence Synthetic oligonucleotide”
ugaguuaaagaauagaguccuca

SEQ ID NO: 354 - RNA, 23 nt, Artificial Sequence
  source: /note=“Description of Artificial Sequence Synthetic oligonucleotide”
acaaaagcguguaaaauuacaag

SEQ ID NO: 355 - RNA, 23 nt, Artificial Sequence
  source: /note=“Description of Artificial Sequence Synthetic oligonucleotide”
uuauuaguaucauccaggacuca

SEQ ID NO: 356 - RNA, 23 nt, Artificial Sequence
  source: /note=“Description of Artificial Sequence Synthetic oligonucleotide”
uucgguaguguguuaacuauaca

SEQ ID NO: 357 - RNA, 23 nt, Artificial Sequence
  source: /note=“Description of Artificial Sequence Synthetic oligonucleotide”
uucuuaacugaucaagauuuugg

SEQ ID NO: 358 - RNA, 23 nt, Artificial Sequence
  source: /note=“Description of Artificial Sequence Synthetic oligonucleotide”
uaauaaucccaugcuuugaguag

SEQ ID NO: 359 - RNA, 23 nt, Artificial Sequence
  source: /note=“Description of Artificial Sequence Synthetic oligonucleotide”
ugccauuacaugguaaugggagu

SEQ ID NO: 360 - RNA, 23 nt, Artificial Sequence
  source: /note=“Description of Artificial Sequence Synthetic oligonucleotide”
acugagacuaaugucaacaaaaa

SEQ ID NO: 361 - RNA, 23 nt, Artificial Sequence
  source: /note=“Description of Artificial Sequence Synthetic oligonucleotide”
uauuaguaucauccaggacucag

SEQ ID NO: 362 - RNA, 23 nt, Artificial Sequence
  source: /note=“Description of Artificial Sequence Synthetic oligonucleotide”
agacacuguacaguuuaaaaaca

SEQ ID NO: 363 - RNA, 23 nt, Artificial Sequence
  source: /note=“Description of Artificial Sequence Synthetic oligonucleotide”
uguaguguguuaacuauacaaaa

SEQ ID NO: 364 - RNA, 23 nt, Artificial Sequence
  source: /note=“Description of Artificial Sequence Synthetic oligonucleotide”
acacauucgguaguguguuaacu

SEQ ID NO: 365 - RNA, 23 nt, Artificial Sequence
  source: /note=“Description of Artificial Sequence Synthetic oligonucleotide”
ugaaaacuaucucaacuuaacuu

SEQ ID NO: 366 - RNA, 23 nt, Artificial Sequence
  source: /note=“Description of Artificial Sequence Synthetic oligonucleotide”
uaucuauagccagcauuuucaua

SEQ ID NO: 367 - RNA, 23 nt, Artificial Sequence
  source: /note=“Description of Artificial Sequence Synthetic oligonucleotide”
uagauguucaguauuccuccuga

SEQ ID NO: 368 - RNA, 23 nt, Artificial Sequence
  source: /note=“Description of Artificial Sequence Synthetic oligonucleotide”
uuaguguguuaacuauacaaaaa

SEQ ID NO: 369 - RNA, 23 nt, Artificial Sequence
  source: /note=“Description of Artificial Sequence Synthetic oligonucleotide”
uguaaagguuagaacauacuuuu

SEQ ID NO: 370 - RNA, 23 nt, Artificial Sequence
  source: /note=“Description of Artificial Sequence Synthetic oligonucleotide”
aaaagcuggcccaauuaauuaaa

SEQ ID NO: 371 - RNA, 23 nt, Artificial Sequence
  source: /note=“Description of Artificial Sequence Synthetic oligonucleotide”
uucuaauaaucccaugcuuugag

SEQ ID NO: 372 - RNA, 23 nt, Artificial Sequence
  source: /note=“Description of Artificial Sequence Synthetic oligonucleotide”
uuauuaguccuucaaggacagac

SEQ ID NO: 373 - RNA, 23 nt, Artificial Sequence
  source: /note=“Description of Artificial Sequence Synthetic oligonucleotide”
uauaacugccauuacaugguaau

SEQ ID NO: 374 - RNA, 23 nt, Artificial Sequence
  source: /note=“Description of Artificial Sequence Synthetic oligonucleotide”
uuccugagcaguccauauuuaga

SEQ ID NO: 375 - RNA, 23 nt, Artificial Sequence
  source: /note=“Description of Artificial Sequence Synthetic oligonucleotide”
ucgguaguguguuaacuauacaa

SEQ ID NO: 376 - RNA, 23 nt, Artificial Sequence
  source: /note=“Description of Artificial Sequence Synthetic oligonucleotide”
agaauagaguccucauguaaagg

SEQ ID NO: 377 - RNA, 23 nt, Artificial Sequence
  source: /note=“Description of Artificial Sequence Synthetic oligonucleotide”
ucauuacaugguaaugggaguua

SEQ ID NO: 378 - RNA, 23 nt, Artificial Sequence
  source: /note=“Description of Artificial Sequence Synthetic oligonucleotide”
uuguguagacaaaagcuuuuuau

SEQ ID NO: 379 - RNA, 23 nt, Artificial Sequence
  source: /note=“Description of Artificial Sequence Synthetic oligonucleotide”
ucaguuauggaugaaaacuaucu

SEQ ID NO: 380 - RNA, 23 nt, Artificial Sequence
  source: /note=“Description of Artificial Sequence Synthetic oligonucleotide”
uguuugauucuaauaaucccaug

SEQ ID NO: 381 - RNA, 23 nt, Artificial Sequence
  source: /note=“Description of Artificial Sequence Synthetic oligonucleotide”
aaagcaaaagacuagcuuccccu

SEQ ID NO: 382 - RNA, 23 nt, Artificial Sequence
  source: /note=“Description of Artificial Sequence Synthetic oligonucleotide”
ugaugaaaacuaucucaacuuaa

SEQ ID NO: 383 - RNA, 23 nt, Artificial Sequence
  source: /note=“Description of Artificial Sequence Synthetic oligonucleotide”
ugggcuaugugaaauuucuuaac

SEQ ID NO: 384 - RNA, 23 nt, Artificial Sequence
  source: /note=“Description of Artificial Sequence Synthetic oligonucleotide”
aagcuggcccaauuaauuaaaaa

SEQ ID NO: 385 - RNA, 23 nt, Artificial Sequence
  source: /note=“Description of Artificial Sequence Synthetic oligonucleotide”
agaaaacgauaaucucgaaaacc

SEQ ID NO: 386 - RNA, 23 nt, Artificial Sequence
  source: /note=“Description of Artificial Sequence Synthetic oligonucleotide”
aucccaugcuuugaguagauuga

SEQ ID NO: 387 - RNA, 23 nt, Artificial Sequence
  source: /note=“Description of Artificial Sequence Synthetic oligonucleotide”
uucguuuccugagcaguccauau

SEQ ID NO: 388 - RNA, 23 nt, Artificial Sequence
  source: /note=“Description of Artificial Sequence Synthetic oligonucleotide”
aguaucauccaggacucagaugu

SEQ ID NO: 389 - RNA, 23 nt, Artificial Sequence
  source: /note=“Description of Artificial Sequence Synthetic oligonucleotide”
ucguauaagcugcaucagagaca

SEQ ID NO: 390 - RNA, 23 nt, Artificial Sequence
  source: /note=“Description of Artificial Sequence Synthetic oligonucleotide”
ugcaaaauauaacugccauuaca

SEQ ID NO: 391 - RNA, 23 nt, Artificial Sequence
  source: /note=“Description of Artificial Sequence Synthetic oligonucleotide”
uucucguuuccugagcaguccau

SEQ ID NO: 392 - RNA, 23 nt, Artificial Sequence
  source: /note=“Description of Artificial Sequence Synthetic oligonucleotide”
uuccucccgacaaguuugcacaa

SEQ ID NO: 393 - RNA, 23 nt, Artificial Sequence
  source: /note=“Description of Artificial Sequence Synthetic oligonucleotide”
uauacgaaauaauuguuguucug

SEQ ID NO: 394 - RNA, 23 nt, Artificial Sequence
  source: /note=“Description of Artificial Sequence Synthetic oligonucleotide”
uuauccuuucauauaguuagcua

SEQ ID NO: 395 - RNA, 23 nt, Artificial Sequence
  source: /note=“Description of Artificial Sequence Synthetic oligonucleotide”
uucacauagcccacuuacauuua

SEQ ID NO: 396 - RNA, 23 nt, Artificial Sequence
  source: /note=“Description of Artificial Sequence Synthetic oligonucleotide”
gaguccuggaugauacuaauaaa

SEQ ID NO: 397 - RNA, 23 nt, Artificial Sequence
  source: /note=“Description of Artificial Sequence Synthetic oligonucleotide”
auuucacauagcccacuuacauu

SEQ ID NO: 398 - RNA, 23 nt, Artificial Sequence
  source: /note=“Description of Artificial Sequence Synthetic oligonucleotide”
cacauagcccacuuacauuuaca

SEQ ID NO: 399 - RNA, 23 nt, Artificial Sequence
  source: /note=“Description of Artificial Sequence Synthetic oligonucleotide”
uuuacaugaggacucuauucuuu

SEQ ID NO: 400 - RNA, 23 nt, Artificial Sequence
  source: /note=“Description of Artificial Sequence Synthetic oligonucleotide”
caugaggacucuauucuuuaacu

SEQ ID NO: 401 - RNA, 23 nt, Artificial Sequence
  source: /note=“Description of Artificial Sequence Synthetic oligonucleotide”
aaaagcgugagcuuaaaauacaa

SEQ ID NO: 402 - RNA, 23 nt, Artificial Sequence
  source: /note=“Description of Artificial Sequence Synthetic oligonucleotide”
gugguuuucgagauuaucguuuu

SEQ ID NO: 403 - RNA, 23 nt, Artificial Sequence
  source: /note=“Description of Artificial Sequence Synthetic oligonucleotide”
caguauuuuaaaauagcguucuu

SEQ ID NO: 404 - RNA, 23 nt, Artificial Sequence
  source: /note=“Description of Artificial Sequence Synthetic oligonucleotide”
uauuguuguccuuuucauagguc

SEQ ID NO: 405 - RNA, 23 nt, Artificial Sequence
  source: /note=“Description of Artificial Sequence Synthetic oligonucleotide”
caggaaacgagacuuuccauuac

SEQ ID NO: 406 - RNA, 23 nt, Artificial Sequence
  source: /note=“Description of Artificial Sequence Synthetic oligonucleotide”
ugguuuucgagauuaucguuuuc

SEQ ID NO: 407 - RNA, 23 nt, Artificial Sequence
  source: /note=“Description of Artificial Sequence Synthetic oligonucleotide”
gauggagugcuguuuuguuauau

SEQ ID NO: 408 - RNA, 23 nt, Artificial Sequence
  source: /note=“Description of Artificial Sequence Synthetic oligonucleotide”
ucucugaugcagcuuauacgaaa

SEQ ID NO: 409 - RNA, 23 nt, Artificial Sequence
  source: /note=“Description of Artificial Sequence Synthetic oligonucleotide”
cucugaugcagcuuauacgaaau

SEQ ID NO: 410 - RNA, 23 nt, Artificial Sequence
  source: /note=“Description of Artificial Sequence Synthetic oligonucleotide”
gagauaguuuucauccauaacug

SEQ ID NO: 411 - RNA, 23 nt, Artificial Sequence
  source: /note=“Description of Artificial Sequence Synthetic oligonucleotide”
uuucacauagcccacuuacauuu

SEQ ID NO: 412 - RNA, 23 nt, Artificial Sequence
  source: /note=“Description of Artificial Sequence Synthetic oligonucleotide”
cuuuacaugaggacucuauucuu

SEQ ID NO: 413 - RNA, 23 nt, Artificial Sequence
  source: /note=“Description of Artificial Sequence Synthetic oligonucleotide”
ugcuggcuauagaugucuuuucc

SEQ ID NO: 414 - RNA, 23 nt, Artificial Sequence
  source: /note=“Description of Artificial Sequence Synthetic oligonucleotide”
guuuucgagauuaucguuuucuu

SEQ ID NO: 415 - RNA, 23 nt, Artificial Sequence
  source: /note=“Description of Artificial Sequence Synthetic oligonucleotide”
cguucuuguaauuuuacacgcuu

SEQ ID NO: 416 - RNA, 23 nt, Artificial Sequence
  source: /note=“Description of Artificial Sequence Synthetic oligonucleotide”
auaguuaacacacuaccgaaugu

SEQ ID NO: 417 - RNA, 23 nt, Artificial Sequence
  source: /note=“Description of Artificial Sequence Synthetic oligonucleotide”
uaaaguuaaguugagauaguuuu

SEQ ID NO: 418 - RNA, 23 nt, Artificial Sequence
  source: /note=“Description of Artificial Sequence Synthetic oligonucleotide”
caaagcaugggauuauuagaauc

SEQ ID NO: 419 - RNA, 23 nt, Artificial Sequence
  source: /note=“Description of Artificial Sequence Synthetic oligonucleotide”
gacucuauucuuuaacucccauu

SEQ ID NO: 420 - RNA, 23 nt, Artificial Sequence
  source: /note=“Description of Artificial Sequence Synthetic oligonucleotide”
guucuuguaauuuuacacgcuuu

SEQ ID NO: 421 - RNA, 23 nt, Artificial Sequence
  source: /note=“Description of Artificial Sequence Synthetic oligonucleotide”
uauaguuaacacacuaccgaaug

SEQ ID NO: 422 - RNA, 23 nt, Artificial Sequence
  source: /note=“Description of Artificial Sequence Synthetic oligonucleotide”
uaguuaacacacuaccgaaugug

SEQ ID NO: 423 - RNA, 23 nt, Artificial Sequence
  source: /note=“Description of Artificial Sequence Synthetic oligonucleotide”
gucucugaugcagcuuauacgaa

SEQ ID NO: 424 - RNA, 23 nt, Artificial Sequence
  source: /note=“Description of Artificial Sequence Synthetic oligonucleotide”
cuuuugucuacacacccugcaua

SEQ ID NO: 425 - RNA, 23 nt, Artificial Sequence
  source: /note=“Description of Artificial Sequence Synthetic oligonucleotide”
ugagauaguuuucauccauaacu

SEQ ID NO: 426 - RNA, 23 nt, Artificial Sequence
  source: /note=“Description of Artificial Sequence Synthetic oligonucleotide”
ucuacucaaagcaugggauuauu

SEQ ID NO: 427 - RNA, 23 nt, Artificial Sequence
  source: /note=“Description of Artificial Sequence Synthetic oligonucleotide”
cuuuaacucccauuaccauguaa

SEQ ID NO: 428 - RNA, 23 nt, Artificial Sequence
  source: /note=“Description of Artificial Sequence Synthetic oligonucleotide”
uuuaacucccauuaccauguaau

SEQ ID NO: 429 - RNA, 23 nt, Artificial Sequence
  source: /note=“Description of Artificial Sequence Synthetic oligonucleotide”
caaaagcgugagcuuaaaauaca

SEQ ID NO: 430 - RNA, 23 nt, Artificial Sequence
  source: /note=“Description of Artificial Sequence Synthetic oligonucleotide”
uuuuguugacauuagucucagug

SEQ ID NO: 431 - RNA, 23 nt, Artificial Sequence
  source: /note=“Description of Artificial Sequence Synthetic oligonucleotide”
uuuugucuacacacccugcauau

SEQ ID NO: 432 - RNA, 23 nt, Artificial Sequence
  source: /note=“Description of Artificial Sequence Synthetic oligonucleotide”
auagcccacuuacauuuacaaac

SEQ ID NO: 433 - RNA, 23 nt, Artificial Sequence
  source: /note=“Description of Artificial Sequence Synthetic oligonucleotide”
uguucuaaccuuuacaugaggac

SEQ ID NO: 434 - RNA, 23 nt, Artificial Sequence
  source: /note=“Description of Artificial Sequence Synthetic oligonucleotide”
uaaccuuuacaugaggacucuau

SEQ ID NO: 435 - RNA, 23 nt, Artificial Sequence
  source: /note=“Description of Artificial Sequence Synthetic oligonucleotide”
ucuuuaacucccauuaccaugua

SEQ ID NO: 436 - RNA, 23 nt, Artificial Sequence
  source: /note=“Description of Artificial Sequence Synthetic oligonucleotide”
acugcucaggaaacgagacuuuc

SEQ ID NO: 437 - RNA, 23 nt, Artificial Sequence
  source: /note=“Description of Artificial Sequence Synthetic oligonucleotide”
guauaguuaacacacuaccgaau

SEQ ID NO: 438 - RNA, 23 nt, Artificial Sequence
  source: /note=“Description of Artificial Sequence Synthetic oligonucleotide”
ucugaugcagcuuauacgaaaua

SEQ ID NO: 439 - RNA, 23 nt, Artificial Sequence
  source: /note=“Description of Artificial Sequence Synthetic oligonucleotide”
agucuguccuugaaggacuaaua

SEQ ID NO: 440 - RNA, 23 nt, Artificial Sequence
  source: /note=“Description of Artificial Sequence Synthetic oligonucleotide”
aggaaacgagacuuuccauuaca

SEQ ID NO: 441 - RNA, 23 nt, Artificial Sequence
  source: /note=“Description of Artificial Sequence Synthetic oligonucleotide”
gcguucuuguaauuuuacacgcu

SEQ ID NO: 442 - RNA, 23 nt, Artificial Sequence
  source: /note=“Description of Artificial Sequence Synthetic oligonucleotide”
uauccuuucauauaguuagcuaa

SEQ ID NO: 443 - RNA, 23 nt, Artificial Sequence
  source: /note=“Description of Artificial Sequence Synthetic oligonucleotide”
ugaaagucuguccuugaaggacu

SEQ ID NO: 444 - RNA, 23 nt, Artificial Sequence
  source: /note=“Description of Artificial Sequence Synthetic oligonucleotide”
cuguccuugaaggacuaauagaa

SEQ ID NO: 445 - RNA, 23 nt, Artificial Sequence
  source: /note=“Description of Artificial Sequence Synthetic oligonucleotide”
agcguucuuguaauuuuacacgc

SEQ ID NO: 446 - RNA, 23 nt, Artificial Sequence
  source: /note=“Description of Artificial Sequence Synthetic oligonucleotide”
uuuguauaguuaacacacuaccg

SEQ ID NO: 447 - RNA, 23 nt, Artificial Sequence
  source: /note=“Description of Artificial Sequence Synthetic oligonucleotide”
ugaggacucuauucuuuaacucc

SEQ ID NO: 448 - RNA, 23 nt, Artificial Sequence
  source: /note=“Description of Artificial Sequence Synthetic oligonucleotide”
cuuguaauuuuacacgcuuuugu

SEQ ID NO: 449 - RNA, 23 nt, Artificial Sequence
  source: /note=“Description of Artificial Sequence Synthetic oligonucleotide”
ugaguccuggaugauacuaauaa

SEQ ID NO: 450 - RNA, 23 nt, Artificial Sequence
  source: /note=“Description of Artificial Sequence Synthetic oligonucleotide”
uguauaguuaacacacuaccgaa

SEQ ID NO: 451 - RNA, 23 nt, Artificial Sequence
  source: /note=“Description of Artificial Sequence Synthetic oligonucleotide”
ccaaaaucuugaucaguuaagaa

SEQ ID NO: 452 - RNA, 23 nt, Artificial Sequence
  source: /note=“Description of Artificial Sequence Synthetic oligonucleotide”
cuacucaaagcaugggauuauua

SEQ ID NO: 453 - RNA, 23 nt, Artificial Sequence
  source: /note=“Description of Artificial Sequence Synthetic oligonucleotide”
acucccauuaccauguaauggca

SEQ ID NO: 454 - RNA, 23 nt, Artificial Sequence
  source: /note=“Description of Artificial Sequence Synthetic oligonucleotide”
uuuuuguugacauuagucucagu

SEQ ID NO: 455 - RNA, 23 nt, Artificial Sequence
  source: /note=“Description of Artificial Sequence Synthetic oligonucleotide”
cugaguccuggaugauacuaaua

SEQ ID NO: 456 - RNA, 23 nt, Artificial Sequence
  source: /note=“Description of Artificial Sequence Synthetic oligonucleotide”
uguuuuuaaacuguacagugucu

SEQ ID NO: 457 - RNA, 23 nt, Artificial Sequence
  source: /note=“Description of Artificial Sequence Synthetic oligonucleotide”
uuuuguauaguuaacacacuacc

SEQ ID NO: 458 - RNA, 23 nt, Artificial Sequence
  source: /note=“Description of Artificial Sequence Synthetic oligonucleotide”
aguuaacacacuaccgaaugugu

SEQ ID NO: 459 - RNA, 23 nt, Artificial Sequence
  source: /note=“Description of Artificial Sequence Synthetic oligonucleotide”
aaguuaaguugagauaguuuuca

SEQ ID NO: 460 - RNA, 23 nt, Artificial Sequence
  source: /note=“Description of Artificial Sequence Synthetic oligonucleotide”
uaugaaaaugcuggcuauagaug

SEQ ID NO: 461 - RNA, 23 nt, Artificial Sequence
  source: /note=“Description of Artificial Sequence Synthetic oligonucleotide”
ucaggaggaauacugaacaucug

SEQ ID NO: 462 - RNA, 23 nt, Artificial Sequence
  source: /note=“Description of Artificial Sequence Synthetic oligonucleotide”
uuuuuguauaguuaacacacuac

SEQ ID NO: 463 - RNA, 23 nt, Artificial Sequence
  source: /note=“Description of Artificial Sequence Synthetic oligonucleotide”
aaaaguauguucuaaccuuuaca

SEQ ID NO: 464 - RNA, 23 nt, Artificial Sequence
  source: /note=“Description of Artificial Sequence Synthetic oligonucleotide”
uuuaauuaauugggccagcuuuu

SEQ ID NO: 465 - RNA, 23 nt, Artificial Sequence
  source: /note=“Description of Artificial Sequence Synthetic oligonucleotide”
cucaaagcaugggauuauuagaa

SEQ ID NO: 466 - RNA, 23 nt, Artificial Sequence
  source: /note=“Description of Artificial Sequence Synthetic oligonucleotide”
gucuguccuugaaggacuaauag

SEQ ID NO: 467 - RNA, 23 nt, Artificial Sequence
  source: /note=“Description of Artificial Sequence Synthetic oligonucleotide”
auuaccauguaauggcaguuaua

SEQ ID NO: 468 - RNA, 23 nt, Artificial Sequence
  source: /note=“Description of Artificial Sequence Synthetic oligonucleotide”
ucuaaauauggacugcucaggaa

SEQ ID NO: 469 - RNA, 23 nt, Artificial Sequence
  source: /note=“Description of Artificial Sequence Synthetic oligonucleotide”
uuguauaguuaacacacuaccga

SEQ ID NO: 470 - RNA, 23 nt, Artificial Sequence
  source: /note=“Description of Artificial Sequence Synthetic oligonucleotide”
ccuuuacaugaggacucuauucu

SEQ ID NO: 471 - RNA, 23 nt, Artificial Sequence
  source: /note=“Description of Artificial Sequence Synthetic oligonucleotide”
uaacucccauuaccauguaaugg

SEQ ID NO: 472 - RNA, 23 nt, Artificial Sequence
  source: /note=“Description of Artificial Sequence Synthetic oligonucleotide”
auaaaaagcuuuugucuacacac

SEQ ID NO: 473 - RNA, 23 nt, Artificial Sequence
  source: /note=“Description of Artificial Sequence Synthetic oligonucleotide”
agauaguuuucauccauaacuga

SEQ ID NO: 474 - RNA, 23 nt, Artificial Sequence
  source: /note=“Description of Artificial Sequence Synthetic oligonucleotide”
caugggauuauuagaaucaaaca

SEQ ID NO: 475 - RNA, 23 nt, Artificial Sequence
  source: /note=“Description of Artificial Sequence Synthetic oligonucleotide”
aggggaagcuagucuuuugcuuu

SEQ ID NO: 476 - RNA, 23 nt, Artificial Sequence
  source: /note=“Description of Artificial Sequence Synthetic oligonucleotide”
uuaaguugagauaguuuucaucc

SEQ ID NO: 477 - RNA, 23 nt, Artificial Sequence
  source: /note=“Description of Artificial Sequence Synthetic oligonucleotide”
guuaagaaauuucacauagccca

SEQ ID NO: 478 - RNA, 23 nt, Artificial Sequence
  source: /note=“Description of Artificial Sequence Synthetic oligonucleotide”
uuuuuaauuaauugggccagcuu

SEQ ID NO: 479 - RNA, 23 nt, Artificial Sequence
  source: /note=“Description of Artificial Sequence Synthetic oligonucleotide”
gguuuucgagauuaucguuuucu

SEQ ID NO: 480 - RNA, 23 nt, Artificial Sequence
  source: /note=“Description of Artificial Sequence Synthetic oligonucleotide”
ucaaucuacucaaagcaugggau

SEQ ID NO: 481 - RNA, 23 nt, Artificial Sequence
  source: /note=“Description of Artificial Sequence Synthetic oligonucleotide”
auauggacugcucaggaaacgag

SEQ ID NO: 482 - RNA, 23 nt, Artificial Sequence
  source: /note=“Description of Artificial Sequence Synthetic oligonucleotide”
acaucugaguccuggaugauacu

SEQ ID NO: 483 - RNA, 23 nt, Artificial Sequence
  source: /note=“Description of Artificial Sequence Synthetic oligonucleotide”
ugucucugaugcagcuuauacga

SEQ ID NO: 484 - RNA, 23 nt, Artificial Sequence
  source: /note=“Description of Artificial Sequence Synthetic oligonucleotide”
uguaauggcaguuauauuuugca

SEQ ID NO: 485 - RNA, 23 nt, Artificial Sequence
  source: /note=“Description of Artificial Sequence Synthetic oligonucleotide”
auggacugcucaggaaacgagac

SEQ ID NO: 486 - RNA, 23 nt, Artificial Sequence
  source: /note=“Description of Artificial Sequence Synthetic oligonucleotide”
uugugcaaacuugucgggaggag

SEQ ID NO: 487 - RNA, 21 nt, Artificial Sequence
  source: /note=“Description of Artificial Sequence Synthetic oligonucleotide”
guaagauuuguuuuuaaacua

SEQ ID NO: 488 - RNA, 23 nt, Artificial Sequence
  source: /note=“Description of Artificial Sequence Synthetic oligonucleotide”
uaguuuaaaaacaaaucuuacac

SEQ ID NO: 489 - RNA, 21 nt, Artificial Sequence
  source: /note=“Description of Artificial Sequence Synthetic oligonucleotide”
acgaagauaauuguuguucua

SEQ ID NO: 490 - RNA, 23 nt, Artificial Sequence
  source: /note=“Description of Artificial Sequence Synthetic oligonucleotide”
uagaacaacaauuaucuucguau

SEQ ID NO: 491 - RNA, 21 nt, Artificial Sequence
  source: /note=“Description of Artificial Sequence Synthetic oligonucleotide”
caucccuaaucuauacauaua

SEQ ID NO: 492 - RNA, 23 nt, Artificial Sequence
  source: /note=“Description of Artificial Sequence Synthetic oligonucleotide”
uauauguauagauuagggaugcu

SEQ ID NO: 493 - RNA, 21 nt, Artificial Sequence
  source: /note=“Description of Artificial Sequence Synthetic oligonucleotide”
uggagugcuguuauauaauua

SEQ ID NO: 494 - RNA, 23 nt, Artificial Sequence
  source: /note=“Description of Artificial Sequence Synthetic oligonucleotide”
uaauuauauaacagcacuccauc

SEQ ID NO: 495 - RNA, 21 nt, Artificial Sequence
  source: /note=“Description of Artificial Sequence Synthetic oligonucleotide”
gcacagcacaaauuaguuaua

SEQ ID NO: 496 - RNA, 23 nt, Artificial Sequence
  source: /note=“Description of Artificial Sequence Synthetic oligonucleotide”
uauaacuaauuugugcugugcac

SEQ ID NO: 497 - RNA, 21 nt, Artificial Sequence
  source: /note=“Description of Artificial Sequence Synthetic oligonucleotide”
cugaaugcuucuaaguaaaua

SEQ ID NO: 498 - RNA, 23 nt, Artificial Sequence
  source: /note=“Description of Artificial Sequence Synthetic oligonucleotide”
uauuuacuuagaagcauucagaa

SEQ ID NO: 499 - RNA, 21 nt, Artificial Sequence
  source: /note=“Description of Artificial Sequence Synthetic oligonucleotide”
guaagauuuguuuuuaaacua

SEQ ID NO: 500 - RNA, 23 nt, Artificial Sequence
  source: /note=“Description of Artificial Sequence Synthetic oligonucleotide”
uaguuuaaaaacaaaucuuacac

SEQ ID NO: 501 - RNA, 21 nt, Artificial Sequence
  source: /note=“Description of Artificial Sequence Synthetic oligonucleotide”
acgaagauaauuguuguucua

SEQ ID NO: 502 - RNA, 23 nt, Artificial Sequence
  source: /note=“Description of Artificial Sequence Synthetic oligonucleotide”
uagaacaacaauuaucuucguau

SEQ ID NO: 503 - RNA, 21 nt, Artificial Sequence
  source: /note=“Description of Artificial Sequence Synthetic oligonucleotide”
caucccuaaucuauacauaua

SEQ ID NO: 504 - RNA, 23 nt, Artificial Sequence
  source: /note=“Description of Artificial Sequence Synthetic oligonucleotide”
uauauguauagauuagggaugcu

SEQ ID NO: 505 - RNA, 21 nt, Artificial Sequence
  source: /note=“Description of Artificial Sequence Synthetic oligonucleotide”
uggagugcuguuauauaauua

SEQ ID NO: 506 - RNA, 23 nt, Artificial Sequence
  source: /note=“Description of Artificial Sequence Synthetic oligonucleotide”
uaauuauauaacagcacuccauc

SEQ ID NO: 507 - RNA, 21 nt, Artificial Sequence
  source: /note=“Description of Artificial Sequence Synthetic oligonucleotide”
gcacagcacaaauuaguuaua

SEQ ID NO: 508 - RNA, 23 nt, Artificial Sequence
  source: /note=“Description of Artificial Sequence Synthetic oligonucleotide”
uauaacuaauuugugcugugcac

SEQ ID NO: 509 - RNA, 21 nt, Artificial Sequence
  source: /note=“Description of Artificial Sequence Synthetic oligonucleotide”
cugaaugcuucuaaguaaaua

SEQ ID NO: 510 - RNA, 23 nt, Artificial Sequence
  source: /note=“Description of Artificial Sequence Synthetic oligonucleotide”
uauuuacuuagaagcauucagaa

SEQ ID NO: 511 - RNA, 21 nt, Artificial Sequence
  source: /note=“Description of Artificial Sequence Synthetic oligonucleotide”
uaaguugguucuagcgcaguu

SEQ ID NO: 512 - RNA, 23 nt, Artificial Sequence
  source: /note=“Description of Artificial Sequence Synthetic oligonucleotide”
aacugcgcuagaaccaacuuauu

SEQ ID NO: 513 - RNA, 21 nt, Artificial Sequence
  source: /note=“Description of Artificial Sequence Synthetic oligonucleotide”
aaguugguucuagcgcaguuu

SEQ ID NO: 514 - RNA, 23 nt, Artificial Sequence
  source: /note=“Description of Artificial Sequence Synthetic oligonucleotide”
aaacugcgcuagaaccaacuuau

SEQ ID NO: 515 - RNA, 21 nt, Artificial Sequence
  source: /note=“Description of Artificial Sequence Synthetic oligonucleotide”
uugaaauguaaggcuguguaa

SEQ ID NO: 516 - RNA, 23 nt, Artificial Sequence
  source: /note=“Description of Artificial Sequence Synthetic oligonucleotide”
uuacacagccuuacauuucaauu

SEQ ID NO: 517 - RNA, 21 nt, Artificial Sequence
  source: /note=“Description of Artificial Sequence Synthetic oligonucleotide”
uauaguuaacacacuaccgaa

SEQ ID NO: 518 - RNA, 23 nt, Artificial Sequence
  source: /note=“Description of Artificial Sequence Synthetic oligonucleotide”
uucgguaguguguuaacuauaca

SEQ ID NO: 519 - RNA, 21 nt, Artificial Sequence
  source: /note=“Description of Artificial Sequence Synthetic oligonucleotide”
aguuaacacacuaccgaaugu

SEQ ID NO: 520 - RNA, 23 nt, Artificial Sequence
  source: /note=“Description of Artificial Sequence Synthetic oligonucleotide”
acauucgguaguguguuaacuau

SEQ ID NO: 521 - RNA, 21 nt, Artificial Sequence
  source: /note=“Description of Artificial Sequence Synthetic oligonucleotide”
ugguauuuucaauagccacua

SEQ ID NO: 522 - RNA, 23 nt, Artificial Sequence
  source: /note=“Description of Artificial Sequence Synthetic oligonucleotide”
uaguggcuauugaaaauaccacc

SEQ ID NO: 523 - RNA, 21 nt, Artificial Sequence
  source: /note=“Description of Artificial Sequence Synthetic oligonucleotide”
ucugaugcagcuuauacgaaa

SEQ ID NO: 524 - RNA, 23 nt, Artificial Sequence
  source: /note=“Description of Artificial Sequence Synthetic oligonucleotide”
uuucguauaagcugcaucagaga

SEQ ID NO: 525 - RNA, 21 nt, Artificial Sequence
  source: /note=“Description of Artificial Sequence Synthetic oligonucleotide”
uaaguugguucuagcgcaguu

SEQ ID NO: 526 - RNA, 23 nt, Artificial Sequence
  source: /note=“Description of Artificial Sequence Synthetic oligonucleotide”
aacugcgcuagaaccaacuuauu

SEQ ID NO: 527 - RNA, 21 nt, Artificial Sequence
  source: /note=“Description of Artificial Sequence Synthetic oligonucleotide”
aaguugguucuagcgcaguuu

SEQ ID NO: 528 - RNA, 23 nt, Artificial Sequence
  source: /note=“Description of Artificial Sequence Synthetic oligonucleotide”
aaacugcgcuagaaccaacuuau

SEQ ID NO: 529 - RNA, 21 nt, Artificial Sequence
  source: /note=“Description of Artificial Sequence Synthetic oligonucleotide”
uugaaauguaaggcuguguaa

SEQ ID NO: 530 - RNA, 23 nt, Artificial Sequence
  source: /note=“Description of Artificial Sequence Synthetic oligonucleotide”
uuacacagccuuacauuucaauu

SEQ ID NO: 531 - RNA, 21 nt, Artificial Sequence
  source: /note=“Description of Artificial Sequence Synthetic oligonucleotide”
uauaguuaacacacuaccgaa

SEQ ID NO: 532 - RNA, 23 nt, Artificial Sequence
  source: /note=“Description of Artificial Sequence Synthetic oligonucleotide”
uucgguaguguguuaacuauaca

SEQ ID NO: 533 - RNA, 21 nt, Artificial Sequence
  source: /note=“Description of Artificial Sequence Synthetic oligonucleotide”
aguuaacacacuaccgaaugu

SEQ ID NO: 534 - RNA, 23 nt, Artificial Sequence
  source: /note=“Description of Artificial Sequence Synthetic oligonucleotide”
acauucgguaguguguuaacuau

SEQ ID NO: 535 - RNA, 21 nt, Artificial Sequence
  source: /note=“Description of Artificial Sequence Synthetic oligonucleotide”
ugguauuuucaauagccacua

SEQ ID NO: 536 - RNA, 23 nt, Artificial Sequence
  source: /note=“Description of Artificial Sequence Synthetic oligonucleotide”
uaguggcuauugaaaauaccacc

SEQ ID NO: 537 - RNA, 21 nt, Artificial Sequence
  source: /note=“Description of Artificial Sequence Synthetic oligonucleotide”
ucugaugcagcuuauacgaaa

SEQ ID NO: 538 - RNA, 23 nt, Artificial Sequence
  source: /note=“Description of Artificial Sequence Synthetic oligonucleotide”
uuucguauaagcugcaucagaga

(19) United States
(12) Patent Application Publication
(10) Pub. No.: US 2020/0308588 A1
(43) Pub. Date: Oct. 1, 2020

(54) HIGH MOBILITY GROUP BOX-1 (HMGB1) IRNA COMPOSITIONS AND METHODS OF USE THEREOF

(71) Applicant: Alnylam Pharmaceuticals, Inc., Cambridge, MA (US)

(72) Inventors: Gregory Hinkle, Plymouth, MA (US); Frederic Tremblay, Boston, MA (US); James D. McIninch, Burlington, MA (US)

(73) Assignees: Alnylam Pharmaceuticals, Inc., Cambridge, MA (US); Alnylam Pharmaceuticals, Inc., Cambridge, MA (US)

(21) Appl. No.: 16/954,625

(22) PCT Filed: Dec. 18, 2018
(86) PCT No.: PCT/US2018/066126

§ 371 Date: Jun. 17, 2020

Related U.S. Application Data
(60) Provisional application No. 62/599,860, filed on Dec. 18, 2017.

(51) Int. Cl. C12N 15/113

(52) U.S. Cl. CPC C12N 15/113; C12N 2310/14; C12N 2310/312; C12N 2310/315; C12N 2310/3183; C12N 2310/3521; C12N 2310/346; C12N 2310/351; C12N 2330/30; C12N 2310/3533; C12N 2310/345

(57) ABSTRACT

The present invention relates to RNAi agents, e.g., double stranded RNA (dsRNA) agents, targeting the HMGB 1 gene. The invention also relates to methods of using such RNAi agents to inhibit expression of a HMGB1 gene and to methods of N preventing and treating an HMGB1-associated disorder, e.g., metabolic disorder or non-alcholic fatty liver disease, e.g., non-alcoholic steatohepatitis (NASH).

### RELATED APPLICATIONS

This application claims the benefit of priority to U.S. Provisional Patent Application No. 62/599,860, filed on Dec. 18, 2017, the entire contents of which are hereby incorporated herein by reference.

### SEQUENCE LISTING

The instant application contains a Sequence Listing which has been submitted electronically in ASCII format and is hereby incorporated by reference in its entirety. Said ASCII copy, created on Dec. 12, 2018, is named 121301-08020_SL.txt and is 192,137 bytes in size.

### BACKGROUND OF THE INVENTION

Metabolic syndrome is a leading cause of mortality and morbidity in industrialized countries and non-alcoholic fatty liver disease (NAFLD) is the most common hepatic disease in developed countries estimated to affect one in three adults in the United States. Its prevalence in children is also increasing in parallel with childhood obesity.

NAFLD includes the entire spectrum of fatty liver disease (FLD) affecting subjects who drink little to no alcohol. As the name implies, the main characteristic of nonalcoholic fatty liver disease is too much fat stored in liver cells. The severity of NAFLD is varied and can range from simple hepatic steatosis or non-alcoholic fatty liver (NAFL) to nonalcoholic steatohepatitis (NASH), a progressive condition that may lead to cirrhosis, hepatocellular cancer or liver failure. NAFLD is commonly associated with metabolic syndrome, a combination of multiple disorders including insulin resistance, abdominal obesity, dyslipidemia, increased blood pressure, hypercholesterolemia, and a pro-inflammatory state, and is considered to be the hepatic manifestation of metabolic syndrome. As with obesity and insulin resistance, NAFLD has been associated with chronic inflammation. In addition to NAFLD, other insults including, but not limited to, hepatic infection, hepatic inflammation, cirrhosis, autoimmune hepatitis, chronic alcohol consumption optionally in association with one or more of fatty liver and elevated serum lipids or cholesterol; hemochromatosis, and chronic use of pharmaceutical agents that cause liver damage have been associated with chronic liver inflammation and liver fibrosis.

Liver inflammation and fibrosis, as a result of NAFLD, metabolic syndrome, and other insults can lead to liver necrosis which results in the release of high mobility group box-1 (HMGB1) protein in passive release from hepatocytes which, in turn, promotes recruitment and activation of inflammatory cells, including neutrophils. HMGB1 can also be secreted actively via a non-canonical secretory pathway in response to cell stress or inflammation. The inflammation response can cause further cell damage and promote fibrosis.

Current treatments for NAFLD and metabolic sysndrome are primarily life-style changes which are often ineffective. Accordingly, there is a need in the art for compositions and methods for the treatment of NAFLD and related disorders.

### SUMMARY OF THE INVENTION

The present invention provides iRNA compositions which affect the RNA-induced silencing complex (RISC)-mediated cleavage of RNA transcripts of a gene encoding high mobility group box-1 (HMGB1). The HMGB1 may be within a cell, e.g., a cell within a subject, such as a human.

In an aspect, the invention provides a double stranded ribonucleic acid (dsRNA) agent for inhibiting expression of HMGB1, wherein the dsRNA agent comprises a sense strand and an antisense strand, wherein the sense strand comprises at least 15 contiguous nucleotides differing by no more than 3 nucleotides from the nucleotide sequence of SEQ ID NO:1 and the antisense strand comprises at least 15 contiguous nucleotides differing by no more than 3 nucleotides from the nucleotide sequence of SEQ ID NO:2.

In certain embodiments, the sense strands and antisense strands comprise sequences selected from any one of the sequences provided in any one of Tables 3, 5, 6, or 7.

In an aspect, the invention provides a double stranded ribonucleic acid (dsRNA) agent for inhibiting expression of HMGB1, wherein the dsRNA agent comprises a sense strand and an antisense strand, the antisense strand comprising a region of complementarity which comprises at least 15 contiguous nucleotides differing by no more than 3 nucleotides from any one of the antisense nucleotide sequences listed in any one of Tables 3, 5, 6, or 7.

In certain embodiments, the dsRNA agent comprises at least one modified nucleotide. In some embodiments, all of the nucleotides of the sense strand and all of the nucleotides of the antisense strand comprise a modification.

In an aspect, the invention provides a double stranded RNA agent for inhibiting expression of HMGB1, wherein the dsRNA agent comprises a sense strand and an antisense strand forming a double stranded region, wherein the sense strand comprises at least 15 contiguous nucleotides differing by no more than 3 nucleotides from the nucleotide sequence of SEQ ID NO:1 and the antisense strand comprises at least 15 contiguous nucleotides differing by no more than 3 nucleotides from the nucleotide sequence of SEQ ID NO:2, wherein substantially all of the nucleotides of the sense strand and substantially all of the nucleotides of the antisense strand are modified nucleotides, and wherein the sense strand is conjugated to a ligand attached at the 3′-terminus.

In an embodiment, the present invention provides double stranded RNA agents for inhibiting expression of HMGB1, which comprise a sense strand and an antisense strand forming a double stranded region, wherein the sense strand comprises at least 15 contiguous nucleotides differing by no more than 3 nucleotides from any of nucleotides 830-851, 830-850, 831-851, 917-937, 944-997, 944-990, 944-964, 968-997, 968-990, 968-995, 968-988, 969-989, 970-990, 971-991, 972-992, 972-995, 973-993, 974-994, 975-995, 976-996, 977-997, 1019-1039, 1158-1194, 1158-1182, 1158-1178, 1159-1179, 1160-1180, 1161-1181, 1162-1182, or 1174-1194 of SEQ ID NO:1 and the antisense strand comprises at least 15 contiguous nucleotides differing by no more than 3 nucleotides from the corresponding position of the nucleotide sequence of SEQ ID NO:2 such that the antisense strand is complementary to the at least 15 contiguous nucleotides differing by no more than 3 nucleotides in the sense strand. In certain embodiments, substantially all of the nucleotides of the sense strand are modified nucleotides. In certain embodiments, substantially all of the nucleotides of the antisense strand are modified nucleotides. In certain embodiments, substantially all of the nucleotides of both strands are modified. In some embodiments, the sense strand is conjugated to a ligand attached at the 3′-terminus.

In an aspect, the present invention also provides dsRNA agents for inhibiting expression of HMGB1, which comprise a sense strand and an antisense strand forming a double stranded region, wherein the sense strand comprises at least 15 contiguous nucleotides from any of nucleotides 830-851, 830-850, 831-851, 917-937, 944-997, 944-990, 944-964, 968-997, 968-990, 968-995, 968-988, 969-989, 970-990, 971-991, 972-992, 972-995, 973-993, 974-994, 975-995, 976-996, 977-997, 1158-1194, 1019-1039, 1158-1182, 1158-1178, 1159-1179, 1160-1180, 1161-1181, 1162-1182, or 1174-1194 of SEQ ID NO:1 and the antisense strand comprises at least 15 contiguous nucleotides from the corresponding position of the nucleotide sequence of SEQ ID NO:2 such that the antisense strand is complementary to the at least 15 contiguous nucleotides in the sense strand.

In certain embodiments, the sense strand comprises at least 15 contiguous nucleotides of the nucleotide sequence 5′-UAAGUUGGUUCUAGCGCAGUU-3′ (SEQ ID NO:511) and the antisense strand comprises at least 15 contiguous nucleotides of the nucleotide sequence 5′-AACUGCGCUAGAACCAACUUAUU-3′ (SEQ ID NO:512). In certain embodiments, the sense strand comprises at least 17 contiguous nucleotides of the nucleotide sequence 5′-UAAGUUGGUUCUAGCGCAGUU-3′ (SEQ ID NO:511) and the antisense strand comprises at least 17 contiguous nucleotides of the nucleotide sequence 5′-AACUGCGCUAGAACCAACUUAUU-3′ (SEQ ID NO:512). In certain embodiments, the sense strand comprises at least 19 contiguous nucleotides of the nucleotide sequence 5′-UAAGUUGGUUCUAGCGCAGUU-3′ (SEQ ID NO:511) and the antisense strand comprises at least 19 contiguous nucleotides of the nucleotide sequence 5′-AACUGCGCUAGAACCAACUUAUU-3′ (SEQ ID NO:512). In certain embodiments, the sense strand comprises the 21 contiguous nucleotides of the nucleotide sequence 5′-UAAGUUGGUUCUAGCGCAGUU-3′ (SEQ ID NO:511) and the antisense strand comprises at least 21 contiguous nucleotides of the nucleotide sequence 5′-AACUGCGCUAGAACCAACUUAUU-3′ (SEQ ID NO:512).

In certain embodiments, the sense strand comprises at least 15 contiguous nucleotides of the nucleotide sequence 5′-AAGUUGGUUCUAGCGCAGUUU-3′ (SEQ ID NO:513) and the antisense strand comprises at least 15 contiguous nucleotides of the nucleotide sequence 5′-AAACUGCGCUAGAACCAACUUAU-3′ (SEQ ID NO:514). In certain embodiments, the sense strand comprises at least 17 contiguous nucleotides of the nucleotide sequence 5′-AAGUUGGUUCUAGCGCAGUUU-3′ (SEQ ID NO:513) and the antisense strand comprises at least 17 contiguous nucleotides of the nucleotide sequence 5′-AAACUGCGCUAGAACCAACUUAU-3′ (SEQ ID NO:514). In certain embodiments, the sense strand comprises at least 19 contiguous nucleotides of the nucleotide sequence 5′-AAGUUGGUUCUAGCGCAGUUU-3′ (SEQ ID NO:513) and the antisense strand comprises at least 19 contiguous nucleotides of the nucleotide sequence 5′-AAACUGCGCUAGAACCAACUUAU-3′ (SEQ ID NO:514). In certain embodiments, the sense strand comprises the 21 contiguous nucleotides of the nucleotide sequence 5′-AAGUUGGUUCUAGCGCAGUUU-3′ (SEQ ID NO:513) and the antisense strand comprises at least 21 contiguous nucleotides of the nucleotide sequence 5′-AAACUGCGCUAGAACCAACUUAU-3′ (SEQ ID NO:514).

In certain embodiments, substantially all of the nucleotides of the sense strand are modified nucleotides. In certain embodiments, substantially all of the nucleotides of the antisense strand are modified nucleotides. In certain embodiments, substantially all of the nucleotides of both strands are modified. In some embodiments, the sense strand is conjugated to a ligand attached at the 3′-terminus.

In certain embodiments, the antisense strand comprises a region of complementarity which comprises at least 15 contiguous nucleotides differing by no more than 3 nucleotides from any one of the antisense sequences listed in any one of Tables 3, 5, 6, or 7. For example, in a certain embodiment, the antisense strand comprises a region of complementarity which comprises at least 15 contiguous nucleotides differing by no more than 3 nucleotides from any one of the antisense sequences of a duplex selected from the group consisting of AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-19318, AD-80651, or AD-80652. In certain embodiment, the antisense strand comprises a region of complementarity which comprises at least 15 contiguous nucleotides of any one of the antisense nucleotide sequences of one of any one of the foregoing duplexes.

In some embodiments, all of the nucleotides of the sense strand and all of the nucleotides of the antisense strand comprise a modification. In one embodiment, at least one of the modified nucleotides is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide (GNA), and a 2-O—(N-methylacetamide) modified nucleotide; and combinations thereof.

In certain embodiments, substantially all of the nucleotides of the sense strand are modified. In certain embodiments, substantially all of the nucleotides of the antisense strand are modified. In certain embodiments, substantially all of the nucleotides of both the sense strand and the antisense strand are modified.

In certain embodiments, the duplex comprises a modified antisense strand selected from the group of antisense sequences provided in any one of Tables 5, 6, or 7. In certain embodiments, the duplex comprises a modified sense strand selected from the group of sense sequence provided in any one of Tables 5, 6, or 7. In certain embodiments, the duplex comprises a modified duplex selected from the group of duplexes provided in any one of Tables 5, 6, or 7. In certain embodiments, the duplex is selected from the group consisting of AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-19318, AD-80651, or AD-80652.

In certain embodiments, the sense strand comprises a modified sequence of 5′-usasaguuGfgUfUfCfuagcgcaguu-3′ (SEQ ID NO:525) and the antisense strand comprises a modified sequence of 5′-asAfscugc(Ggn)cuagaaCfcAfacuuasusu-3′ (SEQ ID NO:526), wherein a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Ggn) is guanosine-glycol nucleic acid (GNA), and s is phosphorothioate linkage; and wherein the 3′end of the sense strand is optionally covalently linked to a ligand, e.g., an N-[tris(GalNAc-alkyl)-amidodecanoyl)]-4-hydroxyprolinol (also referred to as Hyp-(GalNAc-alkyl)3 or L96).

In certain embodiments, the sense strand comprises a modified sequence of 5′-asasguugGfuUfCfUfagcgcaguuu-3′ (SEQ ID NO:527) and the antisense strand comprises a modified sequence of 5′-asAfsacug(Cgn)gcuagaAfcCfaacuusasu-3′ (SEQ ID NO:528), wherein a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Cgn) is cytidine-glycol nucleic acid (GNA), and s is phosphorothioate linkage; and wherein the 3′end of the sense strand is optionally covalently linked to a ligand, e.g., an N-[tris(GalNAc-alkyl)-amidodecanoyl)]-4-hydroxyprolinol (also referred to as Hyp-(GalNAc-alkyl)3 or L96).

In certain embodiments, the region of complementarity between the antisense strand and the target is at least 17 nucleotides in length. For example, the region of complementarity between the antisense strand and the target is 19 to 21 nucleotides in length, for example, the region of complementarity is 21 nucleotides in length. In preferred embodiments, each strand is no more than 30 nucleotides in length. In one embodiment, each strand is independently 21 to 23 nucleotides in length.

In some embodiments, at least one strand comprises a 3′ overhang of at least 1 nucleotide, e.g., at least one strand comprises a 3′ overhang of at least 2 nucleotides.

In many embodiments, the dsRNA agent further comprises a ligand. The ligand can be conjugated to the 3′ end of the sense strand of the dsRNA agent. The ligand can be an N-acetylgalactosamine (GalNAc) derivative including, but not limited to

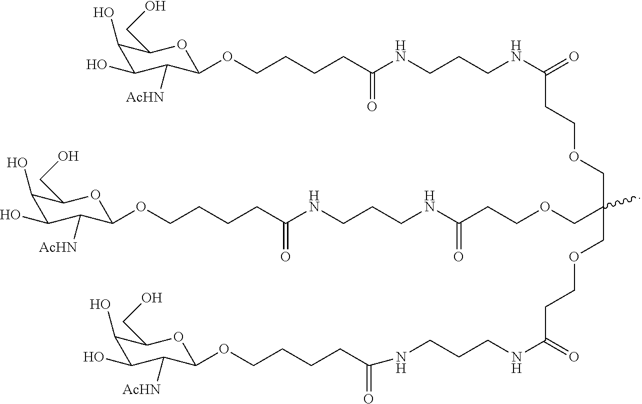 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

An exemplary dsRNA agent conjugated to the ligand as shown in the following schematic:

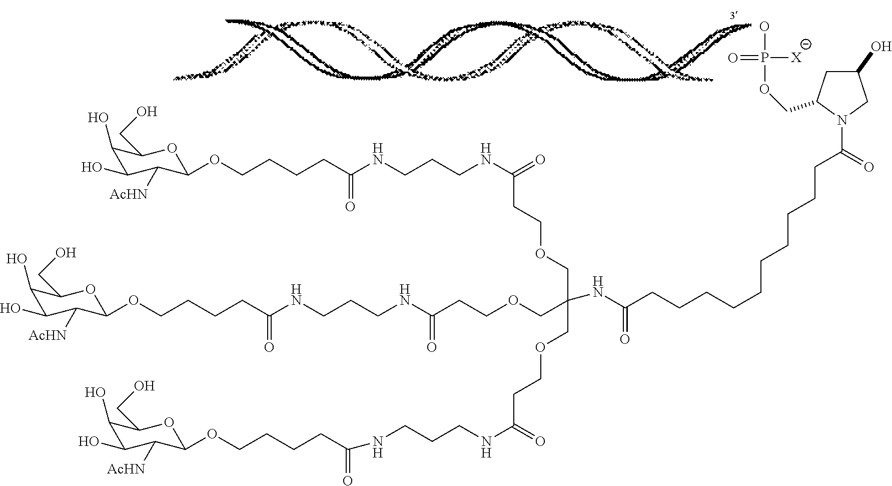

and, wherein X is O or S. In one embodiment, the X is O.

In certain embodiments, the region of complementarity comprises one of the antisense sequences in any one of Tables 3, 5, 6, or 7. In other embodiments, the region of complementarity consists of one of the antisense sequences in any one of Tables 3, 5, 6, or 7. In certain embodiments, the antisense strand is from a duplex selected from the group consisting of AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-19318, AD-80651, and AD-80652.

In an aspect, the invention provides a double stranded RNA (dsRNA) agent for inhibiting expression of HMGB1, wherein the dsRNA agent comprises a sense strand and an antisense strand forming a double stranded region, wherein the sense strand comprises at least 15 contiguous nucleotides differing by no more than 3 nucleotides from the nucleotide sequence of SEQ ID NO:1 and the antisense strand comprises at least 15 contiguous nucleotides differing by no more than 3 nucleotides from the nucleotide sequence of SEQ ID NO:2, wherein substantially all of the nucleotides of the sense strand comprise a modification selected from a 2′-O-methyl modification and a 2′-fluoro modification, wherein the sense strand comprises two phosphorothioate internucleotide linkages at the 5′-terminus, wherein substantially all of the nucleotides of the antisense strand comprise a modification selected from a 2′-O-methyl modification and a 2′-fluoro modification, wherein the antisense strand comprises two phosphorothioate internucleotide linkages at the 5′-terminus and two phosphorothioate internucleotide linkages at the 3′-terminus, and wherein the sense strand is conjugated to one or more GalNAc derivatives attached through a monovalent, a bivalent, or a trivalent branched linker at the 3′-terminus. In certain embodiments, the modifications further include a GNA modification. In certain embodiments, the GNA modification is at a site opposite to the seed region of the antisense strand at positions 2-8 of the 5′-end of the antisense strand.

In certain embodiments, the sense strand comprises at least 15 contiguous nucleotides of any of nucleotides 830-851, 830-850, 831-851, 917-937, 944-997, 944-990, 944-964, 968-997, 968-990, 968-995, 968-988, 969-989, 970-990, 971-991, 972-992, 972-995, 973-993, 974-994, 975-995, 976-996, 977-997, 1158-1194, 1019-1039, 1158-1182, 1158-1178, 1159-1179, 1160-1180, 1161-1181, 1162-1182, or 1174-1194 of SEQ ID NO:1 and the antisense strand comprises at least 15 contiguous nucleotides from the corresponding position of the nucleotide sequence of SEQ ID NO:2 such that the antisense strand is complementary to the at least 15 contiguous nucleotides in the sense strand, wherein substantially all of the nucleotides of the sense strand comprise a modification selected from a 2′-O-methyl modification and a 2′-fluoro modification, wherein the sense strand comprises two phosphorothioate internucleotide linkages at the 5′-terminus, wherein substantially all of the nucleotides of the antisense strand comprise a modification selected from a 2′-O-methyl modification and a 2′-fluoro modification, wherein the antisense strand comprises two phosphorothioate internucleotide linkages at the 5′-terminus and two phosphorothioate internucleotide linkages at the 3′-terminus, and wherein the sense strand is conjugated to one or more GalNAc derivatives attached through a monovalent, a bivalent, or a trivalent branched linker at the 3′-terminus. In certain embodiments, the modifications further include a GNA modification.

In certain embodiments, all of the nucleotides of the sense strand and all of the nucleotides of the antisense strand are modified nucleotides. In certain embodiments, each strand independently has 19-30 nucleotides. In certain embodiments, each strand has 14-40 nucleotides.

In certain embodiments, substantially all of the nucleotides of the sense strand are modified. In certain embodiments, substantially all of the nucleotides of the antisense strand are modified. In certain embodiments, substantially all of the nucleotides of both the sense strand and the antisense strand are modified.

In certain embodiments, the sense strand comprises a thermally destabilizing nucleotide placed at a site opposite to the seed region of the antisense strand at positions 2-8 of the 5′-end of the antisense strand. In certain embodiments, the thermally destabilizing modification is selected from an abasic modification; a mismatch with the opposing nucleotide in the duplex; and destabilizing sugar modification such as 2′-deoxy modification or acyclic nucleotide such as unlocked nucleic acids (UNA) or glycerol nucleic acid (GNA). In certain embodiments, the destabilizing sugar modification is GNA.

In an aspect, the invention provides a cell containing the dsRNA agent as described herein.

In an aspect, the invention provides a vector encoding at least one strand of a dsRNA agent, wherein the dsRNA agent comprises a region of complementarity to at least a part of an mRNA encoding HMGB1, wherein the dsRNA is 30 base pairs or less in length, and wherein the dsRNA agent targets the mRNA for cleavage. In certain embodiments, the region of complementarity is at least 15 nucleotides in length. In certain embodiments, the region of complementarity is 19 to 23 nucleotides in length.

In an aspect, the invention provides a pharmaceutical composition for inhibiting expression of an HMGB1 gene comprising a dsRNA agent of the invention.

In an aspect, the invention provides a pharmaceutical composition comprising the double stranded RNA agent of the invention and a lipid formulation. In certain embodiments, the lipid formulation comprises a LNP. In certain embodiments, the lipid formulation comprises a MC3.

In an aspect, the invention provides a method of inhibiting HMGB1 expression in a cell, the method comprising (a) contacting the cell with the double stranded RNA agent of the invention or a pharmaceutical composition of the invention; and (b) maintaining the cell produced in step (a) for a time sufficient to obtain degradation of the mRNA transcript of a HMGB1 gene, thereby inhibiting expression of the HMGB1 gene in the cell. In certain embodiments, the cell is within a subject, for example, a human subject, for example a female human or a male human. In preferred embodiments, HMGB1 expression is inhibited by at least 30%, 40%, 50%, 60%, 70%, 80%, 90%, 95%, or to below the threshold of detection within the cell. In certain embodiments, reduction of expression of HMGB1 is detected in a subject by measuring the level of HMGB1 in a blood or serum sample from the subject. In certain embodiments, reduction of expression of HMGB1 is detected in a subject by measuring the level of HMGB1 in a urine sample from the subject. In preferred embodiments, the HMGB1 level in the subject sample is reduced by at least 30%, 40%, preferably at least 50%, 60%, 70%, 80%, 90%, or 95%. In certain embodiments, HMGB1 protein is detected. In certain embodiments, HMGB1 RNA is detected, e.g., circulating RNA present in vesicular structures. In certain embodiments, the HMGB1 RNA is assayed for site specific cleavage. In certain embodiments, the subject is suffering from an HMGB1-associated disorder. In certain embodiments, the subject meets at least one diagnostic criterion for metabolic disorder. In certain embodiments, the subject has been diagnosed with metabolic disorder. In certain embodiments, the subject has been diagnosed with NAFLD. In certain embodiments, the subject has been diagnosed with NASH.

In an aspect, the invention provides a method of treating an HMGB1-associated disorder. In certain embodiments, the HMGB1-associated disorder is selected from liver inflammation, liver fibrosis, liver damage associated with an elevated level of HMGB1, metabolic disorder, blood pressure equal to or higher than 130/85 mmHg, large waist circumference (40 inches or more for men and 35 inches or more for women); waist-to-hip ratio <1.0 (for men) or <0.8 (for women); low HDL cholesterol (under 40 mg/dL for men and under 50 mg/dL for women), triglycerides of at least 150 mg/dL, NAFLD, steatohepatitis, NASH, NASH cirrhosis, cryptogenic cirrhosis, hypertension, hypercholesterolemia, hepatic infection, hepatic inflammation, cirrhosis, autoimmune hepatitis, chronic alcohol consumption, alcoholic hepatitis, alcoholic steatohepatitis, hemochromatosis, and chronic use of pharmaceutical agents that cause liver damage. In an aspect, the invention provides a method of treating metabolic disorder. In an aspect, the invention provides a method of treating NAFLD. In an aspect, the invention provides a method of treating NASH.

In an aspect, the invention provides a method of preventing the development of an HMGB1-associated disorder in a subject at risk of developing an HMGB1-associated disorder. In an aspect, the invention provides a method of preventing the development of NASH in a subject at risk of developing NASH, e.g. a subject at risk of developing or diagnosed with NAFLD or metabolic disorder.

In preferred embodiments, the subject diagnosed with or at risk of developing an HMGB1-associated disorder is human. In certain embodiments, the subject has at least one sign of metabolic disorder selected from elevated fasting blood glucose of at least 100 mg/dL, blood pressure equal to or higher than 130/85 mmHg, large waist circumference wherein a large waist circumference is 40 inches or more for men and 35 inches or more for women; low HDL cholesterol wherein low LDH cholesterol is under 40 mg/dL for men and under 50 mg/dL for women; triglycerides equal to or higher than 150 mg/dL. In certain embodiments, the subject has an Hb1Ac of at least 6.5%, type 2 diabetes mellitus, or 2 hour postprandial blood glucose or serum glucose concentration of at least 140 mg/dl. In certain embodiments, the subject is obese. In certain embodiments, the subject has type 2 diabetes mellitus. In certain embodiments, the subject has fatty liver disease (FLD). In certain embodiments, the subject has a NAFLD, e.g., steatohepatitis, NASH, NASH cirrhosis, cryptogenic cirrhosis, or hemochromatosis. In certain embodiments, the subject suffers from liver inflammation or fibrosis. In certain embodiments, the subject suffers from a hepatic infection, cirrhosis, or autoimmune hepatitis. In certain embodiments, the subject has exhibited chronic excessive alcohol consumption. In certain embodiments, the subject has alcoholic hepatitis or alcoholic steatohepatitis. In certain embodiments, the subject is a female human. In certain embodiments, the subject is a male human.

In certain embodiments, the subject with an HMGB1-associated disorder is at risk of developing NAFLD. In certain embodiments, the subject at risk of developing NAFLD is obese. In certain embodiments, the subject at risk of developing NAFLD has at least one sign of metabolic disorder selected from elevated fasting blood glucose of at least 100 mg/dL, blood pressure equal to or higher than 130/85 mmHg, large waist circumference wherein a large waist circumference is 40 inches or more for men and 35 inches or more for women; low HDL cholesterol wherein low LDH cholesterol is under 40 mg/dL for men and under 50 mg/dL for women; triglycerides equal to or higher than 150 mg/dL. In certain embodiments, the subject at risk of developing NAFLD has an Hb1Ac of at least 6.5%, type 2 diabetes mellitus, or 2 hour postprandial blood glucose or serum glucose concentration of at least 140 mg/dl. In certain embodiments, the subject at risk of developing NAFLD has a condition with an established association to NAFLD such as obesity, type 2 diabetes mellitus, dyslipidemia, and polycystic ovarian disease. In certain embodiments, the subject has a condition associated with NAFLD such as hypothyroidism, obstructive sleep apnea, hypopituitarism, hypogonadism, pancreatoduodenal resection, and psoriasis. In certain embodiments, the subject has a NAFLD. In certain embodiments, the subject is a female human. In certain embodiments, the subject is a male human.

In certain embodiments, the dsRNA agent is administered at a dose of about 0.01 mg/kg to about 50 mg/kg. In certain embodiments, the dsRNA agent is administered to the subject subcutaneously.

In certain embodiments, the methods of the invention further comprise monitoring the subject for changes in one or more diagnostic markers of an HMGB1-associated disorder, e.g., NAFLD or metabolic disorder. In certain embodiments, the method further includes measuring level of HMGB1 in the subject, e.g., level of HMGB1 protein or RNA in a subject blood or serum sample or in a subject urine sample. In certain embodiments, the subject has undergone a test for HbA1c level, pre-prandial blood glucose, post-prandial blood glucose, insulin sensitivity, or glucose sensitivity test, a blood pressure test, a cholesterol test, or a serum lipid test; or weight and height determination or a waist circumference determination.

In certain embodiments, the subject has been administered an additional agent or subject to an intervention for the treatment of metabolic disorder, e.g., an additional agent for the treatment of high blood pressure or type 2 diabetes, or gastric bypass surgery.

In various embodiments, the dsRNA agent is administered at a dose of about 0.01 mg/kg to about 10 mg/kg or about 0.5 mg/kg to about 50 mg/kg. In some embodiments, the dsRNA agent is administered at a dose of about 10 mg/kg to about 30 mg/kg. In certain embodiments, the dsRNA agent is administered at a dose selected from the group consisting of 0.5 mg/kg, 1 mg/kg, 1.5 mg/kg, 3 mg/kg, 5 mg/kg, 10 mg/kg, and 30 mg/kg. In certain embodiments, the RNAi agent is administered about once per month, about once every other two months, about once a quarter (i.e., once every three months), or about once every six months at a dose of about 0.1 mg/kg to about 5.0 mg/kg. In certain embodiments, the dsRNA agent is administered no more than about once per month.

In certain embodiments, the dsRNA agent is administered to the subject once a month. In certain embodiments, the dsRNA agent is administered to the subject once every three months. In certain embodiments, the dsRNA agent is administered once every three to six months. In certain embodiments, the RNA agent is administered no more than once per month.

In some embodiments, the dsRNA agent is administered to the subject subcutaneously.

In certain embodiments, the level of HMGB1 is measured in the subject. In certain embodiments, the level of HMGB1 is the level of HMGB1 protein in a subject blood or serum sample, or the level of HMGB1 RNA in a blood or urine sample. In certain embodiments, the RNA in the blood or urine sample is assayed for an siRNA cleavage site.

### DETAILED DESCRIPTION OF THE INVENTION

The present invention provides iRNA compositions which effect the RNA-induced silencing complex (RISC)-mediated cleavage of RNA transcripts of an HMGB1 gene. The gene may be within a cell, e.g., a cell within a subject, such as a human. The use of these iRNAs enables the targeted degradation of mRNAs of the corresponding gene (HMGB1 gene) in mammals.

The iRNAs of the invention have been designed to target the human HMGB1 gene, including portions of the gene that are conserved in the HMGB1 orthologs of other mammalian species. Without intending to be limited by theory, it is believed that a combination or sub-combination of the foregoing properties and the specific target sites or the specific modifications in these iRNAs confer to the iRNAs of the invention improved efficacy, stability, potency, durability, and safety.

Accordingly, the present invention provides methods for treating and preventing an HMGB1-associated disorder, e.g., NAFLD or metabolic disorder, using iRNA compositions which effect the RNA-induced silencing complex (RISC)-mediated cleavage of RNA transcripts of an HMGB1 gene.

The iRNAs of the invention include an RNA strand (the antisense strand) having a region which is about 30 nucleotides or less in length, e.g., 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length, preferably 19-21 nucleotides in length, which region is substantially complementary to at least part of an mRNA transcript of an HMGB1 gene.

In certain embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an HMGB1 gene. In some embodiments, such iRNA agents having longer length antisense strands preferably may include a second RNA strand (the sense strand) of 20-60 nucleotides in length wherein the sense and antisense strands form a duplex of 18-30 contiguous nucleotides.

In some embodiments, one or both of the strands of the double stranded RNAi agents of the invention is up to 66 nucleotides in length, e.g., 36-66, 26-36, 25-36, 31-60, 22-43, 27-53 nucleotides in length, with a region of at least 19 contiguous nucleotides that is substantially complementary to at least a part of an mRNA transcript of an HMGB1 gene. In some embodiments, such iRNA agents having longer length antisense strands may include a second RNA strand (the sense strand) of 20-60 nucleotides in length wherein the sense and antisense strands form a duplex of 18-30 contiguous nucleotides.

The use of iRNAs of the invention enables the targeted degradation of mRNAs of the corresponding gene (HMGB1 gene) in mammals. Using in vitro and in vivo assays, the present inventors have demonstrated that iRNAs targeting an HMGB1 gene can mediate RNAi, resulting in significant inhibition of expression of HMGB1. Thus, methods and compositions including these iRNAs are useful for preventing and treating a subject at risk of developing or diagnosed with an HMGB1-associated disorder, e.g., NAFLD or metabolic disorder. The methods and compositions herein are useful for reducing the level of HMGB1 in a subject, especially in a subject suffering from an HMGB1-associated disorder.

The following detailed description discloses how to make and use compositions containing iRNAs to inhibit the expression of an HMGB1 gene as well as compositions, uses, and methods for treating subjects that would benefit from reduction of the expression of an HMGB1 gene, e.g., subjects at risk of developing or diagnosed with an HMGB1-associated disorder, e.g., NAFLD or metabolic disorder.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21 nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleotides” has a 2, 1, or 0 nucleotide overhang. When “no more than” is present before a series of numbers or a range, it is understood that “no more than” can modify each of the numbers in the series or range.

As used herein, ranges include both the upper and lower limit.

In the event of a conflict between a sequence and its indicated site on a transcript or other sequence, the nucleotide sequence recited in the specification, e.g., in the tables providing duplex sequences, takes precedence.

As used herein, “High Mobility Group Box-1” or “HMGB1” is a protein that belongs to the High Mobility Group-box superfamily. The encoded non-histone, nuclear DNA-binding protein regulates transcription, and is involved in organization of DNA. This protein plays a role in several cellular processes, including inflammation, cell differentiation and tumor cell migration. Multiple pseudogenes of this gene have been identified. Alternative splicing results in multiple transcript variants that encode the same protein. Further information on HMGB1 is provided, for example, in the NCBI Gene database at www.ncbi.nlm.nih.gov/gene/3146 (which is incorporated herein by reference in the version available as of Dec. 18, 2017). HMGB1 is also known as HMG1; HMG3; HMG-1; SBP-1. Unless otherwise clear from context HMGB1, or variations of capitalization thereof, can refer to any of the gene, the RNA, and the protein, including processed and unprocessed forms or fragments of the protein or RNA.

As used herein, “HMGB1” refers to the naturally occurring gene that encodes the HMGB1 protein. The amino acid and complete coding sequences of the reference sequence of the human HMGB1 gene may be found in, for example, GenBank Accession No. NM_002128.5 (SEQ ID NO:1; SEQ ID NO:2). Mammalian orthologs of the human HMGB1 gene may be found in, for example, Accession No. NM_010439.4, mouse (SEQ ID NO:3 and SEQ ID NO:4); Accession No. NM_012963.2, rat (SEQ ID NO:5 and SEQ ID NO:6); and Accession No. NM_001283356.1, cynomolgus monkey (SEQ ID NO:7 and SEQ ID NO:8). Human HMGB1 transcript variants include NM_001313892.1 (SEQ ID NO: 9 and 10) and NM_001313893.1 (SEQ ID NO: 11 and 12).

A number of naturally occurring SNPs are known and can be found, for example, in the SNP database at the NCBI at www.ncbi.nlm.nih.gov/snp?LinkName=gene_snp&from_uid=3146 (which is incorporated herein by reference in the version available as of Dec. 18, 2017) which lists SNPs in human HMGB1. In preferred embodiments, such naturally occurring variants are included within the scope of the HMGB1 gene sequence.

As used herein, an “HMGB1-associated disorder” and the like is understood as a disease or condition associated with liver steatosis, inflammation, fibrosis, or damage associated with an elevated level of HMGB1 or a release of HMGB1 from the liver cells due to cell death (e.g., necrosis or apoptosis) or active secretion. Exemplary HMGB1-associated disorders include metabolic disorder and the diagnostic components thereof, NAFLD (e.g., steatohepatitis, NASH, NASH cirrhosis, or cryptogenic cirrhosis), obesity, hepatic infection, hepatic inflammation, cirrhosis, autoimmune hepatitis, chronic alcohol consumption optionally in association with one or more of fatty liver and elevated serum lipids or cholesterol; alcoholic hepatitis, alcoholic steatohepatitis, hemochromatosis, and chronic use of pharmaceutical agents that cause liver damage. In certain embodiments, HMGB1-associated disorders are disorders associated with chronic insults. In certain embodiments, HMGB1-associated disorders exclude disorders associated with acute insults, e.g., acute liver toxicity due to poisoning, e.g., acute acetaminophen overdose.

As used herein, “target sequence” refers to a contiguous portion of the nucleotide sequence of an mRNA molecule formed during the transcription of an HMGB1 gene, including mRNA that is a product of RNA processing of a primary transcription product. The target portion of the sequence will be at least long enough to serve as a substrate for iRNA-directed cleavage at or near that portion of the nucleotide sequence of an mRNA molecule formed during the transcription of an HMGB1 gene. In one embodiment, the target sequence is within the protein coding region of HMGB1.

The target sequence may be from about 9-36 nucleotides in length, e.g., about 15-30 nucleotides in length. For example, the target sequence can be from about 15-30 nucleotides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. Ranges and lengths intermediate to the above recited ranges and lengths are also contemplated to be part of the invention.

As used herein, the term “strand comprising a sequence” refers to an oligonucleotide comprising a chain of nucleotides that is described by the sequence referred to using the standard nucleotide nomenclature.

“G,” “C,” “A,” “T,” and “U” each generally stand for a nucleotide that contains guanine, cytosine, adenine, thymidine, and uracil as a base, respectively. However, it will be understood that the term “ribonucleotide” or “nucleotide” can also refer to a modified nucleotide, as further detailed below, or a surrogate replacement moiety (see, e.g., Table 2). The skilled person is well aware that guanine, cytosine, adenine, and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

The terms “iRNA”, “RNAi agent,” “iRNA agent,”, “RNA interference agent” as used interchangeably herein, refer to an agent that contains RNA as that term is defined herein, and which mediates the targeted cleavage of an RNA transcript via an RNA-induced silencing complex (RISC) pathway. iRNA directs the sequence-specific degradation of mRNA through a process known as RNA interference (RNAi). The iRNA modulates, e.g., inhibits, the expression of an HMGB1 gene in a cell, e.g., a cell within a subject, such as a mammalian subject.

In one embodiment, an RNAi agent of the invention includes a single stranded RNA that interacts with a target RNA sequence, e.g., an HMGB1 target mRNA sequence, to direct the cleavage of the target RNA. Without wishing to be bound by theory it is believed that long double stranded RNA introduced into cells is broken down into siRNA by a Type III endonuclease known as Dicer (Sharp et al. (2001) Genes Dev. 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleave the target to induce silencing (Elbashir, et al., (2001) Genes Dev. 15:188). Thus, in one aspect the invention relates to a single stranded RNA (siRNA) generated within a cell and which promotes the formation of a RISC complex to effect silencing of the target gene, i.e., an HMGB1 gene. Accordingly, the term “siRNA” is also used herein to refer to an iRNA as described above.

In certain embodiments, the RNAi agent may be a single-stranded siRNA (ssRNAi) that is introduced into a cell or organism to inhibit a target mRNA. Single-stranded RNAi agents bind to the RISC endonuclease, Argonaute 2, which then cleaves the target mRNA. The single-stranded siRNAs are generally 15-30 nucleotides and are chemically modified. The design and testing of single-stranded siRNAs are described in U.S. Pat. No. 8,101,348 and in Lima et al., (2012) Cell 150:883-894, the entire contents of each of which are hereby incorporated herein by reference. Any of the antisense nucleotide sequences described herein may be used as a single-stranded siRNA as described herein or as chemically modified by the methods described in Lima et al., (2012) Cell 150:883-894.

In certain embodiments, an “iRNA” for use in the compositions, uses, and methods of the invention is a double stranded RNA and is referred to herein as a “double stranded RNA agent,” “double stranded RNA (dsRNA) molecule,” “dsRNA agent,” or “dsRNA”. The term “dsRNA”, refers to a complex of ribonucleic acid molecules, having a duplex structure comprising two anti-parallel and substantially complementary nucleic acid strands, referred to as having “sense” and “antisense” orientations with respect to a target RNA, i.e., an HMGB1 gene. In some embodiments of the invention, a double stranded RNA (dsRNA) triggers the degradation of a target RNA, e.g., an mRNA, through a post-transcriptional gene-silencing mechanism referred to herein as RNA interference or RNAi.

In general, the majority of nucleotides of each strand of a dsRNA molecule are ribonucleotides, but as described in detail herein, each or both strands can also include one or more non-ribonucleotides, e.g., a deoxyribonucleotide or a modified nucleotide. In addition, as used in this specification, an “iRNA” may include ribonucleotides with chemical modifications; an iRNA may include substantial modifications at multiple nucleotides. As used herein, the term “modified nucleotide” refers to a nucleotide having, independently, a modified sugar moiety, a modified internucleotide linkage, or modified nucleobase, or any combination thereof. Thus, the term modified nucleotide encompasses substitutions, additions or removal of, e.g., a functional group or atom, to internucleoside linkages, sugar moieties, or nucleobases. The modifications suitable for use in the agents of the invention include all types of modifications disclosed herein or known in the art. Any such modifications, as used in a siRNA type molecule, are encompassed by “iRNA” or “RNAi agent” for the purposes of this specification and claims.

The duplex region may be of any length that permits specific degradation of a desired target RNA through a RISC pathway, and may range from about 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop.” A hairpin loop can comprise at least one unpaired nucleotide. In some embodiments, the hairpin loop can comprise at least 2, 3, 4, 5, 6, 7, 8, 9, 10, 20, 23 or more unpaired nucleotides. In some embodiments, the hairpin loop can be 10 or fewer nucleotides. In some embodiments, the hairpin loop can be 8 or fewer unpaired nucleotides. In some embodiments, the hairpin loop can be 4-10 unpaired nucleotides. In some embodiments, the hairpin loop can be 4-8 nucleotides.

Where the two substantially complementary strands of a dsRNA are comprised by separate RNA molecules, those molecules need not be, but can be covalently connected. Where the two strands are connected covalently by means other than an uninterrupted chain of nucleotides between the 3′-end of one strand and the 5′-end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, an RNAi may comprise one or more nucleotide overhangs.

In certain embodiments, an iRNA agent of the invention is a dsRNA, each strand of which comprises 19-23 nucleotides, that interacts with a target RNA sequence, e.g., an HMGB1 gene, to direct cleavage of the target RNA.

In some embodiments, an iRNA of the invention is a dsRNA of 24-30 nucleotides that interacts with a target RNA sequence, e.g., an HMGB1 target mRNA sequence, to direct the cleavage of the target RNA.

As used herein, the term “nucleotide overhang” refers to at least one unpaired nucleotide that protrudes from the duplex structure of a double stranded iRNA. For example, when a 3′-end of one strand of a dsRNA extends beyond the 5′-end of the other strand, or vice versa, there is a nucleotide overhang. A dsRNA can comprise an overhang of at least one nucleotide. Alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of either an antisense or sense strand of a dsRNA.

In certain embodiments, the antisense strand of a dsRNA has a 1-10 nucleotide, e.g., a 1, 2, 3, 4, 5, 6, 7, 8, 9, or 10 nucleotide overhang at the 3′-end or the 5′-end. In certain embodiments, the overhang on the sense strand or the antisense strand, or both, can include extended lengths longer than 10 nucleotides, e.g., 1-30 nucleotides, 2-30 nucleotides, 10-30 nucleotides, 10-25 nucleotides, 10-20 nucleotides, or 10-15 nucleotides in length. In certain embodiments, an extended overhang is on the sense strand of the duplex. In certain embodiments, an extended overhang is present on the 3′end of the sense strand of the duplex. In certain embodiments, an extended overhang is present on the 5′end of the sense strand of the duplex. In certain embodiments, an extended overhang is on the antisense strand of the duplex. In certain embodiments, an extended overhang is present on the 3′end of the antisense strand of the duplex. In certain embodiments, an extended overhang is present on the 5′end of the antisense strand of the duplex. In certain embodiments, one or more of the nucleotides in the extended overhang is replaced with a nucleoside thiophosphate. In certain embodiments, the overhang includes a self-complementary portion such that the overhang is capable of forming a hairpin structure that is stable under physiological conditions.

“Blunt” or “blunt end” means that there are no unpaired nucleotides at that end of the double stranded RNA agent, i.e., no nucleotide overhang. A “blunt ended” double stranded RNA agent is double stranded over its entire length, i.e., no nucleotide overhang at either end of the molecule. The RNAi agents of the invention include RNAi agents with no nucleotide overhang at one end (i.e., agents with one overhang and one blunt end) or with no nucleotide overhangs at either end. Most often such a molecule will be double-stranded over its entire length.

The term “antisense strand” or “guide strand” refers to the strand of an iRNA, e.g., a dsRNA, which includes a region that is substantially complementary to a target sequence, e.g., an HMGB1 mRNA. As used herein, the term “region of complementarity” refers to the region on the antisense strand that is substantially complementary to a sequence, for example a target sequence, e.g., an HMGB1 nucleotide sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, or 3 nucleotides of the 5′- or 3′-end of the iRNA. In some embodiments, a double stranded RNA agent of the invention includes a nucleotide mismatch in the antisense strand. In some embodiments, a double stranded RNA agent of the invention includes a nucleotide mismatch in the sense strand. In some embodiments, the nucleotide mismatch is, for example, within 5, 4, 3 nucleotides from the 3′-end of the iRNA. In another embodiment, the nucleotide mismatch is, for example, in the 3′-terminal nucleotide of the iRNA.

The term “sense strand” or “passenger strand” as used herein, refers to the strand of an iRNA that includes a region that is substantially complementary to a region of the antisense strand as that term is defined herein.

As used herein, “substantially all of the nucleotides are modified” are largely but not wholly modified and can include not more than 5, 4, 3, 2, or 1 unmodified nucleotides.

As used herein, the term “cleavage region” refers to a region that is located immediately adjacent to the cleavage site. The cleavage site is the site on the target at which cleavage occurs. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12, and 13.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3, or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs or base pairs formed from non-natural and modified nucleotides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of a double stranded RNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding an HMGB1 gene). For example, a polynucleotide is complementary to at least a part of an HMGB1 mRNA if the sequence is substantially complementary to a non-interrupted portion of an mRNA encoding an HMGB1 gene.

Accordingly, in some embodiments, the sense strand polynucleotides disclosed herein are fully complementary to the target HMGB1 sequence. In some embodiments, the antisense polynucleotides disclosed herein are fully complementary to the target HMGB1 sequence. In other embodiments, the sense strand polynucleotides or the antisense polynucleotides disclosed herein are substantially complementary to the reverse complement of the target HMGB1 sequence and comprise a contiguous nucleotide sequence which is at least 80% complementary over its entire length to the equivalent region of the nucleotide sequence of SEQ ID NO:2, or a fragment of SEQ ID NO:2, such as at least 85%, 90%, or 95% complementary; or 100% complementary. In some embodiments, the antisense polynucleotides disclosed herein are substantially complementary to the target HMGB1 sequence and comprise a contiguous nucleotide sequence which is at least 80% complementary over its entire length to the equivalent region of the nucleotide sequence of SEQ ID NO:1, or a fragment of SEQ ID NO:1, such as at least 85%, 90%, or 95% complementary; or 100% complementary. Target sites can be defined by identification of a target site on the target HMGB1 sequence, e.g., SEQ ID NO: 1. A corresponding portion of SEQ ID NO: 2, the reverse complement of SEQ ID NO: 1, is a portion of SEQ ID NO: 2 that would be complementary to the portion of SEQ ID NO: 1 over a sufficient length to provide a double stranded RNAi as disclosed herein.

Accordingly, in some embodiments, the antisense strand polynucleotides disclosed herein are fully complementary to the target HMGB1 sequence. In other embodiments, the antisense strand polynucleotides disclosed herein are substantially complementary to the target HMGB1 sequence and comprise a contiguous nucleotide sequence which is at least about 80% complementary over its entire length to the equivalent region of the nucleotide sequence of SEQ ID NO:1, or a fragment of SEQ ID NO:1, such as about 85%, about 90%, or about 95%, complementary. In certain embodiments, the fragment of SEQ ID NO: 1 is selected from the group of nucleotides 830-851, 830-850, 831-851, 944-997, 944-990, 944-964, 968-997, 968-990, 968-995, 968-988, 969-989, 970-990, 971-991, 972-992, 972-995, 973-993, 974-994, 975-995, 976-996, 977-997, 1019-1039, 1158-1194, 1158-1182, 1158-1178, 1159-1179, 1160-1180, 1161-1181, 1162-1182, or 1174-1194 of SEQ ID NO: 1.

In some embodiments, an iRNA of the invention includes an antisense strand that is substantially complementary to the target HMGB1 sequence and comprises a contiguous nucleotide sequence which is at least about 80% complementary over its entire length to the equivalent region of the nucleotide sequence of any one of the sense strands in any one of Tables 3, 5, 6, or 7, or a fragment of any one of the sense strands in any one of Tables 3, 5, 6, or 7, such as about 85%, 90%, 95%, or 100% complementary.

In some embodiments, an iRNA of the invention includes a sense strand that is substantially complementary to an antisense polynucleotide which, in turn, is complementary to a target HMGB1 sequence, and wherein the sense strand polynucleotide comprises a contiguous nucleotide sequence which is at least about 80% complementary over its entire length to the equivalent region of the nucleotide sequence of any one of the antisense strands in any one of Tables 3, 5, 6, or 7, or a fragment of any one of the antisense strands in any one of Tables 3, 5, 6, or 7, such as about a 85%, 90%, 95%, or 100%.

In certain embodiments, the sense and antisense strands in any one of Tables 3, 5, 6, or 7 are selected from duplexes AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-193181, AD-80651, or AD-80652.

In general, an “iRNA” includes ribonucleotides with chemical modifications. Such modifications may include all types of modifications disclosed herein or known in the art. Any such modifications, as used in a dsRNA molecule, are encompassed by “iRNA” for the purposes of this specification and claims.

In an aspect of the invention, an agent for use in the methods and compositions of the invention is a single-stranded antisense oligonucleotide molecule that inhibits a target mRNA via an antisense inhibition mechanism. The single-stranded antisense oligonucleotide molecule is complementary to a sequence within the target mRNA. The single-stranded antisense oligonucleotides can inhibit translation in a stoichiometric manner by base pairing to the mRNA and physically obstructing the translation machinery, see Dias, N. et al., (2002) Mol Cancer Ther 1:347-355. The single-stranded antisense oligonucleotide molecule may be about 14 to about 30 nucleotides in length and have a sequence that is complementary to a target sequence. For example, the single-stranded antisense oligonucleotide molecule may comprise a sequence that is at least about 14, 15, 16, 17, 18, 19, 20, or more contiguous nucleotides from any one of the antisense sequences described herein.

The phrase “contacting a cell with an iRNA,” such as a dsRNA, as used herein, includes contacting a cell by any possible means. Contacting a cell with an iRNA includes contacting a cell in vitro with the iRNA or contacting a cell in vivo with the iRNA. The contacting may be done directly or indirectly. Thus, for example, the iRNA may be put into physical contact with the cell by the individual performing the method, or alternatively, the iRNA may be put into a situation that will permit or cause it to subsequently come into contact with the cell.

Contacting a cell in vitro may be done, for example, by incubating the cell with the iRNA. Contacting a cell in vivo may be done, for example, by injecting the iRNA into or near the tissue where the cell is located, or by injecting the iRNA into another area, e.g., the bloodstream or the subcutaneous space, such that the agent will subsequently reach the tissue where the cell to be contacted is located. For example, the iRNA may contain or be coupled to a ligand, e.g., GalNAc, that directs the iRNA to a site of interest, e.g., the liver. Combinations of in vitro and in vivo methods of contacting are also possible. For example, a cell may also be contacted in vitro with an iRNA and subsequently transplanted into a subject.

In certain embodiments, contacting a cell with an iRNA includes “introducing” or “delivering the iRNA into the cell” by facilitating or effecting uptake or absorption into the cell. Absorption or uptake of an iRNA can occur through unaided diffusion or active cellular processes, or by auxiliary agents or devices. Introducing an iRNA into a cell may be in vitro or in vivo. For example, for in vivo introduction, iRNA can be injected into a tissue site or administered systemically. In vitro introduction into a cell includes methods known in the art such as electroporation and lipofection. Further approaches are described herein below or are known in the art.

The term “lipid nanoparticle” or “LNP” is a vesicle comprising a lipid layer encapsulating a pharmaceutically active molecule, such as a nucleic acid molecule, e.g., an iRNA or a plasmid from which an iRNA is transcribed. LNPs are described in, for example, U.S. Pat. Nos. 6,858,225, 6,815,432, 8,158,601, and 8,058,069, the entire contents of which are hereby incorporated herein by reference.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird that expresses the target gene, either endogenously or heterologously. In certain embodiments, the subject is an animal that is diagnosed with or at risk of developing an HMGB1-associated disorder. In certain embodiments, the subject is an animal that is diagnosed with NASH or at risk of developing NASH, e.g. a subject at risk of developing or diagnosed with NAFLD or metabolic disorder. In certain embodiments, the subject is a subject meeting at least one diagnostic criterion for metabolic disorder, e.g., blood pressure equal to or higher than 130/85 mmHg, large waist circumference (40 inches or more for men and 35 inches or more for women); waist-to-hip ratio <1.0 (for men) or <0.8 (for women); low HDL cholesterol (under 40 mg/dL for men and under 50 mg/dL for women), or triglycerides of at least 150 mg/dL. In certain embodiments, the subject has one or more of Hb1Ac of at least 6.5%, type 2 diabetes mellitus, elevated fasting blood glucose of at least 100 mg/dL, 2 hour postprandial blood glucose or serum glucose concentration of at least 140 mg/dl. In certain embodiments, the subject is a subject meeting at least one diagnostic criteria for a NAFLD, e.g., for NAFL, steatohepatitis, NASH, NASH cirrhosis, or cryptogenic cirrhosis. In certain embodiments, the subject is a subject meeting at least one diagnositic criteria for NASH. In certain embodiments, the subject has been diagnosed with at least one of hepatic inflammation, cirrhosis, autoimmune hepatitis, chronic alcohol consumption optionally in association with one or more of fatty liver and elevated serum lipids or cholesterol; hemochromatosis, or has or had chronically used of pharmaceutical agents that cause liver damage. The diagnostic criteria for metabolic disorder and NAFLD, e.g., NASH are provided below. In certain embodiments, the subject at risk of developing NASH has been diagnosed with one of hypothyroidism, obstructive sleep apnea, hypopituitarism, hypogonadism, pancreatoduodenal resection, and psoriasis. It is understood that the diagnostic criteria for metabolic syndrome and various NAFLD are overlapping. In certain embodiments, the subject meets the full diagnositic criteria for metabolic disorder or a NAFLD, e.g., NASH. In certain embodiments, the subject has an HMGB1-associated disorder associated with a chronic insult. In some embodiments, the subject is a female human. In other embodiments, the subject is a male human.

As used herein, the terms “treating” or “treatment” refer to a beneficial or desired result, such as reducing at least one sign or symptom of an HMGB1-associated disorder, e.g., metabolic disorder or a NAFLD, e.g., NASH in a subject. “Treatment” can also mean prolonging survival as compared to expected survival in the absence of treatment.

The term “lower” in the context of the level of an HMGB1 gene expression or an HMGB1 protein production in a subject, or a disease marker or symptom refers to a statistically significant decrease in such level. The decrease can be, for example, at least 30%, 35%, 40%, 45%, 50%, 55%, 60%, 65%, 70%, 75%, 80%, 85%, 90%, or 95%, or below the level of detection for the detection method in a relevant cell or tissue, e.g., a liver cell, or other subject sample, e.g., urine, blood or serum derived therefrom. Lowering does not require systemic assessment of protein or RNA level. Lowering may be limited to a tissue, e.g., liver.

As used herein, “prevention” or “preventing,” when used in reference to a disease, disorder, or condition thereof, that would benefit from a reduction in expression of an HMGB1 gene or production of an HMGB1 protein, e.g., a subject at risk of developing an HMGB associated disorder, e.g., metabolic disorder, NAFLD, or NASH. In certain embodiments, a subject at risk of developing NASH has been diagnosed with NAFLD, hypothyroidism, obstructive sleep apnea, hypopituitarism, hypogonadism, pancreatoduodenal resection, or psoriasis. The failure to develop metabolic disorder or a NAFLD, e.g., NASH, or progression to a more severe NAFLD, e.g., from NAFL to NASH, by months or years is considered effective prevention. Prevention may require administration of more than one dose if the iRNA agent.

A “therapeutically-effective amount” or “prophylactically effective amount” also includes an amount of an RNAi agent that produces some desired local or systemic effect at a reasonable benefit/risk ratio applicable to any treatment. The iRNA employed in the methods of the present invention may be administered in a sufficient amount to produce a reasonable benefit/risk ratio applicable to such treatment.

The phrase “pharmaceutically acceptable” is employed herein to refer to those compounds, materials, compositions, or dosage forms which are, within the scope of sound medical judgment, suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated. Pharmaceutically acceptable carriers include carriers for administration by injection. Pharmaceutically acceptable carriers include carriers for administration by inhalation. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium state, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates or polyanhydrides; (22) bulking agents, such as polypeptides and amino acids (23) serum component, such as serum albumin, HDL and LDL; and (22) other non-toxic compatible substances employed in pharmaceutical formulations.

The term “sample,” as used herein, includes a collection of similar fluids, cells, or tissues isolated from a subject, as well as fluids, cells, or tissues present within a subject. Examples of biological fluids include blood, serum and serosal fluids, plasma, cerebrospinal fluid, ocular fluids, lymph, urine, saliva, and the like. Tissue samples may include samples from tissues, organs, or localized regions. For example, samples may be derived from particular organs, parts of organs, or fluids or cells within those organs. In certain embodiments, samples may be derived from the liver (e.g., whole liver or certain segments of liver or certain types of cells in the liver, such as, e.g., hepatocytes). In some embodiments, a “sample derived from a subject” refers to urine obtained from the subject. A “sample derived from a subject” can refer to urine, to blood, or blood derived serum or plasma from the subject.

I. iRNAs of the Invention

The present invention provides iRNAs which inhibit the expression of an HMGB1 gene. In preferred embodiments, the iRNA includes double stranded ribonucleic acid (dsRNA) molecules for inhibiting the expression of an HMGB1 gene in a cell, such as a cell within a subject, e.g., a mammal, such as a human at risk of developing an HMGB1-associated disorder, e.g., metabolic disorder, NAFLD, or NASH. The dsRNAi agent includes an antisense strand having a region of complementarity which is complementary to at least a part of an mRNA formed in the expression of an HMGB1 gene. The region of complementarity is about 30 nucleotides or less in length (e.g., about 30, 29, 28, 27, 26, 25, 24, 23, 22, 21, 20, 19, or 18 nucleotides or less in length). Upon contact with a cell expressing the HMGB1 gene, the iRNA inhibits the expression of the HMGB1 gene (e.g., a human, a primate, a non-primate, or a bird HMGB1 gene) by at least about 30%, preferably at least about 50% as assayed by, for example, a PCR or branched DNA (bDNA)-based method, or by a protein-based method, such as by immunofluorescence analysis, using, for example, western blotting or flow cytometric techniques. In preferred embodiments, inhibition of expression is determined by the qPCR method provided in the examples, especially in Example 2 with the siRNA at a 10 nM concentration in an appropriate organism cell line provided therein. In certain embodiments, inhibition of expression in vivo is determined by knockdown of the human gene in a rodent expressing the human gene, e.g., a mouse or an AAV-infected mouse expressing the human target gene, e.g., when administered a single dose at 3 mg/kg at the nadir of RNA expression. In certain embodiments, inhibition of expression is determined in an appropriate disease model in which the target gene is elevated in response to the disease indication, e.g., a mouse when administered a single dose at 3 mg/kg at the nadir of RNA expression. RNA expression in liver is determined using the PCR methods provided in Example 2. In certain embodiments, RNA expression is determined using a liver sample. In certain embodiments, RNA expression is determined in vesicle associated RNA from a blood or urine sample using methods known in the art as provided in e.g., Sehgal et al., RNA 20:143-149, 2014; Chan et al., Mol. Ther. Nucl. Acids. 4:e263, 2015.

A dsRNA includes two RNA strands that are complementary and hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of an HMGB1 gene. The other strand (the sense strand) includes a region that is complementary to the antisense strand, such that the two strands hybridize and form a duplex structure when combined under suitable conditions. As described elsewhere herein and as known in the art, the complementary sequences of a dsRNA can also be contained as self-complementary regions of a single nucleic acid molecule, as opposed to being on separate oligonucleotides.

Generally, the duplex structure is 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length. In certain embodiments, the duplex length is 19-21 base pairs in length. Ranges and lengths intermediate to the above recited ranges and lengths are also contemplated to be part of the invention.

Similarly, the region of complementarity to the target sequence is 15 to 30 nucleotides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length. In certain embodiments, the region of complementarity is 19-21 nucleotides in length. Ranges and lengths intermediate to the above recited ranges and lengths are also contemplated to be part of the invention.

In some embodiments, the dsRNA is about 15 to about 23 nucleotides in length, or about 25 to about 30 nucleotides in length. In general, the dsRNA is long enough to serve as a substrate for the Dicer enzyme. For example, it is well-known in the art that dsRNAs longer than about 21-23 nucleotides in length may serve as substrates for Dicer. As the ordinarily skilled person will also recognize, the region of an RNA targeted for cleavage will most often be part of a larger RNA molecule, often an mRNA molecule. Where relevant, a “part” of an mRNA target is a contiguous sequence of an mRNA target of sufficient length to allow it to be a substrate for RNAi-directed cleavage (i.e., cleavage through a RISC pathway).

One of skill in the art will also recognize that the duplex region is a primary functional portion of a dsRNA, e.g., a duplex region of about 9 to about 36 base pairs, e.g., about 10-36, 11-36, 12-36, 13-36, 14-36, 15-36, 9-35, 10-35, 11-35, 12-35, 13-35, 14-35, 15-35, 9-34, 10-34, 11-34, 12-34, 13-34, 14-34, 15-34, 9-33, 10-33, 11-33, 12-33, 13-33, 14-33, 15-33, 9-32, 10-32, 11-32, 12-32, 13-32, 14-32, 15-32, 9-31, 10-31, 11-31, 12-31, 13-32, 14-31, 15-31, 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs. Thus, in one embodiment, to the extent that it becomes processed to a functional duplex, of e.g., 15-30 base pairs, that targets a desired RNA for cleavage, an RNA molecule or complex of RNA molecules having a duplex region greater than 30 base pairs is a dsRNA. Thus, an ordinarily skilled artisan will recognize that in one embodiment, a miRNA is a dsRNA. In another embodiment, a dsRNA is not a naturally occurring miRNA. In another embodiment, an iRNA agent useful to target HMGB1 gene expression is not generated in the target cell by cleavage of a larger dsRNA.

A dsRNA as described herein can further include one or more single-stranded nucleotide overhangs e.g., 1-4, 2-4, 1-3, 2-3, 1, 2, 3, or 4 nucleotides. dsRNAs having at least one nucleotide overhang can have superior inhibitory properties relative to their blunt-ended counterparts. A nucleotide overhang can comprise or consist of a nucleotide/nucleoside analog, including a deoxynucleotide/nucleoside. The overhang(s) can be on the sense strand, the antisense strand, or any combination thereof. Furthermore, the nucleotide(s) of an overhang can be present on the 5′-end, 3′-end, or both ends of an antisense or sense strand of a dsRNA.

A dsRNA can be synthesized by standard methods known in the art as further discussed below, e.g., by use of an automated DNA synthesizer, such as are commercially available from, for example, Biosearch, Applied Biosystems™, Inc.

Double stranded RNAi compounds of the invention may be prepared using a two-step procedure. First, the individual strands of the double stranded RNA molecule are prepared separately. Then, the component strands are annealed. The individual strands of the siRNA compound can be prepared using solution-phase or solid-phase organic synthesis or both. Organic synthesis offers the advantage that the oligonucleotide strands comprising unnatural or modified nucleotides can be easily prepared. Similarly, single-stranded oligonucleotides of the invention can be prepared using solution-phase or solid-phase organic synthesis or both.

In an aspect, a dsRNA of the invention includes at least two nucleotide sequences, a sense sequence and an anti-sense sequence. The sense strand is selected from the group of sequences provided in Tables 3 and 5, and the corresponding antisense strand of the sense strand is selected from the group of sequences of any one of Tables 3, 5, 6, or 7. In this aspect, one of the two sequences is complementary to the other of the two sequences, with one of the sequences being substantially complementary to a sequence of an mRNA generated in the expression of an HMGB1 gene. As such, in this aspect, a dsRNA will include two oligonucleotides, where one oligonucleotide is described as the sense strand in any one of Tables 3, 5, 6, or 7, and the second oligonucleotide is described as the corresponding antisense strand of the sense strand in any one of Tables 3, 5, 6, or 7. In certain embodiments, the substantially complementary sequences of the dsRNA are contained on separate oligonucleotides. In other embodiments, the substantially complementary sequences of the dsRNA are contained on a single oligonucleotide. In certain embodiments, the sense or antisense strand from any one of Tables 3, 5, 6, or 7 is selected from a sense or antisense strand of AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-193181, AD-80651, or AD-80652.

It will be understood that, although the sequences in Table 3 are not described as modified or conjugated sequences, the RNA of the iRNA of the invention e.g., a dsRNA of the invention, may comprise any one of the sequences set forth in Table 3, or the sequences of any one of Tables 5, 6, or 7 that are modified, or the sequences of any one of Tables 5, 6, or 7 that are conjugated. In other words, the invention encompasses dsRNA of any one of Tables 3, 5, 6, or 7 which are un-modified, un-conjugated, modified, or conjugated, as described herein.

The skilled person is well aware that dsRNAs having a duplex structure of about 20 to 23 base pairs, e.g., 21, base pairs have been hailed as particularly effective in inducing RNA interference (Elbashir et al., EMBO 2001, 20:6877-6888). However, others have found that shorter or longer RNA duplex structures can also be effective (Chu and Rana (2007) RNA 14:1714-1719; Kim et al. (2005) Nat Biotech 23:222-226). In the embodiments described above, by virtue of the nature of the oligonucleotide sequences provided in any one of any one of Tables 3, 5, 6, or 7, dsRNAs described herein can include at least one strand of a length of minimally 21 nucleotides. It can be reasonably expected that shorter duplexes having one of the sequences of any one of Tables 3, 5, 6, or 7 minus only a few nucleotides on one or both ends can be similarly effective as compared to the dsRNAs described above. Hence, dsRNAs having a sequence of at least 15, 16, 17, 18, 19, 20, or more contiguous nucleotides derived from one of the sequences of any one of Tables 3, 5, 6, or 7, and differing in their ability to inhibit the expression of an HMGB1 gene by not more than about 5, 10, 15, 20, 25, or 30% inhibition from a dsRNA comprising the full sequence, are contemplated to be within the scope of the present invention.

In addition, the RNAs provided in any one of Tables 3, 5, 6, or 7 identify a site(s) in an HMGB1 transcript that is susceptible to RISC-mediated cleavage. As such, the present invention further features iRNAs that target within one of these sites. As used herein, an iRNA is said to target within a particular site of an RNA transcript if the iRNA promotes cleavage of the transcript anywhere within that particular site. Such an iRNA will generally include at least about 15 contiguous nucleotides from one of the sequences provided in any one of Tables 3, 5, 6, or 7 coupled to additional nucleotide sequences taken from the region contiguous to the selected sequence in an HMGB1 gene.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch is not located in the center of the region of complementarity. If the antisense strand of the iRNA contains mismatches to the target sequence, it is preferable that the mismatch be restricted to be within the last 5 nucleotides from either the 5′- or 3′-end of the region of complementarity. For example, for a 23 nucleotide iRNA agent the strand which is complementary to a region of an HMGB1 gene, generally does not contain any mismatch within the central 13 nucleotides. The methods described herein or methods known in the art can be used to determine whether an iRNA containing a mismatch to a target sequence is effective in inhibiting the expression of an HMGB1 gene. Consideration of the efficacy of iRNAs with mismatches in inhibiting expression of an HMGB1 gene is important, especially if the particular region of complementarity in an HMGB1 gene is known to have polymorphic sequence variation within the population.

II. Modified iRNAs of the Invention

In certain embodiments, the RNA of the iRNA of the invention e.g., a dsRNA, is un-modified, and does not comprise, e.g., chemical modifications or conjugations known in the art and described herein. In other embodiments, the RNA of an iRNA of the invention, e.g., a dsRNA, is chemically modified to enhance stability or other beneficial characteristics. In certain embodiments of the invention, substantially all of the nucleotides of an iRNA of the invention are modified. In other embodiments of the invention, all of the nucleotides of an iRNA or substantially all of the nucleotides of an iRNA are modified, i.e., not more than 5, 4, 3, 2, or lunmodified nucleotides are present in a strand of the iRNA.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages. Specific examples of iRNA compounds useful in the embodiments described herein include, but are not limited to, RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments, a modified iRNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments of the invention, the dsRNA agents of the invention are in a free acid form. In other embodiments of the invention, the dsRNA agents of the invention are in a salt form. In one embodiment, the dsRNA agents of the invention are in a sodium salt form. In certain embodiments, when the dsRNA agents of the invention are in the sodium salt form, sodium ions are present in the agent as counterions for substantially all of the phosphodiester or phosphorothiotate groups present in the agent. Agents in which substantially all of the phosphodiester or phosphorothioate linkages have a sodium counterion include not more than 5, 4, 3, 2, or 1 phosphodiester or phosphorothioate linkages without a sodium counterion. In some embodiments, when the dsRNA agents of the invention are in the sodium salt form, sodium ions are present in the agent as counterions for all of the phosphodiester or phosphorothiotate groups present in the agent.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39,464, the entire contents of each of which are hereby incorporated herein by reference.

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

Suitable RNA mimetics are contemplated for use in iRNAs provided herein, in which both the sugar and the internucleoside linkage, i.e., the backbone, of the nucleotide units are replaced with novel groups. The base units are maintained for hybridization with an appropriate nucleic acid target compound. One such oligomeric compound in which an RNA mimetic that has been shown to have excellent hybridization properties is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative US patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the iRNAs of the invention are described in, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂. Further exemplary modifications include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs can also have sugar mimetics such as cyclobutyl moieties in place of the pentofuranosyl sugar. Representative US patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application. The entire contents of each of the foregoing are hereby incorporated herein by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C), and uracil (U). Modified nucleobases include other synthetic and natural nucleobases such as deoxy-thymine (dT), 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

In some embodiments, the RNA of an iRNA can also be modified to include one or more bicyclic sugar moieties. A “bicyclic sugar” is a furanosyl ring modified by the bridging of two atoms. A “bicyclic nucleoside” (“BNA”) is a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleic acids (LNA). A locked nucleic acid is a nucleotide having a modified ribose moiety in which the ribose moiety comprises an extra bridge connecting the 2′ and 4′ carbons. In other words, an LNA is a nucleotide comprising a bicyclic sugar moiety comprising a 4′-CH₂—O-2′ bridge. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Examples of bicyclic nucleosides for use in the polynucleotides of the invention include without limitation nucleosides comprising a bridge between the 4′ and the 2′ ribosyl ring atoms. In certain embodiments, the antisense polynucleotide agents of the invention include one or more bicyclic nucleosides comprising a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and U.S. patent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and 3-D-ribofuranose (see WO 99/14226).

The RNA of an iRNA can also be modified to include one or more constrained ethyl nucleotides. As used herein, a “constrained ethyl nucleotide” or “cEt” is a locked nucleic acid comprising a bicyclic sugar moiety comprising a 4′-CH(CH₃)—O-2′ bridge. In one embodiment, a constrained ethyl nucleotide is in the S conformation referred to herein as “S-cEt.”

An iRNA of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, U.S. Patent Publication No. 2013/0190383; and PCT publication WO 2013/036868, the entire contents of each of which are hereby incorporated herein by reference.

In some embodiments, an iRNA of the invention comprises one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. patent publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference.

Potentially stabilizing modifications to the ends of RNA molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other modifications of the nucleotides of an iRNA of the invention include a 5′ phosphate or 5′ phosphate mimic, e.g., a 5′-terminal phosphate or phosphate mimic on the antisense strand of an iRNA. Suitable phosphate mimics are disclosed in, for example U.S. Patent Publication No. 2012/0157511, the entire contents of which are incorporated herein by reference.

A. Modified iRNAs Comprising Motifs of the Invention

In certain aspects of the invention, the double stranded RNA agents of the invention include agents with chemical modifications as disclosed, for example, in WO2013/075035, the entire contents of each of which are incorporated herein by reference. WO2013/075035 provides motifs of three identical modifications on three consecutive nucleotides into a sense strand or antisense strand of a dsRNAi agent, particularly at or near the cleavage site. In some embodiments, the sense strand and antisense strand of the dsRNAi agent may otherwise be completely modified. The introduction of these motifs interrupts the modification pattern, if present, of the sense or antisense strand. The dsRNAi agent may be optionally conjugated with a GalNAc derivative ligand, for instance on the sense strand.

More specifically, when the sense strand and antisense strand of the double stranded RNA agent are completely modified to have one or more motifs of three identical modifications on three consecutive nucleotides at or near the cleavage site of at least one strand of a dsRNAi agent, the gene silencing activity of the dsRNAi agent was observed.

Accordingly, the invention provides double stranded RNA agents capable of inhibiting the expression of a target gene (i.e., HMGB1 gene) in vivo. The RNAi agent comprises a sense strand and an antisense strand. Each strand of the RNAi agent may be, independently, 12-30 nucleotides in length. For example, each strand may independently be 14-30 nucleotides in length, 17-30 nucleotides in length, 25-30 nucleotides in length, 27-30 nucleotides in length, 17-23 nucleotides in length, 17-21 nucleotides in length, 17-19 nucleotides in length, 19-25 nucleotides in length, 19-23 nucleotides in length, 19-21 nucleotides in length, 21-25 nucleotides in length, or 21-23 nucleotides in length.

The sense strand and antisense strand typically form a duplex double stranded RNA (“dsRNA”), also referred to herein as “dsRNAi agent.” The duplex region of a dsRNAi agent may be 12-30 nucleotide pairs in length. For example, the duplex region can be 14-30 nucleotide pairs in length, 17-30 nucleotide pairs in length, 27-30 nucleotide pairs in length, 17-23 nucleotide pairs in length, 17-21 nucleotide pairs in length, 17-19 nucleotide pairs in length, 19-25 nucleotide pairs in length, 19-23 nucleotide pairs in length, 19-21 nucleotide pairs in length, 21-25 nucleotide pairs in length, or 21-23 nucleotide pairs in length. In another example, the duplex region is selected from 15, 16, 17, 18, 19, 20, 21, 22, 23, 24, 25, 26, and 27 nucleotides in length.

In certain embodiments, the dsRNAi agent may contain one or more overhang regions or capping groups at the 3′-end, 5′-end, or both ends of one or both strands. The overhang can be, independently, 1-6 nucleotides in length, for instance 2-6 nucleotides in length, 1-5 nucleotides in length, 2-5 nucleotides in length, 1-4 nucleotides in length, 2-4 nucleotides in length, 1-3 nucleotides in length, 2-3 nucleotides in length, or 1-2 nucleotides in length. In certain embodiments, the overhang regions can include extended overhang regions as provided above. The overhangs can be the result of one strand being longer than the other, or the result of two strands of the same length being staggered. The overhang can form a mismatch with the target mRNA or it can be complementary to the gene sequences being targeted or can be another sequence. The first and second strands can also be joined, e.g., by additional bases to form a hairpin, or by other non-base linkers.

In certain embodiments, the nucleotides in the overhang region of the dsRNAi agent can each independently be a modified or unmodified nucleotide including, but no limited to 2′-sugar modified, such as, 2′-F, 2′-O-methyl, thymidine (T), 2′-O-methoxyethyl-5-methyluridine (Teo), 2′-O-methoxyethyladenosine (Aeo), 2′-O-methoxyethyl-5-methylcytidine (m5Ceo), and any combinations thereof. For example, TT can be an overhang sequence for either end on either strand. The overhang can form a mismatch with the target mRNA or it can be complementary to the gene sequences being targeted or can be another sequence.

The 5′- or 3′-overhangs at the sense strand, antisense strand, or both strands of the dsRNAi agent may be phosphorylated. In some embodiments, the overhang region(s) contains two nucleotides having a phosphorothioate between the two nucleotides, where the two nucleotides can be the same or different. In some embodiments, the overhang is present at the 3′-end of the sense strand, antisense strand, or both strands. In some embodiments, this 3′-overhang is present in the antisense strand. In some embodiments, this 3′-overhang is present in the sense strand.

The dsRNAi agent may contain only a single overhang, which can strengthen the interference activity of the RNAi, without affecting its overall stability. For example, the single-stranded overhang may be located at the 3′-end of the sense strand or, alternatively, at the 3′-end of the antisense strand. The RNAi may also have a blunt end, located at the 5′-end of the antisense strand (or the 3′-end of the sense strand) or vice versa. Generally, the antisense strand of the dsRNAi agent has a nucleotide overhang at the 3′-end, and the 5′-end is blunt. While not wishing to be bound by theory, the asymmetric blunt end at the 5′-end of the antisense strand and 3′-end overhang of the antisense strand favor the guide strand loading into RISC process.

In certain embodiments, the dsRNAi agent is a double ended bluntmer of 19 nucleotides in length, wherein the sense strand contains at least one motif of three 2′-F modifications on three consecutive nucleotides at positions 7, 8, 9 from the 5′end. The antisense strand contains at least one motif of three 2′-O-methyl modifications on three consecutive nucleotides at positions 11, 12, 13 from the 5′end.

In other embodiments, the dsRNAi agent is a double ended bluntmer of 20 nucleotides in length, wherein the sense strand contains at least one motif of three 2′-F modifications on three consecutive nucleotides at positions 8, 9, 10 from the 5′end. The antisense strand contains at least one motif of three 2′-O-methyl modifications on three consecutive nucleotides at positions 11, 12, 13 from the 5′end.

In yet other embodiments, the dsRNAi agent is a double ended bluntmer of 21 nucleotides in length, wherein the sense strand contains at least one motif of three 2′-F modifications on three consecutive nucleotides at positions 9, 10, 11 from the 5′end. The antisense strand contains at least one motif of three 2′-O-methyl modifications on three consecutive nucleotides at positions 11, 12, 13 from the 5′end.

In certain embodiments, the dsRNAi agent comprises a 21 nucleotide sense strand and a 23 nucleotide antisense strand, wherein the sense strand contains at least one motif of three 2′-F modifications on three consecutive nucleotides at positions 9, 10, 11 from the 5′end; the antisense strand contains at least one motif of three 2′-O-methyl modifications on three consecutive nucleotides at positions 11, 12, 13 from the 5′end, wherein one end of the RNAi agent is blunt, while the other end comprises a 2 nucleotide overhang. Preferably, the 2 nucleotide overhang is at the 3′-end of the antisense strand.

When the 2 nucleotide overhang is at the 3′-end of the antisense strand, there may be two phosphorothioate internucleotide linkages between the terminal three nucleotides, wherein two of the three nucleotides are the overhang nucleotides, and the third nucleotide is a paired nucleotide next to the overhang nucleotide. In one embodiment, the RNAi agent additionally has two phosphorothioate internucleotide linkages between the terminal three nucleotides at both the 5′-end of the sense strand and at the 5′-end of the antisense strand. In certain embodiments, every nucleotide in the sense strand and the antisense strand of the dsRNAi agent, including the nucleotides that are part of the motifs are modified nucleotides. In certain embodiments each residue is independently modified with a 2′-O-methyl or 3′-fluoro, e.g., in an alternating motif. Optionally, the dsRNAi agent further comprises a ligand (preferably GalNAc₃).

In certain embodiments, the dsRNAi agent comprises a sense and an antisense strand, wherein the sense strand is 25-30 nucleotide residues in length, wherein starting from the 5′ terminal nucleotide (position 1) positions 1 to 23 of the first strand comprise at least 8 ribonucleotides; the antisense strand is 36-66 nucleotide residues in length and, starting from the 3′ terminal nucleotide, comprises at least 8 ribonucleotides in the positions paired with positions 1-23 of sense strand to form a duplex; wherein at least the 3′ terminal nucleotide of antisense strand is unpaired with sense strand, and up to 6 consecutive 3′ terminal nucleotides are unpaired with sense strand, thereby forming a 3′ single stranded overhang of 1-6 nucleotides; wherein the 5′ terminus of antisense strand comprises from 10-30 consecutive nucleotides which are unpaired with sense strand, thereby forming a 10-30 nucleotide single stranded 5′ overhang; wherein at least the sense strand 5′ terminal and 3′ terminal nucleotides are base paired with nucleotides of antisense strand when sense and antisense strands are aligned for maximum complementarity, thereby forming a substantially duplexed region between sense and antisense strands; and antisense strand is sufficiently complementary to a target RNA along at least 19 ribonucleotides of antisense strand length to reduce target gene expression when the double stranded nucleic acid is introduced into a mammalian cell; and wherein the sense strand contains at least one motif of three 2′-F modifications on three consecutive nucleotides, where at least one of the motifs occurs at or near the cleavage site. The antisense strand contains at least one motif of three 2′-O-methyl modifications on three consecutive nucleotides at or near the cleavage site.

In certain embodiments, the dsRNAi agent comprises sense and antisense strands, wherein the dsRNAi agent comprises a first strand having a length which is at least 25 and at most 29 nucleotides and a second strand having a length which is at most 30 nucleotides with at least one motif of three 2′-O-methyl modifications on three consecutive nucleotides at position 11, 12, 13 from the 5′ end; wherein the 3′ end of the first strand and the 5′ end of the second strand form a blunt end and the second strand is 1-4 nucleotides longer at its 3′ end than the first strand, wherein the duplex region which is at least 25 nucleotides in length, and the second strand is sufficiently complementary to a target mRNA along at least 19 nucleotide of the second strand length to reduce target gene expression when the RNAi agent is introduced into a mammalian cell, and wherein Dicer cleavage of the dsRNAi agent preferentially results in an siRNA comprising the 3′-end of the second strand, thereby reducing expression of the target gene in the mammal. Optionally, the dsRNAi agent further comprises a ligand.

In certain embodiments, the sense strand of the dsRNAi agent contains at least one motif of three identical modifications on three consecutive nucleotides, where one of the motifs occurs at the cleavage site in the sense strand.

In certain embodiments, the antisense strand of the dsRNAi agent can also contain at least one motif of three identical modifications on three consecutive nucleotides, where one of the motifs occurs at or near the cleavage site in the antisense strand.

For a dsRNAi agent having a duplex region of 17-23 nucleotides in length, e.g., 17-23 basepairs in length, the cleavage site of the antisense strand is typically around the 10, 11, and 12 positions from the 5′-end. Thus the motifs of three identical modifications may occur at the 9, 10, 11 positions; the 10, 11, 12 positions; the 11, 12, 13 positions; the 12, 13, 14 positions; or the 13, 14, 15 positions of the antisense strand, the count starting from the first nucleotide from the 5′-end of the antisense strand, or, the count starting from the first paired nucleotide within the duplex region from the 5′-end of the antisense strand. The cleavage site in the antisense strand may also change according to the length of the duplex region of the dsRNAi agent from the 5′-end.

The sense strand of the dsRNAi agent may contain at least one motif of three identical modifications on three consecutive nucleotides at the cleavage site of the strand; and the antisense strand may have at least one motif of three identical modifications on three consecutive nucleotides at or near the cleavage site of the strand. When the sense strand and the antisense strand form a dsRNA duplex, the sense strand and the antisense strand can be so aligned that one motif of the three nucleotides on the sense strand and one motif of the three nucleotides on the antisense strand have at least one nucleotide overlap, i.e., at least one of the three nucleotides of the motif in the sense strand forms a base pair with at least one of the three nucleotides of the motif in the antisense strand. Alternatively, at least two nucleotides may overlap, or all three nucleotides may overlap.

In some embodiments, the sense strand of the dsRNAi agent may contain more than one motif of three identical modifications on three consecutive nucleotides. The first motif may occur at or near the cleavage site of the strand and the other motifs may be a wing modification. The term “wing modification” herein refers to a motif occurring at another portion of the strand that is separated from the motif at or near the cleavage site of the same strand. The wing modification is either adjacent to the first motif or is separated by at least one or more nucleotides. When the motifs are immediately adjacent to each other than the chemistries of the motifs are distinct from each other, and when the motifs are separated by one or more nucleotide than the chemistries can be the same or different. Two or more wing modifications may be present. For instance, when two wing modifications are present, each wing modification may occur at one end relative to the first motif which is at or near cleavage site or on either side of the lead motif.

Like the sense strand, the antisense strand of the dsRNAi agent may contain more than one motifs of three identical modifications on three consecutive nucleotides, with at least one of the motifs occurring at or near the cleavage site of the strand. This antisense strand may also contain one or more wing modifications in an alignment similar to the wing modifications that may be present on the sense strand.

In some embodiments, the wing modification on the sense strand or antisense strand of the dsRNAi agent typically does not include the first one or two terminal nucleotides at the 3′-end, 5′-end, or both ends of the strand.

In other embodiments, the wing modification on the sense strand or antisense strand of the dsRNAi agent typically does not include the first one or two paired nucleotides within the duplex region at the 3′-end, 5′-end, or both ends of the strand.

When the sense strand and the antisense strand of the dsRNAi agent each contain at least one wing modification, the wing modifications may fall on the same end of the duplex region, and have an overlap of one, two, or three nucleotides.

When the sense strand and the antisense strand of the dsRNAi agent each contain at least two wing modifications, the sense strand and the antisense strand can be so aligned that two modifications each from one strand fall on one end of the duplex region, having an overlap of one, two, or three nucleotides; two modifications each from one strand fall on the other end of the duplex region, having an overlap of one, two or three nucleotides; two modifications one strand fall on each side of the lead motif, having an overlap of one, two or three nucleotides in the duplex region.

In some embodiments, every nucleotide in the sense strand and antisense strand of the dsRNAi agent, including the nucleotides that are part of the motifs, may be modified. Each nucleotide may be modified with the same or different modification which can include one or more alteration of one or both of the non-linking phosphate oxygens or of one or more of the linking phosphate oxygens; alteration of a constituent of the ribose sugar, e.g., of the 2′-hydroxyl on the ribose sugar; wholesale replacement of the phosphate moiety with “dephospho” linkers; modification or replacement of a naturally occurring base; and replacement or modification of the ribose-phosphate backbone.

As nucleic acids are polymers of subunits, many of the modifications occur at a position which is repeated within a nucleic acid, e.g., a modification of a base, or a phosphate moiety, or a non-linking O of a phosphate moiety. In some cases the modification will occur at all of the subject positions in the nucleic acid but in many cases it will not. By way of example, a modification may only occur at a 3′- or 5′-terminal position, may only occur in a terminal region, e.g., at a position on a terminal nucleotide or in the last 2, 3, 4, 5, or 10 nucleotides of a strand. A modification may occur in a double strand region, a single strand region, or in both. A modification may occur only in the double strand region of an RNA or may only occur in a single strand region of an RNA. For example, a phosphorothioate modification at a non-linking O position may only occur at one or both termini, may only occur in a terminal region, e.g., at a position on a terminal nucleotide or in the last 2, 3, 4, 5, or 10 nucleotides of a strand, or may occur in double strand and single strand regions, particularly at termini. The 5′-end or ends can be phosphorylated.

It may be possible, e.g., to enhance stability, to include particular bases in overhangs, or to include modified nucleotides or nucleotide surrogates, in single strand overhangs, e.g., in a 5′- or 3′-overhang, or in both. For example, it can be desirable to include purine nucleotides in overhangs. In some embodiments all or some of the bases in a 3′- or 5′-overhang may be modified, e.g., with a modification described herein. Modifications can include, e.g., the use of modifications at the 2′ position of the ribose sugar with modifications that are known in the art, e.g., the use of deoxyribonucleotides, 2′-deoxy-2′-fluoro (2′-F) or 2′-O-methyl modified instead of the ribosugar of the nucleobase, and modifications in the phosphate group, e.g., phosphorothioate modifications. Overhangs need not be homologous with the target sequence.

In some embodiments, each residue of the sense strand and antisense strand is independently modified with LNA, CRN, cET, UNA, HNA, CeNA, 2′-methoxyethyl, 2′-O-methyl, 2′-O-allyl, 2′-C-allyl, 2′-deoxy, 2′-hydroxyl, or 2′-fluoro. The strands can contain more than one modification. In one embodiment, each residue of the sense strand and antisense strand is independently modified with 2′-O-methyl or 2′-fluoro.

At least two different modifications are typically present on the sense strand and antisense strand. Those two modifications may be the 2′-O-methyl or 2′-fluoro modifications, or others.

In certain embodiments, the Nₐ or N_{b} comprises modifications of an alternating pattern. The term “alternating motif” as used herein refers to a motif having one or more modifications, each modification occurring on alternating nucleotides of one strand. The alternating nucleotide may refer to one per every other nucleotide or one per every three nucleotides, or a similar pattern. For example, if A, B, and C each represent one type of modification to the nucleotide, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleotide, the alternating pattern, i.e., modifications on every other nucleotide, may be the same, but each of the sense strand or antisense strand can be selected from several possibilities of modifications within the alternating motif such as “ABABAB . . . ”, “ACACAC . . . ” “BDBDBD . . . ” or “CDCDCD . . . ,” etc.

In some embodiments, the dsRNAi agent of the invention comprises the modification pattern for the alternating motif on the sense strand relative to the modification pattern for the alternating motif on the antisense strand is shifted. The shift may be such that the modified group of nucleotides of the sense strand corresponds to a differently modified group of nucleotides of the antisense strand and vice versa. For example, the sense strand when paired with the antisense strand in the dsRNA duplex, the alternating motif in the sense strand may start with “ABABAB” from 5′ to 3′ of the strand and the alternating motif in the antisense strand may start with “BABABA” from 5′ to 3′ of the strand within the duplex region. As another example, the alternating motif in the sense strand may start with “AABBAABB” from 5′ to 3′ of the strand and the alternating motif in the antisense strand may start with “BBAABBAA” from 5′ to 3′ of the strand within the duplex region, so that there is a complete or partial shift of the modification patterns between the sense strand and the antisense strand.

In some embodiments, the dsRNAi agent comprises the pattern of the alternating motif of 2′-O-methyl modification and 2′-F modification on the sense strand initially has a shift relative to the pattern of the alternating motif of 2′-O-methyl modification and 2′-F modification on the antisense strand initially, i.e., the 2′-O-methyl modified nucleotide on the sense strand base pairs with a 2′-F modified nucleotide on the antisense strand and vice versa. The 1 position of the sense strand may start with the 2′-F modification, and the 1 position of the antisense strand may start with the 2′-O-methyl modification.

The introduction of one or more motifs of three identical modifications on three consecutive nucleotides to the sense strand or antisense strand interrupts the initial modification pattern present in the sense strand or antisense strand. This interruption of the modification pattern of the sense or antisense strand by introducing one or more motifs of three identical modifications on three consecutive nucleotides to the sense or antisense strand may enhance the gene silencing activity against the target gene.

In some embodiments, when the motif of three identical modifications on three consecutive nucleotides is introduced to any of the strands, the modification of the nucleotide next to the motif is a different modification than the modification of the motif. For example, the portion of the sequence containing the motif is “ . . . NₐYYYN_{b} . . . ,” where “Y” represents the modification of the motif of three identical modifications on three consecutive nucleotide, and “Nₐ” and “N_{b}” represent a modification to the nucleotide next to the motif “YYY” that is different than the modification of Y, and where Nₐ and N_{b} can be the same or different modifications. Alternatively, Nₐ or N_{b} may be present or absent when there is a wing modification present.

The iRNA may further comprise at least one phosphorothioate or methylphosphonate internucleotide linkage. The phosphorothioate or methylphosphonate internucleotide linkage modification may occur on any nucleotide of the sense strand, antisense strand, or both strands in any position of the strand. For instance, the internucleotide linkage modification may occur on every nucleotide on the sense strand or antisense strand; each internucleotide linkage modification may occur in an alternating pattern on the sense strand or antisense strand; or the sense strand or antisense strand may contain both internucleotide linkage modifications in an alternating pattern. The alternating pattern of the internucleotide linkage modification on the sense strand may be the same or different from the antisense strand, and the alternating pattern of the internucleotide linkage modification on the sense strand may have a shift relative to the alternating pattern of the internucleotide linkage modification on the antisense strand. In one embodiment, a double-stranded RNAi agent comprises 6-8 phosphorothioate internucleotide linkages. In some embodiments, the antisense strand comprises two phosphorothioate internucleotide linkages at the 5′-end and two phosphorothioate internucleotide linkages at the 3′-end, and the sense strand comprises at least two phosphorothioate internucleotide linkages at either the 5′-end or the 3′-end.

In some embodiments, the dsRNAi agent comprises a phosphorothioate or methylphosphonate internucleotide linkage modification in the overhang region. For example, the overhang region may contain two nucleotides having a phosphorothioate or methylphosphonate internucleotide linkage between the two nucleotides. Internucleotide linkage modifications also may be made to link the overhang nucleotides with the terminal paired nucleotides within the duplex region. For example, at least 2, 3, 4, or all the overhang nucleotides may be linked through phosphorothioate or methylphosphonate internucleotide linkage, and optionally, there may be additional phosphorothioate or methylphosphonate internucleotide linkages linking the overhang nucleotide with a paired nucleotide that is next to the overhang nucleotide. For instance, there may be at least two phosphorothioate internucleotide linkages between the terminal three nucleotides, in which two of the three nucleotides are overhang nucleotides, and the third is a paired nucleotide next to the overhang nucleotide. These terminal three nucleotides may be at the 3′-end of the antisense strand, the 3′-end of the sense strand, the 5′-end of the antisense strand, or the 5′ end of the antisense strand.

In some embodiments, the 2-nucleotide overhang is at the 3′-end of the antisense strand, and there are two phosphorothioate internucleotide linkages between the terminal three nucleotides, wherein two of the three nucleotides are the overhang nucleotides, and the third nucleotide is a paired nucleotide next to the overhang nucleotide. Optionally, the dsRNAi agent may additionally have two phosphorothioate internucleotide linkages between the terminal three nucleotides at both the 5′-end of the sense strand and at the 5′-end of the antisense strand.

In one embodiment, the dsRNAi agent comprises mismatch(es) with the target, within the duplex, or combinations thereof. The mismatch may occur in the overhang region or the duplex region. The base pair may be ranked on the basis of their propensity to promote dissociation or melting (e.g., on the free energy of association or dissociation of a particular pairing, the simplest approach is to examine the pairs on an individual pair basis, though next neighbor or similar analysis can also be used). In terms of promoting dissociation: A:U is preferred over G:C; G:U is preferred over G:C; and I:C is preferred over G:C (I=inosine). Mismatches, e.g., non-canonical or other than canonical pairings (as described elsewhere herein) are preferred over canonical (A:T, A:U, G:C) pairings; and pairings which include a universal base are preferred over canonical pairings.

In certain embodiments, the dsRNAi agent comprises at least one of the first 1, 2, 3, 4, or 5 base pairs within the duplex regions from the 5′-end of the antisense strand independently selected from the group of: A:U, G:U, I:C, and mismatched pairs, e.g., non-canonical or other than canonical pairings or pairings which include a universal base, to promote the dissociation of the antisense strand at the 5′-end of the duplex.

In certain embodiments, the nucleotide at the 1 position within the duplex region from the 5′-end in the antisense strand is selected from A, dA, dU, U, and dT. Alternatively, at least one of the first 1, 2, or 3 base pair within the duplex region from the 5′-end of the antisense strand is an AU base pair. For example, the first base pair within the duplex region from the 5′-end of the antisense strand is an AU base pair.

In other embodiments, the nucleotide at the 3′-end of the sense strand is deoxy-thymine (dT) or the nucleotide at the 3′-end of the antisense strand is deoxy-thymine (dT). For example, there is a short sequence of deoxy-thymine nucleotides, for example, two dT nucleotides on the 3′-end of the sense, antisense strand, or both strands.

In certain embodiments, the sense strand sequence may be represented by formula (I):

5′ nₚ-Nₐ—(XXX)ᵢ—N_{b}—YYY—N_{b}—(ZZZ)ⱼ—Nₐ-n_{q} 3′ (I)

wherein:

i and j are each independently 0 or 1;

p and q are each independently 0-6;

each Nₐ independently represents an oligonucleotide sequence comprising 0-25 modified nucleotides, each sequence comprising at least two differently modified nucleotides;

each N_{b} independently represents an oligonucleotide sequence comprising 0-10 modified nucleotides;

each nₚ and n_{q} independently represent an overhang nucleotide;

wherein Nb and Y do not have the same modification; and

XXX, YYY, and ZZZ each independently represent one motif of three identical modifications on three consecutive nucleotides. Preferably YYY is all 2′-F modified nucleotides.

In some embodiments, the Nₐ or N_{b} comprises modifications of alternating pattern.

In some embodiments, the YYY motif occurs at or near the cleavage site of the sense strand. For example, when the dsRNAi agent has a duplex region of 17-23 nucleotides in length, the YYY motif can occur at or the vicinity of the cleavage site (e.g.: can occur at positions 6, 7, 8; 7, 8, 9; 8, 9, 10; 9, 10, 11; 10, 11, 12; or 11, 12, 13) of the sense strand, the count starting from the first nucleotide, from the 5′-end; or optionally, the count starting at the first paired nucleotide within the duplex region, from the 5′-end.

In one embodiment, i is 1 and j is 0, or i is 0 and j is 1, or both i and j are 1. The sense strand can therefore be represented by the following formulas:

5′ nₚ-Nₐ—YYY—N_{b}—ZZZ—Nₐ-n_{q} 3′ (Ib);

5′ nₚ-Nₐ—XXX—N_{b}—YYY—Nₐ-n_{q} 3′ (Ic); or

5′ nₚ-Nₐ—XXX—N_{b}—YYY—N_{b}—ZZZ—Nₐ-n_{q} 3′ (Id).

When the sense strand is represented by formula (Ib), N_{b} represents an oligonucleotide sequence comprising 0-10, 0-7, 0-5, 0-4, 0-2, or 0 modified nucleotides. Each Nₐ independently can represent an oligonucleotide sequence comprising 2-20, 2-15, or 2-10 modified nucleotides.

When the sense strand is represented as formula (Ic), N_{b} represents an oligonucleotide sequence comprising 0-10, 0-7, 0-10, 0-7, 0-5, 0-4, 0-2, or 0 modified nucleotides. Each Nₐ can independently represent an oligonucleotide sequence comprising 2-20, 2-15, or 2-10 modified nucleotides.

When the sense strand is represented as formula (Id), each N_{b} independently represents an oligonucleotide sequence comprising 0-10, 0-7, 0-5, 0-4, 0-2, or 0 modified nucleotides. Preferably, N_{b} is 0, 1, 2, 3, 4, 5, or 6 Each Nₐ can independently represent an oligonucleotide sequence comprising 2-20, 2-15, or 2-10 modified nucleotides.

Each of X, Y and Z may be the same or different from each other.

In other embodiments, i is 0 and j is 0, and the sense strand may be represented by the formula:

5′ nₚ-Nₐ—YYY—Nₐ-n_{q} 3′ (Ia).

When the sense strand is represented by formula (Ia), each Nₐ independently can represent an oligonucleotide sequence comprising 2-20, 2-15, or 2-10 modified nucleotides.

In one embodiment, the antisense strand sequence of the RNAi may be represented by formula (II):

5′ n_{q′}-Nₐ′—(Z′Z′Z′)ₖ—N_{b}′—Y′Y′Y′—N_{b}′—(X′X′X′)—N′ₐ-nₚ′ 3′ (II)

wherein:

k and 1 are each independently 0 or 1;

p′ and q′ are each independently 0-6;

each Nₐ′ independently represents an oligonucleotide sequence comprising 0-25 modified nucleotides, each sequence comprising at least two differently modified nucleotides;

each N_{b}′ independently represents an oligonucleotide sequence comprising 0-10 modified nucleotides;

each nₚ′ and n_{q}′ independently represent an overhang nucleotide;

wherein N_{b}′ and Y′ do not have the same modification; and

X′X′X′, Y′Y′Y′, and Z′Z′Z′ each independently represent one motif of three identical modifications on three consecutive nucleotides.

In some embodiments, the Nₐ′ or N_{b}′ comprises modifications of alternating pattern.

The Y′Y′Y′ motif occurs at or near the cleavage site of the antisense strand. For example, when the dsRNAi agent has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense strand, with the count starting from the first nucleotide, from the 5′-end; or optionally, the count starting at the first paired nucleotide within the duplex region, from the 5′-end. Preferably, the Y′Y′Y′ motif occurs at positions 11, 12, 13.

In certain embodiments, Y′Y′Y′ motif is all 2′-OMe modified nucleotides.

In certain embodiments, k is 1 and l is 0, or k is 0 and 1 is 1, or both k and 1 are 1.

The antisense strand can therefore be represented by the following formulas:

5′ n_{q′}-Nₐ′—Z′Z′Z′—N_{b}′—Y′Y′Y′—Nₐ′-n_{p′} 3′ (IIb);

5′ n_{q′}-Nₐ′—Y′Y′Y′—N_{b}′—X′X′X′-n_{p′} 3′ (IIc); or

5′ n_{q′}-Nₐ′— Z′Z′Z′—N_{b}′—Y′Y′Y′—N_{b}′—X′X′X′—Nₐ′-n_{p′} 3′ (IId).

When the antisense strand is represented by formula (IIb), N_{b} represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IIc), N_{b}′ represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides.

When the antisense strand is represented as formula (IId), each N_{b}′ independently represents an oligonucleotide sequence comprising 0-10, 0-7, 0-10, 0-7, 0-5, 0-4, 0-2, or 0 modified nucleotides. Each Nₐ′ independently represents an oligonucleotide sequence comprising 2-20, 2-15, or 2-10 modified nucleotides. Preferably, N_{b} is 0, 1, 2, 3, 4, 5, or 6.

In other embodiments, k is 0 and 1 is 0 and the antisense strand may be represented by the formula:

5′ n_{p′}-N_{a′}—Y′Y′Y′—N_{a′}-n_{q′} 3′ (Ia).

When the antisense strand is represented as formula (IIa), each Nₐ′ independently represents an oligonucleotide sequence comprising 2-20, 2-15, or 2-10 modified nucleotides.

Each of X′, Y′ and Z′ may be the same or different from each other.

Each nucleotide of the sense strand and antisense strand may be independently modified with LNA, CRN, UNA, cEt, HNA, CeNA, 2′-methoxyethyl, 2′-O-methyl, 2′-O-allyl, 2′-C-allyl, 2′-hydroxyl, or 2′-fluoro. For example, each nucleotide of the sense strand and antisense strand is independently modified with 2′-O-methyl or 2′-fluoro. Each X, Y, Z, X′, Y′, and Z′, in particular, may represent a 2′-O-methyl modification or a 2′-fluoro modification.

In some embodiments, the sense strand of the dsRNAi agent may contain YYY motif occurring at 9, 10, and 11 positions of the strand when the duplex region is 21 nt, the count starting from the first nucleotide from the 5′-end, or optionally, the count starting at the first paired nucleotide within the duplex region, from the 5′-end; and Y represents 2′-F modification. The sense strand may additionally contain XXX motif or ZZZ motifs as wing modifications at the opposite end of the duplex region; and XXX and ZZZ each independently represents a 2′-OMe modification or 2′-F modification.

In some embodiments the antisense strand may contain Y′Y′Y′ motif occurring at positions 11, 12, 13 of the strand, the count starting from the first nucleotide from the 5′-end, or optionally, the count starting at the first paired nucleotide within the duplex region, from the 5′-end; and Y′ represents 2′-O-methyl modification. The antisense strand may additionally contain X′X′X′ motif or Z′Z′Z′ motifs as wing modifications at the opposite end of the duplex region; and X′X′X′ and Z′Z′Z′ each independently represents a 2′-OMe modification or 2′-F modification.

The sense strand represented by any one of the above formulas (Ia), (Ib), (Ic), and (Id) forms a duplex with a antisense strand being represented by any one of formulas (IIa), (IIb), (IIc), and (IId), respectively.

Accordingly, the dsRNAi agents for use in the methods of the invention may comprise a sense strand and an antisense strand, each strand having 14 to 30 nucleotides, the iRNA duplex represented by formula (III):

sense: 5′ nₚ-Nₐ—(XXX)ᵢ—N_{b}—YYY—N_{b}—(ZZZ)ⱼ—Nₐ-n_{q} 3′

antisense: 3′ nₚ′-Nₐ′—(X′X′X′)ₖ—N_{b} Y′Y′Y′—N_{b}′—(Z′Z′Z′)ₗ—Nₐ′-n_{q} 5′ (III)

wherein:

i, j, k, and 1 are each independently 0 or 1;

p, p′, q, and q′ are each independently 0-6;

each Nₐ and Nₐ′ independently represents an oligonucleotide sequence comprising 0-25 modified nucleotides, each sequence comprising at least two differently modified nucleotides;

each N_{b} and N_{b}′ independently represents an oligonucleotide sequence comprising 0-10 modified nucleotides;

wherein each nₚ′, nₚ, n_{q}′, and n_{q}, each of which may or may not be present, independently represents an overhang nucleotide; and

XXX, YYY, ZZZ, X′X′X′, Y′Y′Y′, and Z′Z′Z′ each independently represent one motif of three identical modifications on three consecutive nucleotides.

In one embodiment, i is 0 and j is 0; or i is 1 and j is 0; or i is 0 and j is 1; or both i and j are 0; or both i and j are 1. In another embodiment, k is 0 and l is 0; or k is 1 and l is 0; k is 0 and l is 1; or both k and l are 0; or both k and l are 1.

Exemplary combinations of the sense strand and antisense strand forming an iRNA duplex include the formulas below:

5′ nₚ-Nₐ—YYY—Nₐ-n_{q} 3′

3′ nₚ′-Nₐ′—Y′Y′Y′—Nₐ′n_{q}′ 5′ (IIIa)

5′ nₚ-Nₐ—YYY—N_{b}—ZZZ—Nₐ-n_{q} 3′

3′ nₚ′-Nₐ′—Y′Y′Y′—N_{b}—Z′Z′Z′—Nₐ′n_{q}′ 5′ (IIIb)

5′ nₚ-Nₐ—XXX—N_{b}—YYY—Nₐ-n_{q} 3′

3′ nₚ′-Nₐ′—X′X′X′—N_{b}′—Y′Y′Y′—Nₐ′-n_{q}′ 5′ (IIIc)

5′ nₚ-Nₐ—XXX—N_{b}—YYY—N_{b}—ZZZ—Nₐ-n_{q} 3′

3′ nₚ′-Nₐ′—X′X′X′—N_{b}′—Y′Y′Y′—N_{b}′—Z′Z′Z′—Nₐ-n_{q}′ 5′ (IIId)

When the dsRNAi agent is represented by formula (IIIa), each Nₐ independently represents an oligonucleotide sequence comprising 2-20, 2-15, or 2-10 modified nucleotides.

When the dsRNAi agent is represented by formula (IIIb), each N_{b} independently represents an oligonucleotide sequence comprising 1-10, 1-7, 1-5, or 1-4 modified nucleotides. Each Nₐ independently represents an oligonucleotide sequence comprising 2-20, 2-15, or 2-10 modified nucleotides.

When the dsRNAi agent is represented as formula (IIIc), each N_{b}, N_{b}′ independently represents an oligonucleotide sequence comprising 0-10, 0-7, 0-10, 0-7, 0-5, 0-4, 0-2, or 0 modified nucleotides. Each Nₐ independently represents an oligonucleotide sequence comprising 2-20, 2-15, or 2-10 modified nucleotides.

When the dsRNAi agent is represented as formula (IIId), each N_{b}, N_{b}′ independently represents an oligonucleotide sequence comprising 0-10, 0-7, 0-10, 0-7, 0-5, 0-4, 0-2, or 0 modified nucleotides. Each Nₐ, Nₐ independently represents an oligonucleotide sequence comprising 2-20, 2-15, or 2-10 modified nucleotides. Each of Nₐ, Nₐ′, N_{b}, and N_{b}′ independently comprises modifications of alternating pattern.

Each of X, Y, and Z in formulas (III), (IIIa), (IIIb), (IIIc), and (IIId) may be the same or different from each other.

When the dsRNAi agent is represented by formula (III), (IIIa), (IIIb), (IIIc), and (IIId), at least one of the Y nucleotides may form a base pair with one of the Y′ nucleotides. Alternatively, at least two of the Y nucleotides form base pairs with the corresponding Y′ nucleotides; or all three of the Y nucleotides all form base pairs with the corresponding Y′ nucleotides.

When the dsRNAi agent is represented by formula (IIIb) or (IIId), at least one of the Z nucleotides may form a base pair with one of the Z′ nucleotides. Alternatively, at least two of the Z nucleotides form base pairs with the corresponding Z′ nucleotides; or all three of the Z nucleotides all form base pairs with the corresponding Z′ nucleotides.

When the dsRNAi agent is represented as formula (IIIc) or (IIId), at least one of the X nucleotides may form a base pair with one of the X′ nucleotides. Alternatively, at least two of the X nucleotides form base pairs with the corresponding X′ nucleotides; or all three of the X nucleotides all form base pairs with the corresponding X′ nucleotides.

In certain embodiments, the modification on the Y nucleotide is different than the modification on the Y′ nucleotide, the modification on the Z nucleotide is different than the modification on the Z′ nucleotide, or the modification on the X nucleotide is different than the modification on the X′ nucleotide.

In certain embodiments, when the dsRNAi agent is represented by formula (IIId), the Nₐ modifications are 2′-O-methyl or 2′-fluoro modifications. In other embodiments, when the RNAi agent is represented by formula (IIId), the Nₐ modifications are 2′-O-methyl or 2′-fluoro modifications and nₚ′>0 and at least one nₚ′ is linked to a neighboring nucleotide a via phosphorothioate linkage. In yet other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, and the sense strand is conjugated to one or more GalNAc derivatives attached through a bivalent or trivalent branched linker (described below). In other embodiments, when the RNAi agent is represented by formula (IIId), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, when the dsRNAi agent is represented by formula (IIIa), the Nₐ modifications are 2′-O-methyl or 2′-fluoro modifications, nₚ′>0 and at least one nₚ′ is linked to a neighboring nucleotide via phosphorothioate linkage, the sense strand comprises at least one phosphorothioate linkage, and the sense strand is conjugated to one or more GalNAc derivatives attached through a bivalent or trivalent branched linker.

In some embodiments, the dsRNAi agent is a multimer containing at least two duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In some embodiments, the dsRNAi agent is a multimer containing three, four, five, six, or more duplexes represented by formula (III), (IIIa), (IIIb), (IIIc), and (IIId), wherein the duplexes are connected by a linker. The linker can be cleavable or non-cleavable. Optionally, the multimer further comprises a ligand. Each of the duplexes can target the same gene or two different genes; or each of the duplexes can target same gene at two different target sites.

In one embodiment, two dsRNAi agents represented by at least one of formulas (III), (IIIa), (IIIb), (IIIc), and (IIId) are linked to each other at the 5′ end, and one or both of the 3′ ends, and are optionally conjugated to a ligand. Each of the agents can target the same gene or two different genes; or each of the agents can target same gene at two different target sites.

In certain embodiments, an RNAi agent of the invention may contain a low number of nucleotides containing a 2′-fluoro modification, e.g., 10 or fewer nucleotides with 2′-fluoro modification. For example, the RNAi agent may contain 10, 9, 8, 7, 6, 5, 4, 3, 2, 1 or 0 nucleotides with a 2′-fluoro modification. In a specific embodiment, the RNAi agent of the invention contains 10 nucleotides with a 2′-fluoro modification, e.g., 4 nucleotides with a 2′-fluoro modification in the sense strand and 6 nucleotides with a 2′-fluoro modification in the antisense strand. In another specific embodiment, the RNAi agent of the invention contains 6 nucleotides with a 2′-fluoro modification, e.g., 4 nucleotides with a 2′-fluoro modification in the sense strand and 2 nucleotides with a 2′-fluoro modification in the antisense strand.

In other embodiments, an RNAi agent of the invention may contain an ultra low number of nucleotides containing a 2′-fluoro modification, e.g., 2 or fewer nucleotides containing a 2′-fluoro modification. For example, the RNAi agent may contain 2, 1 of 0 nucleotides with a 2′-fluoro modification. In a specific embodiment, the RNAi agent may contain 2 nucleotides with a 2′-fluoro modification, e.g., 0 nucleotides with a 2-fluoro modification in the sense strand and 2 nucleotides with a 2′-fluoro modification in the antisense strand.

Various publications describe multimeric iRNAs that can be used in the methods of the invention. Such publications include WO2007/091269, U.S. Pat. No. 7,858,769, WO2010/141511, WO2007/117686, WO2009/014887, and WO2011/031520 the entire contents of each of which are hereby incorporated herein by reference.

As described in more detail below, the iRNA that contains conjugations of one or more carbohydrate moieties to an iRNA can optimize one or more properties of the iRNA. In many cases, the carbohydrate moiety will be attached to a modified subunit of the iRNA. For example, the ribose sugar of one or more ribonucleotide subunits of a iRNA can be replaced with another moiety, e.g., a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS). A cyclic carrier may be a carbocyclic ring system, i.e., all ring atoms are carbon atoms, or a heterocyclic ring system, i.e., one or more ring atoms may be a heteroatom, e.g., nitrogen, oxygen, sulfur. The cyclic carrier may be a monocyclic ring system, or may contain two or more rings, e.g. fused rings. The cyclic carrier may be a fully saturated ring system, or it may contain one or more double bonds.

The ligand may be attached to the polynucleotide via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a ribonucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide, or polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

The iRNA may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl, and decalin; preferably, the acyclic group is a serinol backbone or diethanolamine backbone.

In another embodiment of the invention, an iRNA agent comprises a sense strand and an antisense strand, each strand having 14 to 40 nucleotides. The RNAi agent may be represented by formula (L):

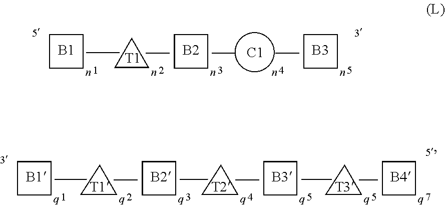

In formula (L), B1, B2, B3, B1′, B2′, B3′, and B4′ each are independently a nucleotide containing a modification selected from the group consisting of 2′-O-alkyl, 2′-substituted alkoxy, 2′-substituted alkyl, 2′-halo, ENA, and BNA/LNA. In one embodiment, B1, B2, B3, B1′, B2′, B3′, and B4′ each contain 2′-OMe modifications. In one embodiment, B1, B2, B3, B1′, B2′, B3′, and B4′ each contain 2′-OMe or 2′-F modifications. In one embodiment, at least one of B1, B2, B3, B1′, B2′, B3′, and B4′ contain 2′-O—N-methylacetamido (2′-O-NMA) modification.

C1 is a thermally destabilizing nucleotide placed at a site opposite to the seed region of the antisense strand (i.e., at positions 2-8 of the 5′-end of the antisense strand). For example, C1 is at a position of the sense strand that pairs with a nucleotide at positions 2-8 of the 5′-end of the antisense strand. In one example, C1 is at position 15 from the 5′-end of the sense strand. C1 nucleotide bears the thermally destabilizing modification which can include abasic modification; mismatch with the opposing nucleotide in the duplex; and sugar modification such as 2′-deoxy modification or acyclic nucleotide e.g., unlocked nucleic acids (UNA) or glycerol nucleic acid (GNA). In one embodiment, C1 has thermally destabilizing modification selected from the group consisting of: i) mismatch with the opposing nucleotide in the antisense strand; ii) abasic modification selected from the group consisting of:

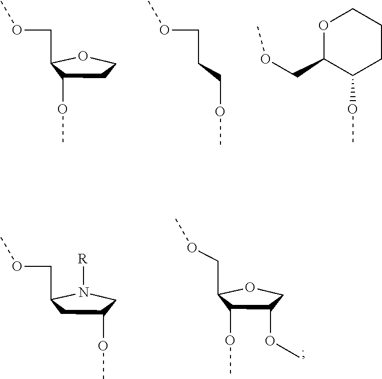

and iii) sugar modification selected from the group consisting of:

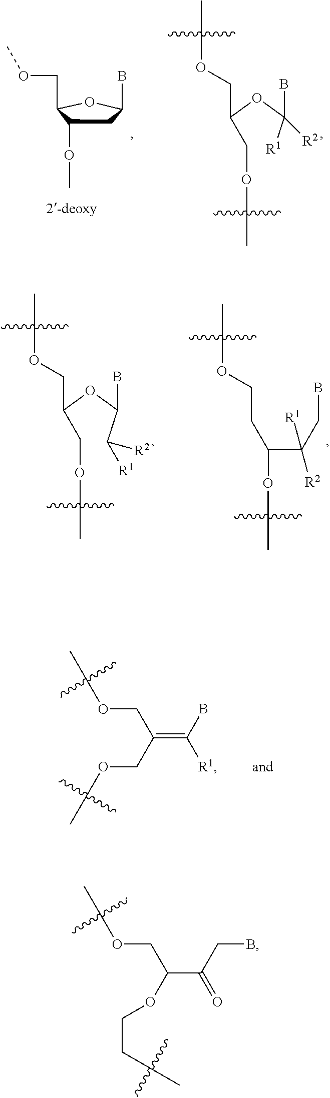

wherein B is a modified or unmodified nucleobase, R¹ and R² independently are H, halogen, OR₃, or alkyl; and R₃ is H, alkyl, cycloalkyl, aryl, aralkyl, heteroaryl or sugar. In one embodiment, the thermally destabilizing modification in C1 is a mismatch selected from the group consisting of G:G, G:A, G:U, G:T, A:A, A:C, C:C, C:U, C:T, U:U, T:T, and U:T; and optionally, at least one nucleobase in the mismatch pair is a 2′-deoxy nucleobase. In one example, the thermally destabilizing modification in C1 is GNA or

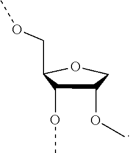

T1, T1′, T2′, and T3′ each independently represent a nucleotide comprising a modification providing the nucleotide a steric bulk that is less or equal to the steric bulk of a 2′-OMe modification. A steric bulk refers to the sum of steric effects of a modification. Methods for determining steric effects of a modification of a nucleotide are known to one skilled in the art. The modification can be at the 2′ position of a ribose sugar of the nucleotide, or a modification to a non-ribose nucleotide, acyclic nucleotide, or the backbone of the nucleotide that is similar or equivalent to the 2′ position of the ribose sugar, and provides the nucleotide a steric bulk that is less than or equal to the steric bulk of a 2′-OMe modification. For example, T1, T1′, T2′, and T3′ are each independently selected from DNA, RNA, LNA, 2′-F, and 2′-F-5′-methyl. In one embodiment, T1 is DNA. In one embodiment, T1′ is DNA, RNA or LNA. In one embodiment, T2′ is DNA or RNA. In one embodiment, T3′ is DNA or RNA.
n¹, n³, and q¹ are independently 4 to 15 nucleotides in length.
n⁵, q³, and q⁷ are independently 1-6 nucleotide(s) in length.
n⁴, q², and q⁶ are independently 1-3 nucleotide(s) in length; alternatively, n⁴ is 0.
q⁵ is independently 0-10 nucleotide(s) in length.
n² and q⁴ are independently 0-3 nucleotide(s) in length.

Alternatively, n⁴ is 0-3 nucleotide(s) in length.

In one embodiment, n⁴ can be 0. In one example, n⁴ is 0, and q² and q⁶ are 1. In another example, n⁴ is 0, and q² and q⁶ are 1, with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴, q², and q⁶ are each 1.

In one embodiment, n², n⁴, q², q⁴, and q⁶ are each 1.

In one embodiment, C1 is at position 14-17 of the 5′-end of the sense strand, when the sense strand is 19-22 nucleotides in length, and n⁴ is 1. In one embodiment, C1 is at position 15 of the 5′-end of the sense strand

In one embodiment, T3′ starts at position 2 from the 5′ end of the antisense strand. In one example, T3′ is at position 2 from the 5′ end of the antisense strand and q⁶ is equal to 1.

In one embodiment, T1′ starts at position 14 from the 5′ end of the antisense strand. In one example, T1′ is at position 14 from the 5′ end of the antisense strand and q² is equal to 1.

In an exemplary embodiment, T3′ starts from position 2 from the 5′ end of the antisense strand and T1′ starts from position 14 from the 5′ end of the antisense strand. In one example, T3′ starts from position 2 from the 5′ end of the antisense strand and q⁶ is equal to 1 and T1′ starts from position 14 from the 5′ end of the antisense strand and q² is equal to 1.

In one embodiment, T1′ and T3′ are separated by 11 nucleotides in length (i.e. not counting the T1′ and T3′ nucleotides).

In one embodiment, T1′ is at position 14 from the 5′ end of the antisense strand. In one example, T1′ is at position 14 from the 5′ end of the antisense strand and q² is equal to 1, and the modification at the 2′ position or positions in a non-ribose, acyclic or backbone that provide less steric bulk than a 2′-OMe ribose.

In one embodiment, T3′ is at position 2 from the 5′ end of the antisense strand. In one example, T3′ is at position 2 from the 5′ end of the antisense strand and q⁶ is equal to 1, and the modification at the 2′ position or positions in a non-ribose, acyclic or backbone that provide less than or equal to steric bulk than a 2′-OMe ribose.

In one embodiment, T1 is at the cleavage site of the sense strand. In one example, T1 is at position 11 from the 5′ end of the sense strand, when the sense strand is 19-22 nucleotides in length, and n² is 1. In an exemplary embodiment, T1 is at the cleavage site of the sense strand at position 11 from the 5′ end of the sense strand, when the sense strand is 19-22 nucleotides in length, and n² is 1,

In one embodiment, T2′ starts at position 6 from the 5′ end of the antisense strand. In one example, T2′ is at positions 6-10 from the 5′ end of the antisense strand, and q⁴ is 1.

In an exemplary embodiment, T1 is at the cleavage site of the sense strand, for instance, at position 11 from the 5′ end of the sense strand, when the sense strand is 19-22 nucleotides in length, and n² is 1; T1′ is at position 14 from the 5′ end of the antisense strand, and q² is equal to 1, and the modification to T1′ is at the 2′ position of a ribose sugar or at positions in a non-ribose, acyclic or backbone that provide less steric bulk than a 2′-OMe ribose; T2′ is at positions 6-10 from the 5′ end of the antisense strand, and q⁴ is 1; and T3′ is at position 2 from the 5′ end of the antisense strand, and q⁶ is equal to 1, and the modification to T3′ is at the 2′ position or at positions in a non-ribose, acyclic or backbone that provide less than or equal to steric bulk than a 2′-OMe ribose.

In one embodiment, T2′ starts at position 8 from the 5′ end of the antisense strand. In one example, T2′ starts at position 8 from the 5′ end of the antisense strand, and q⁴ is 2.

In one embodiment, T2′ starts at position 9 from the 5′ end of the antisense strand. In one example, T2′ is at position 9 from the 5′ end of the antisense strand, and q⁴ is 1.

In one embodiment, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 6, T1 is 2′F, n² is 3, B2 is 2′-OMe, n³ is 7, n⁴ is 0, B3 is 2′OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1.

In one embodiment, B1 is 2′-OMe or 2′-F, n¹ is 6, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 7, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 1, B3′ is 2′-OMe or 2′-F, q⁵ is 6, T3′ is 2′-F, q⁶ is 1, B4′ is 2′-OMe, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 5, T2′ is 2′-F, q⁴ is 1, B3′ is 2′-OMe or 2′-F, q⁵ is 5, T3′ is 2′-F, q⁶ is 1, B4′ is 2′-OMe, and q⁷ is 1; optionally with at least 2 additional TT at the 3′-end of the antisense strand; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within positions 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end).

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within positions 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand).

The RNAi agent can comprise a phosphorus-containing group at the 5′-end of the sense strand or antisense strand. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos), or 5′-deoxy-5′-C-malonyl

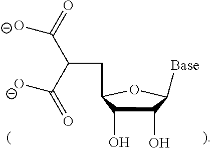

When the 5′-end phosphorus-containing group is 5′-end vinylphosphonate (5′-VP), the 5′-VP can be either 5′-E-VP isomer (i.e., trans-vinylphosate,

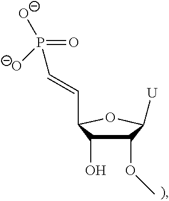

5′-Z-VP isomer (i.e., cis-vinylphosphate,

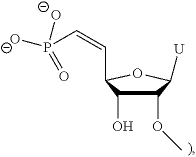

or mixtures thereof.

In one embodiment, the RNAi agent comprises a phosphorus-containing group at the 5′-end of the sense strand. In one embodiment, the RNAi agent comprises a phosphorus-containing group at the 5′-end of the antisense strand.

In one embodiment, the RNAi agent comprises a 5′-P. In one embodiment, the RNAi agent comprises a 5′-P in the antisense strand.

In one embodiment, the RNAi agent comprises a 5′-PS. In one embodiment, the RNAi agent comprises a 5′-PS in the antisense strand.

In one embodiment, the RNAi agent comprises a 5′-VP. In one embodiment, the RNAi agent comprises a 5′-VP in the antisense strand. In one embodiment, the RNAi agent comprises a 5′-E-VP in the antisense strand. In one embodiment, the RNAi agent comprises a 5′-Z-VP in the antisense strand.

In one embodiment, the RNAi agent comprises a 5′-PS₂. In one embodiment, the RNAi agent comprises a 5′-PS₂ in the antisense strand.

In one embodiment, the RNAi agent comprises a 5′-PS₂. In one embodiment, the RNAi agent comprises a 5′-deoxy-5′-C-malonyl in the antisense strand.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The dsRNA agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The dsRNAi RNA agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1. The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP. The 5′-VP may be 5′-E-VP, 5′-Z-VP, or combination thereof.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof), and a targeting ligand.

In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-OMe, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-OMe, and q⁷ is 1; with two phosphorothioate internucleotide linkage modifications within position 1-5 of the sense strand (counting from the 5′-end), and two phosphorothioate internucleotide linkage modifications at positions 1 and 2 and two phosphorothioate internucleotide linkage modifications within positions 18-23 of the antisense strand (counting from the 5′-end). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, T2′ is 2′-F, q⁴ is 2, B3′ is 2′-OMe or 2′-F, q⁵ is 5, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-P and a targeting ligand. In one embodiment, the 5′-P is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS and a targeting ligand. In one embodiment, the 5′-PS is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-VP (e.g., a 5′-E-VP, 5′-Z-VP, or combination thereof) and a targeting ligand. In one embodiment, the 5′-VP is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-PS₂ and a targeting ligand. In one embodiment, the 5′-PS₂ is at the 5′-end of the antisense strand, and the targeting ligand is at the 3′-end of the sense strand.

In one embodiment, B1 is 2′-OMe or 2′-F, n¹ is 8, T1 is 2′F, n² is 3, B2 is 2′-OMe, n³ is 7, n⁴ is 0, B3 is 2′-OMe, n⁵ is 3, B1′ is 2′-OMe or 2′-F, q¹ is 9, T1′ is 2′-F, q² is 1, B2′ is 2′-OMe or 2′-F, q³ is 4, q⁴ is 0, B3′ is 2′-OMe or 2′-F, q⁵ is 7, T3′ is 2′-F, q⁶ is 1, B4′ is 2′-F, and q⁷ is 1; with two phosphorothioate internucleotide linkage modifications within position 1-5 of the sense strand (counting from the 5′-end of the sense strand), and two phosphorothioate internucleotide linkage modifications at positions 1 and 2 and two phosphorothioate internucleotide linkage modifications within positions 18-23 of the antisense strand (counting from the 5′-end of the antisense strand). The RNAi agent also comprises a 5′-deoxy-5′-C-malonyl and a targeting ligand. In one embodiment, the 5′-deoxy-5′-C-malonyl is at the 5′-end of the antisense strand, and the targeting ligand is at the 3′-end of the sense strand.

In a particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense strand having:
   (i) a length of 23 nucleotides;
   (ii) 2′-OMe modifications at positions 1, 3, 5, 9, 11 to 13, 15, 17, 19, 21, and 23, and 2′F modifications at positions 2, 4, 6 to 8, 10, 14, 16, 18, 20, and 22 (counting from the 5′ end); and
   (iii) phosphorothioate internucleotide linkages between nucleotide positions 21 and 22, and between nucleotide positions 22 and 23 (counting from the 5′ end);
wherein the dsRNA agents have a two nucleotide overhang at the 3′-end of the antisense strand, and a blunt end at the 5′-end of the antisense strand.

In another particular embodiment, an RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, 13, 15, 17, 19, and 21, and 2′-OMe modifications at positions 2, 4, 6, 8, 12, 14, 16, 18, and 20 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, and 12 to 21, 2′-F modifications at positions 7, and 9, and a deoxy-nucleotide (e.g. dT) at position 11 (counting from the 5′ end); and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, 10, 12, 14, and 16 to 21, and 2′-F modifications at positions 7, 9, 11, 13, and 15; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 5, 7, 9, 11, 13, 15, 17, 19, and 21 to 23, and 2′-F modifications at positions 2 to 4, 6, 8, 10, 12, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 9, and 12 to 21, and 2′-F modifications at positions 10, and 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5, 7, 9, 11 to 13, 15, 17, 19, and 21 to 23, and 2′-F modifications at positions 2, 4, 6, 8, 10, 14, 16, 18, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-F modifications at positions 1, 3, 5, 7, 9 to 11, and 13, and 2′-OMe modifications at positions 2, 4, 6, 8, 12, and 14 to 21; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3, 5 to 7, 9, 11 to 13, 15, 17 to 19, and 21 to 23, and 2′-F modifications at positions 2, 4, 8, 10, 14, 16, and 20 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1, 2, 4, 6, 8, 12, 14, 15, 17, and 19 to 21, and 2′-F modifications at positions 3, 5, 7, 9 to 11, 13, 16, and 18; and
   (iv) phosphorothioate internucleotide linkages between nucleotide positions 1 and 2, and between nucleotide positions 2 and 3 (counting from the 5′ end);
and
(b) an antisense strand having:
   (i) a length of 25 nucleotides;
   (ii) 2′-OMe modifications at positions 1, 4, 6, 7, 9, 11 to 13, 15, 17, and 19 to 23, 2′-F modifications at positions 2, 3, 5, 8, 10, 14, 16, and 18, and desoxy-nucleotides (e.g. dT) at positions 24 and 25 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a four nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 21 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 6, 8, and 12 to 21, and 2′-F modifications at positions 7, and 9 to 11; and
   (iv) phosphorothioate internucleotide linkages between nucleotide positions 1 and 2, and between nucleotide positions 2 and 3 (counting from the 5′ end);
and
(b) an antisense strand having:
   (i) a length of 23 nucleotides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 21 and 22, and between nucleotide positions 22 and 23 (counting from the 5′ end);
      wherein the RNAi agents have a two nucleotide overhang at the 3′-end of the antisense strand, and a blunt end at the 5′-end of the antisense strand.

In another particular embodiment, a RNAi agent of the present invention comprises:
(a) a sense strand having:
   (i) a length of 19 nucleotides;
   (ii) an ASGPR ligand attached to the 3′-end, wherein said ASGPR ligand comprises three GalNAc derivatives attached through a trivalent branched linker;
   (iii) 2′-OMe modifications at positions 1 to 4, 6, and 10 to 19, and 2′-F modifications at positions 5, and 7 to 9; and
   (iv) phosphorothioate internucleotide linkages between nucleotide positions 1 and 2, and between nucleotide positions 2 and 3 (counting from the 5′ end);
and
(b) an antisense strand having:
   (i) a length of 21 nucleotides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleotide linkages between nucleotide positions 1 and 2, between nucleotide positions 2 and 3, between nucleotide positions 19 and 20, and between nucleotide positions 20 and 21 (counting from the 5′ end);
      wherein the RNAi agents have a two nucleotide overhang at the 3′-end of the antisense strand, and a blunt end at the 5′-end of the antisense strand.

In certain embodiments, the iRNA for use in the methods of the invention is an agent selected from agents listed in any one of Tables 3, 5, 6, or 7. These agents may further comprise a ligand.

III. iRNAs Conjugated to Ligands

Another modification of the RNA of an iRNA of the invention involves chemically linking to the iRNA one or more ligands, moieties or conjugates that enhance the activity, cellular distribution, or cellular uptake of the iRNA e.g., into a cell. Such moieties include but are not limited to lipid moieties such as a cholesterol moiety (Letsinger et al., Proc. Natl. Acid. Sci. USA, 1989, 86: 6553-6556). In other embodiments, the ligand is cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In certain embodiments, a ligand alters the distribution, targeting, or lifetime of an iRNA agent into which it is incorporated. In preferred embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Preferred ligands do not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-glucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic. In certain embodiments, the ligand is a multivalent galactose, e.g., an N-acetyl-galactosamine.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-icB.

The ligand can be a substance, e.g., a drug, which can increase the uptake of the iRNA agent into the cell, for example, by disrupting the cell's cytoskeleton, e.g., by disrupting the cell's microtubules, microfilaments, or intermediate filaments. The drug can be, for example, taxol, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic modulator (PK modulator). PK modulators include lipophiles, bile acids, steroids, phospholipid analogues, peptides, protein binding agents, PEG, vitamins, etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin. Oligonucleotides that comprise a number of phosphorothioate linkages are also known to bind to serum protein, thus short oligonucleotides, e.g., oligonucleotides of about 5 bases, 10 bases, 15 bases, or 20 bases, comprising multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated iRNAs of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems® (Foster City, Calif.). Any other methods for such synthesis known in the art may additionally or alternatively be employed. It is also known to use similar techniques to prepare other oligonucleotides, such as the phosphorothioates and alkylated derivatives.

In the ligand-conjugated iRNAs and ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

A. Lipid Conjugates

In certain embodiments, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, naproxen or aspirin can be used. A lipid or lipid-based ligand can (a) increase resistance to degradation of the conjugate, (b) increase targeting or transport into a target cell or cell membrane, or (c) can be used to adjust binding to a serum protein, e.g., HSA.

A lipid based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In certain embodiments, the lipid based ligand binds HSA. Preferably, it binds HSA with a sufficient affinity such that the conjugate will be preferably distributed to a non-kidney tissue. However, it is preferred that the affinity not be so strong that the HSA-ligand binding cannot be reversed.

In other embodiments, the lipid based ligand binds HSA weakly or not at all, such that the conjugate will be preferably distributed to the kidney. Other moieties that target to kidney cells can also be used in place of, or in addition to, the lipid based ligand.

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by target cells such as liver cells. Also included are HSA and low density lipoprotein (LDL).

B. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to iRNA agents can affect pharmacokinetic distribution of the iRNA, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO: 13). An RFGF analogue (e.g., amino acid sequence AALLPVLLAAP (SEQ ID NO:14) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ (SEQ ID NO:15) and the Drosophila antennapedia protein (RQIKIWFQNRRMKWKK (SEQ ID NO:16) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to a dsRNA agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidiomimemtics may include D-amino acids, as well as synthetic RGD mimics. In addition to RGD, one can use other moieties that target the integrin ligand. Preferred conjugates of this ligand target PECAM-1 or VEGF.

A “cell permeation peptide” is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

C. Carbohydrate Conjugates

In some embodiments of the compositions and methods of the invention, an iRNA further comprises a carbohydrate. The carbohydrate conjugated iRNA is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri-, and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In certain embodiments, a carbohydrate conjugate for use in the compositions and methods of the invention is a monosaccharide.

In one embodiment, a carbohydrate conjugate for use in the compositions and methods of the invention is selected from the group consisting of:

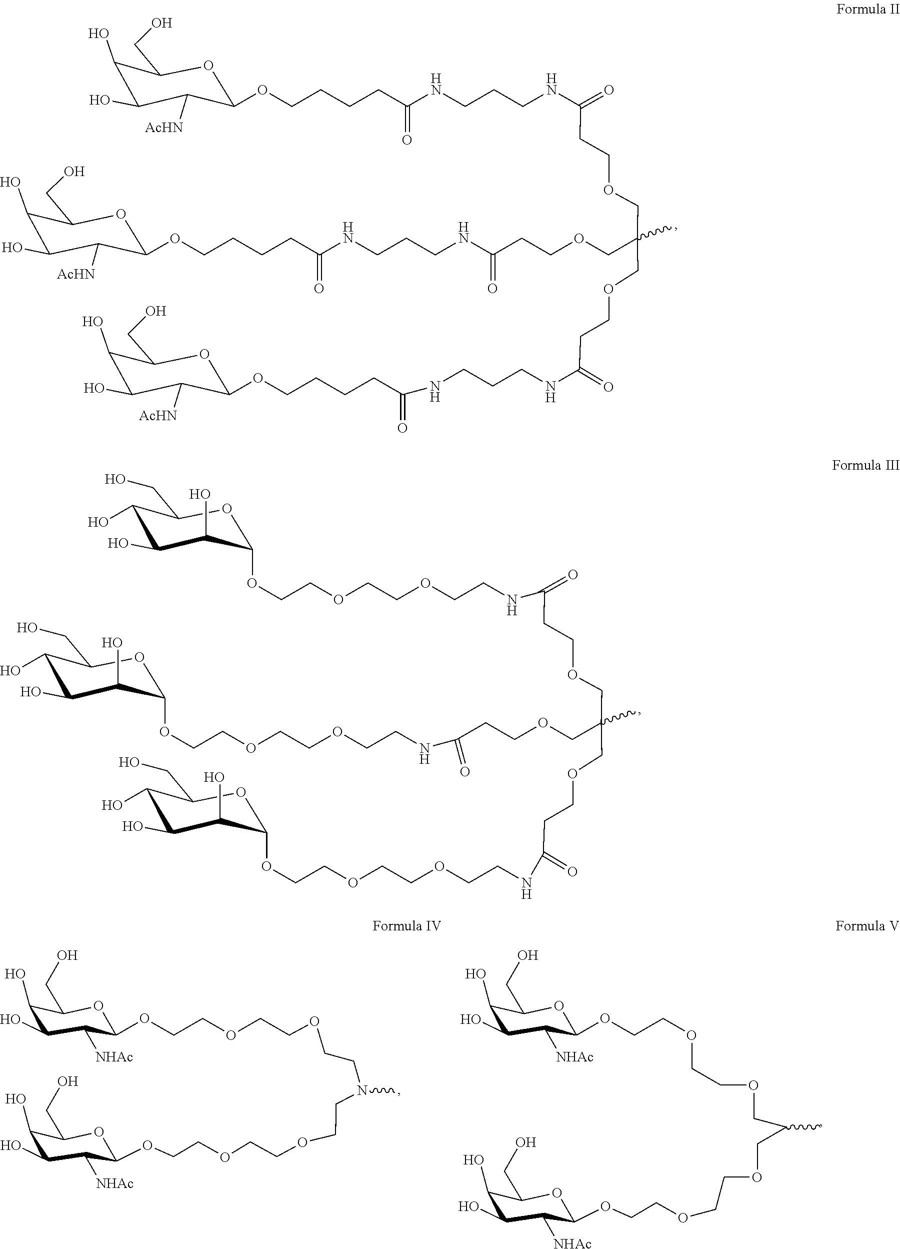

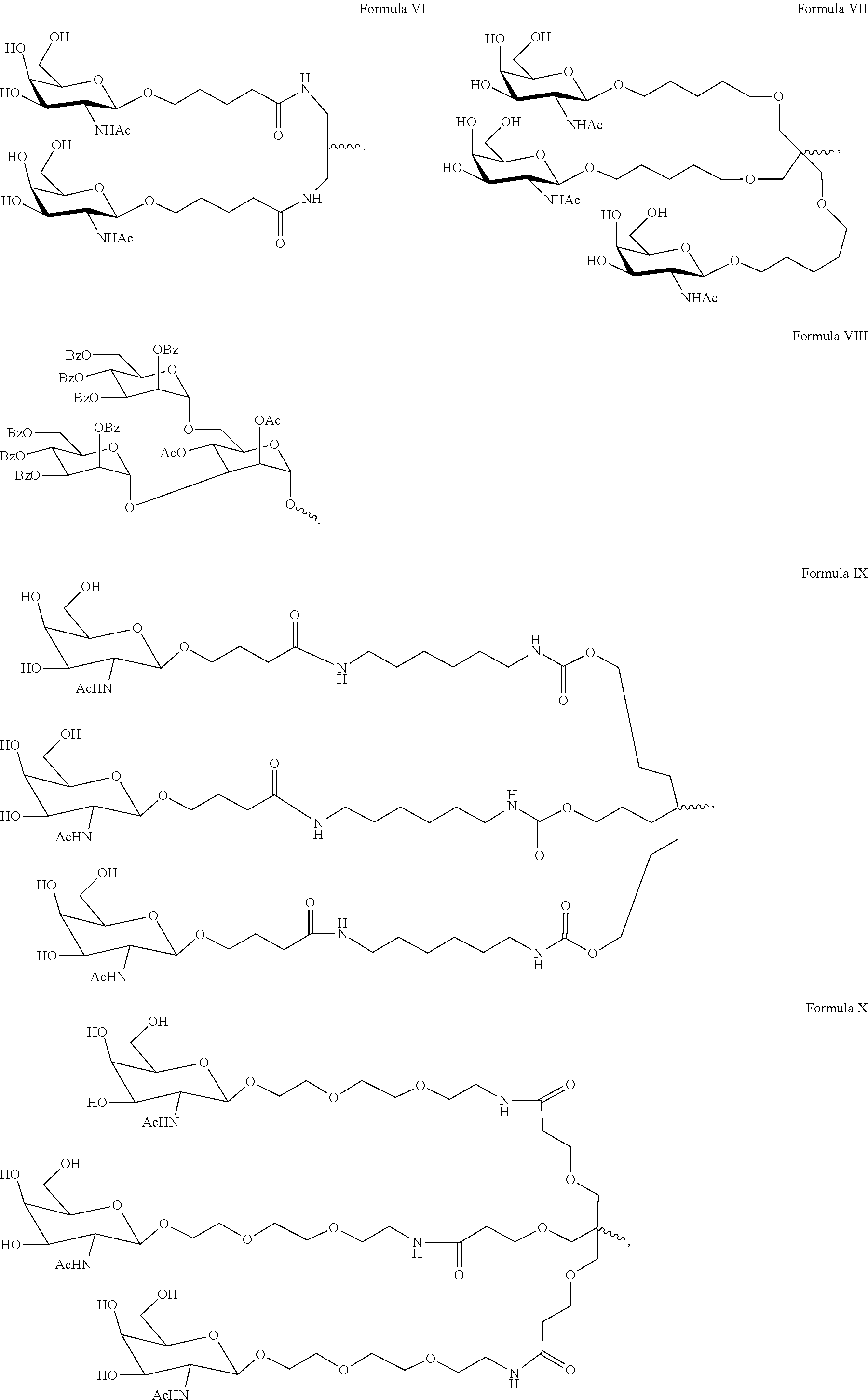

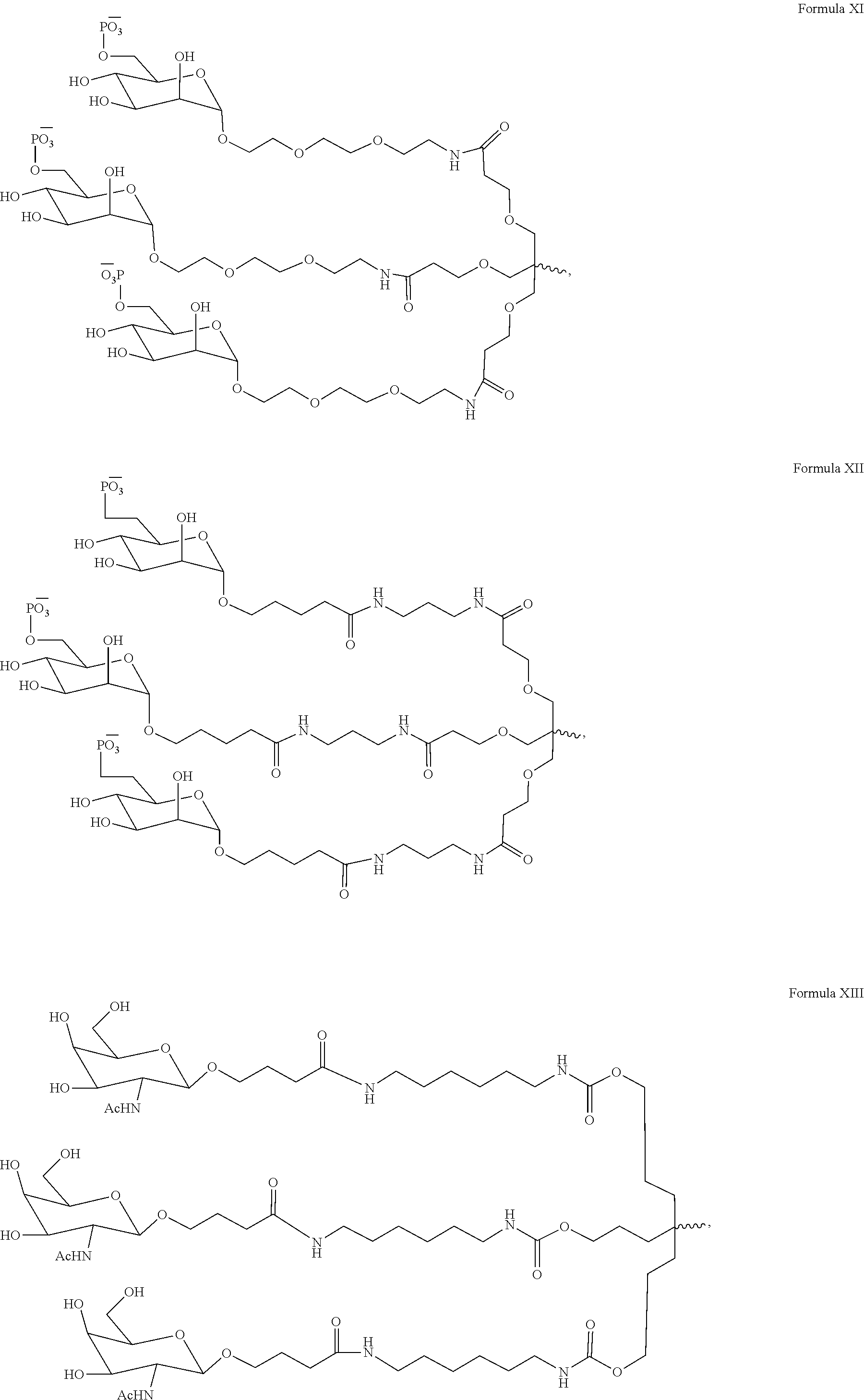

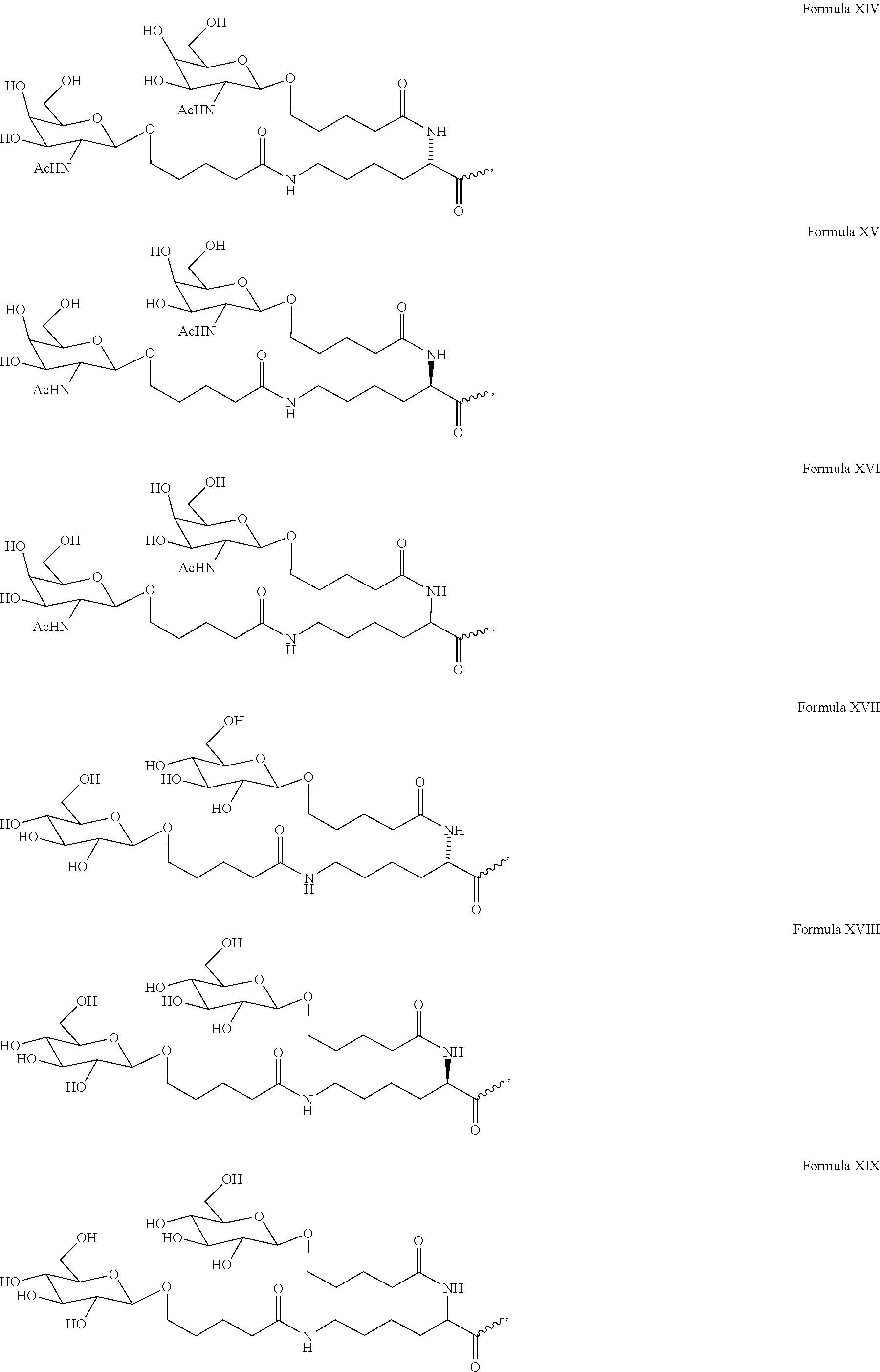

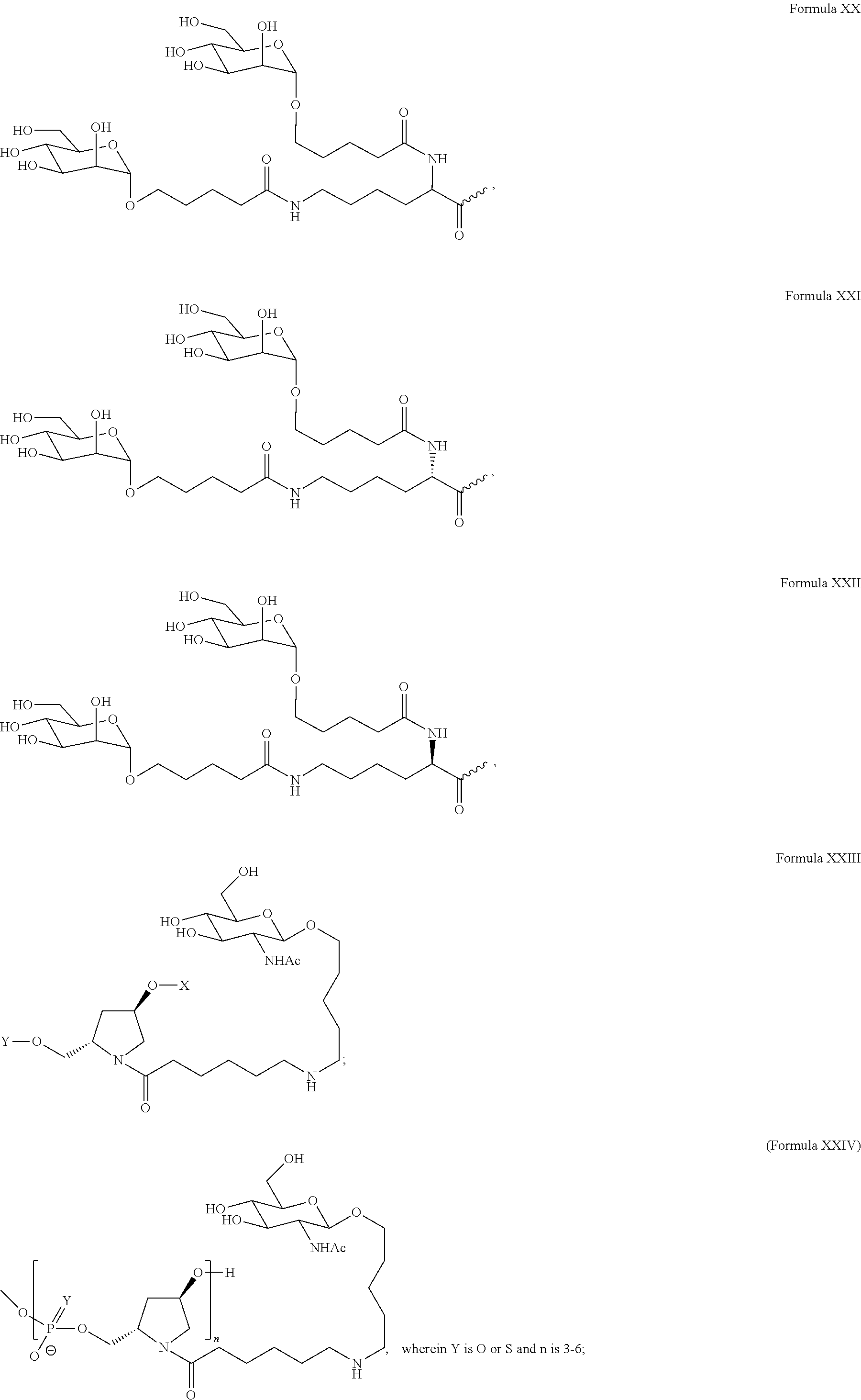

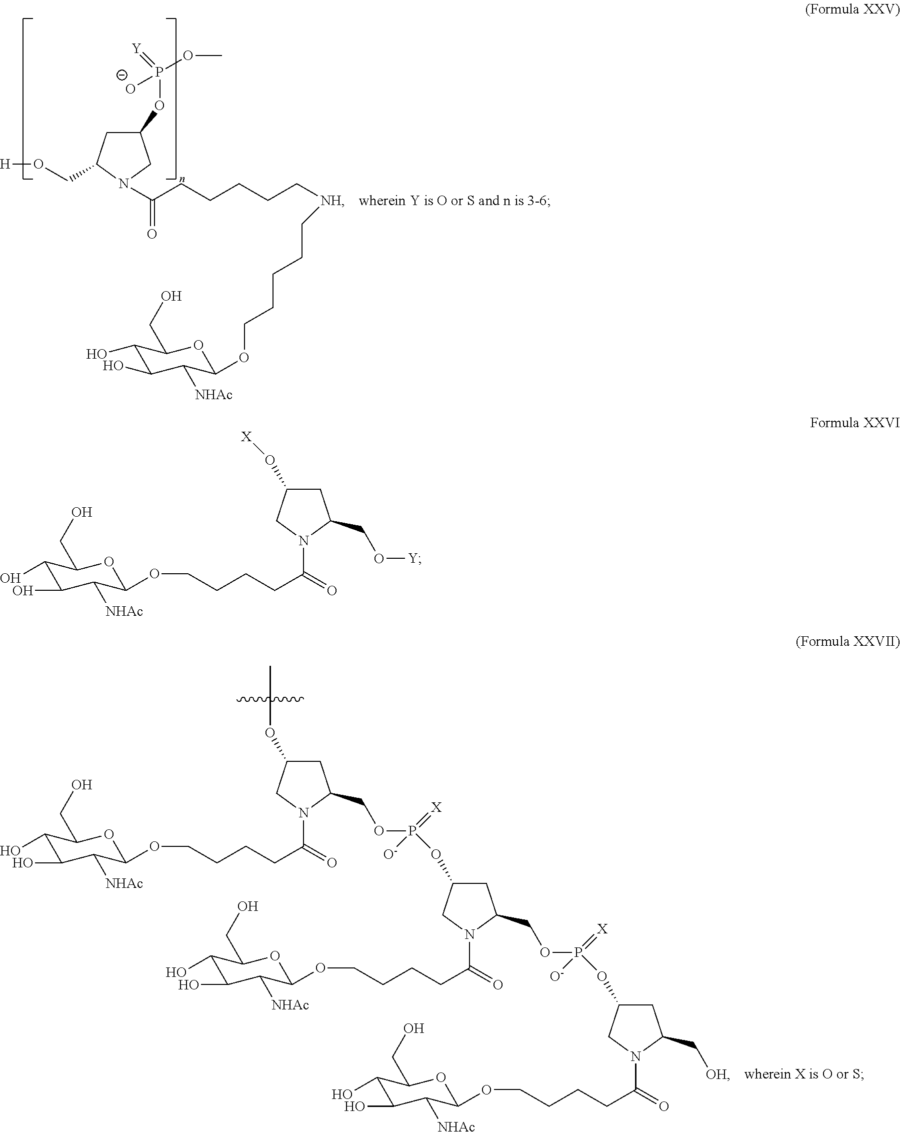

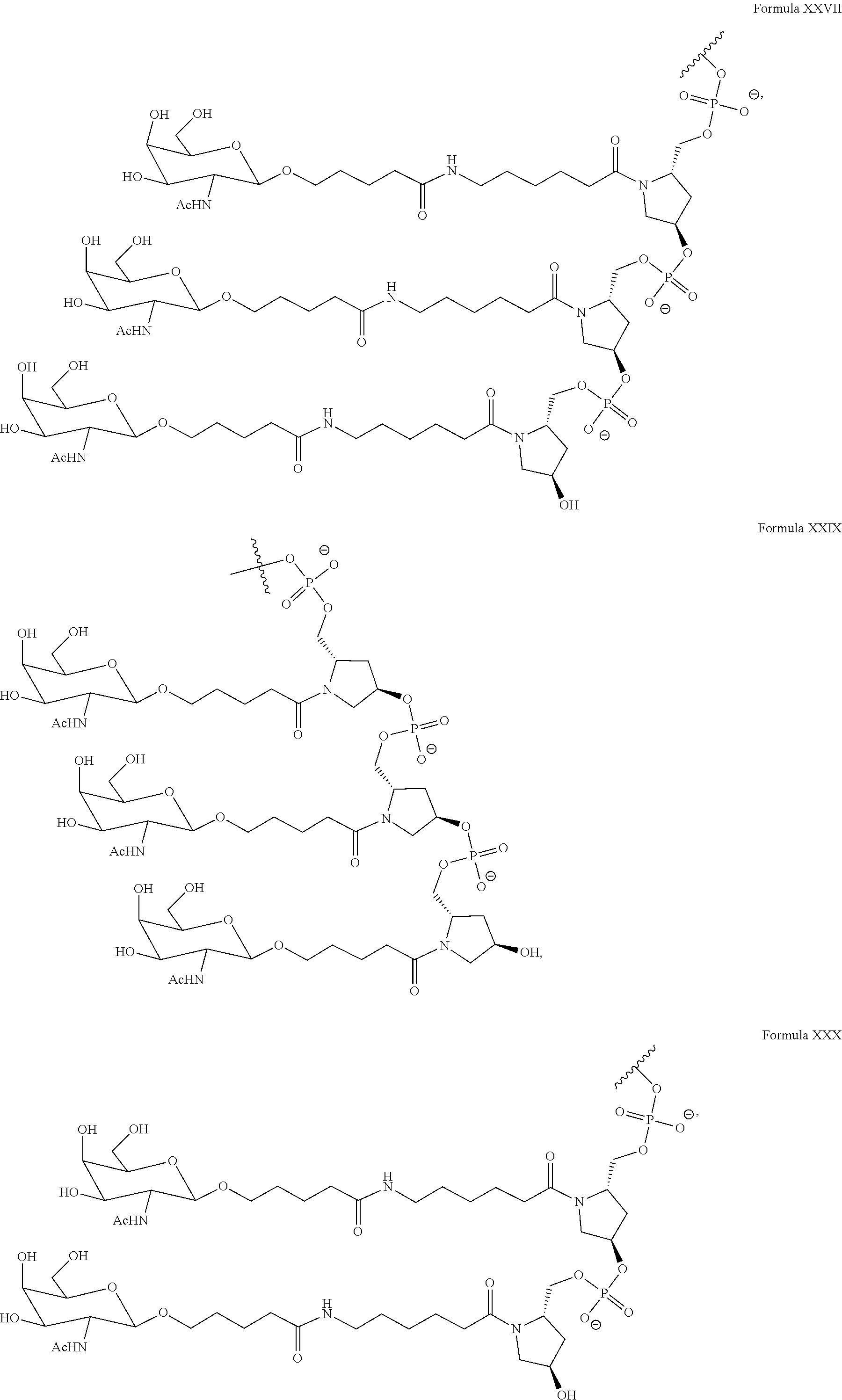

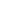

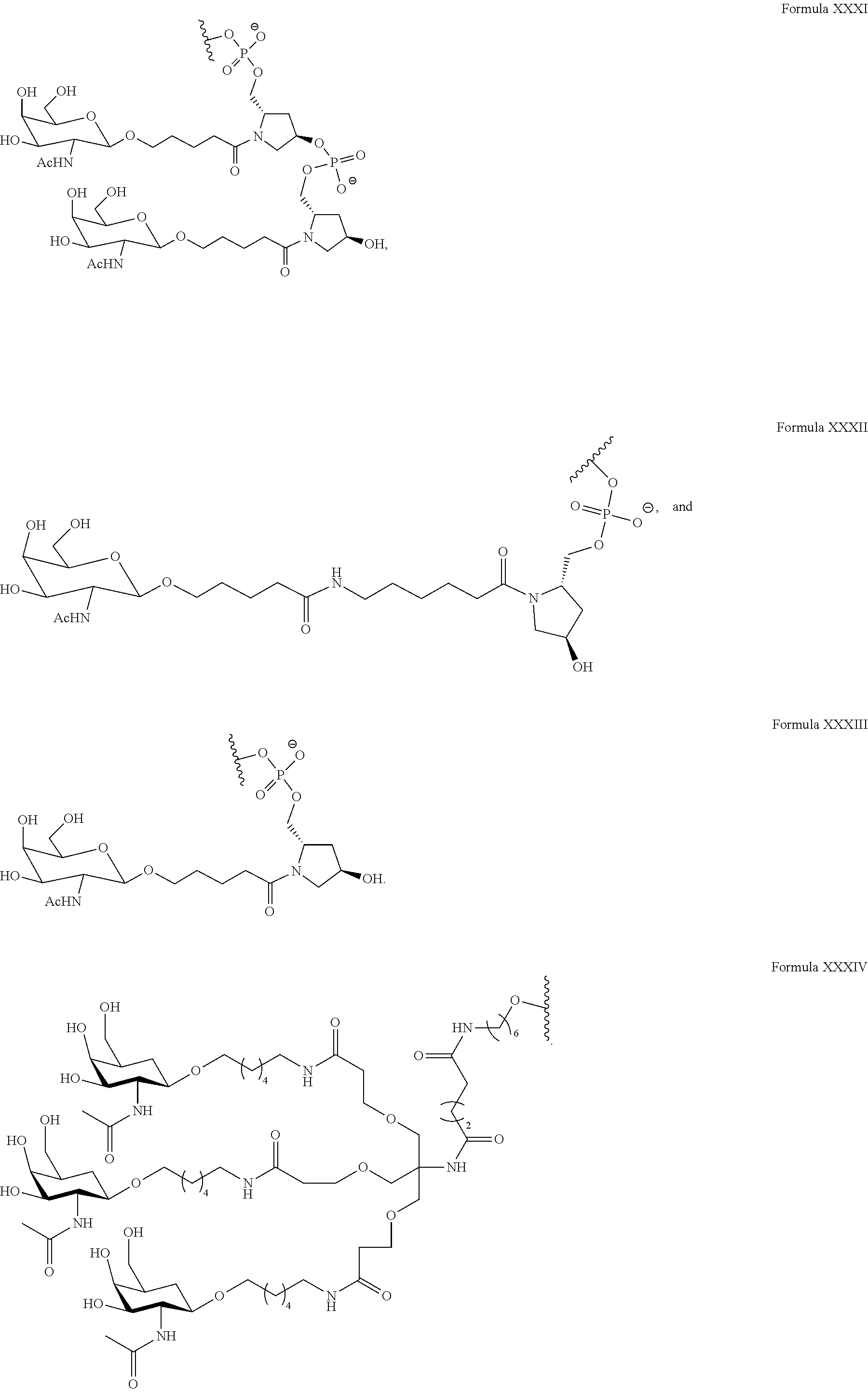

In another embodiment, a carbohydrate conjugate for use in the compositions and methods of the invention is a monosaccharide. In one embodiment, the monosaccharide is an N-acetylgalactosamine, such as

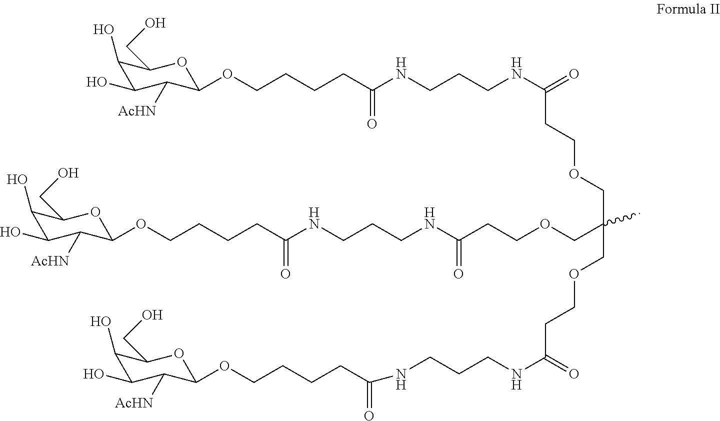 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

Another representative carbohydrate conjugate for use in the embodiments described herein includes, but is not limited to,

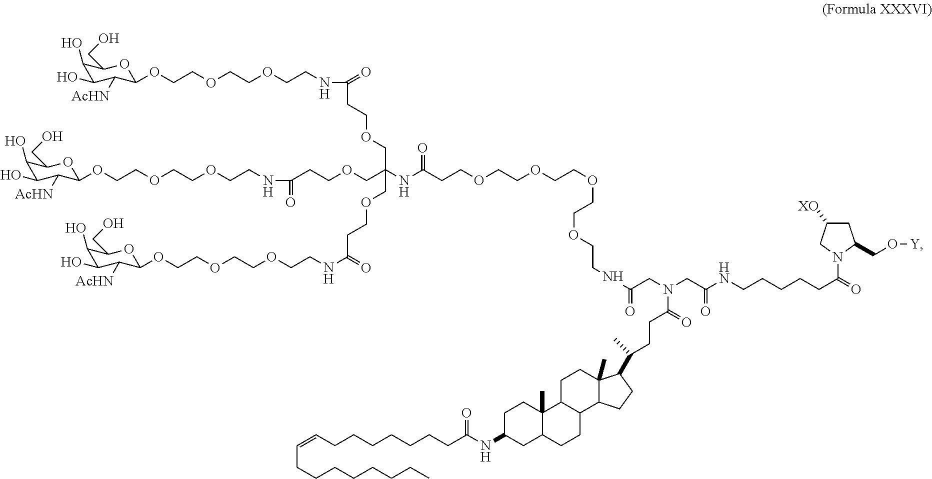

when one of X or Y is an oligonucleotide, the other is a hydrogen.

In certain embodiments of the invention, the GalNAc or GalNAc derivative is attached to an iRNA agent of the invention via a monovalent linker. In some embodiments, the GalNAc or GalNAc derivative is attached to an iRNA agent of the invention via a bivalent linker. In yet other embodiments of the invention, the GalNAc or GalNAc derivative is attached to an iRNA agent of the invention via a trivalent linker.

In one embodiment, the double stranded RNAi agents of the invention comprise one GalNAc or GalNAc derivative attached to the iRNA agent, e.g., the 5′end of the sense strand of a dsRNA agent, or the 5′ end of one or both sense strands of a dual targeting RNAi agent as described herein. In another embodiment, the double stranded RNAi agents of the invention comprise a plurality (e.g., 2, 3, 4, 5, or 6) GalNAc or GalNAc derivatives, each independently attached to a plurality of nucleotides of the double stranded RNAi agent through a plurality of monovalent linkers.

In some embodiments, for example, when the two strands of an iRNA agent of the invention are part of one larger molecule connected by an uninterrupted chain of nucleotides between the 3′-end of one strand and the 5′-end of the respective other strand forming a hairpin loop comprising, a plurality of unpaired nucleotides, each unpaired nucleotide within the hairpin loop may independently comprise a GalNAc or GalNAc derivative attached via a monovalent linker.

In some embodiments, the carbohydrate conjugate further comprises one or more additional ligands as described above, such as, but not limited to, a PK modulator or a cell permeation peptide.

Additional carbohydrate conjugates and linkers suitable for use in the present invention include those described in PCT Publication Nos. WO 2014/179620 and WO 2014/179627, the entire contents of each of which are incorporated herein by reference.

D. Linkers

In some embodiments, the conjugate or ligand described herein can be attached to an iRNA oligonucleotide with various linkers that can be cleavable or non-cleavable.

The term “linker” or “linking group” means an organic moiety that connects two parts of a compound, e.g., covalently attaches two parts of a compound. Linkers typically comprise a direct bond or an atom such as oxygen or sulfur, a unit such as NR8, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R8), C(O), substituted or unsubstituted aryl, substituted or unsubstituted heteroaryl, or substituted or unsubstituted heterocyclic; where R⁸ is hydrogen, acyl, aliphatic, or substituted aliphatic. In one embodiment, the linker is about 1-24 atoms, 2-24, 3-24, 4-24, 5-24, 6-24, 6-18, 7-18, 8-18, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, 30 times, 40 times, 50 times, 60 times, 70 times, 80 times, 90 times, or more, or at least 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissue. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or 100 times faster in the cell (or under in vitro conditions selected to mimic intracellular conditions) as compared to blood or serum (or under in vitro conditions selected to mimic extracellular conditions).

i. Redox Cleavable Linking Groups

In certain embodiments, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular iRNA moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

ii. Phosphate-Based Cleavable Linking Groups

In other embodiments, a cleavable linker comprises a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(ORk)-O—, —O—P(S)(ORk)-O—, —O—P(S)(SRk)-O—, —S—P(O)(ORk)-O—, —O—P(O)(ORk)-S—, —S—P(O)(ORk)-S—, —O—P(S)(ORk)-S—, —S—P(S)(ORk)-O—, —O—P(O)(Rk)-O—, —O—P(S)(Rk)-O—, —S—P(O)(Rk)-O—, —S—P(S)(Rk)-O—, —S—P(O)(Rk)-S—, —O—P(S)(Rk)-S—. Preferred embodiments are —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O, —S—P(S)(H)—O—, —S—P(O)(H)—S—, and —O—P(S)(H)—S—. A preferred embodiment is —O—P(O)(OH)—O—. These candidates can be evaluated using methods analogous to those described above.

iii. Acid Cleavable Linking Groups

In other embodiments, a cleavable linker comprises an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.5, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

iv. Ester-Based Linking Groups

In other embodiments, a cleavable linker comprises an ester-based cleavable linking group. An ester-based cleavable linking group is cleaved by enzymes such as esterases and amidases in cells. Examples of ester-based cleavable linking groups include, but are not limited to, esters of alkylene, alkenylene and alkynylene groups. Ester cleavable linking groups have the general formula —C(O)O—, or —OC(O)—. These candidates can be evaluated using methods analogous to those described above.

v. Peptide-Based Cleaving Groups

In yet other embodiments, a cleavable linker comprises a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In some embodiments, an iRNA of the invention is conjugated to a carbohydrate through a linker. Non-limiting examples of iRNA carbohydrate conjugates with linkers of the compositions and methods of the invention include, but are not limited to,

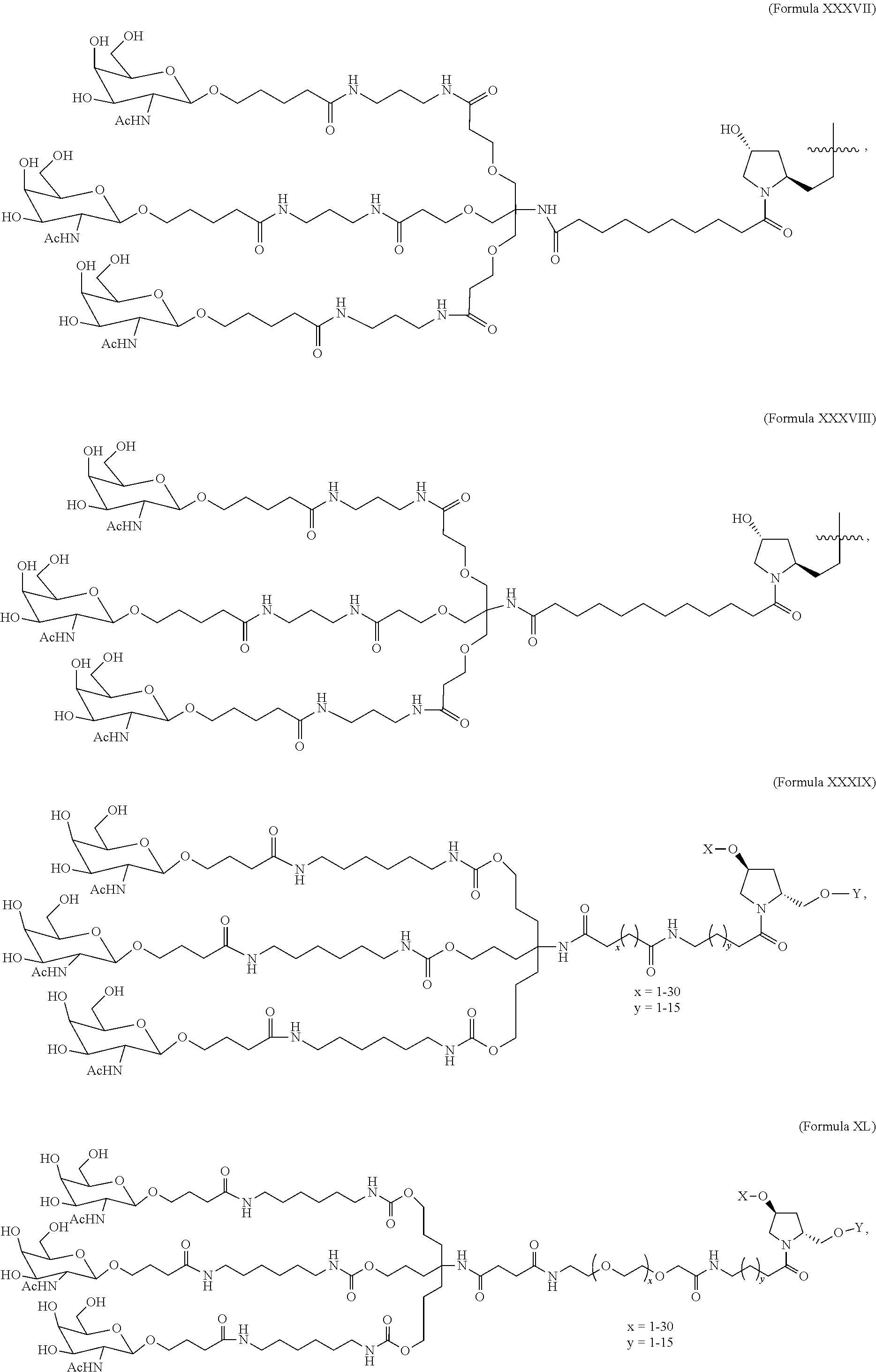

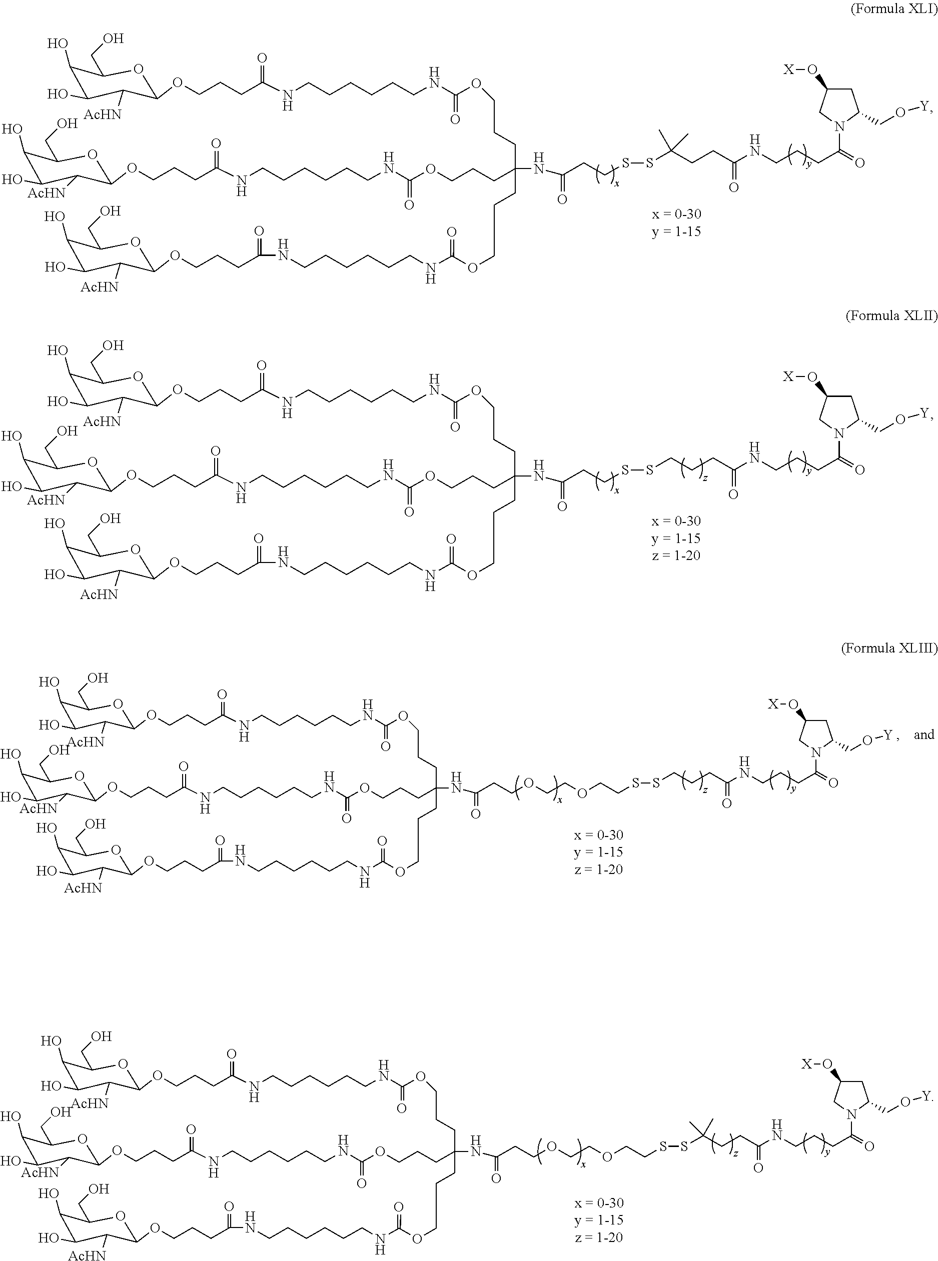

(Formula XLIV), when one of X or Y is an oligonucleotide, the other is a hydrogen.

In certain embodiments of the compositions and methods of the invention, a ligand is one or more “GalNAc” (N-acetylgalactosamine) derivatives attached through a bivalent or trivalent branched linker.

In one embodiment, a dsRNA of the invention is conjugated to a bivalent or trivalent branched linker selected from the group of structures shown in any of formula (XLV)-(XLVI):

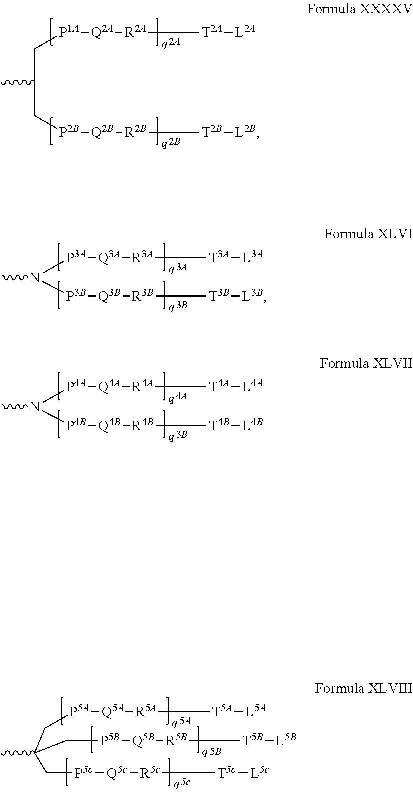

wherein:
q2A, q2B, q3A, q3B, q4A, q4B, q5A, q5B and q5C represent independently for each occurrence 0-20 and wherein the repeating unit can be the same or different;
P^{2A}, P^{2B}, P^{3A}, P^{3B}, P^{4A}, P^{4B}, P^{5A}, P^{5B}, P^{5C}, T^{2A}, T^{2B}, T^{3A}, T^{3B}, T^{4A}, T^{4B}, T^{4A}, T^{5B}, T^{5C} are each independently for each occurrence absent, CO, NH, O, S, OC(O), NHC(O), CH₂, CH₂NH or CH₂O;
Q^{2A}, Q^{2B}, Q^{3A}, Q^{3B}, Q^{4A}, Q^{4B}, Q^{5A}, Q^{5B}, Q^{5C} are independently for each occurrence absent, alkylene, substituted alkylene wherein one or more methylenes can be interrupted or terminated by one or more of O, S, S(O), SO₂, N(R^{N}), C(R′)═C(R″), C≡C or C(O);
R^{2A}, R^{2B}, R^{3A}, R^{3B}, R^{4A}, R^{4B}, R^{4B}, R^{5A}, R^{5B}, R^{5C} are each independently for each occurrence absent, NH, O, S, CH₂, C(O)O, C(O)NH, NHCH(R^{a})C(O), —C(O)—CH(R^{a})—NH—, CO, CH═N—

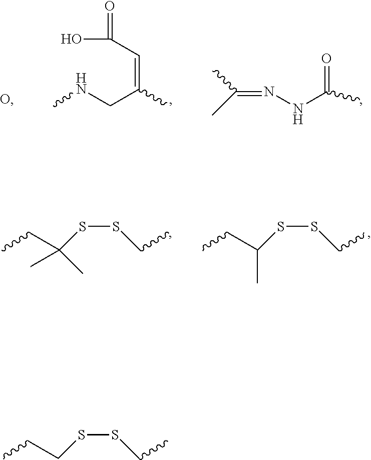

L^{2A}, L^{2B}, L^{3A}, L^{3B}, L^{4A}, L^{4B}, L^{5A}, L^{5B} and L^{5C} represent the ligand; i.e. each independently for each occurrence a monosaccharide (such as GalNAc), disaccharide, trisaccharide, tetrasaccharide, oligosaccharide, or polysaccharide; and R^{a} is H or amino acid side chain. Trivalent conjugating GalNAc derivatives are particularly useful for use with RNAi agents for inhibiting the expression of a target gene, such as those of formula (XLIX):

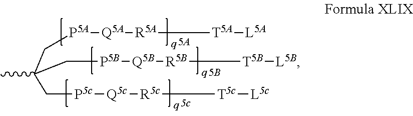

wherein L^{5A}, L^{5B} and L^{5C} represent a monosaccharide, such as GalNAc derivative.

Examples of suitable bivalent and trivalent branched linker groups conjugating GalNAc derivatives include, but are not limited to, the structures recited above as formulas II, VII, XI, X, and XIII.

Representative U.S. patents that teach the preparation of RNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,688,941; 6,294,664; 6,320,017; 6,576,752; 6,783,931; 6,900,297; 7,037,646; and 8,106,022, the entire contents of each of which are hereby incorporated herein by reference.

It is not necessary for all positions in a given compound to be uniformly modified, and in fact more than one of the aforementioned modifications can be incorporated in a single compound or even at a single nucleoside within an iRNA. The present invention also includes iRNA compounds that are chimeric compounds.

“Chimeric” iRNA compounds or “chimeras,” in the context of this invention, are iRNA compounds, preferably dsRNAi agents, that contain two or more chemically distinct regions, each made up of at least one monomer unit, i.e., a nucleotide in the case of a dsRNA compound. These iRNAs typically contain at least one region wherein the RNA is modified so as to confer upon the iRNA increased resistance to nuclease degradation, increased cellular uptake, or increased binding affinity for the target nucleic acid. An additional region of the iRNA can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of iRNA inhibition of gene expression. Consequently, comparable results can often be obtained with shorter iRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the RNA of an iRNA can be modified by a non-ligand group. A number of non-ligand molecules have been conjugated to iRNAs in order to enhance the activity, cellular distribution or cellular uptake of the iRNA, and procedures for performing such conjugations are available in the scientific literature. Such non-ligand moieties have included lipid moieties, such as cholesterol (Kubo, T. et al., Biochem. Biophys. Res. Comm., 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such RNA conjugates have been listed above. Typical conjugation protocols involve the synthesis of RNAs bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

IV. Delivery of an iRNA of the Invention

The delivery of an iRNA of the invention to a cell e.g., a cell within a subject, such as a human subject (e.g., a subject in need thereof, such as a subject at risk of developing or diagnosed with a metabolic disorder, e.g., a deficiency in glycemic control) can be achieved in a number of different ways. For example, delivery may be performed by contacting a cell with an iRNA of the invention either in vitro or in vivo. In vivo delivery may also be performed directly by administering a composition comprising an iRNA, e.g., a dsRNA, to a subject. Alternatively, in vivo delivery may be performed indirectly by administering one or more vectors that encode and direct the expression of the iRNA. These alternatives are discussed further below.

In general, any method of delivering a nucleic acid molecule (in vitro or in vivo) can be adapted for use with an iRNA of the invention (see e.g., Akhtar S. and Julian R L. (1992) Trends Cell. Biol. 2(5):139-144 and WO94/02595, which are incorporated herein by reference in their entireties). For in vivo delivery, factors to consider in order to deliver an iRNA molecule include, for example, biological stability of the delivered molecule, prevention of non-specific effects, and accumulation of the delivered molecule in the target tissue. The non-specific effects of an iRNA can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the iRNA molecule to be administered. Several studies have shown successful knockdown of gene products when a dsRNAi agent is administered locally. For example, intraocular delivery of a VEGF dsRNA by intravitreal injection in cynomolgus monkeys (Tolentino, M J, et al (2004) Retina 24:132-138) and subretinal injections in mice (Reich, S J., et al (2003) Mol. Vis. 9:210-216) were both shown to prevent neovascularization in an experimental model of age-related macular degeneration. In addition, direct intratumoral injection of a dsRNA in mice reduces tumor volume (Pille, J., et al (2005) Mol. Ther. 11:267-274) and can prolong survival of tumor-bearing mice (Kim, W J., et al (2006) Mol. Ther. 14:343-350; Li, S., et al (2007) Mol. Ther. 15:515-523). RNA interference has also shown success with local delivery to the CNS by direct injection (Dorn, G., et al. (2004) Nucleic Acids 32:e49; Tan, P H., et al (2005) Gene Ther. 12:59-66; Makimura, H., et al (2002) BMC Neurosci. 3:18; Shishkina, G T., et al (2004) Neuroscience 129:521-528; Thakker, E R., et al (2004) Proc. Natl. Acad. Sci. U.S.A. 101:17270-17275; Akaneya, Y., et al (2005) J. Neurophysiol. 93:594-602) and to the lungs by intranasal administration (Howard, K A., et al (2006) Mol. Ther. 14:476-484; Zhang, X., et al (2004) J. Biol. Chem. 279:10677-10684; Bitko, V., et al (2005) Nat. Med. 11:50-55). For administering an iRNA systemically for the prevention of an infection, the RNA can be modified or alternatively delivered using a drug delivery system; both methods act to prevent the rapid degradation of the dsRNA by endo- and exo-nucleases in vivo. Modification of the RNA or the pharmaceutical carrier can also permit targeting of the iRNA to the target tissue and avoid undesirable off-target effects. iRNA molecules can be modified by chemical conjugation to lipophilic groups such as cholesterol to enhance cellular uptake and prevent degradation. For example, an iRNA directed against ApoB conjugated to a lipophilic cholesterol moiety was injected systemically into mice and resulted in knockdown of apoB mRNA in both the liver and jejunum (Soutschek, J., et al (2004) Nature 432:173-178). Conjugation of an iRNA to an aptamer has been shown to inhibit tumor growth and mediate tumor regression in a mouse model of prostate cancer (McNamara, J O, et al (2006) Nat. Biotechnol. 24:1005-1015). In an alternative embodiment, the iRNA can be delivered using drug delivery systems such as a nanoparticle, a dendrimer, a polymer, liposomes, or a cationic delivery system. Positively charged cationic delivery systems facilitate binding of an iRNA molecule (negatively charged) and also enhance interactions at the negatively charged cell membrane to permit efficient uptake of an iRNA by the cell. Cationic lipids, dendrimers, or polymers can either be bound to an iRNA, or induced to form a vesicle or micelle (see e.g., Kim S H, et al (2008) Journal of Controlled Release 129(2):107-116) that encases an iRNA. The formation of vesicles or micelles further prevents degradation of the iRNA when administered systemically. Methods for making and administering cationic-iRNA complexes are well within the abilities of one skilled in the art (see e.g., Sorensen, D R, et al (2003) J. Mol. Biol 327:761-766; Verma, U N, et al (2003) Clin. Cancer Res. 9:1291-1300; Arnold, A S et al (2007) J. Hypertens. 25:197-205, which are incorporated herein by reference in their entirety). Some non-limiting examples of drug delivery systems useful for systemic delivery of iRNAs include DOTAP (Sorensen, D R., et al (2003), supra; Verma, U N, et al (2003), supra), Oligofectamine, “solid nucleic acid lipid particles” (Zimmermann, T S, et al (2006) Nature 441:111-114), cardiolipin (Chien, P Y, et al (2005) Cancer Gene Ther. 12:321-328; Pal, A, et al (2005) Int J. Oncol. 26:1087-1091), polyethyleneimine (Bonnet M E, et al (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A, et al (2007) Biochem. Soc. Trans. 35:61-67; Yoo, H., et al (1999) Pharm. Res. 16:1799-1804). In some embodiments, an iRNA forms a complex with cyclodextrin for systemic administration. Methods for administration and pharmaceutical compositions of iRNAs and cyclodextrins can be found in U.S. Pat. No. 7,427,605, which is herein incorporated by reference in its entirety.

A. Vector encoded iRNAs of the Invention

iRNA targeting the HMGB1 gene can be expressed from transcription units inserted into DNA or RNA vectors (see, e.g., Couture, A, et al., TIG. (1996), 12:5-10; Skillern, A, et al., PCT Publication No. WO 00/22113, PCT Publication No. WO 00/22114, and U.S. Pat. No. 6,054,299). Expression can be transient (on the order of hours to weeks) or sustained (weeks to months or longer), depending upon the specific construct used and the target tissue or cell type. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

The individual strand or strands of an iRNA can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as inverted repeat polynucleotides joined by a linker polynucleotide sequence such that the dsRNA has a stem and loop structure.

iRNA expression vectors are generally DNA plasmids or viral vectors. Expression vectors compatible with eukaryotic cells, preferably those compatible with vertebrate cells, can be used to produce recombinant constructs for the expression of an iRNA as described herein. Eukaryotic cell expression vectors are well known in the art and are available from a number of commercial sources. Typically, such vectors are provided containing convenient restriction sites for insertion of the desired nucleic acid segment. Delivery of iRNA expressing vectors can be systemic, such as by intravenous or intramuscular administration, by administration to target cells ex-planted from the patient followed by reintroduction into the patient, or by any other means that allows for introduction into a desired target cell.

Viral vector systems which can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g. EPV and EBV vectors. Constructs for the recombinant expression of an iRNA will generally require regulatory elements, e.g., promoters, enhancers, etc., to ensure the expression of the iRNA in target cells. Other aspects to consider for vectors and constructs are known in the art.

### V. Pharmaceutical Compositions of the Invention

The present invention also includes pharmaceutical compositions and formulations which include the iRNAs of the invention. In one embodiment, provided herein are pharmaceutical compositions containing an iRNA, as described herein, and a pharmaceutically acceptable carrier. The pharmaceutical compositions containing the iRNA are useful for preventing or treating an HMGB1-associated disorder, e.g., metabolic disorder or NAFLD, e.g., NASH. Such pharmaceutical compositions are formulated based on the mode of delivery. One example is compositions that are formulated for systemic administration via parenteral delivery, e.g., by subcutaneous (SC), intramuscular (IM), or intravenous (IV) delivery. The pharmaceutical compositions of the invention may be administered in dosages sufficient to inhibit expression of an HMGB1 gene.

The pharmaceutical compositions of the invention may be administered in dosages sufficient to inhibit expression of an HMGB1 gene. In general, a suitable dose of an iRNA of the invention will be in the range of about 0.001 to about 200.0 milligrams per kilogram body weight of the recipient per day, generally in the range of about 1 to 50 mg per kilogram body weight per day. Typically, a suitable dose of an iRNA of the invention will be in the range of about 0.1 mg/kg to about 5.0 mg/kg, preferably about 0.3 mg/kg and about 3.0 mg/kg. A repeat-dose regimen may include administration of a therapeutic amount of iRNA on a regular basis, such as every month, once every 3-6 months, or once a year. In certain embodiments, the iRNA is administered about once per month to about once per six months.

After an initial treatment regimen, the treatments can be administered on a less frequent basis. Duration of treatment can be determined based on the severity of disease.

In other embodiments, a single dose of the pharmaceutical compositions can be long lasting, such that doses are administered at not more than 1, 2, 3, or 4 month intervals. In some embodiments of the invention, a single dose of the pharmaceutical compositions of the invention is administered about once per month. In other embodiments of the invention, a single dose of the pharmaceutical compositions of the invention is administered quarterly (i.e., about every three months). In other embodiments of the invention, a single dose of the pharmaceutical compositions of the invention is administered twice per year (i.e., about once every six months).

The skilled artisan will appreciate that certain factors can influence the dosage and timing required to effectively treat a subject, including but not limited to mutations present in the subject, previous treatments, the general health or age of the subject, and other diseases present. Moreover, treatment of a subject with a prophylactically or therapeutically effective amount, as appropriate, of a composition can include a single treatment or a series of treatments.

The pharmaceutical compositions of the present invention can be administered in a number of ways depending upon whether local or systemic treatment is desired and upon the area to be treated. Administration can be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal, or intramuscular injection or infusion; subdermal, e.g., via an implanted device; or intracranial, e.g., by intraparenchymal, intrathecal or intraventricular administration.

The iRNA can be delivered in a manner to target a particular tissue (e.g., hepatocytes).

Pharmaceutical compositions and formulations for topical or transdermal administration can include transdermal patches, ointments, lotions, creams, gels, drops, suppositories, sprays, liquids, and powders. Conventional pharmaceutical carriers, aqueous, powder or oily bases, thickeners and the like can be necessary or desirable. Coated condoms, gloves and the like can also be useful. Suitable topical formulations include those in which the iRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). iRNAs featured in the invention can be encapsulated within liposomes or can form complexes thereto, in particular to cationic liposomes. Alternatively, iRNAs can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₂₀ alkyl ester (e.g., isopropylmyristate IPM), monoglyceride, diglyceride or pharmaceutically acceptable salt thereof). Topical formulations are described in detail in U.S. Pat. No. 6,747,014, which is incorporated herein by reference.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids, or binders can be desirable. In some embodiments, oral formulations are those in which dsRNAs featured in the invention are administered in conjunction with one or more penetration enhancer surfactants and chelators. Suitable surfactants include fatty acids or esters or salts thereof, bile acids or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. DsRNAs featured in the invention can be delivered orally, in granular form including sprayed dried particles, or complexed to form micro or nanoparticles. DsRNA complexing agents include polyamino acids; polyimines; polyacrylates; polyalkylacrylates, polyoxethanes, polyalkylcyanoacrylates; cationized gelatins, albumins, starches, acrylates, polyethyleneglycols (PEG), and starches; polyalkylcyanoacrylates; DEAE-derivatized polyimines, pollulans, celluloses, and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, U.S. Publn. No. 20030027780, and U.S. Pat. No. 6,747,014, each of which is incorporated herein by reference.

Compositions and formulations for parenteral, intraparenchymal (into the brain), intrathecal, intraventricular or intrahepatic administration can include sterile aqueous solutions which can also contain buffers, diluents, and other suitable additives such as, but not limited to, penetration enhancers, carrier compounds, and other pharmaceutically acceptable carriers or excipients.

Pharmaceutical compositions of the present invention include, but are not limited to, solutions, emulsions, and liposome-containing formulations. These compositions can be generated from a variety of components that include, but are not limited to, preformed liquids, self-emulsifying solids, and self-emulsifying semisolids. Formulations include those that target the liver.

The pharmaceutical formulations of the present invention, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention can be formulated into any of many possible dosage forms such as, but not limited to, tablets, capsules, gel capsules, liquid syrups, soft gels, suppositories, and enemas. The compositions of the present invention can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

A. Additional Formulations

i. Emulsions

The compositions of the present invention can be prepared and formulated as emulsions. Emulsions are typically heterogeneous systems of one liquid dispersed in another in the form of droplets usually exceeding 0.1 μm in diameter (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution either in the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic, and amphoteric (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y. Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin, and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate, and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives, and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols, and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral, and parenteral routes, and methods for their manufacture have been reviewed in the literature (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins, and high fat nutritive preparations are among the materials that have commonly been administered orally as o/w emulsions.

ii. Microemulsions

In one embodiment of the present invention, the compositions of iRNAs and nucleic acids are formulated as microemulsions. A microemulsion can be defined as a system of water, oil, and amphiphile which is a single optically isotropic and thermodynamically stable liquid solution (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (see e.g., Ansel's Pharmaceutical Dosage Forms and Drug Delivery Systems, Allen, L V., Popovich N G., and Ansel H C., 2004, Lippincott Williams & Wilkins (8th ed.), New York, N.Y.; Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij® 96, polyoxyethylene oleyl ethers, polyglycerol fatty acid esters, tetraglycerol monolaurate (ML310), tetraglycerol monooleate (M0310), hexaglycerol monooleate (P0310), hexaglycerol pentaoleate (P0500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex® 300, Captex® 355, Capmul® MCM, fatty acid esters, medium chain (C8-C12) mono, di, and tri-glycerides, polyoxyethylated glyceryl fatty acid esters, fatty alcohols, polyglycolized glycerides, saturated polyglycolized C₈-C₁₀ glycerides, vegetable oils, and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (see e.g., U.S. Pat. Nos. 6,191,105; 7,063,860; 7,070,802; 7,157,099; Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (see e.g., U.S. Pat. Nos. 6,191,105; 7,063,860; 7,070,802; 7,157,099; Constantinides et al., Pharmaceutical Research, 1994, 11, 1385; Ho et al., J. Pharm. Sci., 1996, 85, 138-143). Often microemulsions can form spontaneously when their components are brought together at ambient temperature. This can be particularly advantageous when formulating thermolabile drugs, peptides or iRNAs. Microemulsions have also been effective in the transdermal delivery of active components in both cosmetic and pharmaceutical applications. It is expected that the microemulsion compositions and formulations of the present invention will facilitate the increased systemic absorption of iRNAs and nucleic acids from the gastrointestinal tract, as well as improve the local cellular uptake of iRNAs and nucleic acids.

Microemulsions of the present invention can also contain additional components and additives such as sorbitan monostearate (Grill® 3), Labrasol@, and penetration enhancers to improve the properties of the formulation and to enhance the absorption of the iRNAs and nucleic acids of the present invention. Penetration enhancers used in the microemulsions of the present invention can be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

iii. Microparticles

An iRNA of the invention may be incorporated into a particle, e.g., a microparticle. Microparticles can be produced by spray-drying, but may also be produced by other methods including lyophilization, evaporation, fluid bed drying, vacuum drying, or a combination of these techniques.

iv. Penetration Enhancers

In one embodiment, the present invention employs various penetration enhancers to effect the efficient delivery of nucleic acids, particularly iRNAs, to the skin of animals. Most drugs are present in solution in both ionized and nonionized forms. However, usually only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories, i.e., surfactants, fatty acids, bile salts, chelating agents, and non-chelating non-surfactants (see e.g., Malmsten, M. Surfactants and polymers in drug delivery, Informa Health Care, New York, N.Y., 2002; Lee et al., Critical Reviews in Therapeutic Drug Carrier Systems, 1991, p. 92).

Each of the above mentioned classes of penetration enhancers are described below in greater detail.

Surfactants (or “surface-active agents”) are chemical entities which, when dissolved in an aqueous solution, reduce the surface tension of the solution or the interfacial tension between the aqueous solution and another liquid, with the result that absorption of iRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (see e.g., Malmsten, M. Surfactants and polymers in drug delivery, Informa Health Care, New York, N.Y., 2002; Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₂₀ alkyl esters thereof (e.g., methyl, isopropyl and t-butyl), and mono- and di-glycerides thereof (i.e., oleate, laurate, caprate, myristate, palmitate, stearate, linoleate, etc.) (see e.g., Touitou, E., et al. Enhancement in Drug Delivery, CRC Press, Danvers, Mass., 2006; Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

The physiological role of bile includes the facilitation of dispersion and absorption of lipids and fat-soluble vitamins (see e.g., Malmsten, M. Surfactants and polymers in drug delivery, Informa Health Care, New York, N.Y., 2002; Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, M. Surfactants and polymers in drug delivery, Informa Health Care, New York, N.Y., 2002; Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating agents, as used in connection with the present invention, can be defined as compounds that remove metallic ions from solution by forming complexes therewith, with the result that absorption of iRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(see e.g., Katdare, A. et al., Excipient development for pharmaceutical, biotechnology, and drug delivery, CRC Press, Danvers, Mass., 2006; Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

As used herein, non-chelating non-surfactant penetration enhancing compounds can be defined as compounds that demonstrate insignificant activity as chelating agents or as surfactants but that nonetheless enhance absorption of iRNAs through the alimentary mucosa (see e.g., Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of iRNAs at the cellular level can also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs. Examples of commercially available transfection reagents include, for example Lipofectamine™ (Invitrogen; Carlsbad, Calif.), Lipofectamine 2000™ (Invitrogen; Carlsbad, Calif.), 293fectin™ (Invitrogen; Carlsbad, Calif.), Cellfectin™ (Invitrogen; Carlsbad, Calif.), DMRIE-C™ (Invitrogen; Carlsbad, Calif.), FreeStyle™ MAX (Invitrogen; Carlsbad, Calif.), Lipofectamine™ 2000 CD (Invitrogen; Carlsbad, Calif.), Lipofectamine™ (Invitrogen; Carlsbad, Calif.), RNAiMAX (Invitrogen; Carlsbad, Calif.), Oligofectamine™ (Invitrogen; Carlsbad, Calif.), Optifect™ (Invitrogen; Carlsbad, Calif.), X-tremeGENE Q2 Transfection Reagent (Roche; Grenzacherstrasse, Switzerland), DOTAP Liposomal Transfection Reagent (Grenzacherstrasse, Switzerland), DOSPER Liposomal Transfection Reagent (Grenzacherstrasse, Switzerland), or Fugene (Grenzacherstrasse, Switzerland), Transfectam® Reagent (Promega; Madison, Wis.), TransFast™ Transfection Reagent (Promega; Madison, Wis.), Tfx™-20 Reagent (Promega; Madison, Wis.), Tfx™-50 Reagent (Promega; Madison, Wis.), DreamFect™ (OZ Biosciences; Marseille, France), EcoTransfect (OZ Biosciences; Marseille, France), TransPass^{a} D1 Transfection Reagent (New England Biolabs; Ipswich, Mass., USA), LyoVec™/LipoGen™ (Invitrogen; San Diego, Calif., USA), PerFectin Transfection Reagent (Genlantis; San Diego, Calif., USA), NeuroPORTER Transfection Reagent (Genlantis; San Diego, Calif., USA), GenePORTER Transfection reagent (Genlantis; San Diego, Calif., USA), GenePORTER 2 Transfection reagent (Genlantis; San Diego, Calif., USA), Cytofectin Transfection Reagent (Genlantis; San Diego, Calif., USA), BaculoPORTER Transfection Reagent (Genlantis; San Diego, Calif., USA), TroganPORTER™ transfection Reagent (Genlantis; San Diego, Calif., USA), RiboFect (Bioline; Taunton, Mass., USA), PlasFect (Bioline; Taunton, Mass., USA), UniFECTOR (B-Bridge International; Mountain View, Calif., USA), SureFECTOR (B-Bridge International; Mountain View, Calif., USA), or HiFect™ (B-Bridge International, Mountain View, Calif., USA), among others.

Other agents can be utilized to enhance the penetration of the administered nucleic acids, including glycols such as ethylene glycol and propylene glycol, pyrrols such as 2-pyrrol, azones, and terpenes such as limonene and menthone.

v. Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate dsRNA in hepatic tissue can be reduced when it is coadministered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

vi. Excipients

In contrast to a carrier compound, a “pharmaceutical carrier” or “excipient” is a pharmaceutically acceptable solvent, suspending agent, or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone, and the like.

vii. Other Components

The compositions of the present invention can additionally contain other adjunct components conventionally found in pharmaceutical compositions, at their art-established usage levels. Thus, for example, the compositions can contain additional, compatible, pharmaceutically-active materials such as, for example, antipruritics, astringents, local anesthetics or anti-inflammatory agents, or can contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings, or aromatic substances, and the like which do not deleteriously interact with the nucleic acid(s) of the formulation.

Aqueous suspensions can contain substances which increase the viscosity of the suspension including, for example, sodium carboxymethylcellulose, sorbitol, or dextran. The suspension can also contain stabilizers.

In some embodiments, pharmaceutical compositions featured in the invention include (a) one or more iRNA and (b) one or more agents which function by a non-iRNA mechanism and which are useful in treating a metabolic disorder, e.g., a deficiency in glycemic control.

Toxicity and prophylactic efficacy of such compounds can be determined by standard pharmaceutical procedures in cell cultures or experimental animals, e.g., for determining the LD50 (the dose lethal to 50% of the population) and the ED50 (the dose prophylactically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured herein in the invention lies generally within a range of circulating concentrations that include the ED50, preferably an ED80 or ED90, with little or no toxicity. The dosage can vary within this range depending upon the dosage form employed and the route of administration utilized. For any compound used in the methods featured in the invention, the prophylactically effective dose can be estimated initially from cell culture assays. A dose can be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) or higher levels of inhibition as determined in cell culture. Such information can be used to more accurately determine useful doses in humans. Levels in plasma can be measured, for example, by high performance liquid chromatography.

In addition to their administration, as discussed above, the iRNAs featured in the invention can be administered in combination with other known agents used for the prevention or treatment of a metabolic disorder, e.g., a deficiency in glycemic control. In any event, the administering physician can adjust the amount and timing of iRNA administration on the basis of results observed using standard measures of efficacy known in the art or described herein.

### VI. Methods for Inhibiting HMGB1 Expression

The present invention also provides methods of inhibiting expression of an HMGB1 gene in a cell. The methods include contacting a cell with an RNAi agent, e.g., double stranded RNA agent, in an amount effective to inhibit expression of HMGB1 in the cell, thereby inhibiting expression of HMGB1 in the cell.

Contacting of a cell with an iRNA, e.g., a double stranded RNA agent, may be done in vitro or in vivo. Contacting a cell in vitro includes contacting a group of cells in culture. Contacting a cell in vivo with the iRNA includes contacting a cell or group of cells within a subject, e.g., a human subject, with the iRNA. Combinations of in vitro and in vivo methods of contacting a cell are also possible. Contacting a cell may be direct or indirect, as discussed above. Furthermore, contacting a cell may be accomplished via a targeting ligand, including any ligand described herein or known in the art. In preferred embodiments, the targeting ligand is a carbohydrate moiety, e.g., a GalNAc₃ ligand, or any other ligand that directs the RNAi agent to a site of interest.

The term “inhibiting,” as used herein, is used interchangeably with “reducing,” “silencing,” “downregulating”, “suppressing”, and other similar terms, and includes any level of inhibition.

The phrase “inhibiting expression of an HMGB1” is intended to refer to inhibition of expression of any HMGB1 gene (such as, e.g., a mouse HMGB1 gene, a rat HMGB1 gene, a monkey HMGB1 gene, or a human HMGB1 gene) as well as variants or mutants of an HMGB1 gene. Thus, the HMGB1 gene may be a wild-type HMGB1 gene, a mutant HMGB1 gene, or a transgenic HMGB1 gene in the context of a genetically manipulated cell, group of cells, or organism.

“Inhibiting expression of an HMGB1 gene” includes any level of inhibition of an HMGB1 gene, e.g., at least partial suppression of the expression of an HMGB1 gene. The expression of the HMGB1 gene may be assessed based on the level, or the change in the level, of any variable associated with HMGB1 gene expression, e.g., HMGB1 mRNA level or HMGB1 protein level. This level may be assessed in an individual cell or in a group of cells, including, for example, a sample derived from a subject. It is understood that HMGB1 is expressed in a number of tissue types in the body, e.g., liver, adrenal, appendix, bone marrow, brain, colon, endometrium, esophagus, fat, gall bladder, heart, kidney, lung, lymph node, ovary, prostate, skin, small intestine, stomach, testis, thyroid, and urinary bladder. HMGB1 RNA associated with vesicular structures is expected to be present in blood, urine, and other body fluids. The level of RNA in blood and urine has been demonstrated to correlate with RNA levels in liver (see, e.g., Sehgal et al., RNA 20:143-149, 2014; Chan et al., Mol. Ther. Nucl. Acids. 4:e263, 2015; both incorporated herein by reference). Further, methods to confirm RNA interference by detection of predicted site specific siRNA cleavage products are known in the art and have been used to demonstrate RNA cleavage in clinical trials (see, e.g., Zimermann et al., Nature. 441:111-114, 2006; Davis et al., Nature. 464:1067-1070, 2010; both incorporated herein by reference). Therefore, the level of knockdown of HMGB1 gene and gene expression in the liver may be greater than the level of knockdown of HMGB1 protein in the blood or HMGB1 RNA associated with vesicular structures in the blood or urine. HMGB1 is released from liver cells, either actively or passively due to leakage from damaged cells, during liver inflammation. Therefore, a reduction in HMGB1 after treatment may only be observed in comparison to an earlier time point in the same individual, e.g., prior to treatment, or as compared to a level from a diseased population. Such considerations are well understood by those of skill in the art.

Inhibition may be assessed by a decrease in an absolute or relative level of one or more variables that are associated with HMGB1 expression compared with a control level. The control level may be any type of control level that is utilized in the art, e.g., a pre-dose baseline level, or a level determined from a similar subject, cell, or sample that is untreated or treated with a control (such as, e.g., buffer only control or inactive agent control).

In some embodiments of the methods of the invention, expression of an HMGB1 gene is inhibited by at least 30%, 35%, 40%, 45%, 50%, 55%, 60%, 65%, 70%, 75%, 80%, 85%, 90%, or 95%, or to below the level of detection of the assay. In preferred embodiments, expression of an HMGB1 gene is inhibited by at least 50%. It is understood that complete or near complete inhibition of HMGB1 may be undesirable. It is further understood that inhibition of HMGB1 expression in certain tissues, e.g., in liver, without a significant inhibition of expression in other tissues, e.g., brain, may be desirable. In preferred embodiments, expression level is determined using the assay method provided in Example 2 with a 10 nM siRNA concentration in the appropriate species matched cell line.

In certain embodiments, inhibition of expression in vivo is determined by knockdown of the human gene in a rodent expressing the human gene, e.g., an AAV-infected mouse expressing the human target gene (i.e., HMGB1), e.g., when administered a single dose at 3 mg/kg at the nadir of RNA expression. Knockdown of expression of an endogenous gene in a model animal system can also be determined, e.g., after administration of a single dose at 3 mg/kg at the nadir of RNA expression. Such systems are useful when the nucleic acid sequence of the human gene and the model animal gene are sufficiently close such that the human iRNA provides effective knockdown of the model animal gene. RNA expression in liver is determined using the PCR methods provided in Example 2.

Inhibition of the expression of an HMGB1 gene may be manifested by a reduction of the amount of mRNA expressed by a first cell or group of cells (such cells may be present, for example, in a sample derived from a subject) in which an HMGB1 gene is transcribed and which has or have been treated (e.g., by contacting the cell or cells with an iRNA of the invention, or by administering an iRNA of the invention to a subject in which the cells are or were present) such that the expression of an HMGB1 gene is inhibited, as compared to a second cell or group of cells substantially identical to the first cell or group of cells but which has not or have not been so treated (control cell(s) not treated with an iRNA or not treated with an iRNA targeted to the gene of interest). In preferred embodiments, the inhibition is assessed by the method provided in Example 2 using a 10 nM siRNA concentration in the species matched cell line and expressing the level of mRNA in treated cells as a percentage of the level of mRNA in control cells, using the following formula:

$$\frac{( mRNA in control cells ) - ( mRNA in treated cells )}{( mRNA in control cells )} • 100 \%$$

In other embodiments, inhibition of the expression of an HMGB1 gene may be assessed in terms of a reduction of a parameter that is functionally linked to an HMGB1 gene expression, e.g., HMGB1 protein level in blood or serum from a subject, RNA level in a blood or urine sample from a subject. HMGB1 gene silencing may be determined in any cell expressing HMGB1, either endogenous or heterologous from an expression construct, and by any assay known in the art.

Inhibition of the expression of an HMGB1 protein may be manifested by a reduction in the level of the HMGB1 protein that is expressed by a cell or group of cells or in a subject sample (e.g., the level of protein in a blood sample derived from a subject). As explained above, for the assessment of mRNA suppression, the inhibition of protein expression levels in a treated cell or group of cells may similarly be expressed as a percentage of the level of protein in a control cell or group of cells, or the change in the level of protein in a subject sample, e.g., urine or blood, or serum derived therefrom.

A control cell, a group of cells, or subject sample that may be used to assess the inhibition of the expression of an HMGB1 gene includes a cell, group of cells, or subject sample that has not yet been contacted with an RNAi agent of the invention. For example, the control cell, group of cells, or subject sample may be derived from an individual subject (e.g., a human or animal subject) prior to treatment of the subject with an RNAi agent or an appropriately matched population control.

The level of HMGB1 mRNA that is expressed by a cell or group of cells may be determined using any method known in the art for assessing mRNA expression. In one embodiment, the level of expression of HMGB1 in a sample is determined by detecting a transcribed polynucleotide, or portion thereof, e.g., mRNA of the HMGB1 gene. RNA may be extracted from cells using RNA extraction techniques including, for example, using acid phenol/guanidine isothiocyanate extraction (RNAzol B; Biogenesis), RNeasy™ RNA preparation kits (Qiagen®) or PAXgene™ (PreAnalytix™, Switzerland). Typical assay formats utilizing ribonucleic acid hybridization include nuclear run-on assays, RT-PCR, RNase protection assays, northern blotting, in situ hybridization, and microarray analysis.

In some embodiments, the level of expression of HMGB1 is determined using a nucleic acid probe. The term “probe”, as used herein, refers to any molecule that is capable of selectively binding to a specific HMGB1. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses and probe arrays. One method for the determination of mRNA levels involves contacting the isolated mRNA with a nucleic acid molecule (probe) that can hybridize to HMGB1 mRNA. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix® gene chip array. A skilled artisan can readily adapt known mRNA detection methods for use in determining the level of HMGB1 mRNA.

An alternative method for determining the level of expression of HMGB1 in a sample involves the process of nucleic acid amplification or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of HMGB1 is determined by quantitative fluorogenic RT-PCR (i.e., the TaqMan™ System). In certain embodiments, methods to confirm RNA interference by detection of predicted site specific siRNA cleavage products are known in the art and have been used to demonstrate RNA cleavage in clinical trials (see, e.g., Zimermann et al., Nature. 441:111-114, 2006; Davis et al., Nature. 464:1067-1070, 2010; both incorporated herein by reference). In preferred embodiments, expression level is determined by the method provided in Example 2 using a 10 nM siRNA concentration in the species matched cell line.

The expression levels of HMGB1 mRNA may be monitored using a membrane blot (such as used in hybridization analysis such as northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are incorporated herein by reference. The determination of HMGB1 expression level may also comprise using nucleic acid probes in solution.

In preferred embodiments, the level of mRNA expression is assessed using branched DNA (bDNA) assays or real time PCR (qPCR). The use of these methods is described and exemplified in the Examples presented herein. In preferred embodiments, expression level is determined by the method provided in Example 2 using a 10 nM siRNA concentration in the species matched cell line.

The level of HMGB1 protein expression may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like.

In some embodiments, the efficacy of the methods of the invention are assessed by a decrease in HMGB1 mRNA or protein level (e.g., in a liver biopsy, blood, or urine).

In some embodiments of the methods of the invention, the iRNA is administered to a subject such that the iRNA is delivered to a specific site within the subject. The inhibition of expression of HMGB1 may be assessed using measurements of the level or change in the level of HMGB1 mRNA or HMGB1 protein in a sample derived from fluid or tissue from the specific site within the subject (e.g., liver, blood, or urine).

As used herein, the terms detecting or determining a level of an analyte are understood to mean performing the steps to determine if a material, e.g., protein, RNA, is present. As used herein, methods of detecting or determining include detection or determination of an analyte level that is below the level of detection for the method used.

VII. Methods of Preventing and Treating HMGB1-Associated Disorders with an HMGB1 iRNA

The present invention also provides methods of using an iRNA of the invention or a composition containing an iRNA of the invention to inhibit expression of HMGB1, thereby preventing or treating an HMGB1-associated disorder, e.g., metabolic disorder or NAFLD, e.g., NASH.

In the methods of the invention the cell may be contacted with the siRNA in vitro or in vivo, i.e., the cell may be within a subject.

A cell suitable for treatment using the methods of the invention may be any cell that expresses an HMGB1 gene, e.g., adrenal, appendix, bone marrow, brain, colon, endometrium, esophagus, fat, gall bladder, heart, kidney, lung, lymph node, ovary, prostate, skin, small intestine, stomach, testis, thyroid, and urinary bladder. In preferred embodiments, a suitable cell is a liver cell. A cell suitable for use in the methods of the invention may be a mammalian cell, e.g., a primate cell (such as a human cell, including human cell in a chimeric non-human animal, or a non-human primate cell, e.g., a monkey cell or a chimpanzee cell), or a non-primate cell. In certain embodiments, the cell is a human cell, e.g., a human liver cell. In the methods of the invention, HMGB1 expression is inhibited in the cell by at least 30, 35, 40, 45, 50, 55, 60, 65, 70, 75, 80, 85, 90, or 95, or to a level below the level of detection of the assay. In preferred embodiments, HMGB1 expression is inhibited by at least 50%. In certain embodiments, expression is determined in a liver cell. In certain embodiments, expression is determined by detecting vesicle associated RNA in a body fluid, e.g., blood or urine.

The in vivo methods of the invention may include administering to a subject a composition containing an iRNA, where the iRNA includes a nucleotide sequence that is complementary to at least a part of an RNA transcript of the HMGB1 gene of the mammal to which the RNAi agent is to be administered. The composition can be administered by any means known in the art including, but not limited to oral, intraperitoneal, or parenteral routes, including intracranial (e.g., intraventricular, intraparenchymal, and intrathecal), intravenous, intramuscular, subcutaneous, transdermal, airway (aerosol), nasal, rectal, and topical (including buccal and sublingual) administration. In certain embodiments, the compositions are administered by intravenous infusion or injection. In certain embodiments, the compositions are administered by subcutaneous injection. In certain embodiments, the compositions are administered by intramuscular injection. In certain embodiments, the compositions are administered by inhalation.

In some embodiments, the administration is via a depot injection. A depot injection may release the iRNA in a consistent way over a prolonged time period. Thus, a depot injection may reduce the frequency of dosing needed to obtain a desired effect, e.g., a desired inhibition of HMGB1 or prophylactic or treatment effect. A depot injection may also provide more consistent serum concentrations. Depot injections may include subcutaneous injections or intramuscular injections. In preferred embodiments, the depot injection is a subcutaneous injection.

In some embodiments, the administration is via a pump. The pump may be an external pump or a surgically implanted pump. In certain embodiments, the pump is a subcutaneously implanted osmotic pump. In other embodiments, the pump is an infusion pump. An infusion pump may be used for intravenous, subcutaneous, arterial, or epidural infusions. In preferred embodiments, the infusion pump is a subcutaneous infusion pump. In other embodiments, the pump is a surgically implanted pump that delivers the iRNA to the liver.

The mode of administration may be chosen based upon whether local or systemic treatment is desired and based upon the area to which the iRNA agent is to be administered. The route and site of administration may be chosen to enhance targeting.

In one aspect, the present invention also provides methods for inhibiting the expression of an HMGB1 gene in a mammal. The methods include administering to the mammal a composition comprising a dsRNA that targets an HMGB1 gene in a cell of the mammal and maintaining the mammal for a time sufficient to obtain degradation of the mRNA transcript of the HMGB1 gene, thereby inhibiting expression of the HMGB1 gene in the cell. Reduction in gene expression can be assessed by any methods known in the art and by methods, e.g. qRT-PCR, described herein, e.g., in Example 2. Reduction in protein production can be assessed by any methods known it the art, e.g. ELISA, and by methods described herein. In one embodiment, a puncture liver biopsy sample serves as the tissue material for monitoring the reduction in the HMGB1 gene or protein expression. In other embodiments, a blood sample serves as the subject sample for monitoring the reduction in the HMGB1 protein expression.

The present invention further provides methods of treatment in a subject in need thereof, e.g., a subject diagnosed with metabolic disorder or NAFLD, e.g., NASH.

The present invention further provides methods of prophylaxis in a subject in need thereof. The treatment methods of the invention include administering an iRNA of the invention to a subject, e.g., a subject that would benefit from a reduction of HMGB1 expression, in a prophylactically effective amount of an iRNA targeting an HMGB1 gene or a pharmaceutical composition comprising an iRNA targeting an HMGB1 gene.

An iRNA of the invention may be administered as a “free iRNA.” A free iRNA is administered in the absence of a pharmaceutical composition. The naked iRNA may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution containing the iRNA can be adjusted such that it is suitable for administering to a subject.

Alternatively, an iRNA of the invention may be administered as a pharmaceutical composition, such as a dsRNA liposomal formulation.

Subjects that would benefit from an inhibition of an HMGB1 gene expression are subjects at risk of developing or diagnosed with metabolic disorder or a NAFLD, e.g., NASH, or other condition associated with NAFL, e.g., obesity, type 2 diabetes mellitus, dyslipidemia, polycystic ovarian disease, hypothyroidism, obstructive sleep apnea, hypopituitarism, hypogonadism, pancreatoduodenal resection, and psoriasis.

In an embodiment, the method includes administering a composition featured herein such that expression of the target HMGB1 gene is decreased, such as for about 1, 2, 3, 4, 5, 6, 1-6, 1-3, or 3-6 months per dose.

Preferably, the iRNAs useful for the methods and compositions featured herein specifically target RNAs (primary or processed) of the target HMGB1 gene. Compositions and methods for inhibiting the expression of these genes using iRNAs can be prepared and performed as described herein.

Administration of the iRNA according to the methods of the invention may result prevention or treatment of metabolic disorder or a NAFLD, e.g., NASH. Diagnostic criteria for metabolic disorder and various NAFLDs, e.g., NASH are provided below.

Subjects can be administered a therapeutic amount of iRNA, such as about 0.01 mg/kg to about 200 mg/kg.

The iRNA can be administered by intravenous infusion over a period of time, on a regular basis. In certain embodiments, after an initial treatment regimen, the treatments can be administered on a less frequent basis. Administration of the iRNA can reduce HMGB1 levels, e.g., in a cell, tissue, blood, or other compartment of the patient by at least 30%, 35%, 40%, 45%, 50%, 55%, 60%, 65%, 70%, 75%, 80%, 85%, 90%, or 95%, or below the level of detection of the assay method used. Preferably administration of the iRNA can reduce HMGB1 levels, e.g., in a cell, tissue, blood, or other compartment of the patient by at least 50%.

Alternatively, the iRNA can be administered subcutaneously, i.e., by subcutaneous injection. One or more injections may be used to deliver the desired dose of iRNA to a subject. The injections may be repeated over a period of time.

The administration may be repeated on a regular basis. In certain embodiments, after an initial treatment regimen, the treatments can be administered on a less frequent basis. A repeat-dose regimen may include administration of a therapeutic amount of iRNA on a regular basis, such as once per month to once a year. In certain embodiments, the iRNA is administered about once per month to about once every three months, or about once every three months to about once every six months.

### VIII. Characteristics and Diagnostic Criteria for a Metabolic Disorder and NAFLD

A. Metabolic Syndrome and Related Conditions

Metabolic syndrome (Syndrome X) is a name for a group of risk factors that occur together and increase the risk for coronary artery disease, stroke, and type 2 diabetes (www.ncbi.nlm.nih.gov/pubmedhealth/PMH0004546/). The diagnostic criteria for metabolic syndrome are defined by the American Heart Association and the National Heart, Lung, and Blood Institute, as individuals having three or more of the following signs:

1. Blood pressure equal to or higher than 130/85 mmHg;

2. Fasting blood sugar (glucose) equal to or higher than 100 mg/dL;

3. Large waist circumference (length around the waist): Men—40 inches or more, Women—35 inches or more;

4. Low HDL cholesterol: Men—under 40 mg/dL, Women—under 50 mg/dL; and

5. Triglycerides equal to or higher than 150 mg/dL

The two most important risk factors for metabolic syndrome are extra weight around the middle and upper parts of the body (central obesity) and insulin resistance, in which the body cannot use insulin effectively. In individuals who do not produce enough insulin or respond to the level of insulin that is produced, blood sugar and fat levels rise. Other risk factors for metabolic syndrome include aging, genetic factors, hormone changes, and a sedentary lifestyle. Individuals with metabolic syndrome frequently suffer from one or both of excessive blood clotting and low levels of systemic inflammation, both of which can exacerbate the condition. In addition to having an increased long-term risk for developing cardiovascular disease and type 2 diabetes, complications of metabolic syndrome further include atherosclerosis, heart attack, kidney disease, non-alcoholic fatty liver disease, peripheral artery disease, and stroke, as well as complications typically associated with diabetes.

Although metabolic syndrome is formally defined by the five criteria above, metabolic imbalances can be defined by other measures. For example, insulin resistance or pre-diabetes can be indicated by elevated fasting blood glucose of at least 100 mg/dL, 2 hour postprandial blood glucose or serum glucose concentration of at least 140 mg/dl. Diagnostic criteria for type 2 diabetes mellitus are any one of hemoglobin Alc (HbA1c) level of 6.5% or higher; fasting blood glucose of at least 126 mg/dL; 2 hour postprandial blood glucose or serum glucose concentration of at least 200 mg/dl; or random plasma glucose of at least 200 mg/dL in a patient with classic symptoms of hyperglycemia (i.e., polyuria, polydipsia, polyphagia, weight loss) or hyperglycemic crisis.

Obesity (excessive body mass index [BMI] and visceral obesity) is the most common and well-documented risk factor for NAFLD. In fact, the entire spectrum of obesity, ranging from overweight (BMI 25.0 to <30) to obese (BMI>30) and severely obese (Class 1: BMI 30 to <35; Class 2: 35 to <40; Class 3: >40).

Hypertension, high blood pressure, can be defined by severity with stage 1 hypertension is a systolic pressure ranging from 140 to 159 mm Hg or a diastolic pressure ranging from 90 to 99 mm Hg. More severe hypertension, stage 2 hypertension is a systolic pressure of 160 mm Hg or higher or a diastolic pressure of 100 mm Hg or higher.

Similarly, hypercholesterolemia is considered to have various severities based on levels. For LDL-cholesterol, 130 to 159 mg/dL is considered borderline high; 160 to 189 mg/dL is considered high; and more than 190 mg/dL is considered very high. For total cholesterol, 200 to 239 mg/dL is considered borderline high and more than 240 mg/dl is considered high. For HDL-cholesterol, less than 40 mg/dl is considered too low.

Treatment of metabolic syndrome and related disorders includes lifestyle changes or medicines to help reduce blood pressure, LDL cholesterol, and blood sugar, e.g., lose weight, increase exercise. Blood pressure and cholesterol may also be regulated using appropriate drugs.

B. NAFLD

NAFLD is formally defined by (1) evidence of hepatic steatosis (HS), either by imaging or histology, and (2) lack of secondary causes of hepatic fat accumulation such as significant alcohol consumption, longterm use of a steatogenic medication, or monogenic hereditary disorders (Chalasani et al., 2017. Hepatology. DOI: 10.1002/hep.29367, incorporated herein by reference). NAFLD is understood to encompass the entire spectrum of fatty liver disease in individuals without significant alcohol consumption, including, e.g., fatty liver, steatohepatitis, and cirrhosis.

NAFL is considered to include the presence of at least 5% HS without evidence of hepatocellular injury in the form of ballooning of the hepatocytes or evidence of fibrosis. Typically, the risk of progression to cirrhosis and liver failure is considered minimal.

NASH is considered to include the presence of at least 5% HS with inflammation and hepatocyte injury (ballooning) with or without fibrosis. NASH can progress to cirrhosis, liver failure, and sometimes liver cancer.

Both unweighted and semiquantitiative scoring methods for grading NAFLD are known. The NAFLD activity score (NAS), developed by the NASH Clinical Research Network, is an unweighted composite of steatosis, lobular inflammation, and ballooning scores. In theory, NAS is a useful tool to measure changes in liver histology in patients with NAFLD in clinical trials, but the cost and risks to study subjects of serial liver biopsy make the scoring system less practical for monitoring. Fibrosis is scored separately (Kleiner et al., 2005. Hepatology. 41:1313-1321, also see tpis.upmc.com/changebody.cfm?url=/tpis/schema/NAFLD2006.jsp, both of which are incorporated by reference).

Steatosis Activity Fibrosis (SAF), developed by the European Fatty Liver Inhibition of Progression Consortium is a semiquantitative score consisting of steatosis amount, activity (lobular inflammation plus ballooning), and fibrosis (Bedossa, 2014. Hepatology, 60:565-575, incorporated herein by reference). There is growing evidence that patients with histological NASH, especially those with some degree of fibrosis, are at higher risk for adverse outcomes such as cirrhosis and liver-related mortality, such that the inclusion of fibrosis in the assessment may be useful. Types of fibrosis most closely associated with long-term mortality are zone 3 sinusoidal fibrosis plus periportal fibrosis (stage 2) to advanced bridging fibrosis (stage 3) or cirrhosis (stage 4). However, as with NAS, the need to perform a liver biopsy makes the scoring method more useful for confirming diagnosis rather than monitoring.

In the majority of patients, NAFLD is associated with metabolic comorbidities such as obesity, diabetes mellitus, and dyslipidemia. NAFLD can also be associated with, polycystic ovarian disease, hypothyroidism, obstructive sleep apnea, hypopituitarism, hypogonadism, pancreatoduodenal resection, and psoriasis. Although definitive diagnosis of NAFLD requires histological confirmation, as there are no specific treatments of NAFLD and obtaining a liver biopsy is costly, presents a risk to the patient, and can be subject to sampling “error” due to potential heterogeneity in histology throughout the liver. Therefore, liver biopsy should only be performed in those who will likely benefit from the screening, by including or excluding NAFLD as a diagnosis and providing information on the severity of disease. Routine screening of the general patient population by liver biopsy is not advised.

The presence of metabolic syndrome is a strong predictor for the presence of SH in patients with NAFLD. Although NAFLD is highly associated with components of metabolic syndrome, the presence of increasing number of metabolic diseases, such as insulin resistance, type 2 diabetes, hypertension dyslipidemia, and visceral obesity, seems to increase the risk of progressive liver disease. Therefore, patients with NAFLD and multiple risk factors such as type 2 diabetes mellitus and hypertension are at the highest risk for adverse outcomes.

Non-invasive methods to assess SH and fibrosis are known, but such methods have their limitations. Serum aminotransferase levels and imaging tests, such as ultrasound, including transient elastography (TE); computed tomography (CT); and magnetic resonance imaging (MRI), do not reliably reflect the spectrum of liver histology in patients with NAFLD. Despite such limitations, MRI, either by spectroscopy or by proton density fat fraction, is an excellent noninvasive modality for quantifying HS and is being widely used in NAFLD clinical trials. The use of TE to obtain continuous attenuation parameters is a promising tool for quantifying hepatic fat in an ambulatory setting. However, the utility of noninvasively quantifying HS in patients with NAFLD in routine clinical care is limited.

There has been significant interest in developing clinical prediction rules and noninvasive biomarkers for identifying SH in patients with NAFLD. Circulating levels of cytokeratin-18 fragments have been investigated extensively as novel biomarkers for the presence of SH in patients with NAFLD. This test is currently not available in a clinical care setting. Commonly investigated noninvasive tools for the presence of advanced fibrosis in NAFLD include aspartate aminotransferase (AST) to platelet ratio index (APRI), and serum biomarkers (Enhanced Liver Fibrosis [ELF] panel). However, to date, no fully satisfactory assay methods or biomarkers have been identified (Musso et al., 2011. Ann. Med. 43:617-649).

C. Cirrhosis

Cirrhosis is defined histologically as a diffuse hepatic process characterized by fibrosis and conversion of the normal liver architecture into structurally abnormal nodules. The progression of liver injury to cirrhosis may occur over weeks to years. Cirrhosis may arise due to any of a number of insults including autoimmune hepatitis, viral hepatitis (e.g., hepatitis B or C), hemochromatosis, primary biliary cirrhosis, or chronic or excessive consumption of alcohol or other drugs that may cause liver damage.

Cirrhosis may be asymptomatic or may be accompanied by multiple symptoms and result in end stage liver disease. Common signs and symptoms may stem from decreased hepatic synthetic function (eg, coagulopathy), portal hypertension (eg, variceal bleeding), or decreased detoxification capabilities of the liver (eg, hepatic encephalopathy). Patients with cirrhosis experience fatigue, anorexia, weight loss, and muscle wasting. Cutaneous manifestations of cirrhosis include jaundice, spider angiomata, skin telangiectasias (“paper money skin”), palmar erythema, white nails, disappearance of lunulae, and finger clubbing, especially in the setting of hepatopulmonary syndrome. Liver fibrosis can be monitored by the methods discussed above for monitoring NAFLD.

The treatment of cirrhosis depends on the underlying etiology of the disease.

This invention is further illustrated by the following examples which should not be construed as limiting. The entire contents of all references, patents and published patent applications cited throughout this application, as well as the Sequence Listing, are hereby incorporated herein by reference.

### EXAMPLES

### Example 1. iRNA Synthesis

### Source of Reagents

Where the source of a reagent is not specifically given herein, such reagent can be obtained from any supplier of reagents for molecular biology at a quality/purity standard for application in molecular biology.

siRNA Design

A set of siRNAs targeting the human high mobility group box 1 gene (HMGB1; human NCBI refseqID NM_002128.5; NCBI GeneID: 3146) as well the toxicology-species HMGB1 ortholog from cynomolgus monkey: NM_001283356) was designed using custom R and Python scripts. All the siRNA designs have a perfect match to the human HMGB1 transcript and a subset either perfect or near-perfect matches to the cynomolgus monkey ortholog. The human NM_002128 REFSEQ mRNA, version 5, has a length of 4273 bases.

A detailed list of the unmodified HMGB1 sense and antisense strand sequences is shown in Table 3. A detailed list of the modified HMGB1 sense and antisense strand sequences is shown in Tables 5, 6, and 7.

siRNA Synthesis

siRNAs were synthesized and annealed using routine methods known in the art.

### Example 2—In Vitro Screening Methods

### Cell Culture and Transfections:

Hep3b cells (ATCC) were transfected by adding 4.9 μl of Opti-MEM plus 0.1 μl of Lipofectamine RNAiMax per well (Invitrogen, Carlsbad Calif. cat #13778-150) to 5 μl of siRNA duplexes per well, with 4 replicates of each SiRNA duplex, into a 384-well plate, and incubated at room temperature for 15 minutes. Forty μl of Eagle's Minimal Essential Medium (Life Tech) containing ˜5×10³ cells were then added to the siRNA mixture. Cells were incubated for 24 hours prior to RNA purification. Single dose experiments were performed at 10 nM.

Total RNA Isolation Using DYNABEADS mRNA Isolation Kit:

RNA was isolated using an automated protocol on a BioTek-EL406 platform using DYNABEADs (Invitrogen, cat #61012). Briefly, 70 μl of Lysis/Binding Buffer and 10 ul of lysis buffer containing 3 μl of magnetic beads were added to the plate with cells. Plates were incubated on an electromagnetic shaker for 10 minutes at room temperature and then magnetic beads were captured and the supernatant was removed. Bead-bound RNA was then washed 2 times with 150 μl Wash Buffer A and once with Wash Buffer B. Beads were then washed with 150 μl Elution Buffer, re-captured and supernatant removed.

cDNA Synthesis Using ABI High Capacity cDNA Reverse Transcription Kit (Applied Biosystems, Foster City, Calif., Cat #4368813):

Ten μl of a master mix containing 1 μl 10× Buffer, 0.4 μl 25× dNTPs, 1 μl 10× Random primers, 0.5 μl Reverse Transcriptase, 0.5 μl RNase inhibitor and 6.6 μl of H₂O per reaction was added to RNA isolated above. Plates were sealed, mixed, and incubated on an electromagnetic shaker for 10 minutes at room temperature, followed by 2 h 37° C.

### Real Time PCR:

Two μl of cDNA were added to a master mix containing 0.5 μl of Human GAPDH TaqMan Probe (4326317E), and 0.5 μl HMGB1 human probe (Hs.PT.58.2259017, IDT) and 5 μl Lightcycler 480 probe master mix (Roche Cat #04887301001) per well in a 384 well plates (Roche cat #04887301001). Real time PCR was done in a LightCycler480 Real Time PCR system (Roche). Each duplex was tested at least two times and data were normalized to cells transfected with a non-targeting control siRNA. To calculate relative fold change, real time data were analyzed using the ΔΔCt method and normalized to assays performed with cells transfected with a non-targeting control siRNA.

TABLE 2
Abbreviations of nucleotide monomers used in nucleic acid sequence representation
when modifications are indicated. It will be understood that these
monomers, when present in an oligonucleotide, are mutually linked by
5′-3′-phosphodiester bonds unless otherwise indicated.

| Abbreviation | Nucleotide(s) |
|---|---|
| A | Adenosine-3′-phosphate |
| Ab | beta-L-adenosine-3′-phosphate |
| Abs | beta-L-adenosine-3′-phosphorothioate |
| Af | 2′-fluoroadenosine-3′-phosphate |
| Afs | 2′-fluoroadenosine-3′-phosphorothioate |
| As | adenosine-3′-phosphorothioate |
| C | cytidine-3′-phosphate |
| Cb | beta-L-cytidine-3′-phosphate |
| Cbs | beta-L-cytidine-3′-phosphorothioate |
| Cf | 2′-fluorocytidine-3′-phosphate |
| Cfs | 2′-fluorocytidine-3′-phosphorothioate |
| Cs | cytidine-3′-phosphorothioate |
| G | guanosine-3′-phosphate |
| Gb | beta-L-guanosine-3′-phosphate |
| Gbs | beta-L-guanosine-3′-phosphorothioate |
| Gf | 2′-fluoroguanosine-3′-phosphate |
| Gfs | 2′-fluoroguanosine-3′-phosphorothioate |
| Gs | guanosine-3′-phosphorothioate |
| T | 5′-methyluridine-3′-phosphate |
| Tf | 2′-fluoro-5-methyluridine-3′-phosphate |
| Tfs | 2′-fluoro-5-methyluridine-3′-phosphorothioate |
| Ts | 5-methyluridine-3′-phosphorothioate |
| U | Uridine-3′-phosphate |
| Uf | 2′-fluorouridine-3′-phosphate |
| Ufs | 2′-fluorouridine-3′-phosphorothioate |
| Us | uridine-3′-phosphorothioate |
| N | any nucleotide, modified or unmodified |
| a | 2′-O-methyladenosine-3′-phosphate |
| as | 2′-O-methyladenosine-3′-phosphorothioate |
| c | 2′-O-methylcytidine-3′-phosphate |
| cs | 2′-O-methylcytidine-3′-phosphorothioate |
| g | 2′-O-methylguanosine-3′-phosphate |
| gs | 2′-O-methylguanosine-3′-phosphorothioate |
| t | 2′-O-methyl-5-methyluridine-3′-phosphate |
| ts | 2′-O-methyl-5-methyluridine-3′-phosphorothioate |
| u | 2′-O-methyluridine-3′-phosphate |
| us | 2′-O-methyluridine-3′-phosphorothioate |
| s | phosphorothioate linkage |
| L96 | N-[tris(GalNAc-alkyl)-amidodecanoyl)]-4-hydroxyprolinol |
| | Hyp-(GalNAc-alkyl)3 |
| Y34 | 2-hydroxymethyl-tetrahydrofurane-4-methoxy-3-phosphate (abasic 2′-OMe |
| | furanose) |
| Y44 | inverted abasic DNA (2-hydroxymethyl-tetrahydrofurane-5-phosphate) |
| (Agn) | Adenosine-glycol nucleic acid (GNA) |
| (Cgn) | Cytidine-glycol nucleic acid (GNA) |
| (Ggn) | Guanosine-glycol nucleic acid (GNA) |
| (Tgn) | Thymidine-glycol nucleic acid (GNA) S-Isomer |
| P | Phosphate |
| VP | Vinyl-phosphate |
| (Aam) | 2′-O-(N-methylacetamide)adenosine-3′-phosphate |
| (Aams) | 2′-O-(N-methylacetamide)adenosine-3′-phosphorothioate |
| (Gam) | 2′-O-(N-methylacetamide)guanosine-3′-phosphate |
| (Gams) | 2′-O-(N-methylacetamide)guanosine-3′-phosphorothioate |
| (Tam) | 2′-O-(N-methylacetamide)thymidine-3′-phosphate |
| (Tams) | 2′-O-(N-methylacetamide)thymidine-3′-phosphorothioate |
| dA | 2′-deoxyadenosine-3′-phosphate |
| dAs | 2′-deoxyadenosine-3′-phosphorothioate |
| dC | 2′-deoxycytidine-3′-phosphate |
| dCs | 2′-deoxycytidine-3′-phosphorothioate |
| dG | 2′-deoxyguanosine-3′-phosphate |
| dGs | 2′-deoxyguanosine-3′-phosphorothioate |
| dT | 2′-deoxythymidine-3′-phosphate |
| dTs | 2′-deoxythymidine-3′-phosphorothioate |
| dU | 2′-deoxyuridine |
| dUs | 2′-deoxyuridine-3′-phosphorothioate |
| (Aeo) | 2′-O-methoxyethyladenosine-3′-phosphate |
| (Aeos) | 2′-O-methoxyethyladenosine-3′-phosphorothioate |
| (Geo) | 2′-O-methoxyethylguanosine-3′-phosphate |
| (Geos) | 2′-O-methoxyethylguanosine-3′-phosphorothioate |
| (Teo) | 2′-O-methoxyethyl-5-methyluridine-3′-phosphate |
| (Teos) | 2′-O-methoxyethyl-5-methyluridine-3′-phosphorothioate |
| (m5Ceo) | 2′-O-methoxyethyl-5-methylcytidine-3′-phosphate |
| (m5Ceos) | 2′-O-methoxyethyl-5-methylcytidine-3′-phosphorothioate |
| (A3m) | 3′-O-methyladenosine-2′-phosphate |
| (A3mx) | 3′-O-methyl-xylofuranosyladenosine-2′-phosphate |
| (G3m) | 3′-O-methylguanosine-2′-phosphate |
| (G3mx) | 3′-O-methyl-xylofuranosylguanosine-2′-phosphate |
| (C3m) | 3′-O-methylcytidine-2′-phosphate |
| (C3mx) | 3′-O-methyl-xylofuranosylcytidine-2′-phosphate |
| (U3m) | 3′-O-methyluridine-2′-phosphate |
| U3mx) | 3′-O-methyl-xylofuranosyluridine-2′-phosphate |
| (m5Cam) | 2′-O-(N-methylacetamide)-5-methylcytidine-3′-phosphate |
| (m5Cams) | 2′-O-(N-methylacetamide)-5-methylcytidine-3′-phosphorothioate |
| (Chd) | 2′-O-hexadecyl-cytidine-3′-phosphate |
| (Chds) | 2′-O-hexadecyl-cytidine-3′-phosphorothioate |
| (Uhd) | 2′-O-hexadecyl-uridine-3′-phosphate |
| (Uhds) | 2′-O-hexadecyl-uridine-3′-phosphorothioate |
| (pshe) | Hydroxyethylphosphorothioate |

TABLE 3
Unmodified Sense and Antisense Strand Sequences of HMGB1 dsRNAs

| | Sense | | | | Antisense | | | |
|---|---|---|---|---|---|---|---|---|
| Duplex | Oligo | Sense Strand Sequence | SEQ ID | Position in | Oligo | Antisense Strand Sequence | SEQ ID | Position in |
| Name | Name | 5′ to 3′ | NO | NM_002128.5 | Name | 5′ to 3′ | NO | NM_002128.5 |
| AD-192647 | A-380909 | GUGCAAACUUGUCGGGAGGAA | 17 | 242-262 | A-380910 | UUCCUCCCGACAAGUUUGCACAA | 111 | 240-262 |
| AD-193168 | A-381951 | UUUUUAAACUGUACAGUGUCU | 18 | 944-964 | A-381952 | AGACACUGUACAGUUUAAAAACA | 112 | 942-964 |
| AD-193173 | A-381961 | UUUGUAUAGUUAACACACUAA | 19 | 968-988 | A-381962 | UUAGUGUGUUAACUAUACAAAAA | 113 | 966-988 |
| AD-193174 | A-381963 | UUGUAUAGUUAACACACUACA | 20 | 969-989 | A-381964 | UGUAGUGUGUUAACUAUACAAAA | 114 | 967-989 |
| AD-193175 | A-381965 | UGUAUAGUUAACACACUACCA | 21 | 970-990 | A-381966 | UGGUAGUGUGUUAACUAUACAAA | 115 | 968-990 |
| AD-193176 | A-381967 | GUAUAGUUAACACACUACCGA | 22 | 971-991 | A-381968 | UCGGUAGUGUGUUAACUAUACAA | 116 | 969-991 |
| AD-193177 | A-381969 | UAUAGUUAACACACUACCGAA | 23 | 972-992 | A-381970 | UUCGGUAGUGUGUUAACUAUACA | 117 | 970-992 |
| AD-193178 | A-381971 | AUAGUUAACACACUACCGAAU | 24 | 973-993 | A-381972 | AUUCGGUAGUGUGUUAACUAUAC | 118 | 971-993 |
| AD-193179 | A-381973 | UAGUUAACACACUACCGAAUA | 25 | 974-994 | A-381974 | UAUUCGGUAGUGUGUUAACUAUA | 119 | 972-994 |
| AD-193180 | A-381975 | AGUUAACACACUACCGAAUGU | 26 | 975-995 | A-381976 | ACAUUCGGUAGUGUGUUAACUAU | 120 | 973-995 |
| AD-193181 | A-381977 | GUUAACACACUACCGAAUGUA | 27 | 976-996 | A-381978 | UACAUUCGGUAGUGUGUUAACUA | 121 | 974-996 |
| AD-193182 | A-381979 | UUAACACACUACCGAAUGUGU | 28 | 977-997 | A-381980 | ACACAUUCGGUAGUGUGUUAACU | 122 | 975-997 |
| AD-193311 | A-382236 | UCUCUGAUGCAGCUUAUACGA | 29 | 1158-1178 | A-382237 | UCGUAUAAGCUGCAUCAGAGACA | 123 | 1156-1178 |
| AD-193312 | A-382238 | CUCUGAUGCAGCUUAUACGAA | 30 | 1159-1179 | A-382239 | UUCGUAUAAGCUGCAUCAGAGAC | 124 | 1157-1179 |
| AD-193313 | A-382240 | UCUGAUGCAGCUUAUACGAAA | 31 | 1160-1180 | A-382241 | UUUCGUAUAAGCUGCAUCAGAGA | 125 | 1158-1180 |
| AD-193314 | A-382242 | CUGAUGCAGCUUAUACGAAAU | 32 | 1161-1181 | A-382243 | AUUUCGUAUAAGCUGCAUCAGAG | 126 | 1159-1181 |
| AD-193315 | A-382244 | UGAUGCAGCUUAUACGAAAUA | 33 | 1162-1182 | A-382245 | UAUUUCGUAUAAGCUGCAUCAGA | 127 | 1160-1182 |
| AD-193326 | A-382266 | UACGAAAUAAUUGUUGUUCUA | 34 | 1174-1194 | A-382267 | UAGAACAACAAUUAUUUCGUAUA | 128 | 1172-1194 |
| AD-193400 | A-382413 | UUGUUGUCCUUUUCAUAGGUA | 35 | 1298-1318 | A-382414 | UACCUAUGAAAAGGACAACAAUA | 129 | 1296-1318 |
| AD-193422 | A-382457 | GGGAAGCUAGUCUUUUGCUUU | 36 | 1338-1358 | A-382458 | AAAGCAAAAGACUAGCUUCCCCU | 130 | 1336-1358 |
| AD-193478 | A-382569 | AUCCUUUCAUAUAGUUAGCUA | 37 | 1395-1415 | A-382570 | UAGCUAACUAUAUGAAAGGAUAA | 131 | 1393-1415 |
| AD-193479 | A-382571 | UCCUUUCAUAUAGUUAGCUAA | 38 | 1396-1416 | A-382572 | UUAGCUAACUAUAUGAAAGGAUA | 132 | 1394-1416 |
| AD-193498 | A-382609 | AAAAAGCUUUUGUCUACACAA | 39 | 1418-1438 | A-382610 | UUGUGUAGACAAAAGCUUUUUAU | 133 | 1416-1438 |
| AD-193506 | A-382625 | UUUGUCUACACACCCUGCAUA | 40 | 1426-1446 | A-382626 | UAUGCAGGGUGUGUAGACAAAAG | 134 | 1424-1446 |
| AD-193507 | A-382627 | UUGUCUACACACCCUGCAUAU | 41 | 1427-1447 | A-382628 | AUAUGCAGGGUGUGUAGACAAAA | 135 | 1425-1447 |
| AD-193522 | A-382657 | AAGUUAAGUUGAGAUAGUUUU | 42 | 1461-1481 | A-382658 | AAAACUAUCUCAACUUAACUUUA | 136 | 1459-1481 |
| AD-193524 | A-382661 | GUUAAGUUGAGAUAGUUUUCA | 43 | 1463-1483 | A-382662 | UGAAAACUAUCUCAACUUAACUU | 137 | 1461-1483 |
| AD-193527 | A-382667 | AAGUUGAGAUAGUUUUCAUCA | 44 | 1466-1486 | A-382668 | UGAUGAAAACUAUCUCAACUUAA | 138 | 1464-1486 |
| AD-193533 | A-382679 | AGAUAGUUUUCAUCCAUAACU | 45 | 1472-1492 | A-382680 | AGUUAUGGAUGAAAACUAUCUCA | 139 | 1470-1492 |
| AD-193534 | A-382681 | GAUAGUUUUCAUCCAUAACUA | 46 | 1473-1493 | A-382682 | UAGUUAUGGAUGAAAACUAUCUC | 140 | 1471-1493 |
| AD-193535 | A-382683 | AUAGUUUUCAUCCAUAACUGA | 47 | 1474-1494 | A-382684 | UCAGUUAUGGAUGAAAACUAUCU | 141 | 1472-1494 |
| AD-193562 | A-382737 | AAAAUCUUGAUCAGUUAAGAA | 48 | 1501-1521 | A-382738 | UUCUUAACUGAUCAAGAUUUUGG | 142 | 1499-1521 |
| AD-193577 | A-382767 | UAAGAAAUUUCACAUAGCCCA | 49 | 1516-1536 | A-382768 | UGGGCUAUGUGAAAUUUCUUAAC | 143 | 1514-1536 |
| AD-193585 | A-382783 | UUCACAUAGCCCACUUACAUU | 50 | 1524-1544 | A-382784 | AAUGUAAGUGGGCUAUGUGAAAU | 144 | 1522-1544 |
| AD-193586 | A-382785 | UCACAUAGCCCACUUACAUUU | 51 | 1525-1545 | A-382786 | AAAUGUAAGUGGGCUAUGUGAAA | 145 | 1523-1545 |
| AD-193587 | A-382787 | CACAUAGCCCACUUACAUUUA | 52 | 1526-1546 | A-382788 | UAAAUGUAAGUGGGCUAUGUGAA | 146 | 1524-1546 |
| AD-193589 | A-382791 | CAUAGCCCACUUACAUUUACA | 53 | 1528-1548 | A-382792 | UGUAAAUGUAAGUGGGCUAUGUG | 147 | 1526-1548 |
| AD-193592 | A-382797 | AGCCCACUUACAUUUACAAAC | 54 | 1531-1551 | A-382798 | GUUUGUAAAUGUAAGUGGGCUAU | 148 | 1529-1551 |
| AD-193625 | A-382863 | AAUCUACUCAAAGCAUGGGAU | 55 | 1564-1584 | A-382864 | AUCCCAUGCUUUGAGUAGAUUGA | 149 | 1562-1584 |
| AD-193629 | A-382871 | UACUCAAAGCAUGGGAUUAUU | 56 | 1568-1588 | A-382872 | AAUAAUCCCAUGCUUUGAGUAGA | 150 | 1566-1588 |
| AD-193630 | A-382873 | ACUCAAAGCAUGGGAUUAUUA | 57 | 1569-1589 | A-382874 | UAAUAAUCCCAUGCUUUGAGUAG | 151 | 1567-1589 |
| AD-193633 | A-382879 | CAAAGCAUGGGAUUAUUAGAA | 58 | 1572-1592 | A-382880 | UUCUAAUAAUCCCAUGCUUUGAG | 152 | 1570-1592 |
| AD-193635 | A-382883 | AAGCAUGGGAUUAUUAGAAUA | 59 | 1574-1594 | A-382884 | UAUUCUAAUAAUCCCAUGCUUUG | 153 | 1572-1594 |
| AD-193640 | A-382893 | UGGGAUUAUUAGAAUCAAACA | 60 | 1579-1599 | A-382894 | UGUUUGAUUCUAAUAAUCCCAUG | 154 | 1577-1599 |
| AD-193662 | A-382937 | AAAGUCUGUCCUUGAAGGACU | 61 | 1605-1625 | A-382938 | AGUCCUUCAAGGACAGACUUUCA | 155 | 1603-1625 |
| AD-193666 | A-382945 | UCUGUCCUUGAAGGACUAAUA | 62 | 1609-1629 | A-382946 | UAUUAGUCCUUCAAGGACAGACU | 156 | 1607-1629 |
| AD-193667 | A-382947 | CUGUCCUUGAAGGACUAAUAA | 63 | 1610-1630 | A-382948 | UUAUUAGUCCUUCAAGGACAGAC | 157 | 1608-1630 |
| AD-193669 | A-382951 | GUCCUUGAAGGACUAAUAGAA | 64 | 1612-1632 | A-382952 | UUCUAUUAGUCCUUCAAGGACAG | 158 | 1610-1632 |
| AD-193690 | A-382993 | AAGUAUGUUCUAACCUUUACA | 65 | 1633-1653 | A-382994 | UGUAAAGGUUAGAACAUACUUUU | 159 | 1631-1653 |
| AD-193697 | A-383007 | UUCUAACCUUUACAUGAGGAA | 66 | 1640-1660 | A-383008 | UUCCUCAUGUAAAGGUUAGAACA | 160 | 1638-1660 |
| AD-193702 | A-383017 | ACCUUUACAUGAGGACUCUAU | 67 | 1645-1665 | A-383018 | AUAGAGUCCUCAUGUAAAGGUUA | 161 | 1643-1665 |
| AD-193705 | A-383023 | UUUACAUGAGGACUCUAUUCU | 68 | 1648-1668 | A-383024 | AGAAUAGAGUCCUCAUGUAAAGG | 162 | 1646-1668 |
| AD-193706 | A-383025 | UUACAUGAGGACUCUAUUCUU | 69 | 1649-1669 | A-383026 | AAGAAUAGAGUCCUCAUGUAAAG | 163 | 1647-1669 |
| AD-193707 | A-383027 | UACAUGAGGACUCUAUUCUUU | 70 | 1650-1670 | A-383028 | AAAGAAUAGAGUCCUCAUGUAAA | 164 | 1648-1670 |
| AD-193711 | A-383035 | UGAGGACUCUAUUCUUUAACU | 71 | 1654-1674 | A-383036 | AGUUAAAGAAUAGAGUCCUCAUG | 165 | 1652-1674 |
| AD-193713 | A-383039 | AGGACUCUAUUCUUUAACUCA | 72 | 1656-1676 | A-383040 | UGAGUUAAAGAAUAGAGUCCUCA | 166 | 1654-1676 |
| AD-193717 | A-383047 | CUCUAUUCUUUAACUCCCAUU | 73 | 1660-1680 | A-383048 | AAUGGGAGUUAAAGAAUAGAGUC | 167 | 1658-1680 |
| AD-193725 | A-383063 | UUUAACUCCCAUUACCAUGUA | 74 | 1668-1688 | A-383064 | UACAUGGUAAUGGGAGUUAAAGA | 168 | 1666-1688 |
| AD-193726 | A-383065 | UUAACUCCCAUUACCAUGUAA | 75 | 1669-1689 | A-383066 | UUACAUGGUAAUGGGAGUUAAAG | 169 | 1667-1689 |
| AD-193727 | A-383067 | UAACUCCCAUUACCAUGUAAU | 76 | 1670-1690 | A-383068 | AUUACAUGGUAAUGGGAGUUAAA | 170 | 1668-1690 |
| AD-193729 | A-383071 | ACUCCCAUUACCAUGUAAUGA | 77 | 1672-1692 | A-383072 | UCAUUACAUGGUAAUGGGAGUUA | 171 | 1670-1692 |
| AD-193731 | A-383075 | UCCCAUUACCAUGUAAUGGCA | 78 | 1674-1694 | A-383076 | UGCCAUUACAUGGUAAUGGGAGU | 172 | 1672-1694 |
| AD-193737 | A-383087 | UACCAUGUAAUGGCAGUUAUA | 79 | 1680-1700 | A-383088 | UAUAACUGCCAUUACAUGGUAAU | 173 | 1678-1700 |
| AD-193744 | A-383101 | UAAUGGCAGUUAUAUUUUGCA | 80 | 1687-1707 | A-383102 | UGCAAAAUAUAACUGCCAUUACA | 174 | 1685-1707 |
| AD-193782 | A-383177 | AAAGCGUGAGCUUAAAAUACA | 81 | 1745-1765 | A-383178 | UGUAUUUUAAGCUCACGCUUUUG | 175 | 1743-1765 |
| AD-193783 | A-383179 | AAGCGUGAGCUUAAAAUACAA | 82 | 1746-1766 | A-383180 | UUGUAUUUUAAGCUCACGCUUUU | 176 | 1744-1766 |
| AD-193804 | A-383221 | UUUGUUGACAUUAGUCUCAGU | 83 | 1785-1805 | A-383222 | ACUGAGACUAAUGUCAACAAAAA | 177 | 1783-1805 |
| AD-193805 | A-383223 | UUGUUGACAUUAGUCUCAGUA | 84 | 1786-1806 | A-383224 | UACUGAGACUAAUGUCAACAAAA | 178 | 1784-1806 |
| AD-193833 | A-383279 | UGAAAAUGCUGGCUAUAGAUA | 85 | 1814-1834 | A-383280 | UAUCUAUAGCCAGCAUUUUCAUA | 179 | 1812-1834 |
| AD-193841 | A-383295 | CUGGCUAUAGAUGUCUUUUCA | 86 | 1822-1842 | A-383296 | UGAAAAGACAUCUAUAGCCAGCA | 180 | 1820-1842 |
| AD-193870 | A-383353 | UAAAUAUGGACUGCUCAGGAA | 87 | 1851-1871 | A-383354 | UUCCUGAGCAGUCCAUAUUUAGA | 181 | 1849-1871 |
| AD-193875 | A-383363 | AUGGACUGCUCAGGAAACGAA | 88 | 1856-1876 | A-383364 | UUCGUUUCCUGAGCAGUCCAUAU | 182 | 1854-1876 |
| AD-193877 | A-383367 | GGACUGCUCAGGAAACGAGAA | 89 | 1858-1878 | A-383368 | UUCUCGUUUCCUGAGCAGUCCAU | 183 | 1856-1878 |
| AD-193881 | A-383375 | UGCUCAGGAAACGAGACUUUA | 90 | 1862-1882 | A-383376 | UAAAGUCUCGUUUCCUGAGCAGU | 184 | 1860-1882 |
| AD-193887 | A-383387 | GGAAACGAGACUUUCCAUUAA | 91 | 1868-1888 | A-383388 | UUAAUGGAAAGUCUCGUUUCCUG | 185 | 1866-1888 |
| AD-193888 | A-383389 | GAAACGAGACUUUCCAUUACA | 92 | 1869-1889 | A-383390 | UGUAAUGGAAAGUCUCGUUUCCU | 186 | 1867-1889 |
| AD-193905 | A-383423 | UUUAAUUAAUUGGGCCAGCUU | 93 | 1896-1916 | A-383424 | AAGCUGGCCCAAUUAAUUAAAAA | 187 | 1894-1916 |
| AD-193907 | A-383427 | UAAUUAAUUGGGCCAGCUUUU | 94 | 1898-1918 | A-383428 | AAAAGCUGGCCCAAUUAAUUAAA | 188 | 1896-1918 |
| AD-194087 | A-383787 | GGUUUUCGAGAUUAUCGUUUU | 95 | 2131-2151 | A-383788 | AAAACGAUAAUCUCGAAAACCAC | 189 | 2129-2151 |
| AD-194088 | A-383789 | GUUUUCGAGAUUAUCGUUUUA | 96 | 2132-2152 | A-383790 | UAAAACGAUAAUCUCGAAAACCA | 190 | 2130-2152 |
| AD-194089 | A-383791 | UUUUCGAGAUUAUCGUUUUCU | 97 | 2133-2153 | A-383792 | AGAAAACGAUAAUCUCGAAAACC | 191 | 2131-2153 |
| AD-194090 | A-383793 | UUUCGAGAUUAUCGUUUUCUU | 98 | 2134-2154 | A-383794 | AAGAAAACGAUAAUCUCGAAAAC | 192 | 2132-2154 |
| AD-194120 | A-383853 | GUAUUUUAAAAUAGCGUUCUU | 99 | 2164-2184 | A-383854 | AAGAACGCUAUUUUAAAAUACUG | 193 | 2162-2184 |
| AD-194134 | A-383881 | CGUUCUUGUAAUUUUACACGA | 100 | 2178-2198 | A-383882 | UCGUGUAAAAUUACAAGAACGCU | 194 | 2176-2198 |
| AD-194135 | A-383883 | GUUCUUGUAAUUUUACACGCU | 101 | 2179-2199 | A-383884 | AGCGUGUAAAAUUACAAGAACGC | 195 | 2177-2199 |
| AD-194136 | A-383885 | UUCUUGUAAUUUUACACGCUU | 102 | 2180-2200 | A-383886 | AAGCGUGUAAAAUUACAAGAACG | 196 | 2178-2200 |
| AD-194137 | A-383887 | UCUUGUAAUUUUACACGCUUU | 103 | 2181-2201 | A-383888 | AAAGCGUGUAAAAUUACAAGAAC | 197 | 2179-2201 |
| AD-194140 | A-383893 | UGUAAUUUUACACGCUUUUGU | 104 | 2184-2204 | A-383894 | ACAAAAGCGUGUAAAAUUACAAG | 198 | 2182-2204 |
| AD-194163 | A-383939 | UGGAGUGCUGUUUUGUUAUAU | 105 | 2207-2227 | A-383940 | AUAUAACAAAACAGCACUCCAUC | 199 | 2205-2227 |
| AD-194218 | A-384049 | AGGAGGAAUACUGAACAUCUA | 106 | 2274-2294 | A-384050 | UAGAUGUUCAGUAUUCCUCCUGA | 200 | 2272-2294 |
| AD-194234 | A-384081 | AUCUGAGUCCUGGAUGAUACU | 107 | 2290-2310 | A-384082 | AGUAUCAUCCAGGACUCAGAUGU | 201 | 2288-2310 |
| AD-194238 | A-384089 | GAGUCCUGGAUGAUACUAAUA | 108 | 2294-2314 | A-384090 | UAUUAGUAUCAUCCAGGACUCAG | 202 | 2292-2314 |
| AD-194239 | A-384091 | AGUCCUGGAUGAUACUAAUAA | 109 | 2295-2315 | A-384092 | UUAUUAGUAUCAUCCAGGACUCA | 203 | 2293-2315 |
| AD-194240 | A-384093 | GUCCUGGAUGAUACUAAUAAA | 110 | 2296-2316 | A-384094 | UUUAUUAGUAUCAUCCAGGACUC | 204 | 2294-2316 |

TABLE 4
HMGB1 Single 10 nM Dose Screen in Hep3B cells

| | % Message | |
|---|---|---|
| Duplex Name | Remaining | STDEV |
| AD-192647 | 81.57 | 3.56 |
| AD-193168 | 20.43 | 2.54 |
| AD-193173 | 24.09 | 1.23 |
| AD-193174 | 26.53 | 3.25 |
| AD-193175 | 27.90 | 4.30 |
| AD-193176 | 34.01 | 6.20 |
| AD-193177 | 15.73 | 2.06 |
| AD-193178 | 26.24 | 4.01 |
| AD-193179 | 25.73 | 9.39 |
| AD-193180 | 21.18 | 2.01 |
| AD-193181 | 38.63 | 7.53 |
| AD-193182 | 21.56 | 5.17 |
| AD-193311 | 25.70 | 4.33 |
| AD-193312 | 18.47 | 3.13 |
| AD-193313 | 20.82 | 10.27 |
| AD-193314 | 22.41 | 5.21 |
| AD-193315 | 27.80 | 8.56 |
| AD-193326 | 29.11 | 7.73 |
| AD-193400 | 82.08 | 4.18 |
| AD-193422 | 79.51 | 7.23 |
| AD-193478 | 79.52 | 8.18 |
| AD-193479 | 70.42 | 3.83 |
| AD-193498 | 83.25 | 2.42 |
| AD-193506 | 80.09 | 6.36 |
| AD-193507 | 66.94 | 6.17 |
| AD-193522 | 74.92 | 4.81 |
| AD-193524 | 79.70 | 0.98 |
| AD-193527 | 74.59 | 4.47 |
| AD-193533 | 74.98 | 2.84 |
| AD-193534 | 82.24 | 7.32 |
| AD-193535 | 90.81 | 17.72 |
| AD-193562 | 69.12 | 4.29 |
| AD-193577 | 82.35 | 9.79 |
| AD-193585 | 69.16 | 7.65 |
| AD-193586 | 84.34 | 8.42 |
| AD-193587 | 73.06 | 2.96 |
| AD-193589 | 68.30 | 0.31 |
| AD-193592 | 71.65 | 9.71 |
| AD-193625 | 85.01 | 3.19 |
| AD-193629 | 75.42 | 6.58 |
| AD-193630 | 70.89 | 3.30 |
| AD-193633 | 76.98 | 1.12 |
| AD-193635 | 73.79 | 8.93 |
| AD-193640 | 83.67 | 10.30 |
| AD-193662 | 79.32 | 2.93 |
| AD-193666 | 69.14 | 3.47 |
| AD-193667 | 71.79 | 4.27 |
| AD-193669 | 74.43 | 6.47 |
| AD-193690 | 82.84 | 3.36 |
| AD-193697 | 95.48 | 14.41 |
| AD-193702 | 99.68 | 11.04 |
| AD-193705 | 65.56 | 5.34 |
| AD-193706 | 92.00 | 13.15 |
| AD-193707 | 62.04 | 4.15 |
| AD-193711 | 62.39 | 4.64 |
| AD-193713 | 65.37 | 3.62 |
| AD-193717 | 85.08 | 8.81 |
| AD-193725 | 120.19 | 10.38 |
| AD-193726 | 114.41 | 11.32 |
| AD-193727 | 87.63 | 6.87 |
| AD-193729 | 94.59 | 11.02 |
| AD-193731 | 97.95 | 9.65 |
| AD-193737 | 84.99 | 8.68 |
| AD-193744 | 78.18 | 5.00 |
| AD-193782 | 69.62 | 5.67 |
| AD-193783 | 81.44 | 7.84 |
| AD-193804 | 54.01 | 2.50 |
| AD-193805 | 78.66 | 9.48 |
| AD-193833 | 81.66 | 5.79 |
| AD-193841 | 87.16 | 16.71 |
| AD-193870 | 85.76 | 3.40 |
| AD-193875 | 86.06 | 16.09 |
| AD-193877 | 79.32 | 3.03 |
| AD-193881 | 100.68 | 12.73 |
| AD-193887 | 84.55 | 12.29 |
| AD-193888 | 76.99 | 10.96 |
| AD-193905 | 74.52 | 2.74 |
| AD-193907 | 86.49 | 6.58 |
| AD-194087 | 86.44 | 13.74 |
| AD-194088 | 74.32 | 13.04 |
| AD-194089 | 86.18 | 21.81 |
| AD-194090 | 92.04 | 16.15 |
| AD-194120 | 84.68 | 18.31 |
| AD-194134 | 63.14 | 9.93 |
| AD-194135 | 80.83 | 2.56 |
| AD-194136 | 75.00 | 9.48 |
| AD-194137 | 74.80 | 8.75 |
| AD-194140 | 79.80 | 8.97 |
| AD-194163 | 71.16 | 5.21 |
| AD-194218 | 80.36 | 8.98 |
| AD-194234 | 70.23 | 0.72 |
| AD-194238 | 65.81 | 15.61 |
| AD-194239 | 75.27 | 10.77 |
| AD-194240 | 67.08 | 6.69 |

TABLE 5
HMGB1 Modified Sequences

| | Sense | | SEQ | Antisense | | | | |
|---|---|---|---|---|---|---|---|---|
| Duplex | Oligo | Sense Strand Sequence | ID | Oligo | Antisense Strand Sequence | SEQ ID | Position in | SEQ ID |
| Name | Name | 5′ to 3′ | NO | Name | 5′ to 3′ | NO | NM_002128.5 | NO |
| AD-193326 | A-382266 | usascgaaAfuAfAfUfuguuguucuaL96 | 205 | A-382267 | usAfsgaaCfaAfCfaauuAfuUfucguasusa | 299 | UAUACGAAAUAAUUGUUGUUCUG | 393 |
| AD-193478 | A-382569 | asusccuuUfcAfUfAfuaguuagcuaL96 | 206 | A-382570 | usAfsgcuAfaCfUfauauGfaAfaggausasa | 300 | UUAUCCUUUCAUAUAGUUAGCUA | 394 |
| AD-193587 | A-382787 | csascauaGfcCfCfAfcuuacauuuaL96 | 207 | A-382788 | usAfsaauGfuAfAfguggGfcUfaugugsasa | 301 | UUCACAUAGCCCACUUACAUUUA | 395 |
| AD-194240 | A-384093 | gsusccugGfaUfGfAfuacuaauaaaL96 | 208 | A-384094 | usUfsuauUfaGfUfaucaUfcCfaggacsusc | 302 | GAGUCCUGGAUGAUACUAAUAAA | 396 |
| AD-193585 | A-382783 | ususcacaUfaGfCfCfcacuuacauuL96 | 209 | A-382784 | asAfsuguAfaGfUfgggcUfaUfgugaasasu | 303 | AUUUCACAUAGCCCACUUACAUU | 397 |
| AD-193589 | A-382791 | csasuagcCfcAfCfUfuacauuuacaL96 | 210 | A-382792 | usGfsuaaAfuGfUfaaguGfgGfcuaugsusg | 304 | CACAUAGCCCACUUACAUUUACA | 398 |
| AD-193707 | A-383027 | usascaugAfgGfAfCfucuauucuuuL96 | 211 | A-383028 | asAfsagaAfuAfGfagucCfuCfauguasasa | 305 | UUUACAUGAGGACUCUAUUCUUU | 399 |
| AD-193711 | A-383035 | usgsaggaCfuCfUfAfuucuuuaacuL96 | 212 | A-383036 | asGfsuuaAfaGfAfauagAfgUfccucasusg | 306 | CAUGAGGACUCUAUUCUUUAACU | 400 |
| AD-193783 | A-383179 | asasgcguGfaGfCfUfuaaaauacaaL96 | 213 | A-383180 | usUfsguaUfuUfUfaagcUfcAfcgcuususu | 307 | AAAAGCGUGAGCUUAAAAUACAA | 401 |
| AD-194087 | A-383787 | gsgsuuuuCfgAfGfAfuuaucguuuuL96 | 214 | A-383788 | asAfsaacGfaUfAfaucuCfgAfaaaccsasc | 308 | GUGGUUUUCGAGAUUAUCGUUUU | 402 |
| AD-194120 | A-383853 | gsusauuuUfaAfAfAfuagcguucuuL96 | 215 | A-383854 | asAfsgaaCfgCfUfauuuUfaAfaauacsusg | 309 | CAGUAUUUUAAAAUAGCGUUCUU | 403 |
| AD-193400 | A-382413 | ususguugUfcCfUfUfuucauagguaL96 | 216 | A-382414 | usAfsccuAfuGfAfaaagGfaCfaacaasusa | 310 | UAUUGUUGUCCUUUUCAUAGGUC | 404 |
| AD-193887 | A-383387 | gsgsaaacGfaGfAfCfuuuccauuaaL96 | 217 | A-383388 | usUfsaauGfgAfAfagucUfcGfuuuccsusg | 311 | CAGGAAACGAGACUUUCCAUUAC | 405 |
| AD-194088 | A-383789 | gsusuuucGfaGfAfUfuaucguuuuaL96 | 218 | A-383790 | usAfsaaaCfgAfUfaaucUfcGfaaaacscsa | 312 | UGGUUUUCGAGAUUAUCGUUUUC | 406 |
| AD-194163 | A-383939 | usgsgaguGfcUfGfUfuuuguuauauL96 | 219 | A-383940 | asUfsauaAfcAfAfaacaGfcAfcuccasusc | 313 | GAUGGAGUGCUGUUUUGUUAUAU | 407 |
| AD-193313 | A-382240 | uscsugauGfcAfGfCfuuauacgaaaL96 | 220 | A-382241 | usUfsucgUfaUfAfagcuGfcAfucagasgsa | 314 | UCUCUGAUGCAGCUUAUACGAAA | 408 |
| AD-193314 | A-382242 | csusgaugCfaGfCfUfuauacgaaauL96 | 221 | A-382243 | asUfsuucGfuAfUfaagcUfgCfaucagsasg | 315 | CUCUGAUGCAGCUUAUACGAAAU | 409 |
| AD-193534 | A-382681 | gsasuaguUfuUfCfAfuccauaacuaL96 | 222 | A-382682 | usAfsguuAfuGfGfaugaAfaAfcuaucsusc | 316 | GAGAUAGUUUUCAUCCAUAACUG | 410 |
| AD-193586 | A-382785 | uscsacauAfgCfCfCfacuuacauuuL96 | 223 | A-382786 | asAfsaugUfaAfGfugggCfuAfugugasasa | 317 | UUUCACAUAGCCCACUUACAUUU | 411 |
| AD-193706 | A-383025 | ususacauGfaGfGfAfcucuauucuuL96 | 224 | A-383026 | asAfsgaaUfaGfAfguccUfcAfuguaasasg | 318 | CUUUACAUGAGGACUCUAUUCUU | 412 |
| AD-193841 | A-383295 | csusggcuAfuAfGfAfugucuuuucaL96 | 225 | A-383296 | usGfsaaaAfgAfCfaucuAfuAfgccagscsa | 319 | UGCUGGCUAUAGAUGUCUUUUCC | 413 |
| AD-194090 | A-383793 | ususucgaGfaUfUfAfucguuuucuuL96 | 226 | A-383794 | asAfsgaaAfaCfGfauaaUfcUfcgaaasasc | 320 | GUUUUCGAGAUUAUCGUUUUCUU | 414 |
| AD-194136 | A-383885 | ususcuugUfaAfUfUfuuacacgcuuL96 | 227 | A-383886 | asAfsgcgUfgUfAfaaauUfaCfaagaascsg | 321 | CGUUCUUGUAAUUUUACACGCUU | 415 |
| AD-193180 | A-381975 | asgsuuaaCfaCfAfCfuaccgaauguL96 | 228 | A-381976 | asCfsauuCfgGfUfagugUfgUfuaacusasu | 322 | AUAGUUAACACACUACCGAAUGU | 416 |
| AD-193522 | A-382657 | asasguuaAfgUfUfGfagauaguuuuL96 | 229 | A-382658 | asAfsaacUfaUfCfucaaCfuUfaacuususa | 323 | UAAAGUUAAGUUGAGAUAGUUUU | 417 |
| AD-193635 | A-382883 | asasgcauGfgGfAfUfuauuagaauaL96 | 230 | A-382884 | usAfsuucUfaAfUfaaucCfcAfugcuususg | 324 | CAAAGCAUGGGAUUAUUAGAAUC | 418 |
| AD-193717 | A-383047 | csuscuauUfcUfUfUfaacucccauuL96 | 231 | A-383048 | asAfsuggGfaGfUfuaaaGfaAfuagagsusc | 325 | GACUCUAUUCUUUAACUCCCAUU | 419 |
| AD-194137 | A-383887 | uscsuuguAfaUfUfUfuacacgcuuuL96 | 232 | A-383888 | asAfsagcGfuGfUfaaaaUfuAfcaagasasc | 326 | GUUCUUGUAAUUUUACACGCUUU | 420 |
| AD-193179 | A-381973 | usasguuaAfcAfCfAfcuaccgaauaL96 | 233 | A-381974 | usAfsuucGfgUfAfguguGfuUfaacuasusa | 327 | UAUAGUUAACACACUACCGAAUG | 421 |
| AD-193181 | A-381977 | gsusuaacAfcAfCfUfaccgaauguaL96 | 234 | A-381978 | usAfscauUfcGfGfuaguGfuGfuuaacsusa | 328 | UAGUUAACACACUACCGAAUGUG | 422 |
| AD-193312 | A-382238 | csuscugaUfgCfAfGfcuuauacgaaL96 | 235 | A-382239 | usUfscguAfuAfAfgcugCfaUfcagagsasc | 329 | GUCUCUGAUGCAGCUUAUACGAA | 423 |
| AD-193506 | A-382625 | ususugucUfaCfAfCfacccugcauaL96 | 236 | A-382626 | usAfsugcAfgGfGfugugUfaGfacaaasasg | 330 | CUUUUGUCUACACACCCUGCAUA | 424 |
| AD-193533 | A-382679 | asgsauagUfuUfUfCfauccauaacuL96 | 237 | A-382680 | asGfsuuaUfgGfAfugaaAfaCfua ucuscsa | 331 | UGAGAUAGUUUUCAUCCAUAACU | 425 |
| AD-193629 | A-382871 | usascucaAfaGfCfAfugggauuauuL96 | 238 | A-382872 | asAfsuaaUfcCfCfaugcUfuUfgaguasgsa | 332 | UCUACUCAAAGCAUGGGAUUAUU | 426 |
| AD-193726 | A-383065 | ususaacuCfcCfAfUfuaccauguaaL96 | 239 | A-383066 | usUfsacaUfgGfUfaaugGfgAfguuaasasg | 333 | CUUUAACUCCCAUUACCAUGUAA | 427 |
| AD-193727 | A-383067 | usasacucCfcAfUfUfaccauguaauL96 | 240 | A-383068 | asUfsuacAfuGfGfuaauGfgGfaguuasasa | 334 | UUUAACUCCCAUUACCAUGUAAU | 428 |
| AD-193782 | A-383177 | asasagcgUfgAfGfCfuuaaaauacaL96 | 241 | A-383178 | usGfsuauUfuUfAfagcuCfaCfgcuuususg | 335 | CAAAAGCGUGAGCUUAAAAUACA | 429 |
| AD-193805 | A-383223 | ususguugAfcAfUfUfagucucaguaL96 | 242 | A-383224 | usAfscugAfgAfCfuaauGfuCfaacaasasa | 336 | UUUUGUUGACAUUAGUCUCAGUG | 430 |
| AD-193507 | A-382627 | ususgucuAfcAfCfAfcccugcauauL96 | 243 | A-382628 | asUfsaugCfaGfGfguguGfuAfgacaasasa | 337 | UUUUGUCUACACACCCUGCAUAU | 431 |
| AD-193592 | A-382797 | asgscccaCfuUfAfCfauuuacaaacL96 | 244 | A-382798 | gsUfsuugUfaAfAfuguaAfgUfgggcusasu | 338 | AUAGCCCACUUACAUUUACAAAC | 432 |
| AD-193697 | A-383007 | ususcuaaCfcUfUfUfacaugaggaaL96 | 245 | A-383008 | usUfsccuCfaUfGfuaaaGfgUfuagaascsa | 339 | UGUUCUAACCUUUACAUGAGGAC | 433 |
| AD-193702 | A-383017 | ascscuuuAfcAfUfGfaggacucuauL96 | 246 | A-383018 | asUfsagaGfuCfCfucauGfuAfaaggususa | 340 | UAACCUUUACAUGAGGACUCUAU | 434 |
| AD-193725 | A-383063 | ususuaacUfcCfCfAfuuaccauguaL96 | 247 | A-383064 | usAfscauGfgUfAfauggGfaGfuuaaasgsa | 341 | UCUUUAACUCCCAUUACCAUGUA | 435 |
| AD-193881 | A-383375 | usgscucaGfgAfAfAfcgagacuuuaL96 | 248 | A-383376 | usAfsaagUfcUfCfguuuCfcUfgagcasgsu | 342 | ACUGCUCAGGAAACGAGACUUUC | 436 |
| AD-193178 | A-381971 | asusaguuAfaCfAfCfacuaccgaauL96 | 249 | A-381972 | asUfsucgGfuAfGfugugUfuAfacuausasc | 343 | GUAUAGUUAACACACUACCGAAU | 437 |
| AD-193315 | A-382244 | usgsaugcAfgCfUfUfauacgaaauaL96 | 250 | A-382245 | usAfsuuuCfgUfAfuaagCfuGfcaucasgsa | 344 | UCUGAUGCAGCUUAUACGAAAUA | 438 |
| AD-193666 | A-382945 | uscsugucCfuUfGfAfaggacuaauaL96 | 251 | A-382946 | usAfsuuaGfuCfCfuucaAfgGfacagascsu | 345 | AGUCUGUCCUUGAAGGACUAAUA | 439 |
| AD-193888 | A-383389 | gsasaacgAfgAfCfUfuuccauuacaL96 | 252 | A-383390 | usGfsuaaUfgGfAfaaguCfuCfguuucscsu | 346 | AGGAAACGAGACUUUCCAUUACA | 440 |
| AD-194135 | A-383883 | gsusucuuGfuAfAfUfuuuacacgcuL96 | 253 | A-383884 | asGfscguGfuAfAfaauuAfcAfagaacsgsc | 347 | GCGUUCUUGUAAUUUUACACGCU | 441 |
| AD-193479 | A-382571 | uscscuuuCfaUfAfUfaguuagcuaaL96 | 254 | A-382572 | usUfsagcUfaAfCfuauaUfgAfaaggasusa | 348 | UAUCCUUUCAUAUAGUUAGCUAA | 442 |
| AD-193662 | A-382937 | asasagucUfgUfCfCfuugaaggacuL96 | 255 | A-382938 | asGfsuccUfuCfAfaggaCfaGfacuuuscsa | 349 | UGAAAGUCUGUCCUUGAAGGACU | 443 |
| AD-193669 | A-382951 | gsusccuuGfaAfGfGfacuaauagaaL96 | 256 | A-382952 | usUfscuaUfuAfGfuccuUfcAfaggacsasg | 350 | CUGUCCUUGAAGGACUAAUAGAA | 444 |
| AD-194134 | A-383881 | csgsuucuUfgUfAfAfuuuuacacgaL96 | 257 | A-383882 | usCfsgugUfaAfAfauuaCfaAfgaacgscsu | 351 | AGCGUUCUUGUAAUUUUACACGC | 445 |
| AD-193175 | A-381965 | usgsuauaGfuUfAfAfcacacuaccaL96 | 258 | A-381966 | usGfsguaGfuGfUfguuaAfcUfauacasasa | 352 | UUUGUAUAGUUAACACACUACCG | 446 |
| AD-193713 | A-383039 | asgsgacuCfuAfUfUfcuuuaacucaL96 | 259 | A-383040 | usGfsaguUfaAfAfgaauAfgAfguccuscsa | 353 | UGAGGACUCUAUUCUUUAACUCC | 447 |
| AD-194140 | A-383893 | usgsuaauUfuUfAfCfacgcuuuuguL96 | 260 | A-383894 | asCfsaaaAfgCfGfuguaAfaAfuuacasasg | 354 | CUUGUAAUUUUACACGCUUUUGU | 448 |
| AD-194239 | A-384091 | asgsuccuGfgAfUfGfauacuaauaaL96 | 261 | A-384092 | usUfsauuAfgUfAfucauCfcAfggacuscsa | 355 | UGAGUCCUGGAUGAUACUAAUAA | 449 |
| AD-193177 | A-381969 | usasuaguUfaAfCfAfcacuaccgaaL96 | 262 | A-381970 | usUfscggUfaGfUfguguUfaAfcuauascsa | 356 | UGUAUAGUUAACACACUACCGAA | 450 |
| AD-193562 | A-382737 | asasaaucUfuGfAfUfcaguuaagaaL96 | 263 | A-382738 | usUfscuuAfaCfUfgaucAfaGfauuuusgsg | 357 | CCAAAAUCUUGAUCAGUUAAGAA | 451 |
| AD-193630 | A-382873 | ascsucaaAfgCfAfUfgggauuauuaL96 | 264 | A-382874 | usAfsauaAfuCfCfcaugCfuUfugagusasg | 358 | CUACUCAAAGCAUGGGAUUAUUA | 452 |
| AD-193731 | A-383075 | uscsccauUfaCfCfAfuguaauggcaL96 | 265 | A-383076 | usGfsccaUfuAfCfauggUfaAfugggasgsu | 359 | ACUCCCAUUACCAUGUAAUGGCA | 453 |
| AD-193804 | A-383221 | ususuguuGfaCfAfUfuagucucaguL96 | 266 | A-383222 | asCfsugaGfaCfUfaaugUfcAfacaaasasa | 360 | UUUUUGUUGACAUUAGUCUCAGU | 454 |
| AD-194238 | A-384089 | gsasguccUfgGfAfUfgauacuaauaL96 | 267 | A-384090 | usAfsuuaGfuAfUfcaucCfaGfgacucsasg | 361 | CUGAGUCCUGGAUGAUACUAAUA | 455 |
| AD-193168 | A-381951 | ususuuuaAfaCfUfGfuacagugucuL96 | 268 | A-381952 | asGfsacaCfuGfUfacagUfuUfaaaaascsa | 362 | UGUUUUUAAACUGUACAGUGUCU | 456 |
| AD-193174 | A-381963 | ususguauAfgUfUfAfacacacuacaL96 | 269 | A-381964 | usGfsuagUfgUfGfuuaaCfuAfuacaasasa | 363 | UUUUGUAUAGUUAACACACUACC | 457 |
| AD-193182 | A-381979 | ususaacaCfaCfUfAfccgaauguguL96 | 270 | A-381980 | asCfsacaUfuCfGfguagUfgUfguuaascsu | 364 | AGUUAACACACUACCGAAUGUGU | 458 |
| AD-193524 | A-382661 | gsusuaagUfuGfAfGfauaguuuucaL96 | 271 | A-382662 | usGfsaaaAfcUfAfucucAfaCfuuaacsusu | 365 | AAGUUAAGUUGAGAUAGUUUUCA | 459 |
| AD-193833 | A-383279 | usgsaaaaUfgCfUfGfgcuauagauaL96 | 272 | A-383280 | usAfsucuAfuAfGfccagCfaUfuuucasusa | 366 | UAUGAAAAUGCUGGCUAUAGAUG | 460 |
| AD-194218 | A-384049 | asgsgaggAfaUfAfCfugaacaucuaL96 | 273 | A-384050 | usAfsgauGfuUfCfaguaUfuCfcuccusgsa | 367 | UCAGGAGGAAUACUGAACAUCUG | 461 |
| AD-193173 | A-381961 | ususuguaUfaGfUfUfaacacacuaaL96 | 274 | A-381962 | usUfsaguGfuGfUfuaacUfaUfacaaasasa | 368 | UUUUUGUAUAGUUAACACACUAC | 462 |
| AD-193690 | A-382993 | asasguauGfuUfCfUfaaccuuuacaL96 | 275 | A-382994 | usGfsuaaAfgGfUfuagaAfcAfuacuususu | 369 | AAAAGUAUGUUCUAACCUUUACA | 463 |
| AD-193907 | A-383427 | usasauuaAfuUfGfGfgccagcuuuuL96 | 276 | A-383428 | asAfsaagCfuGfGfcccaAfuUfaauuasasa | 370 | UUUAAUUAAUUGGGCCAGCUUUU | 464 |
| AD-193633 | A-382879 | csasaagcAfuGfGfGfauuauuagaaL96 | 277 | A-382880 | usUfscuaAfuAfAfucccAfuGfcuuugsasg | 371 | CUCAAAGCAUGGGAUUAUUAGAA | 465 |
| AD-193667 | A-382947 | csusguccUfuGfAfAfggacuaauaaL96 | 278 | A-382948 | usUfsauuAfgUfCfcuucAfaGfgacagsasc | 372 | GUCUGUCCUUGAAGGACUAAUAG | 466 |
| AD-193737 | A-383087 | usasccauGfuAfAfUfggcaguuauaL96 | 279 | A-383088 | usAfsuaaCfuGfCfcauuAfcAfugguasasu | 373 | AUUACCAUGUAAUGGCAGUUAUA | 467 |
| AD-193870 | A-383353 | usasaauaUfgGfAfCfugcucaggaaL96 | 280 | A-383354 | usUfsccuGfaGfCfagucCfaUfauuuasgsa | 374 | UCUAAAUAUGGACUGCUCAGGAA | 468 |
| AD-193176 | A-381967 | gsusauagUfuAfAfCfacacuaccgaL96 | 281 | A-381968 | usCfsgguAfgUfGfuguuAfaCfuauacsasa | 375 | UUGUAUAGUUAACACACUACCGA | 469 |
| AD-193705 | A-383023 | ususuacaUfgAfGfGfacucuauucuL96 | 282 | A-383024 | asGfsaauAfgAfGfuccuCfaUfguaaasgsg | 376 | CCUUUACAUGAGGACUCUAUUCU | 470 |
| AD-193729 | A-383071 | ascsucccAfuUfAfCfcauguaaugaL96 | 283 | A-383072 | usCfsauuAfcAfUfgguaAfuGfggagususa | 377 | UAACUCCCAUUACCAUGUAAUGG | 471 |
| AD-193498 | A-382609 | asasaaagCfuUfUfUfgucuacacaaL96 | 284 | A-382610 | usUfsgugUfaGfAfcaaaAfgCfuuuuusasu | 378 | AUAAAAAGCUUUUGUCUACACAC | 472 |
| AD-193535 | A-382683 | asusaguuUfuCfAfUfccauaacugaL96 | 285 | A-382684 | usCfsaguUfaUfGfgaugAfaAfacuauscsu | 379 | AGAUAGUUUUCAUCCAUAACUGA | 473 |
| AD-193640 | A-382893 | usgsggauUfaUfUfAfgaaucaaacaL96 | 286 | A-382894 | usGfsuuuGfaUfUfcuaaUfaAfucccasusg | 380 | CAUGGGAUUAUUAGAAUCAAACA | 474 |
| AD-193422 | A-382457 | gsgsgaagCfuAfGfUfcuuuugcuuuL96 | 287 | A-382458 | asAfsagcAfaAfAfgacuAfgCfuucccscsu | 381 | AGGGGAAGCUAGUCUUUUGCUUU | 475 |
| AD-193527 | A-382667 | asasguugAfgAfUfAfguuuucaucaL96 | 288 | A-382668 | usGfsaugAfaAfAfcuauCfuCfaacuusasa | 382 | UUAAGUUGAGAUAGUUUUCAUCC | 476 |
| AD-193577 | A-382767 | usasagaaAfuUfUfCfacauagcccaL96 | 289 | A-382768 | usGfsggcUfaUfGfugaaAfuUfucuuasasc | 383 | GUUAAGAAAUUUCACAUAGCCCA | 477 |
| AD-193905 | A-383423 | ususuaauUfaAfUfUfgggccagcuuL96 | 290 | A-383424 | asAfsgcuGfgCfCfcaauUfaAfuuaaasasa | 384 | UUUUUAAUUAAUUGGGCCAGCUU | 478 |
| AD-194089 | A-383791 | ususuucgAfgAfUfUfaucguuuucuL96 | 291 | A-383792 | asGfsaaaAfcGfAfuaauCfuCfgaaaascsc | 385 | GGUUUUCGAGAUUAUCGUUUUCU | 479 |
| AD-193625 | A-382863 | asasucuaCfuCfAfAfagcaugggauL96 | 292 | A-382864 | asUfscccAfuGfCfuuugAfgUfagauusgsa | 386 | UCAAUCUACUCAAAGCAUGGGAU | 480 |
| AD-193875 | A-383363 | asusggacUfgCfUfCfaggaaacgaaL96 | 293 | A-383364 | usUfscguUfuCfCfugagCfaGfuccausasu | 387 | AUAUGGACUGCUCAGGAAACGAG | 481 |
| AD-194234 | A-384081 | asuscugaGfuCfCfUfggaugauacuL96 | 294 | A-384082 | asGfsuauCfaUfCfcaggAfcUfcagausgsu | 388 | ACAUCUGAGUCCUGGAUGAUACU | 482 |
| AD-193311 | A-382236 | uscsucugAfuGfCfAfgcuuauacgaL96 | 295 | A-382237 | usCfsguaUfaAfGfcugcAfuCfagagascsa | 389 | UGUCUCUGAUGCAGCUUAUACGA | 483 |
| AD-193744 | A-383101 | usasauggCfaGfUfUfauauuuugcaL96 | 296 | A-383102 | usGfscaaAfaUfAfuaacUfgCfcauuascsa | 390 | UGUAAUGGCAGUUAUAUUUUGCA | 484 |
| AD-193877 | A-383367 | gsgsacugCfuCfAfGfgaaacgagaaL96 | 297 | A-383368 | usUfscucGfuUfUfccugAfgCfaguccsasu | 391 | AUGGACUGCUCAGGAAACGAGAC | 485 |
| AD-192647 | A-380909 | gsusgcaaAfcUfUfGfucgggaggaaL96 | 298 | A-380910 | usUfsccuCfcCfGfacaaGfuUfugcacsasa | 392 | UUGUGCAAACUUGUCGGGAGGAG | 486 |

### Example 3—Knockdown of HMGB1 Expression with a Single Dose of HMGB1 siRNA

A series of dsRNA agents targeting mouse HMGB1 were designed and tested for the ability to knockdown expression of HMGB1 mRNA in 6-8 week old C57Bl/6 female mice. The duplexes are listed in Table 6.

Duplex AD-80644 includes a single mismatch at position 1 in the antisense strand as compared to the human mRNA sequence. Duplexes AD-80652 and AD-80653 are a perfect match in the antisense strand as compared to the human mRNA sequence. Duplexes AD-80646, AD-80651, and AD80652 include multiple mismatches between the mouse and human sequences.

A single dose of six selected dsRNA agents; or PBS control, were administered subcutaneously on day 1 as a dose of 3 mg/kg (n=3 per group). At days 7 and 21 post-dosing, mice were fasted for 5 hours prior to sacrifice to clear glycogen from the liver in order to facilitate liver imaging, livers were harvested, and HMGB1 expression was analyzed.

RNA was isolated using the RNeasy plus kit (Qiagen, Cat #74136). 1 μg of total RNA was transcribed to cDNA using ABI® High capacity cDNA reverse transcription kit (Applied Biosystems, Cat #4368813), both following manufacturer's protocol.

One μl of cDNA was added to a master mix containing 0.5 μl of GAPDH TaqMan Probe (4352339E), 0.5 μl mouse HMGB1 probe (Mm00849805_gH), 5 μl Lightcycler 480 probe master mix (Roche Cat #04887301001) and 3 μl of nuclease free water per well in a 384-well plate (Roche Cat #04887301001). Reactions were run in triplicate. Real time PCR was done in a LightCycler480 Real Time PCR system (Roche). To calculate relative fold change, real time data were analyzed using the ΔΔCt method and normalized to PBS treated animals. Results are shown in the table below.

| | Day 7 | stdev | Day 21 | stdev |
|---|---|---|---|---|
| PBS | 101.6365 | 21.35079 | 100.0892 | 5.139943 |
| AD-80644 | 17.72006 | 0.911535 | 21.8557 | 4.188224 |
| AD-80646 | 16.06716 | 3.34729 | 19.21384 | 3.47529 |
| AD-80648 | 67.76202 | 7.184302 | 103.9695 | 16.18052 |
| AD-80651 | 18.60321 | 0.45585 | 30.74869 | 8.934711 |
| AD-80652 | 11.1124 | 1.73809 | 14.60145 | 1.346553 |
| AD-80653 | 10.98006 | 1.528939 | 14.96498 | 3.3726 |

Based on these data, duplex AD-80653 was selected for further studies.

TABLE 6
HMGB1 Modified Sequences

| | Strand | | | | | |
|---|---|---|---|---|---|---|
| | (S = | | | | | |
| | Sense; | | | SEQ | | SEQ |
| Duplex | AS = | Oligo | Oligo Sequence | ID | Oligo Sequence | ID |
| Name | Antisense) | Name | (modified) 5′ to 3′ | NO: | (unmodified) 5′ to 3′ | NO: |
| AD-80644 | S | A-161392 | gsusaagaUfuUfGfUfuuuuaaacuaL96 | 487 | GUAAGAUUUGUUUUUAAACUA | 499 |
| | AS | A-161393 | VPusAfsguuUfaAfAfaacaAfaUfcuuacsasc | 488 | UAGUUUAAAAACAAAUCUUACAC | 500 |
| AD-80646 | S | A-161396 | ascsgaagAfuAfAfUfuguuguucuaL96 | 489 | ACGAAGAUAAUUGUUGUUCUA | 501 |
| | AS | A-161397 | VPusAfsgaaCfaAfCfaauuAfuCfuucgusasu | 490 | UAGAACAACAAUUAUCUUCGUAU | 502 |
| AD-80648 | S | A-161400 | csasucccUfaAfUfCfuauacauauaL96 | 491 | CAUCCCUAAUCUAUACAUAUA | 503 |
| | AS | A-161401 | VPusAfsuauGfuAfUfagauUfaGfggaugscsu | 492 | UAUAUGUAUAGAUUAGGGAUGCU | 504 |
| AD-80651 | S | A-161406 | usgsgaguGfcUfGfUfuauauaauuaL96 | 493 | UGGAGUGCUGUUAUAUAAUUA | 505 |
| | AS | A-161407 | VPusAfsauuAfuAfUfaacaGfcAfcuccasusc | 494 | UAAUUAUAUAACAGCACUCCAUC | 506 |
| AD-80652 | S | A-161408 | gscsacagCfaCfAfAfauuaguuauaL96 | 495 | GCACAGCACAAAUUAGUUAUA | 507 |
| | AS | A-161409 | VPusAfsuaaCfuAfAfuuugUfgCfugugcsasc | 496 | UAUAACUAAUUUGUGCUGUGCAC | 508 |
| AD-80653 | S | A-161410 | csusgaauGfcUfUfCfuaaguaaauaL96 | 497 | CUGAAUGCUUCUAAGUAAAUA | 509 |
| | AS | A-161411 | VPusAfsuuuAfcUfUfagaaGfcAfuucagsasa | 498 | UAUUUACUUAGAAGCAUUCAGAA | 510 |

### Example 4—Treatment of HMGB1-Associated Disorder with an HMGB1 siRNA

A high fat-high fructose (HF HFr) fed mouse model of NASH (Softic et al. J Clin Invest 127 (11):4059-4074, 2017, incorporated herein by reference) was used to demonstrate the efficacy of HMGB1 siRNA to treat NASH and signs of metabolic disorder.

Six to eight weeks-old C57BL/6 male mice obtained from Jackson Laboratories were fed a high fat diet containing 60% of calories as fat plus 30% fructose in water (Hf Hfr diet) for 12 weeks prior to treatment with a dsRNA agent in order to induce NASH or fed a standard chow and water diet. Food and water were provided ad libitum. As expected, body weight and liver weight were significantly higher in the HF HFr fed mice. Liver injury was indicated by a significant increase in serum glutamate dehydrogenase (GLDH), alanine aminotransferase (ALT), and aspartate aminotransferase (AST) levels, in the Hf Hfr mice. Liver histology was assessed to confirm the development of NASH. Vacuolation, inflammation, ballooning degeneration, and fibrosis were observed in mice fed the Hf Hfr diet, but in none of the mice fed standard chow. These data demonstrate that the Hf Hfr diet was effective in establishing a NASH phenotype. A number of signs related to metabolic syndrome were also observed as a result of the Hf Hfr diet including increased serum insulin and glucose and increased serum cholesterol.

Starting at week 12, HF HFr fed mice were subcutaneously administered a 10 mg/kg dose of an siRNA targeted to HMGB1 (AD-80653) every other week for a total of four doses. Two weeks after the final dose (at week 20), livers were harvested, RNA was isolated, and HMGB1 knockdown was determined by rtPCR using the method described above. A 94% decrease in liver HMGB1 mRNA was observed in the HMGB1 siRNA treated Hf Hfr fed mice as compared to the PBS treated HF HFr fed mice.

Treatment with the HMGB1 siRNA ameliorated some of the adverse effects of the Hf Hfr diet by decreasing liver weight as a percent of body weight and decreasing liver triglyceride levels. The results are shown in the table below.

| | | | | | % decrease in HF |
|---|---|---|---|---|---|
| | LFD + PBS | HF HFr + PBS | HF HFr + HMGB1 | | HFr + HMGB1 vs |
| Parameters at week 20 | (N = 5) | (N = 9) | (N = 9) | p values | HF HFr + PBS |
| Liver weight (% BW) | 4.16 +/− 0.19 | 6.35 +/− 0.46 | 5.10 +/− 0.24 | 0.08 | 20 |
| Liver Triglycerides | 21.96 +/− 3.02 | 77.21 +/− 5.41 | 36.39 +/− 3.84 | 0.001 | 53 |
| (mg/g of liver) | | | | | |

Each value represents the mean+/−SEM. The p values are derived from statistical analysis using one-way ANOVA and depicts comparison between HF HFr+PBS and HF HFr+HMGB1.

Serum cholesterol was also significantly decreased (56%, p=0.0001) in the HMGB1 siRNA treated HF HFr mice as compared to the HF HFr PBS treated mice.

Treatment with the HMGB1 siRNA also normalized the expression of genes related to lipid metabolism as shown in the table below. Values are expressed as a relative fold change from HF HFr+PBS.

| | LFD + PBS | HF HFr + PBS | HF HFr + HMGB1 | |
|---|---|---|---|---|
| Genes | (N = 5) | (N = 9) | (N = 9) | p values |
| SREBP2 | 1.3 +/− 0.08 | 1.03 +/− 0.08 | 0.76 +/− 0.05 | 0.01 |
| ACC2 | 1.06 +/− 0.08 | 1.02 +/− 0.07 | 0.46 +/− 0.02 | 0.0001 |
| SCD1 | 0.72 +/− 0.13 | 1.03 +/− 0.09 | 0.73 +/− 0.06 | 0.03 |
| HMG Co-A reductase | 0.63 +/− 0.1 | 1.04 +/− 0.1 | 0.58 +/− 0.02 | 0.0005 |
| PCSK9 | 0.96 +/− 0.2 | 1.09 +/− 0.17 | 0.42 +/− 0.04 | 0.0026 |

Each value represents the mean+/−SEM. The p values are derived from statistical analysis using one-way ANOVA and depicts comparison between HF HFr+PBS and HF HFr+HMGB1.

Treatment with the HMGB1 siRNA also improved some mean NAS and fibrosis scores based on histopathological data as shown below.

| Histopathology | | | | |
|---|---|---|---|---|
| and NAS score at | LFD + PBS | HF HFr + PBS | HF HFr + HMGB1 | |
| week 20 | (N = 5) | (N = 9) | (N = 9) | p values |
| Steatosis score | 0 | 2.88 +/− 0.1 | 2.44 +/− 0.2 | 0.03 |
| Lobular inflammation | 0 | 1.7 +/− 0.2 | 1.0 +/− 0 | 0.0001 |
| Ballooning | 0 | 1.0 +/− 0 | 1.0 +/− 0 | 1 |
| Mean NAS score | 0 | 5.6 +/− 0.2 | 4.4 +/− 0.2 | 0.003 |
| Fibrosis score | 0 | 1 +/− 0.2 | 0.56 +/− 0.2 | 0.3 |

Each value represents the mean+/−SEM. The p values are derived from statistical analysis using one way ANOVA and depicts comparison between HF HFr+PBS and HF HFr+HMGB1.

No significant changes in expression of inflammation or fibrosis genes, serum biomarkers of liver injury, or serum insulin or glucose were observed.

A second experiment was performed using the same mouse model and dosing regimen described above. Some of the endpoints assayed were different in the second study. The results of this study are provided below.

Briefly, as described above, 6-8 weeks-old C57BL/6 male mice obtained from Jackson Laboratories were fed a high fat diet containing 60% of calories as fat plus 30% fructose in water (Hf Hfr diet) for 12 weeks prior to treatment in order to induce NASH, or fed a standard chow and water diet.

Starting at week 12, HF HFr fed mice were subcutaneously administered a 10 mg/kg dose of an siRNA targeted to HMGB1 (AD-80653) every other week for a total of four doses. Two weeks after the final dose (at week 20), livers were harvested, RNA was isolated, and HMGB1 knockdown was determined by rtPCR using the method described above. A 90% decrease in liver HMGB1 mRNA was observed in the HMGB1 siRNA treated Hf Hfr fed mice as compared to the PBS treated HF HFr fed mice.

Treatment with the HMGB1 siRNA ameliorated some of the adverse effects of the Hf Hfr diet by decreasing liver weight as a percent of body weight, liver triglyceride, liver free-cholesterol, and liver free fatty acid levels. The results are shown in the table below.

| | | | | | % decrease in HF |
|---|---|---|---|---|---|
| Parameters at | LFD + PBS | HF HFr + PBS | HF HFr + HMGB1 | | HFr + HMGB1 vs HF |
| week 20 | (N = 5) | (N = 9) | (N = 9) | P values | HFr + PBS |
| Liver weight | 4.17 +/− 0.21 | 6.37 +/− 0.23 | 5.07 +/− 0.19 | 0.01 | 20.5 |
| (% BW) | | | | | |
| Liver | 22.25 +/− 1.74 | 110.95 +/− 3.27 | 50.78 +/− 5.28 | 0.0001 | 54.2 |
| Triglycerides | | | | | |
| (mg/g of liver) | | | | | |
| Liver Free- | 2.30 +/− 0.03 | 3.10 +/− 0.08 | 2.50 +/− 0.1 | 0.0006 | 19.4 |
| Cholesterol | | | | | |
| (mg/g of liver) | | | | | |
| Liver Free Fatty | 1.86 +/− 0.06 | 2.55 +/− 0.09 | 1.98 +/− 0.09 | 0.0002 | 22.3 |
| Acids | | | | | |

Each value represents the mean+/−SEM. The p values are derived from statistical analysis using one-way ANOVA and depicts comparison between HF HFr+PBS and HF HFr+HMGB1.

Treatment with HMGB1 siRNA also resulted in a decrease in serum cholesterol, LDL cholesterol and serum non-esterified fatty acids (NEFA). There was an increase in the serum triglyceride levels seen with HMGB1 siRNA treatment. The results are shown in the table below.

| | | | | | % decrease in HF |
|---|---|---|---|---|---|
| Parameters at | LFD + PBS | HF HFr + PBS | HF HFr + HMGB1 | | HFr + HMGB1 vs |
| week 20 | (N = 5) | (N = 9) | (N = 9) | p values | HF HFr + PBS |
| Serum | 80.36 +/− 1.75 | 229.1 +/− 17.65 | 102.6 +/− 6.72 | 0.0001 | 55.2 |
| Cholesterol | | | | | |
| (mg/dL) | | | | | |
| Serum | 73.68 +/− 4.73 | 71.79 +/− 5.8 | 94.44 +/− 5.35 | 0.008 | −31.9 |
| Triglycerides | | | | | |
| (mg/dL) | | | | | |
| Serum NEFA | 0.88 +/− 0.06 | 0.92 +/− 0.06 | 0.74 +/− 0.04 | 0.04 | 19 |
| (mM) | | | | | |
| Serum LDL | 7 +/− 0 | 12.62 +/− 1.57 | 7.71 +/− 0.32 | 0.0001 | 20.4 |
| Cholesterol | | | | | |
| (mg/dL) | | | | | |

Each value represents the mean+/−SEM. The p values are derived from statistical analysis using one-way ANOVA and depicts comparison between HF HFr+PBS and HF HFr+HMGB1.

Treatment with the HMGB1 siRNA also improved mean NAS and fibrosis scores based on histopathological data as shown in the Table below.

| Histopathology | | | | |
|---|---|---|---|---|
| and NAS score | LFD + PBS | HF HFr + PBS | HF HFr + HMGB1 | |
| at week 20 | (N = 5) | (N = 9) | (N = 9) | p value |
| Steatosis score | 0 | 2.9 +/− 0.1 | 1.5 +/− 0.24 | <0.0001 |
| Lobular inflammation | 0 | 1.0 +/− 0 | 0.4 +/− 0.18 | 0.02 |
| Ballooning | 0 | 0.3 +/− 0.17 | 0 +/− 0 | 0.16 |
| Mean NAS score | 0 | 4.2 +/− 0.22 | 2 +/− 0.37 | 0.0001 |

Each value represents the mean+/−SEM. The p values are derived from statistical analysis using one way ANOVA and depicts comparison between HF HFr+PBS and HF HFr+HMGB1.

Treatment with the HMGB1 siRNA also mildly reduced serum ALT, AST, and glutamate dehydrogenase (GLDH).

These data demonstrate that HMGB1 siRNA is effective in the treatment of HMGB1-associated disorders, e.g., metabolic disorder and NAFLD, e.g., NASH.

### Example 5—Knockdown of HMGB1 Expression with a Single Dose of HMGB1 siRNA

A series of dsRNA agents targeting mouse HMGB1 were designed and tested for the ability to knockdown expression of HMGB1 mRNA in 6-8 week old C57Bl/6 female mice. The duplexes that were assessed are listed in Table 7 below.

TABLE 7
HMGB1 Unmodified and Modified Sequences

| | | | SEQ | | SEQ | Target site |
|---|---|---|---|---|---|---|
| Duplex | | Unmodified | ID | | ID | in |
| Name | Strand | sequence 5′ to 3′ | NO | Modified sequence5′ to 3′ | NO | NM_002128.5 |
| AD-245281 | sense | UAAGUUGGUUCUAGCGCAGUU | 511 | usasaguuGfgUfUfCfuagcgcaguuL96 | 525 | 830-850 |
| | antisense | AACUGCGCUAGAACCAACUUAUU | 512 | asAfscugc(Ggn)cuagaaCfcAfacuuasusu | 526 | 828-850 |
| AD-245282 | sense | AAGUUGGUUCUAGCGCAGUUU | 513 | asasguugGfuUfCfUfagcgcaguuuL96 | 527 | 831-851 |
| | antisense | AAACUGCGCUAGAACCAACUUAU | 514 | asAfsacug(Cgn)gcuagaAfcCfaacuusasu | 528 | 829-851 |
| AD-245305 | sense | UUGAAAUGUAAGGCUGUGUAA | 515 | ususgaaaUfgUfAfAfggcuguguaaL96 | 529 | 917-937 |
| | antisense | UUACACAGCCUUACAUUUCAAUU | 516 | usUfsacac(Agn)gccuuaCfaUfuucaasusu | 530 | 915-937 |
| AD-245336 | sense | UAUAGUUAACACACUACCGAA | 517 | usasuaguUfaAfCfAfcacuaccgaaL96 | 531 | 972-992 |
| | antisense | UUCGGUAGUGUGUUAACUAUACA | 518 | usUfscggu(Agn)guguguUfaAfcuauascsa | 532 | 970-992 |
| AD-245339 | sense | AGUUAACACACUACCGAAUGU | 519 | asgsuuaaCfaCfAfCfuaccgaauguL96 | 533 | 975-995 |
| | antisense | ACAUUCGGUAGUGUGUUAACUAU | 520 | asCfsauuc(Ggn)guagugUfgUfuaacusasu | 534 | 973-995 |
| AD-245383 | sense | UGGUAUUUUCAAUAGCCACUA | 521 | usgsguauUfuUfCfAfauagccacuaL96 | 535 | 1019-1039 |
| | antisense | UAGUGGCUAUUGAAAAUACCACC | 522 | usAfsgugg(Cgn)uauugaAfaAfuaccascsc | 536 | 1017-1039 |
| AD-245472 | sense | UCUGAUGCAGCUUAUACGAAA | 523 | uscsugauGfcAfGfCfuuauacgaaaL96 | 537 | 1160-1180 |
| | antisense | UUUCGUAUAAGCUGCAUCAGAGA | 524 | usUfsucgu(Agn)uaagcuGfcAfucagasgsa | 538 | 1158-1180 |

A single dose of one of the dsRNA agents; or PBS control, was administered subcutaneously on day 1 at a dose of 10 mg/kg (n=3 per group). At day 14 post dosing, mice were sacrificed, livers were harvested, and HMGB1 expression was analyzed.

RNA was isolated using the RNeasy plus kit (Qiagen, Cat #74136). One g of total RNA was transcribed to cDNA using ABI® High capacity cDNA reverse transcription kit (Applied Biosystems, Cat #4368813), both following manufacturer's protocol.

One μl of cDNA was added to a master mix containing 0.5 μl of GAPDH TaqMan Probe (4352339E), 0.5 μl mouse HMGB1 probe (Mm00849805_gH), 5 μl Lightcycler 480 probe master mix (Roche Cat #04887301001) and 3 μl of nuclease free water per well in a 384-well plate (Roche Cat #04887301001). Reactions were run in triplicate. Real time PCR was done in a LightCycler480 Real Time PCR system (Roche). To calculate relative fold change, real time data were analyzed using the ΔΔCt method and normalized to PBS treated animals. Results are shown in Table 8.

TABLE 8

| Duplex Name | Average | SEM |
|---|---|---|
| PBS | 1.00 | 0.06 |
| AD-245281 | 0.15 | 0.02 |
| AD-245282 | 0.13 | 0.02 |
| AD-245305 | 0.26 | 0.02 |
| AD-245336 | 0.23 | 0.01 |
| AD-245339 | 0.19 | 0.03 |
| AD-245383 | 0.28 | 0.02 |
| AD-245472 | 0.20 | 0.02 |

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

We claim:

1. A double stranded ribonucleic acid (dsRNA) agent for inhibiting expression of high mobility group box-1 (HMGB1), wherein the dsRNA agent comprises a sense strand and an antisense strand forming a double stranded region, wherein the sense strand comprises at least 15 contiguous nucleotides differing by no more than 3 nucleotides from the nucleotide sequence of SEQ ID NO:1 and the antisense strand comprises at least 15 contiguous nucleotides differing by no more than 3 nucleotides from the nucleotide sequence of SEQ ID NO:2.

2. The dsRNA agent of claim 1, wherein the sense strand comprises at least 15 contiguous nucleotides differing by no more than 3 nucleotides from any one of the nucleotide sequences of nucleotides 830-851, 830-850, 831-851, 917-937, 944-997, 944-990, 944-964, 968-997, 968-990, 968-995, 968-988, 969-989, 970-990, 971-991, 972-992, 972-995, 973-993, 974-994, 975-995, 976-996, 977-997, 1019-1039, 1158-1194, 1158-1182, 1158-1178, 1159-1179, 1160-1180, 1161-1181, 1162-1182, or 1174-1194 of SEQ ID NO:1.

3. A double stranded ribonucleic acid (dsRNA) agent for inhibiting expression of HMGB1, wherein the dsRNA agent comprises a sense strand and an antisense strand, the antisense strand comprising a region of complementarity to an mRNA encoding HMGB1 comprising at least 15 contiguous nucleotides differing by no more than 3 nucleotides from any one of the antisense nucleotide sequences listed in any one of Table 3, Table 5, Table 6, or Table 7.

4. The dsRNA agent of claim 3, wherein the antisense strand comprises at least 15 contiguous nucleotides differing by no more than 3 nucleotides from any one of the antisense nucleotide sequences of a duplex selected from the group consisting of AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-193181, AD-80651, and AD-80652.

5. The dsRNA agent of claim 4, wherein the sense strand comprises at least 15 contiguous nucleotides of the sense strand nucleotide sequence of AD-245281 (5′-UAAGUUGGUUCUAGCGCAGUU-3′) (SEQ ID NO:511) and the antisense strand comprises at least 15 contiguous nucleotides from the antisense strand nucleotide sequence of AD-245281 (5′-AACUGCGCUAGAACCAACUUAUU-3′) (SEQ ID NO:512).

6. The dsRNA agent of claim 5, wherein the sense strand comprises at least 17 contiguous nucleotides of the nucleotide sequence 5′-UAAGUUGGUUCUAGCGCAGUU-3′ (SEQ ID NO:511) and the antisense strand comprises at least 17 contiguous nucleotides of the nucleotide sequence 5′-AACUGCGCUAGAACCAACUUAUU-3′(SEQ ID NO:512).

7. The dsRNA agent of claim 5, wherein the strand comprises at least 19 contiguous nucleotides of the nucleotide sequence 5′-UAAGUUGGUUCUAGCGCAGUU-3′ (SEQ ID NO:511) and the antisense strand comprises at least 19 contiguous nucleotides of the nucleotide sequence 5′-AACUGCGCUAGAACCAACUUAUU-3′(SEQ ID NO:512).

8. The dsRNA agent of claim 5, wherein the sense strand comprises 21 contiguous nucleotides of the nucleotide sequence 5′-UAAGUUGGUUCUAGCGCAGUU-3′ (SEQ ID NO:511) and the antisense strand comprises at least 21 contiguous 7 nucleotides of the nucleotide sequence 5′-AACUGCGCUAGAACCAACUUAUU-3′(SEQ ID NO:512).

9. The dsRNA agent of claim 4, wherein the sense strand comprises at least 15 contiguous nucleotides of the sense strand nucleotide sequence of AD-245282 (5′-AAGUUGGUUCUAGCGCAGUUU-3′) (SEQ ID NO:513) and the antisense strand comprises at least 15 contiguous nucleotides of the antisense strand nucleotide sequence of AD-245282 (5′-AAACUGCGCUAGAACCAACUUAU-3′) (SEQ ID NO:514).

10. The dsRNA agent of claim 9, wherein the sense strand comprises at least 17 contiguous nucleotides of the nucleotide sequence 5′-AAGUUGGUUCUAGCGCAGUUU-3′ (SEQ ID NO:513) and the antisense strand comprises at least 17 contiguous nucleotides of the nucleotide sequence 5′-AAACUGCGCUAGAACCAACUUAU-3′ (SEQ ID NO:514).

11. The dsRNA agent of claim 9, wherein the sense strand comprises at least 19 contiguous nucleotides of the nucleotide sequence 5′-AAGUUGGUUCUAGCGCAGUUU-3′ (SEQ ID NO:513) and the antisense strand comprises at least 19 contiguous nucleotides of the nucleotide sequence 5′-AAACUGCGCUAGAACCAACUUAU-3′ (SEQ ID NO:514).

12. The dsRNA agent of claim 9, wherein the sense strand comprises 21 contiguous nucleotides of the nucleotide sequence 5′-AAGUUGGUUCUAGCGCAGUUU-3′ (SEQ ID NO:513) and the antisense strand comprises at least 21 contiguous nucleotides of the nucleotide sequence 5′-AAACUGCGCUAGAACCAACUUAU-3′ (SEQ ID NO:514).

13. The dsRNA agent of claim 3, wherein the sense strand and antisense strand comprise nucleotide sequences selected from the group consisting of any of the nucleotide sequences in any one of Table 3, Table 5, Table 6, or Table 7.

14. The dsRNA agent of any one of claims 1-13, wherein the dsRNA agent comprises at least one modified nucleotide.

15. The dsRNA agent of any one of claims 1-13, wherein all of the nucleotides of the sense strand and all of the nucleotides of the antisense strand comprise a nucleotide modification.

16. The dsRNA agent of claim 15, wherein the sense strand comprises a modified nucleotide sequence of 5′-usasaguuGfgUfUfCfuagcgcaguu-3′ (SEQ ID NO:525) and the antisense strand comprises a modified nucleotide sequence of 5′-asAfscugc(Ggn)cuagaaCfcAfacuuasusu-3′ (SEQ ID NO:526), wherein a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Ggn) is guanosine-glycol nucleic acid (GNA), and s is phosphorothioate linkage.

17. The dsRNA agent of claim 16, further comprising an N-[tris(GalNAc-alkyl)-amidodecanoyl)]-4-hydroxyprolinol covalently linked to the 3′-end of the sense strand.

18. The dsRNA agent of claim 15, wherein the sense strand comprises a modified nucleotide sequence of 5′-asasguugGfuUfCfUfagcgcaguuu-3′ (SEQ ID NO:527) and the antisense strand comprises a modified nucleotide sequence of 5′-asAfsacug(Cgn)gcuagaAfcCfaacuusasu-3′ (SEQ ID NO:528), wherein a is 2′-O-methyladenosine-3′-phosphate, c is 2′-O-methylcytidine-3′-phosphate, g is 2′-O-methylguanosine-3′-phosphate, u is 2′-O-methyluridine-3′-phosphate, Af is 2′-fluoroadenosine-3′-phosphate, Cf is 2′-fluorocytidine-3′-phosphate, Gf is 2′-fluoroguanosine-3′-phosphate, Uf is 2′-fluorouridine-3′-phosphate, (Cgn) is cytidine-glycol nucleic acid (GNA), and s is phosphorothioate linkage.

19. The dsRNA agent of claim 18, further comprising an N-[tris(GalNAc-alkyl)-amidodecanoyl)]-4-hydroxyprolinol covalently linked to the 3′-end of the sense strand.

20. A double stranded RNA (dsRNA) agent for inhibiting expression of HMGB1, wherein the double stranded RNA agent comprises a sense strand and an antisense strand forming a double stranded region,
wherein the sense strand comprises at least 15 contiguous nucleotides differing by no more than 3 nucleotides from the nucleotide sequence of SEQ ID NO:1 and the antisense strand comprises at least 15 contiguous nucleotides differing by no more than 3 nucleotides from the nucleotide sequence of SEQ ID NO:2,
wherein substantially all of the nucleotides of the sense strand and substantially all of the nucleotides of the antisense strand are modified nucleotides, and
wherein the sense strand is conjugated to a ligand attached at the 3′-terminus.

21. The dsRNA agent of claim 20, wherein the sense strand comprises at least 15 contiguous nucleotides differing by no more than 3 nucleotides from the nucleotide sequence of any one of nucleotides 830-851, 830-850, 831-851, 917-937, 944-997, 944-990, 944-964, 968-997, 968-990, 968-995, 968-988, 969-989, 970-990, 971-991, 972-992, 972-995, 973-993, 974-994, 975-995, 976-996, 977-997, 1019-1039, 1158-1194, 1158-1182, 1158-1178, 1159-1179, 1160-1180, 1161-1181, 1162-1182, or 1174-1194 of SEQ ID NO:1.

22. The dsRNA agent of claim 20 or 21, wherein all of the nucleotides of the sense strand and all of the nucleotides of the antisense strand comprise a nucleotide modification.

23. The dsRNA agent of claim 20 or 21, wherein at least one of the modified nucleotides is selected from the group consisting of a deoxy-nucleotide, a 3′-terminal deoxy-thymine (dT) nucleotide, a 2′-O-methyl modified nucleotide, a 2′-fluoro modified nucleotide, a 2′-deoxy-modified nucleotide, a locked nucleotide, an unlocked nucleotide, a conformationally restricted nucleotide, a constrained ethyl nucleotide, an abasic nucleotide, a 2′-amino-modified nucleotide, a 2′-O-allyl-modified nucleotide, 2′-C-alkyl-modified nucleotide, 2′-hydroxly-modified nucleotide, a 2′-methoxyethyl modified nucleotide, a 2′-O-alkyl-modified nucleotide, a morpholino nucleotide, a phosphoramidate, a non-natural base comprising nucleotide, a tetrahydropyran modified nucleotide, a 1,5-anhydrohexitol modified nucleotide, a cyclohexenyl modified nucleotide, a nucleotide comprising a phosphorothioate group, a nucleotide comprising a methylphosphonate group, a nucleotide comprising a 5′-phosphate, a nucleotide comprising a 5′-phosphate mimic, a glycol modified nucleotide (GNA), and a 2-O—(N-methylacetamide) modified nucleotide; and combinations thereof.

24. The dsRNA agent of claim 23, wherein the modified nucleotide comprises a short sequence of 3′-terminal deoxy-thymine nucleotides (dT).

25. The dsRNA agent of claim 3, wherein the region of complementarity is at least 17 nucleotides in length.

26. The dsRNA agent of claim 3, wherein the region of complementarity is 19-21 nucleotides in length.

27. The dsRNA agent of claim 26, wherein the region of complementarity is 19 nucleotides in length.

28. The dsRNA agent of any one of claims 1-27, wherein each strand is independently no more than 30 nucleotides in length.

29. The dsRNA agent of any one of claims 1-27, wherein at least one strand comprises a 3′ overhang of at least 1 nucleotide.

30. The dsRNA agent of any one of claims 1-27, wherein at least one strand comprises a 3′ overhang of at least 2 nucleotides.

31. The dsRNA agent of any one of claims 1-16 and 18, further comprising a ligand.

32. The dsRNA agent of claim 31, wherein the ligand is conjugated to the 3′ end of the sense strand of the dsRNA agent.

33. The dsRNA agent of claim 20 or 31, wherein the ligand is an N-acetylgalactosamine (GalNAc) derivative.

34. The dsRNA agent of claim 33, wherein the ligand is

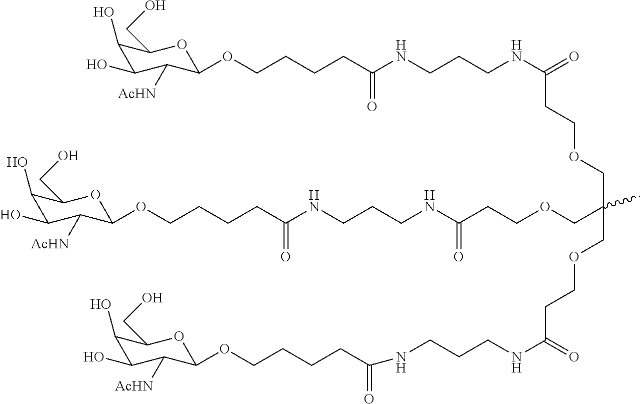 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

35. The dsRNA agent of claim 34, wherein the dsRNA agent is conjugated to the ligand as shown in the following schematic 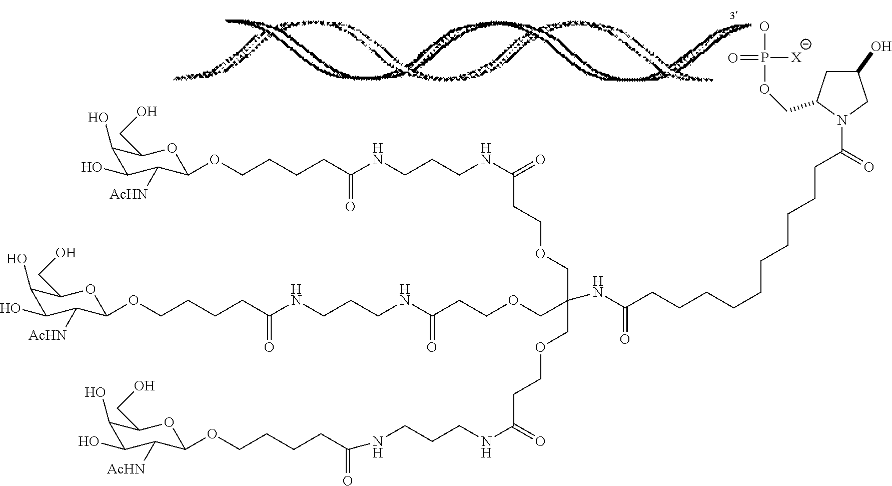
and, wherein X is O or S.

36. The dsRNA agent of claim 35, wherein the X is O.

37. The dsRNA agent of claim 3 or 4, wherein the region of complementarity comprises any one of the antisense nucleotide sequences of any one of Table 3, Table 5, Table 6, or Table 7.

38. The dsRNA agent of claim 3 or 4, wherein the region of complementarity consists of any one of the antisense nucleotide sequences of any one of Table 3, Table 5, Table 6, or Table 7.

39. The dsRNA agent of claim 37 or 38, wherein the antisense nucleotide sequence is the antisense sequence of a duplex selected from the group consisting of AD-245281, AD-245282, AD-245305, AD-245336, AD-245339, AD-245383, AD-245472, AD-193177, AD-193312, AD-193168, AD-193313, AD-193180, AD-193182, AD-193314, AD-193173, AD-193311, AD-193179, AD-193178, AD-193174, AD-193315, AD-193175, AD-193326, AD-193176, AD-193181, AD-80651, and AD-80652.

40. The dsRNA agent of any one of claims 1-39, wherein the double stranded region is 15-30 nucleotide pairs in length.

41. The dsRNA agent of claim 40, wherein the double stranded region is 17-23 nucleotide pairs in length.

42. The dsRNA agent of claim 40, wherein the double stranded region is 17-25 nucleotide pairs in length.

43. The dsRNA agent of claim 40, wherein the double stranded region is 23-27 nucleotide pairs in length.

44. The dsRNA agent of claim 40, wherein the double stranded region is 19-21 nucleotide pairs in length.

45. The dsRNA agent of claim 20, wherein the double stranded region is 21-23 nucleotide pairs in length.

46. The dsRNA agent of any one of claims 1-45, wherein each strand independently has 19-30 nucleotides.

47. The dsRNA agent of claim 20, wherein the modifications on the nucleotides are selected from the group consisting of LNA, HNA, CeNA, 2′-methoxyethyl, 2′-O-alkyl, 2′-O-allyl, 2′-C-allyl, 2′-fluoro, 2′-deoxy, 2′-hydroxyl, GNA, and combinations thereof.

48. The dsRNA agent of claim 47, wherein the modifications on the nucleotides are 2′-O-methyl or 2′-fluoro modifications.

49. The dsRNA agent of claim 20, wherein the ligand is one or more GalNAc derivatives attached through a monovalent, bivalent, or trivalent branched linker.

50. The dsRNA agent of claim 20, wherein the agent further comprises at least one phosphorothioate or methylphosphonate internucleotide linkage.

51. The dsRNA agent of claim 50, wherein the phosphorothioate or methylphosphonate internucleotide linkage is at the 3′-terminus of one strand.

52. The dsRNA agent of claim 51, wherein the strand is the antisense strand.

53. The dsRNA agent of claim 51, wherein the strand is the sense strand.

54. The dsRNA agent of claim 50, wherein the phosphorothioate or methylphosphonate internucleotide linkage is at the 5′-terminus of one strand.

55. The dsRNA agent of claim 54, wherein the strand is the antisense strand.

56. The dsRNA agent of claim 54, wherein the strand is the sense strand.

57. The dsRNA agent of claim 50, wherein the phosphorothioate or methylphosphonate internucleotide linkage is at the both 5′- and 3′-terminus of one strand.

58. The dsRNA agent of claim 57, wherein the strand is the antisense strand.

59. The dsRNA agent of claim 20, wherein the base pair at the 1 position of the 5′-end of the antisense strand of the double stranded region is an AU base pair.

60. The dsRNA agent of claim 56, wherein the sense strand has a total of 21 nucleotides and the antisense strand has a total of 23 nucleotides.

61. A double stranded RNA (dsRNA) agent for inhibiting the expression of HMGB1,
wherein the dsRNA agent comprises a sense strand and an antisense strand forming a double stranded region,
wherein the sense strand comprises at least 15 contiguous nucleotides differing by no more than 3 nucleotides from the nucleotide sequence of SEQ ID NO:1 and the antisense strand comprises at least 15 contiguous nucleotides differing by no more than 3 nucleotides from the nucleotide sequence of SEQ ID NO:2,
wherein substantially all of the nucleotides of the sense strand comprise a nucleotide modification selected from a 2′-O-methyl modification and a 2′-fluoro modification,
wherein the sense strand comprises two phosphorothioate internucleotide linkages at the 5′-terminus,
wherein substantially all of the nucleotides of the antisense strand comprise a nucleotide modification selected from the group consisting of a 2′-O-methyl modification and a 2′-fluoro modification,
wherein the antisense strand comprises two phosphorothioate internucleotide linkages at the 5′-terminus and two phosphorothioate internucleotide linkages at the 3′-terminus, and
wherein the sense strand is conjugated to one or more GalNAc derivatives attached through a monovalent, bivalent or trivalent branched linker at the 3′-terminus.

62. The dsRNA agent of claim 61, wherein the sense strand comprises at least 15 contiguous nucleotides differing by no more than 3 nucleotides from the nucleotide sequence of any one of nucleotides 830-851, 830-850, 831-851, 917-937, 944-997, 944-990, 944-964, 968-997, 968-990, 968-995, 968-988, 969-989, 970-990, 971-991, 972-992, 972-995, 973-993, 974-994, 975-995, 976-996, 977-997, 1019-1039, 1158-1194, 1158-1182, 1158-1178, 1159-1179, 1160-1180, 1161-1181, 1162-1182, or 1174-1194 of SEQ ID NO:1.

63. The dsRNA agent of claim 61, wherein all of the nucleotides of the sense strand and all of the nucleotides of the antisense strand are modified nucleotides.

64. The dsRNA agent of claim 61, wherein each strand independently has 19-30 nucleotides.

65. The dsRNA agent of claim 61, wherein each strand independently has 14-40 nucleotides.

66. The dsRNA agent of claim 61, wherein the sense strand comprises a thermally destabilizing nucleotide placed at a site opposite to a seed region of the antisense strand at positions 2-8 of the 5′-end of the antisense strand.

67. The dsRNA agent of claim 66, wherein the thermally destabilizing modification is selected from an abasic modification; a mismatch with the opposing nucleotide in the duplex; and a destabilizing sugar modification.

68. An isolated cell containing the dsRNA agent of any one of claims 1-67.

69. A pharmaceutical composition for inhibiting expression of a gene encoding HMGB1 comprising the dsRNA agent of any one of claims 1-67.

70. A pharmaceutical composition comprising the dsRNA agent of any one of claims 1-67, and a lipid.

71. A method of inhibiting expression of an HMGB1 gene in a cell, the method comprising contacting the cell with the dsRNA agent of any one of claims 1-67 or the pharmaceutical composition of claim 69 or 70, thereby inhibiting expression of the HMGB1 gene in the cell.

72. The method of claim 71, wherein the cell is within a subject.

73. The method of claim 72, wherein the subject is a human subject.

74. The method of claim 73, wherein the subject has an HMGB1-associated disorder.

75. The method of claim 74, wherein the HMGB1-associated disorder is selected from the group consisting of liver inflammation, liver fibrosis, liver damage associated with an elevated level of HMGB1, metabolic disorder, blood pressure equal to or higher than 130/85 mmHg, elevated fasting blood glucose of at least 100 mg/dL, large waist circumference (40 inches or more for men and 35 inches or more for women); waist-to-hip ratio <1.0 (for men) or <0.8 (for women); low HDL cholesterol (under 40 mg/dL for men and under 50 mg/dL for women), triglycerides of at least 150 mg/dL, NAFLD, steatohepatitis, NASH, NASH cirrhosis, cryptogenic cirrhosis, hypertension, hypercholesterolemia, hepatic infection, hepatic inflammation, cirrhosis, autoimmune hepatitis, chronic alcohol consumption, alcoholic hepatitis, alcoholic steatohepatitis, hemochromatosis, and chronic use of pharmaceutical agents that cause liver damage.

76. The method of claim 74, wherein the HMGB1-associated disorder is metabolic disorder.

77. The method of claim 74, wherein the HMGB1-associated disorder is NAFLD.

78. The method of any one of claims 71-77, wherein the expression of HMGB1 is inhibited by at least 30%, 40%, 50%, 60%, 70%, 80%, 90%, 95%, 98% or to below the level of detection of the assay as compared to not contacting the cell with the dsRNA agent.

79. The method of any one of claims 73-77, wherein inhibiting the expression of HMGB1 decreases an HMGB1 protein level in the serum of the subject by at least 30%, 40%, 50%, 60%, 70%, 80%, 90%, or 95%.

80. A method of treating an HMGB1-associated disorder in a subject, comprising administering to the subject the dsRNA agent of any one of claims 1-67 or the pharmaceutical composition of claim 69 or 70, thereby treating the HMGB1-associated disorder in the subject.

81. The method of claim 80, wherein the HMGB1-associated disorder is selected from the group consisting of liver inflammation, liver fibrosis, liver damage associated with an elevated level of HMGB1, metabolic disorder, blood pressure equal to or higher than 130/85 mmHg, elevated fasting blood glucose of at least 100 mg/dL, large waist circumference (40 inches or more for men and 35 inches or more for women); waist-to-hip ratio <1.0 (for men) or <0.8 (for women); low HDL cholesterol (under 40 mg/dL for men and under 50 mg/dL for women), triglycerides of at least 150 mg/dL, NAFLD, steatohepatitis, NASH, NASH cirrhosis, cryptogenic cirrhosis, hypertension, hypercholesterolemia, hepatic infection, hepatic inflammation, cirrhosis, autoimmune hepatitis, chronic alcohol consumption, alcoholic hepatitis, alcoholic steatohepatitis, hemochromatosis, and chronic use of pharmaceutical agents that cause liver damage.

82. The method of claim 81, wherein the HMGB1-associated disorder is a metabolic disorder.

83. The method of claim 81, wherein the HMGB1-associated disorder is NAFLD.

84. The method of claim 80, wherein the subject is a human subject.

85. The method of any one of claims 80-84, wherein the dsRNA agent is administered to the subject at a dose of about 0.01 mg/kg to about 50 mg/kg.

86. The method of any one of claims 80-84, wherein the dsRNA agent is administered to the subject subcutaneously.

87. The method of any one of claims 80-85, wherein the level of HMGB1 is measured in the subject.

88. The method of claim 86, where the level of HMGB1 is the level of HMGB1 protein in a subject blood or serum sample, or the level of HMGB1 RNA in a blood or urine sample.

89. The method of claim 88, wherein the RNA in the blood or urine sample is assayed for an siRNA cleavage site.
